(19) United States
(12) Patent Application Publication
(10) Pub. No.: US 2024/0148803 A1
(43) Pub. Date: May 9, 2024

(54) USE OF IMMUNOTHERAPY AND MICROBIOME MODULATION TO TREAT CANCER

(71) Applicant: Seres Therapeutics, Inc., Cambridge, MA (US)

(72) Inventors: Christopher B. FORD, Swampscott, MA (US); Elizabethh HALVORSEN, Malden, MA (US); Christopher DESJARDINS, Somerville, MA (US); Kankana BARDHAN, Lynn, MA (US); Swarma Deepa PANDIAN, Arlington, MA (US)

(73) Assignee: Seres Therapeutics, Inc., Cambridge, MA (US)

(21) Appl. No.: 18/546,922

(22) PCT Filed: Feb. 18, 2022
(86) PCT No.: PCT/US2022/016885

§ 371 Date: Aug. 17, 2023

Related U.S. Application Data
(60) Provisional application No. 63/150,554, filed on Feb. 17, 2021.

(51) Int. Cl. A61K 35/742; A61K 35/745; A61K 45/06; A61P 35/04

(52) U.S. Cl. CPC A61K 35/742; A61K 35/745; A61K 45/06; A61P 35/04

(57) ABSTRACT

Provided herein are methods and compositions for the treatment of various types of cancers by modulating the microbiome to effect or enhance the efficacy of immunotherapy. Also provided herein are methods of determining a response to an immunotherapy by identifying if a subject has a favorable microbial profile. Further provided herein are methods of determining a donor whose feces are useful for fecal matter transfer.

### CROSS-REFERENCE TO RELATED APPLICATIONS

This application claims the priority benefit of U.S. Provisional Application No. 63/150,554, filed Feb. 17, 2021, which is incorporated by reference herein in its entirety.

### REFERENCE TO SEQUENCE LISTING SUBMITTED ELECTRONICALLY

The content of the electronically submitted sequence listing in ASCII text file (Name 4268_061PC01_Seqlisting_ST25.txt; Size: 3.58 MB (3,749,696 bytes); and Date of Creation: Feb. 17, 2022) filed with the application is incorporated herein by reference in its entirety.

### FIELD OF THE DISCLOSURE

The present disclosure relates to methods and therapeutic compositions for the treatment of various types of cancers that are useful for modulating the microbiome to effect or enhance the efficacy of immunotherapy, as well as for identifying subjects having or donors whose feces are useful for enhancing the efficacy of immunotherapy.

### BACKGROUND

Growing evidence suggests that the gut microbiome can affect outcome in cancer patients treated with immune checkpoint inhibitors (CPI), though most data is from patients with metastatic melanoma. The relationship of the gastrointestinal microbiome to treatment outcome in other cancers is poorly understood. The assessment of the gastrointestinal microbiome composition in correlation to CPI response in patients with metastatic lung, urothelial, or renal cell carcinoma, or other types of cancers is lacking.

Fecal matter transplant and designed compositions with certain individual species or consortia of species have been proposed as treatments in modulating gastrointestinal microbiomes, and thus effect or improve therapeutic responses of cancer treatments in animal models and human patients. The growing appreciation of the role of the gastrointestinal microbiomes in shaping immune responses in health and disease parallels an unmet need for microbiome therapeutic strategies to effect or enhance therapeutic responses to immunotherapies in various types of cancer.

### BRIEF SUMMARY

The disclosure relates to a therapeutic composition comprising an effective amount of a purified or isolated population of bacteria selected from Intestinimonas butyriciproducens and Anaerotruncus colihominis or bacteria having a 16S rDNA sequence having at least 97%, 98%, 98.7% or 99% sequence identity to any one 16S rDNA sequence thereof. In some aspects, the 16S rDNA sequence of Intestinimonas butyriciproducens is selected from SEQ ID NOs: 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, and 1394, and the 16S rDNA sequence of Anaerotruncus colihominis is selected from SEQ ID NOs: 1066, 1067, 1068, 1069, 1070, 1071, 1072, 1073, 1074, 1075, 1076, 1077, 1078, 1079, 1080, 1081, 1082, 1083, 1084, and 1085.

The disclosure also relates to a therapeutic composition comprising an effective amount of a purified or isolated population of bacteria selected from Murimonas intestini and Faecalicatena orotica, or bacteria having a 16S rDNA sequence having at least 97%, 98%, 98.7% or 99% sequence identity to any one 16S rDNA sequence thereof. In some aspects, the 16S rDNA sequence of Murimonas intestini is SEQ ID NO: 1621 and the 16S rDNA sequence of Faecalicatena orotica is selected from SEQ ID NOs: 800, 801, 802, 803, 804, 805, 806, 807, 808, 809, and 810.

The disclosure also relates to a therapeutic composition comprising an effective amount of a purified or isolated population of bacteria comprising one or more bacteria selected from Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans, or bacteria having 16S rDNA having at least 94.5%, 95%, 97%, 98%, 98.7% or 99% sequence identity to any one of the 16S rDNA thereof. In some aspects, the 16S rDNA sequence of Anaerostipes hadrus is selected from SEQ ID NOs: 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, and 1057, the 16S rDNA sequence of Christensenella timonensis is selected from SEQ ID NOs: 1283, 1284, and 1285, the 16S rDNA sequence of Desulfovibrio desulfuricans is selected from SEQ ID NOs: 1293, 1294, 1295, and 1296, the 16S rDNA sequence of Dialister invisus is selected from SEQ ID NOs: 1297, 1298, and 1299, the 16S rDNA sequence of Lachnoclostridium edouardi is selected from SEQ ID NOs: 1405, 1406, 1407, 1408, 1409, and 1410, the 16S rDNA sequence of Lachnospira pectinoschiza is selected from SEQ ID NOs: 1411, 1412, 1413, 1414, 1415, and 1416, the 16S rDNA sequence of Lachnospiraceae NG18_sp7 is selected from SEQ ID NOs: 1419, 1420, 1421, 1422, 1423, 1424, 1425, and 1426, the 16S rDNA sequence of Lawsonibacter sp7 is selected from SEQ ID NOs: 1608, 1609, 1610, 1611, 1612, 1613, 1614, and 1615, the 16S rDNA sequence of Merdimonas faecis is SEQ ID NO: 1010, the 16S rDNA sequence of Odoribacter splanchnicus is selected from SEQ ID NOs: 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, and 1648, the 16S rDNA sequence of Roseburia faecis is selected from SEQ ID NOs: 1696, 1697, 1698, 1699, 1700, 1701, 1702, 1703, and 1704, the 16S rDNA sequence of Roseburia intestinalis is selected from SEQ ID NOs: 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732, or the 16S rDNA sequence of Ruthenibacterium lactatiformans is selected from SEQ ID NOs: 1733, 1734, 1735, 1736, 1737, 1738, 1739, 1740, 1741, 1742, 1743, 1744, 1745, 1746, 1747, 1748, and 1749.

In some aspects, the therapeutic composition comprises one or more bacteria selected from Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof. In some aspects, the 16S rDNA sequence of Anaerostipes hadrus is selected from SEQ ID NOs: 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, and 1057, the 16S rDNA sequence of Dialister invisus is selected from SEQ ID NOs: 1297, 1298, and 1299, the 16S rDNA sequence of Merdimonas faecis is SEQ ID NO: 1010, the 16S rDNA sequence of Roseburia faecis is selected from SEQ ID NOs: 1696, 1697, 1698, 1699, 1700, 1701, 1702, 1703, and 1704, or the 16S rDNA sequence of Roseburia intestinalis is selected from SEQ ID NOs: 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects, the therapeutic composition comprises one or more bacteria selected from Desulfovibrio desulfuricans, Roseburia intestinalis, or bacteria having 16S rDNA having at least 94.5%, 95%, 97%, 98%, 98.7% or 99% sequence identity to any one of the 16S rDNA thereof. In some aspects, the 16S rDNA sequence of Desulfovibrio desulfuricans is selected from SEQ ID NOs: 1293, 1294, 1295, and 1296 or the 16S rDNA sequence of Roseburia intestinalis is selected from SEQ ID NOs: 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects, the therapeutic composition comprises one or more bacteria selected from Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof. In some aspects, the 16S rDNA sequence of Lachnoclostridium edouardi is selected from SEQ ID NOs: 1405, 1406, 1407, 1408, 1409, and 1410, the 16S rDNA sequence of Lachnospira pectinoschiza is selected from SEQ ID NOs: 1411, 1412, 1413, 1414, 1415, and 1416, the 16S rDNA sequence of Lawsonibacter sp7 is selected from SEQ ID NOs: 1608, 1609, 1610, 1611, 1612, 1613, 1614, and 1615, the 16S rDNA sequence of Odoribacter splanchnicus is selected from SEQ ID NOs: 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, and 1648, or the 16S rDNA sequence of Ruthenibacterium lactatiformans is selected from SEQ ID NOs: 1733, 1734, 1735, 1736, 1737, 1738, 1739, 1740, 1741, 1742, 1743, 1744, 1745, 1746, 1747, 1748, and 1749.

The disclosure also relates to a therapeutic composition comprising an effective amount of a purified or isolated population of bacteria comprising one or more bacteria selected from Anaerotignum lactatifermentans, Anaerotruncus colihominis, Angelakisella sp1, Bacteroides cellulosilyticus, Bacteroides gallinarum, Bacteroides plebeius, Blautia coccoides group, Christensenella minuta, Christensenellaceae NG16_sp1, Clostridium hylemonae, Collinsella bouchesdurhonensis, Coprobacter secundus group, Eisenbergiella massiliensis, Emergencia timonensis, Faecalicatena orotica, Fusicatenibacter sp1, Intestinimonas butyriciproducens, Intestinimonas timonensis, Lachnospiraceae NG17_sp3, Lawsonibacter sp3, Oxalobacter formigenes, Peptoniphilaceae NG4_sp2, Prochlorotrichaceae NG20_sp1, or bacteria having a 16S rDNA sequence having at least 97%, 98%, 98.7% or 99% sequence identity to any one 16S rDNA sequence thereof. In some aspects, the 16S rDNA sequence of Anaerotignum lactatifermentans is selected from SEQ ID NOs: 1058, 1059, 1060, 1061, 1062, 1063, 1064, and 1065, the 16S rDNA sequence of Anaerotruncus colihominis is selected from SEQ ID NOs: 1066, 1067, 1068, 1069, 1070, 1071, 1072, 1073, 1074, 1075, 1076, 1077, 1078, 1079, 1080, 1081, 1082, 1083, 1084, and 1085, the 16S rDNA sequence of Bacteroides cellulosilyticus is selected from SEQ ID NOs: 1086, 1087, 1088, 1089, 1090, 1091, 1092, 1093, 1094, 1095, 1096, 1097, 1098, 1099, 1100, 1101, 1102, 1103, 1104, 1105, and 1106, the 16S rDNA sequence of Bacteroides gallinarum is selected from SEQ ID NOs: 1128 and 1129, the 16S rDNA sequence of Bacteroides plebeius is selected from SEQ ID NOs: 1136, 1137, 1138, 1139, 1140, 1141, 1142, 1143, 1144, 1145, 1146, 1147, 1148, 1149, 1150, 1151, 1152, 1153, 1154, and 1155, the 16S rDNA sequence of Blautia coccoides group is selected from SEQ ID NOs: 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, and 1272, the 16S rDNA sequence of Christensenella minuta is selected from SEQ ID NOs: 1275, 1276, 1277, 1278, 1279, 1280, 1281, and 1282, the 16S rDNA sequence of Christensenellaceae NG16_sp1 is selected from SEQ ID NOs: 1286, 1287, and 1288, the 16S rDNA sequence of Clostridium hylemonae is selected from SEQ ID NOs: 790, 791, 792, 793, 794, 795, and 796, the 16S rDNA sequence of Collinsella bouchesdurhonensis is selected from SEQ ID NOs: 1289, 1290, 1291, and 1292, the 16S rDNA sequence of Coprobacter secundus group is selected from SEQ ID NOs: 797, 798, and 799, the 16S rDNA sequence of Eisenbergiella massiliensis is selected from SEQ ID NOs: 1300, 1301, 1302, 1303, 1304, 1305, 1306, 1307, and 1308, the 16S rDNA sequence of Emergencia timonensis is selected from SEQ ID NOs: 1309, 1310, 1311, 1312, 1313, 1314, 1315, 1316, 1317, 1318, 1319, and 1320, the 16S rDNA sequence of Faecalicatena orotica is selected from SEQ ID NOs: 800, 801, 802, 803, 804, 805, 806, 807, 808, 809, and 810, the 16S rDNA sequence of Fusicatenibacter sp1 is selected from SEQ ID NOs: 1350, 1351, 1352, 1353, 1354, and 1355, the 16S rDNA sequence of Intestinimonas butyriciproducens is selected from SEQ ID NOs: 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, and 1394, the 16S rDNA sequence of Intestinimonas timonensis is selected from SEQ ID NOs: 1403 and, 1404, the 16S rDNA sequence of Lachnospiraceae NG17_sp3 is selected from SEQ ID NOs: 1417 and 1418, the 16S rDNA sequence of Lawsonibacter sp3 is selected from SEQ ID NOs: 1604, 1605, 1606, and 1607, the 16S rDNA sequence of Oxalobacter formigenes is selected from SEQ ID NOs: 1649, 1650, 1651, 1652, and 1653, the 16S rDNA sequence of Peptoniphilaceae NG4_sp2 is SEQ ID NO: 172, or the 16S rDNA sequence of Prochlorotrichaceae NG20_sp1 is SEQ ID NO: 1661.

In some aspects, the therapeutic composition comprises two, three, four, five, six, seven, eight, nine, ten, eleven, twelve, thirteen, fourteen, fifteen, sixteen, seventeen, eighteen or more bacterial species.

In some aspects, the therapeutic composition does not comprise one or more bacteria selected from Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof. In some aspects, the 16S rDNA sequence of Actinomycetaceae_NG7 sp1 is selected from SEQ ID NOs: 1011, 1012, 1013, 1014, 1015, 1016, and 1017, the 16S rDNA sequence of Allisonella histaminiformans is SEQ ID NO: 774, the 16S rDNA sequence of Bacteroides coprocola is selected from SEQ ID NOs: 1107, 1108, 1109, 1110, 1111, and 1112, the 16S rDNA sequence of Bacteroides faecis is selected from SEQ ID NOs: 1113, 1114, 1115, 1116, 1117, 1118, 1119, 1120, 1121, 1122, 1123, 1124, 1125, 1126, and 1127, the 16S rDNA sequence of Bacteroides massiliensis is selected from SEQ ID NOs: 1130, 1131, 1132, 1133, 1134, and 1135, the 16S rDNA sequence of Bifidobacterium animalis is selected from SEQ ID NOs: 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, and 1218, the 16S rDNA sequence of Clostridium asparagiforme group is selected from SEQ ID NOs: 775, 776, 777, 778, 779, 780, 781, 782, 783, 784, 785, 786, 787, 788, and 789, the 16S rDNA sequence of Faecalibacterium prausnitzii is selected from SEQ ID NOs: 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349, the 16S rDNA sequence of Gemmiger sp2 is selected from SEQ ID NOs: 1356, 1357, 1358, 1359, 1360, 1361, and 1362, the 16S rDNA sequence of Granulicatella adiacens is selected from SEQ ID NOs: 1363, 1364, 1365, 1366, 1367, and 1368, the 16S rDNA sequence of Intestinimonas massiliensis is selected from SEQ ID NOs: 1395, 1396, 1397, 1398, 1399, 1400, 1401, and 1402, the 16S rDNA sequence of Lactobacillus delbrueckii is selected from SEQ ID NOs: 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, and 1505, the 16S rDNA sequence of Lactobacillus reuteri is selected from SEQ ID NOs: 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, and 1009, the 16S rDNA sequence of Lactococcus lactis is selected from SEQ ID NOs: 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, and 1603, the 16S rDNA sequence of Marvinbryantia sp5 is selected from SEQ ID NOs: 1616, 1617, 1618, 1619, and 1620, the 16S rDNA sequence of Prevotella buccalis is selected from SEQ ID NOs: 1654, 1655, 1656, 1657, 1658, 1659, and 1660, the 16S rDNA sequence of Propionibacterium freudenreichii is selected from SEQ ID NOs: 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, and 1695, the 16S rDNA sequence of Scardovia wiggsiae is selected from SEQ ID NOs: 1750, 1751, 1752, 1753, and 1754, or the 16S rDNA sequence of Slackia isoflavoniconvertens is selected from SEQ ID NOs: 1755 and 1756.

In some aspects, the therapeutic composition does not comprise Allisonella histaminiformans, Bacteroides faecis, Bacteroides massiliensis, Desulfovibrionaceae NG5_sp1, Gemmiger sp2, Intestinimonas massiliensis, Marvinbryantia sp5, Propionibacterium freudenreichii, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof. In some aspects, the 16S rDNA sequence of Allisonella histaminiformans is SEQ ID NO: 774, the 16S rDNA sequence of Bacteroides faecis is selected from SEQ ID NOs: 1113, 1114, 1115, 1116, 1117, 1118, 1119, 1120, 1121, 1122, 1123, 1124, 1125, 1126, and 1127, the 16S rDNA sequence of Bacteroides massiliensis is selected from SEQ ID NOs: 1130, 1131, 1132, 1133, 1134, and 1135, the 16S rDNA sequence of Gemmiger sp2 is selected from SEQ ID NOs: 1356, 1357, 1358, 1359, 1360, 1361, and 1362, the 16S rDNA sequence of Intestinimonas massiliensis is selected from SEQ ID NOs: 1395, 1396, 1397, 1398, 1399, 1400, 1401, and 1402, the 16S rDNA sequence of Marvinbryantia sp5 is selected from SEQ ID NOs: 1616, 1617, 1618, 1619, and 1620, the 16S rDNA sequence of Propionibacterium freudenreichii is selected from SEQ ID NOs: 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, and 1695, or the 16S rDNA sequence of Slackia isoflavoniconvertens is selected from SEQ ID NOs: 1755 and 1756.

In some aspects, the therapeutic composition does not comprise Bifidobacterium animalis, Faecalibacterium prausnitzii, or bacteria having 16S rDNA having at least 94.5%, 95%, 97%, 98%, 98.7% or 99% sequence identity to any one of the 16S rDNA thereof. In some aspects, the 16S rDNA sequence of Bifidobacterium animalis is selected from SEQ ID NOs: 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, and 1218, or the 16S rDNA sequence of Faecalibacterium prausnitzii is selected from SEQ ID NOs: 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349.

In some aspects, the therapeutic composition does not comprise Granulicatella adiacens, Lactococcus lactis, Scardovia wiggsiae, or bacteria having 16S rDNA having at least 94.5%, 95%, 97%, 98%, 98.7% or 99% sequence identity to any one of the 16S rDNA thereof. In some aspects, the 16S rDNA sequence of Granulicatella adiacens is selected from SEQ ID NOs: 1363, 1364, 1365, 1366, 1367, and 1368, the 16S rDNA sequence of Lactococcus lactis is selected from SEQ ID NOs: 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, and 1603, or the 16S rDNA sequence of Scardovia wiggsiae is selected from SEQ ID NOs: 1750, 1751, 1752, 1753, and 1754.

The disclosure also relates to a therapeutic composition comprising an effective amount of a purified or isolated population of bacteria that have 16S rDNA sequence identity of at least 94.5% to 16S rDNA sequences of species belonging to one or more of the families Sutterellaceae, Coriobacteriaceae, Akkermansiaceae, Desulfovibrionaceae, Odoribacteraceae, or Rikenellaceae. In some aspects, the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Desulfovibrionaceae, Odoribacteraceae, or Rikenellaceae. In some aspects, the therapeutic composition comprises bacteria belonging to two, three, four or more families.

The disclosure also relates to a therapeutic composition comprising an effective amount of a purified or isolated population of bacteria comprising one or more bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans, or bacteria having 16S rDNA having at least 94.5%, 95%, 97%, 98%, 98.7% or 99% sequence identity to any one of the 16S rDNA thereof.

In some aspects of the therapeutic composition, the bacteria comprises one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof. In some aspects, the bacteria comprises one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

In some aspects of the therapeutic composition, the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof.

In some aspects of the therapeutic composition, the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, Desulfovibrio desulfuricans, Roseburia intestinalis, or bacteria having 16S rDNA having at least 94.5%, 95%, 97%, 98%, 98.7% or 99% sequence identity to any one of the 16S rDNA thereof.

In some aspects of the therapeutic composition, the bacteria comprises one or more bacteria selected from [Adlercreutzia] equolifaciens group, [Anaerotruncus]rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof.

The disclosure also relates to a therapeutic composition comprising a purified population of bacteria, wherein the purified or isolated population of bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772, 773, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1283, 1284, 1285, 1293, 1294, 1295, 1296, 1297, 1298, 1299, 1405, 1406, 1407, 1408, 1409, 1410, 1411, 1412, 1413, 1414, 1415, 1416, 1419, 1420, 1421, 1422, 1423, 1424, 1425, 1426, 1608, 1609, 1610, 1611, 1612, 1613, 1614, 1615, 1010, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, and 1749.

In some aspects of the therapeutic composition, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

In some aspects of the therapeutic composition, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

In some aspects of the therapeutic composition, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180, 182, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1297, 1298, 1299, 1010, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects of the therapeutic composition, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179, 184, 1293, 1294, 1295, 1296, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects of the therapeutic composition, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183, 186, 1405, 1406, 1407, 1408, 1409, 1410, 1411, 1412, 1413, 1414, 1415, 1416, 1608, 1609, 1610, 1611, 1612, 1613, 1614, 1615, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1733, 1734, 1735, 1736, 1737, 1738, 1739, 1740, 1741, 1742, 1743, 1744, 1745, 1746, 1747, 1748, and 1749.

In some aspects, the therapeutic composition comprises two, three, four, five, six, seven, eight, nine, ten, eleven, twelve, thirteen, fourteen, fifteen, sixteen, seventeen, eighteen or more bacterial species.

In some aspects of the therapeutic composition, the therapeutic composition does not comprise one or more bacteria selected from Holdemania massiliensis, Rothia mucilaginosa, Hungatella sp2, Prevotella copri, Campylobacter concisus, [Atopobium]parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus] bromii, [Ruminococcus] gauvreaui, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects of the therapeutic composition, the therapeutic composition does not comprise Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof.

In some aspects of the therapeutic composition, the therapeutic composition does not comprise Rothia mucilaginosa, [Atopobium]parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof.

In some aspects of the therapeutic composition, the therapeutic composition does not comprise Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, Bifidobacterium animalis, Faecalibacterium prausnitzii, or bacteria having 16S rDNA having at least 94.5%, 95%, 97%, 98%, 98.7% or 99% sequence identity to any one of the 16S rDNA thereof.

In some aspects of the therapeutic composition, the therapeutic composition does not comprise [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, Allisonella histaminiformans, Bacteroides faecis, Bacteroides massiliensis, Desulfovibrionaceae NG5_sp1, Gemmiger sp2, Intestinimonas massiliensis, Marvinbryantia sp5, Propionibacterium freudenreichii, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects of the therapeutic composition, the therapeutic composition does not comprise [Atopobium] parvulum, [Ruminococcus] gnavus, Bacteroides dorei group, Blautia sp1, Schaalia sp2, Granulicatella adiacens, Lactococcus lactis, Scardovia wiggsiae, or bacteria having 16S rDNA having at least 94.5%, 95%, 97%, 98%, 98.7% or 99% sequence identity to any one of the 16S rDNA thereof.

In some aspects of the therapeutic composition, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734, 764, 1011, 1012, 1013, 1014, 1015, 1016, 1017, 774, 1107, 1108, 1109, 1110, 1111, 1112, 1113, 1114, 1115, 1116, 1117, 1118, 1119, 1120, 1121, 1122, 1123, 1124, 1125, 1126, 1127, 1130, 1131, 1132, 1133, 1134, 1135, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 775, 776, 777, 778, 779, 780, 781, 782, 783, 784, 785, 786, 787, 788, 789, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1356, 1357, 1358, 1359, 1360, 1361, 1362, 1363, 1364, 1365, 1366, 1367, 1368, 1395, 1396, 1397, 1398, 1399, 1400, 1401, 1402, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1616, 1617, 1618, 1619, 1620, 1654, 1655, 1656, 1657, 1658, 1659, 1660, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1750, 1751, 1752, 1753, 1754, 1755, and 1756.

In some aspects of the therapeutic composition, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

In some aspects of the therapeutic composition, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

In some aspects of the therapeutic composition, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538, 582, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, o1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349.

In some aspects of the therapeutic composition, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599, 600, 774, 1113, 1114, 1115, 1116, 1117, 1118, 1119, 1120, 1121, 1122, 1123, 1124, 1125, 1126, 1127, 1130, 1131, 1132, 1133, 1134, 1135, 1356, 1357, 1358, 1359, 1360, 1361, 1362, 1395, 1396, 1397, 1398, 1399, 1400, 1401, 1402, 1616, 1617, 1618, 1619, 1620, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1755, and 1756.

In some aspects of the therapeutic composition, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590, 593, 1363, 1364, 1365, 1366, 1367, 1368, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1750, 1751, 1752, 1753, and 1754.

In some aspects the therapeutic composition further comprises at least one anticancer agent. In some aspects, the anticancer agent is a checkpoint inhibitor. In some aspects, the checkpoint inhibitor is selected from an anti-PD-1 antibody, an anti-CTLA-4 antibody, an anti-PD-L1 antibody or combinations thereof. In some aspects, the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT O11, MPDL3280A, MEDI-4736, MSB-0020718C, AUR-012, LAG-3, OX40 inhibitors, OX40L inhibitors, TIGIT inhibitors or STI-A1010, or combinations thereof. In some aspects, the anticancer agent is a chemotherapy agent. In some aspects, the anticancer agent is a combination of a checkpoint inhibitor and a chemotherapy agent.

In some aspects of the therapeutic composition, each of the purified or isolated populations of bacteria is present in the composition at a concentration of at least about 1×10² viable colony forming units (CFU). In some aspects, each purified or isolated population of bacteria is present in the composition at a concentration of about 1×10² to 1×10⁹ viable CFU.

In some aspects of the therapeutic composition, a fraction of the purified or isolated population of bacteria comprises a spore-forming bacteria. In some aspects, a fraction of the purified or isolated population of bacteria is in spore form. In some aspects of the therapeutic composition, a fraction of the purified or isolated population of bacteria is in vegetative form.

In some aspects, the therapeutic composition further comprises a pharmaceutically acceptable excipient. In some aspects, the therapeutic composition is formulated for delivery to the intestine. In some aspects, the therapeutic composition is enterically coated. In some aspects, the therapeutic composition is formulated for oral administration. In some aspects, the therapeutic composition is formulated into a food or beverage.

In some aspects, the therapeutic composition is capable of or can reduce the rate of tumor growth in an animal model or in a mammalian subject. In some aspects, the therapeutic composition is capable of or can reduce the rate of tumor growth in an animal model or in a mammalian subject when administered concomitantly with, prior to, or subsequent to the administration of at least one anticancer agent.

In some aspects, the therapeutic composition is capable of or can increase the number of active CD8 T cells in a tumor of an animal model or of a mammalian subject when administered concomitantly with, prior to, or subsequent to the administration of at least one anticancer agent.

In some aspects, the therapeutic composition is formulated for multiple administrations.

In some aspects of the therapeutic composition, each of the populations of bacteria is present in the composition at a concentration of at least 1×10³ viable CFU.

The disclosure also relates to a method of treating or preventing or reducing risk of a cancer in a mammalian subject in need thereof comprising administering to the subject a therapeutic composition as described herein.

In some aspects, the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof. In some aspects, the cancer is a bladder cancer.

In some aspects, the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, Desulfovibrio desulfuricans, Roseburia intestinalis, or bacteria having 16S rDNA having at least 94.5%, 95%, 97%, 98%, 98.7% or 99% sequence identity to any one of the 16S rDNA thereof. In some aspects, the cancer is a non-small cell lung cancer.

In some aspects, the bacteria comprises one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof. In some aspects, the cancer is a metastatic melanoma.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180, 182, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1297, 1298, 1299, 1010, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732. In some aspects, the cancer is a bladder cancer.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179, 184, 1293, 1294, 1295, 1296, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732. In some aspects, the cancer is a non-small cell lung cancer.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183, 186, 1405, 1406, 1407, 1408, 1409, 1410, 1411, 1412, 1413, 1414, 1415, 1416, 1608, 1609, 1610, 1611, 1612, 1613, 1614, 1615, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1733, 1734, 1735, 1736, 1737, 1738, 1739, 1740, 1741, 1742, 1743, 1744, 1745, 1746, 1747, 1748, and 1749. In some aspects, the cancer is a metastatic melanoma.

In some aspects of the method of treating or preventing or reducing risk of a cancer, the therapeutic composition does not comprise Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, Bifidobacterium animalis, Faecalibacterium prausnitzii, or bacteria having 16S rDNA having at least 94.5%, 95%, 97%, 98%, 98.7% or 99% sequence identity to any one of the 16S rDNA thereof. In some aspects, the cancer is a bladder cancer.

In some aspects of the method of treating or preventing or reducing risk of a cancer, the therapeutic composition does not comprise [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, Allisonella histaminiformans, Bacteroides faecis, Bacteroides massiliensis, Desulfovibrionaceae NG5_sp1, Gemmiger sp2, Intestinimonas massiliensis, Marvinbryantia sp5, Propionibacterium freudenreichii, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof. In some aspects, the cancer is a non-small cell lung cancer.

In some aspects of the method of treating or preventing or reducing risk of a cancer, the therapeutic composition does not comprise [Atopobium] parvulum, [Ruminococcus] gnavus, Bacteroides dorei group, Blautia sp1, Schaalia sp2, Granulicatella adiacens, Lactococcus lactis, Scardovia wiggsiae, or bacteria having 16S rDNA having at least 94.5%, 95%, 97%, 98%, 98.7% or 99% sequence identity to any one of the 16S rDNA thereof. In some aspects, the cancer is a metastatic melanoma.

In some aspects of the method of treating or preventing or reducing risk of a cancer, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538, 582, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, o1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349. In some aspects, the cancer is a bladder cancer.

In some aspects of the method of treating or preventing or reducing risk of a cancer, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599, 600, 774, 1113, 1114, 1115, 1116, 1117, 1118, 1119, 1120, 1121, 1122, 1123, 1124, 1125, 1126, 1127, 1130, 1131, 1132, 1133, 1134, 1135, 1356, 1357, 1358, 1359, 1360, 1361, 1362, 1395, 1396, 1397, 1398, 1399, 1400, 1401, 1402, 1616, 1617, 1618, 1619, 1620, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1755, and 1756. In some aspects, the cancer is non-small cell lung cancer.

In some aspects of the method of treating or preventing or reducing risk of a cancer, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590, 593, 1363, 1364, 1365, 1366, 1367, 1368, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1750, 1751, 1752, 1753, and 1754. In some aspects, the cancer is metastatic melanoma.

In some aspects of the method of treating or preventing or reducing risk of a cancer, the method further comprises administering at least one anticancer agent to the subject. In some aspects, the anticancer agent is a checkpoint inhibitor. In some aspects, the checkpoint inhibitor is selected from an anti-PD-1 antibody, an anti-CTLA-4 antibody, an anti-PD-L1 antibody or combinations thereof. In some aspects, the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT O11, MPDL3280A, MEDI-4736, MSB-0020718C, AUR-012, LAG-3, OX40 inhibitors, OX40L inhibitors, TIGIT inhibitors, STI-A1010 or combinations thereof. In some aspects, the anticancer agent comprises a chemotherapy agent. In some aspects, the anticancer agent is a combination of a checkpoint inhibitor and a chemotherapy agent.

In some aspects of the method of treating or preventing or reducing risk of a cancer, the purified or isolated population of bacteria is administered at a concentration of at least about 1×10² viable colony forming units. In some aspects of the method of treating or preventing or reducing risk of a cancer, the purified or isolated population of bacteria is administered at a concentration of at a concentration of about 1×10² to 1×10⁹ viable colony forming units.

In some aspects of the method of treating or preventing or reducing risk of a cancer, a fraction of the purified or isolated population of bacteria comprises a spore-forming bacteria. In some aspects of the method of treating or preventing or reducing risk of a cancer, a fraction of the purified or isolated population of bacteria is in spore form. In some aspects of the method of treating or preventing or reducing risk of a cancer, a fraction of the purified or isolated population of bacteria is in vegetative form.

In some aspects of the method of treating or preventing or reducing risk of a cancer, the composition further comprises a pharmaceutically acceptable excipient. In some aspects of the method of treating or preventing or reducing risk of a cancer, the composition is formulated for delivery to the intestine. In some aspects of the method of treating or preventing or reducing risk of a cancer, the composition is enterically coated. In some aspects of the method of treating or preventing or reducing risk of a cancer, the composition is formulated for oral administration. In some aspects of the method of treating or preventing or reducing risk of a cancer, the composition is formulated into a food or beverage.

In some aspects of the method of treating or preventing or reducing risk of a cancer, the mammalian subject is a human.

In some aspects of the method of treating or preventing or reducing risk of a cancer, the cancer is selected from a metastatic melanoma, melanoma of the skin, non-small cell lung cancer, a kidney cancer, a bladder cancer, a head and neck cancer, a Merkel cell skin cancer (Merkel cell carcinoma), or a Hodgkin lymphoma. In some aspects, the cancer is a metastatic melanoma. In some aspects, the cancer is a non-small cell lung cancer. In some aspects, the cancer is a bladder cancer.

In some aspects of the method of treating or preventing or reducing risk of a cancer, prior to administration of the population of bacteria the subject is subjected to antibiotic treatment and/or a bowel cleanse.

In some aspects of the method of treating or preventing or reducing risk of a cancer, the subject has previously been treated for the cancer. In some aspects, the subject has been determined to be a non-responder to the previous treatment. In some aspects, the subject has been determined to have a toxic response to the previous treatment. In some aspects, the previous treatment comprises immune checkpoint blockade monotherapy or immune checkpoint blockade combination therapy. In some aspects, the cancer is recurrent cancer.

The disclosure also relates to a method of identifying a mammalian subject as a candidate for immune checkpoint therapy in combination with adjuvant microbiome therapy to treat a cancer, the method comprising:
a) obtaining a microbiome sample from the subject,
b) determining the prevalence and/or abundance of the family of bacteria in the microbiome sample, and
c) determining that the subject is a candidate for treatment if the microbiome sample comprises the bacteria as described herein.

In some aspects, the subject is determined to be a candidate for immune checkpoint inhibitor therapy. In some aspects, the immune checkpoint therapy comprises immune checkpoint blockade monotherapy or immune checkpoint blockade combination therapy.

In some aspects of the method of identifying a mammalian subject as a candidate for immune checkpoint therapy in combination with adjuvant microbiome therapy to treat a cancer, the treatment further comprises a chemotherapy.

In some aspects of the method of identifying a mammalian subject as a candidate for immune checkpoint therapy in combination with adjuvant microbiome therapy to treat a cancer, the mammalian subject is a human.

In some aspects of the method of identifying a mammalian subject as a candidate for immune checkpoint therapy in combination with adjuvant microbiome therapy to treat a cancer, the cancer is selected from a metastatic melanoma, a melanoma of the skin, a non-small cell lung cancer, a kidney cancer, a bladder cancer, a head and neck cancer, a Merkel cell skin cancer (Merkel cell carcinoma), or a Hodgkin lymphoma. In some aspects, the cancer is a metastatic melanoma. In some aspects, the cancer is a non-small cell lung cancer. In some aspects, the cancer is a bladder cancer.

The disclosure also relates to a method of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises the bacteria as described herein.

The disclosure also relates to a therapeutic composition derived from fecal matter from a donor identified using the method of identifying a mammalian subject as a donor whose feces are useful for fecal matter transfer. In some aspects, the composition further comprises a pharmaceutically acceptable excipient. In some aspects, the therapeutic composition comprises bacteria that are in vegetative and/or spore form. In some aspects, the therapeutic composition further comprises a checkpoint inhibitor. In some aspects, the checkpoint inhibitor is selected from anti-PD-1 antibody, an anti-CTLA-4 antibody, an anti-PD-L1 antibody or combinations thereof. In some aspects, the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT O11, MPDL3280A, MEDI-4736, MSB-0020718C, AUR-012, LAG-3, OX40 inhibitors, OX40L inhibitors, TIGIT inhibitors, STI-A1010 or combinations thereof.

The disclosure also relates to a method of treating or preventing or reducing risk of a cancer in a mammalian subject comprising administering to the subject a therapeutic composition derived from fecal matter from a donor identified using the method of identifying a mammalian subject as a donor whose feces are useful for fecal matter transfer.

The disclosure also relates to a method of treating or preventing or reducing risk of a cancer comprising administering at least one anticancer treatment to a subject determined to have a microbiome sample comprising the bacteria disclosed herein.

The disclosure also relates to a method comprising evaluating a microbiome profile in a sample from a subject for the bacteria disclosed herein. In some aspects, the method further comprises comparing the microbiome profile to a control microbiome. In some aspects, the control microbiome comprises a microbiome sample from a subject determined to be a responder to an anticancer treatment. In some aspects, comparing comprises comparison of the bacteria disclosed herein to the control microbiome from a subject determined to be a responder to an anticancer treatment. In some aspects, the control microbiome comprises a microbiome sample from a subject determined to be a non-responder to an anticancer treatment. In some aspects, comparing comprises comparison of the bacteria disclosed herein to the control microbiome from a subject determined to be a non-responder to an anticancer treatment.

### BRIEF DESCRIPTION OF THE FIGURES

The following figures form part of the present specification and are included to further demonstrate certain aspects of the present disclosure. The disclosure may be better understood by reference to one or more of these figures in combination with the detailed description of specific aspects presented herein.

FIG. 1 is a graph depicting the alpha diversity distribution (i.e., the inverse Simpson index and the spore species richness) of the microbiome samples analyzed. The samples are grouped by responders (R) or non-responders (NR).

FIGS. 2A, 2B, 2C, 2D, 2E and 2F are graphs showing the microbial communities dissimilarity analysis results of the microbiome samples. FIGS. 2A and 2B show the non-metric multidimensional scaling (NMDS) plots of Bray-Curtis (abundance-based) dissimilarity and binary Jaccard (prevalence-based) distance, respectively. Each patient has only one microbiome sample included. FIGS. 2C and 2D show the NMDS plots of Bray-Curtis (abundance-based) dissimilarity and binary Jaccard (prevalence-based) distance, respectively. Each patient has up to two microbiome samples included and the samples of each patient are linked by solid lines. FIG. 2E is the NMDS plot of FIG. 2C faceted by different time points, i.e., post-treatment and pre-treatment groupings. FIG. 2F is the NMDS plot of FIG. 2E further faceted by specific cancer types, i.e., bladder cancer, lung cancer, melanoma, and RCC.

FIGS. 3A, 3B, 3C, 3D, 3E, 3F and 3G are graphs showing the weighted UniFrac distance analysis of the microbiome samples. FIG. 3A shows a principal coordinates analysis (PCoA) plot of the weighted UniFrac distance. Each patient has one microbiome sample included. FIG. 3B compares Axis 1 values distribution of the weighted Unifrac distance in the PCoA analysis between R and NR groups. FIG. 3C is a PCoA plot of the weighted UniFrac distance faceted by specific cancer types, i.e., bladder cancer, lung cancer, melanoma, and RCC. FIG. 3D is a PCoA plot of the weighted UniFrac distance faceted by specific bacterial phylum, i.e., Actinobacteria, Bacteroidetes, Firmicutes, and Proteobacteria. FIG. 3E shows a PCoA plot of the weighted UniFrac distance grouped by best overall responses, i.e., complete response (CR), progressive disease (PD), and partial response (PR). CR and PR are shaded similarly in the figure to capture overall response. FIG. 3F is FIG. 3E faceted by specific cancer types, i.e., bladder cancer, lung cancer, melanoma, and RCC. FIG. 3G is FIG. 3C overlaid with supervised clustering, indicated by the ovals.

FIGS. 4A, 4B, 4C, 4D, 4E and 4F are graphs showing the unweighted UniFrac distance analysis results of the microbiome samples. Each patient has one microbiome sample included. FIG. 4A shows a PCoA plot of the unweighted UniFrac distance. FIG. 4B compares Axis 2 values distribution of the unweighted Unifrac distance in the PCoA analysis between R and NR groups. FIG. 4C is FIG. 4A faceted by specific cancer types, i.e., bladder cancer, lung cancer, melanoma, and RCC. FIG. 4D is a PCoA plot of the unweighted UniFrac distance faceted by specific bacterial phylum, i.e., Actinobacteria, Bacteroidetes, Firmicutes, and Proteobacteria. FIG. 4E shows a PCoA plot of the unweighted UniFrac distance grouped by best overall responses, i.e., complete response (CR), progressive disease (PD), and partial response (PR). CR and PR are shaded similarly in the figure to capture overall response. FIG. 4F is FIG. 4E faceted by specific cancer types, i.e., bladder cancer, lung cancer, melanoma, and RCC.

FIGS. 5A, 5B, 5C and 5D are schematics and graphs showing the in vivo tests in tumor models of the non-responder fecal matters. FIG. 5A shows the treatment schedule of CT26 models. All of the animals were colonized with the NR patient stool. Some of the animals additionally received the anti-PD-L1+anti-CTLA4 antibodies, while the control animals received isotype control antibodies. FIG. 5B shows a comparison of tumor volume in the CT26 model animals from the different antibody treatment groups from days 9 to 21 post tumor inoculation. FIG. 5C shows the treatment schedule of MC38 models. All of the animals were colonized with the NR patient stool. Some of the animals additionally received the anti-PD-1 antibody, while the control animals received isotype control antibody. FIG. 5D shows a comparison of tumor volume in the MC38 model animals from the different antibody treatment groups from days 5 to 19 post tumor inoculation.

FIG. 6 is a schematic illustrating the CD8 activation nanostring assay. Briefly, primary human CD8 T cells were thawed at 37C for 24 hours, then activated using three-dimensional beads conjugated to α-CD3 and α-CD28 antibodies, mimicking antigen-presenting cells for 48 hours. Then, the cells were treated either with supernatants from FMTs, single bacterial strain cultures or with SCFAs for 24 hours. The cells were lysed and the cell lysates were used for a downstream gene expression analysis.

FIGS. 7A-Q are graphs illustrating individual CD8 expression profiles for the following genes: CCL3 (FIG. 7A), CCL4 (FIG. 7B), CCL5 (FIG. 7C), CD69 (FIG. 7D), CTLA4 (FIG. 7E), GZMA (FIG. 7F), GZMB (FIG. 7G), GZMH (FIG. 7H), TIM-3 (HAVCR2, (FIG. 7I), IFNG (FIG. 7J), IL2RA (FIG. 7K), LAG3 (FIG. 7L), PD-1 (FIG. 7M), PRF1 (FIG. 7N), TIGIT (FIG. 7O), TNFα (FIG. 7P), XCL1/2 (FIG. 7Q). Each “BP200xxx” sample represents an FMT prepared from an individual cancer patient.

FIG. 8A is a graph showing responder (R) FMTs compared to non-responders (NR) FMTs induce higher expression of the CD8 activation marker CD69.

FIG. 8B is a graph showing CD69 gene expression in individual cancer groups (i.e., bladder, lung, melanoma, and renal cell carcinoma (RCC) cancer groups).

FIG. 9A is a graph showing T/NK cell recruiting and activating chemokine CCL5 is correlated with outcome (R, NR).

FIG. 9B is a graph showing inhibitory receptor TIM3 is inversely correlated with outcome (R, NR).

FIG. 10A is a graph showing CD8 effector cytokines TNFα expression by patient outcome (R, NR) for each cancer type (bladder, lung, melanoma, RCC).

FIG. 10B is a graph showing CD8 interferon gamma expression by patient outcome (R, NR) for each cancer type (bladder, lung, melanoma, RCC).

FIG. 11 is a graph showing the correspondence of gene-level signals between experiment 1 and experiment 2 of CD8 gene profiles from bacterial FMT supernatants.

FIGS. 12A-Q are graphs showing CD8 expression profiles for the following genes: CCL3 (FIG. 12A), CCL4 (FIG. 12B), CCL5 (FIG. 12C), CD69 (FIG. 12D), CTLA4 (FIG. 12E), GZMA (FIG. 12F), GZMB (FIG. 12G), GZMH (FIG. 12H), HAVCR2 (TIM-3, FIG. 12I), IFNG (FIG. 12J), IL2RA (FIG. 12K), LAG3 (FIG. 12L), PDCD1 (PD-1, FIG. 12M), PRF1 (FIG. 12N), TIGIT (FIG. 12O), TNFα (FIG. 12P), XCL 1/2 (FIG. 12), for individual strains.

FIG. 13A is a graph showing CD8 cytotoxicity data for 34 single strains.

FIG. 13B is a graph showing CD8 cytotoxicity data for 7 single strains.

FIGS. 14A-Q are graphs showing data from CD8 T cell donor 1, showing a dose-dependent effect on CD8 gene expression [CCL3 (FIG. 14A), CCL4 (FIG. 14B), CCL5 (FIG. 14C), CD69 (FIG. 14D), CTLA4 (FIG. 14E), GZMA (FIG. 14F), GZMB (FIG. 14G), GZMH (FIG. 14H), HAVCR2 (TIM-3, FIG. 14I), IFNG (FIG. 14J), IL2RA (FIG. 14K), LAG3 (FIG. 14L), PDCD1 (PD-1, FIG. 14M), PRF1 (FIG. 14N), TIGIT (FIG. 14O), TNFα (FIG. 14P), XCL 1/2 (FIG. 14Q)] after treatment with three SCFAs propionate, butyrate and valerate.

FIGS. 15A-Q are graphs showing data from CD8 T cell donor 2, showing a dose-dependent effect on CD8 gene expression [CCL3 (FIG. 15A), CCL4 (FIG. 15B), CCL5 (FIG. 15C), CD69 (FIG. 15D), CTLA4 (FIG. 15E), GZMA (FIG. 15F), GZMB (FIG. 15G), GZMH (FIG. 15H), HAVCR2 (TIM-3, FIG. 15I), IFNG (FIG. 15J), IL2RA (FIG. 15K), LAG3 (FIG. 15L), PDCD1 (PD-1, FIG. 15M), PRF1 (FIG. 15N), TIGIT (FIG. 15O), TNFα (FIG. 15P), XCL 1/2 (FIG. 15Q)] after treatment with three SCFAs propionate, butyrate and valerate.

FIG. 16 is a graph showing the loading of individual genes into PC1 composite score where genes with larger absolute magnitudes (larger positive or negative scores) contribute more to the PC1 composite score. Genes with positive values are positively correlated with PC1 and bars with negative values are negatively correlated with PC1.

FIG. 17 is a graph showing PC1 was positively associated with responder status.

FIG. 18 is a graph showing PC1 by outcome separated by cancer type.

FIG. 19 is a graph showing PC1 by outcome with bladder samples removed.

FIG. 20A is a graph showing Intestinimonas butyriciproducens correlation with PC1.

FIG. 20B is a graph showing Anaerotruncus colihominis correlation with PC1.

### DETAILED DESCRIPTION

### I. Definitions

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Throughout this application, the term “about” is used according to its plain and ordinary meaning in the area of cell biology to indicate that a value includes the standard deviation of error for the device or method being employed to determine the value. When the term “about” is used in conjunction with a numerical range, it modifies that range by extending the boundaries above and below the numerical values set forth. In general, the term “about” can modify a numerical value above and below the stated value by a variance of, e.g., 10 percent, 5 percent, 3 percent, 2 percent, or 1 percent; up or down (higher or lower).

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

Unless defined otherwise, all technical and scientific terms used herein have the same meaning as commonly understood by one of ordinary skill in the art to which this disclosure is related.

“Microbiota” refers to the communities of microbes that live in or on an individual's body, both sustainably and transiently, including eukaryotes, archaea, bacteria, and viruses (including bacterial viruses (i.e., phage)).

“Microbiome” refers to the genetic content of the communities of microbes that live in and on the human body, both sustainably and transiently, including eukaryotes, archaea, bacteria, and viruses (including bacterial viruses (i.e., phage)), wherein “genetic content” includes genomic DNA, RNA such as micro RNA and ribosomal RNA, the epigenome, plasmids, and all other types of genetic information.

“Dysbiosis” refers to a state of the microbiota or microbiome of the GI tract or other body area, including mucosal or skin surfaces in which the normal diversity and/or function of the ecological network is disrupted. Any disruption from the preferred (e.g., ideal) state of the microbiota can be considered a dysbiosis, even if such dysbiosis does not result in a detectable decrease in health. This state of dysbiosis may be unhealthy, it may be unhealthy under only certain conditions, or it may prevent a subject from becoming healthier. Dysbiosis may be due to a decrease in diversity, the overgrowth of one or more pathogens or pathobionts, symbiotic organisms able to cause disease only when certain genetic and/or environmental conditions are present in a patient, or the shift to an ecological network that no longer provides a beneficial function to the host and therefore no longer promotes health.

A “spore” or a population of “spores” includes bacteria (or other single-celled organisms) that are generally viable, more resistant to environmental influences such as heat and bacteriocidal agents than vegetative forms of the same bacteria, and typically are capable of germination and out-growth. “Spore-formers” or bacteria “capable of forming spores” are those bacteria containing the genes and other necessary features to produce spores under suitable environmental conditions.

The terms “pathogen”, “pathobiont” and “pathogenic” in reference to a bacterium or any other organism or entity includes any such organism or entity that is capable of causing or affecting a disease, disorder or condition of a host organism containing the organism or entity.

The term “isolated” encompasses a bacterium or other entity or substance that has been (1) separated from at least some of the components with which it was associated when initially produced (whether in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or more of the other components with which they were initially associated. In some embodiments, isolated bacteria are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a bacterium or other material that has been separated from at least some of the components with which it was associated either when initially produced or generated (e.g., whether in nature or in an experimental setting), or during any time after its initial production. A bacterium or a bacterial population may be considered purified if it is isolated at or after production, such as from a material or environment containing the bacterium or bacterial population, and a purified bacterium or bacterial population may contain other materials up to about 10%, about 20%, about 30%, about 40%, about 50%, about 60%, about 70%, about 80%, about 90%, or above about 90% and still be considered “isolated.” In some embodiments, purified bacteria and bacterial populations are more than about 80%, about 85%, about 90%, about 91%, about 92%, about 93%, about 94%, about 95%, about 96%, about 97%, about 98%, about 99%, or more than about 99% pure. In the instance of bacterial compositions provided herein, the one or more bacterial types present in the composition can be independently purified from one or more other bacteria produced and/or present in the material or environment containing the bacterial type. Bacterial compositions and the bacterial components thereof are generally purified from residual habitat products.

“Inhibition” of a pathogen encompasses the inhibition of any desired function or activity of the bacterial compositions of the present invention. Demonstrations of pathogen inhibition, such as decrease in the growth of a pathogenic bacterium or reduction in the level of colonization of a pathogenic bacterium are provided herein and otherwise recognized by one of ordinary skill in the art. Inhibition of a pathogenic bacterium's “growth” may include inhibiting the increase in size of the pathogenic bacterium and/or inhibiting the proliferation (or multiplication) of the pathogenic bacterium. Inhibition of colonization of a pathogenic bacterium may be demonstrated by measuring the amount or burden of a pathogen before and after a treatment. An “inhibition” or the act of “inhibiting” includes the total cessation and partial reduction of one or more activities of a pathogen, such as growth, proliferation, colonization, and function.

The “colonization” of a host organism includes the transitory (e.g., for 1 day, 2 days, 3 days, 4 days, 5 days, 6 days, or 1 week) or non-transitory (e.g., greater than one week, at least two weeks, at least three weeks, at least 4 weeks, at least 6 weeks, at least 8 weeks, at least 3 month, at least 4 months, at least 6 months) residence of a bacterium or other microscopic organism. As used herein, “reducing colonization” of a host subject's gastrointestinal tract (or any other microbiotal niche) by a pathogenic bacterium includes a reduction in the residence time of the pathogen in the gastrointestinal tract as well as a reduction in the number (or concentration) of the pathogen in the lumen of the gastrointestinal tract or adhered to the mucosal surface of the gastrointestinal tract. Measuring reductions of adherent pathogens may be demonstrated, e.g., by a biopsy sample, or luminal reductions may be measured indirectly, e.g., indirectly by measuring the pathogenic burden in the stool of a mammalian host.

A “combination” of two or more bacteria includes the physical co-existence of the two bacteria, either in the same material or product or in physically connected products, as well as the temporal co-administration or co-localization of the two bacteria.

A “cytotoxic” activity or bacterium includes the ability to kill another bacterial cell, such as a pathogenic bacterial cell or a closely related species of strain. A “cytostatic” activity or bacterium includes the ability to inhibit, partially or fully, growth, metabolism, and/or proliferation of a bacterial cell, such as a pathogenic bacterial cell.

To be free of “non-comestible products” means that a bacterial composition or other material provided herein does not have a substantial amount of a non-comestible product, e.g., a product or material that is inedible, harmful or otherwise undesired in a product suitable for administration, e.g., oral administration, to a human subject.

“Residual habitat products” refers to material derived from the habitat for microbiota within or on a human or animal. For example, microbiota live in feces in the gastrointestinal tract, on the skin itself, in saliva, mucus of the respiratory tract, or secretions of the genitourinary tract (i.e., biological matter associated with the microbial community). Substantially free of residual habitat products means that the bacterial composition no longer contains the biological matter associated with the microbial environment on or in the human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms and/or fragments of microorganisms. Substantially free of residual habitat products may also mean that the bacterial composition contains no detectable cells from a human or animal and that only microbial cells are detectable. In one embodiment, substantially free of residual habitat products may also mean that the bacterial composition contains no detectable viral (including bacterial viruses (i.e., phage) or human viruses), fungal, or mycoplasmal contaminants. In another embodiment, it means that fewer than 1×10⁻²%, 1×10⁻³%, 1×10⁻⁴%, 1×10⁻⁵%, 1×10⁻⁶%, 1×10⁻⁷%, 1×10⁻⁸% of the viable cells in the bacterial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate purity include genetic analysis (e.g. PCR, DNA sequencing), serology and antigen analysis, enzymatic and metabolic analysis, and methods using instrumentation such as flow cytometry with reagents that distinguish desired constituents from contaminants.

“Phylogenetic tree” refers to a graphical representation of the evolutionary relationships of one genetic sequence to another that is generated using a defined set of phylogenetic reconstruction algorithms (e.g., parsimony, maximum likelihood, or Bayesian). Nodes in the tree represent distinct ancestral sequences and the confidence of any node is provided by a bootstrap or Bayesian posterior probability, which measures branch uncertainty.

“Operational taxonomic unit (OTU, plural OTUs)”, in some embodiments, refers to a terminal leaf in a phylogenetic tree and is defined by a specific genetic sequence and all sequences that share sequence identity to this sequence at the level of species. A “type” or a plurality of “types” of bacteria includes an OTU or a plurality of different OTUs, and also encompasses a strain, species, genus, family or order of bacteria. The specific genetic sequence may be the 16S rDNA sequence or a portion of the 16S rDNA sequence or it may be a functionally conserved housekeeping gene found broadly across the eubacterial kingdom. OTUs share at least 95%, 96%, 97%, 98%, or 99/a sequence identity. OTUs generally defined by comparing sequences between organisms. Sequences with less than 95% sequence identity are not considered to form part of the same OTU. In some embodiments, metagenomics methods, known in the art, are used to identify species and/or OTUs.

“Clade” refers to the set of OTUs or members of a phylogenetic tree downstream of a statistically valid node in a phylogenetic tree. A clade is a group of related organisms representing all of the phylogenetic descendants of a common ancestor. The clade comprises a set of terminal leaves in the phylogenetic tree that is a distinct monophyletic evolutionary unit.

The terms “subject” or “patient” refers to any animal subject including humans, laboratory animals (e.g., primates, rats, mice), livestock (e.g., cows, sheep, goats, pigs, turkeys, chickens), and household pets (e.g., dogs, cats, rodents, etc.). The subject or patient may be healthy, or may be suffering from an infection due to a gastrointestinal pathogen or may be at risk of developing or transmitting to others an infection due to a gastrointestinal pathogen.

The term “pathobiont” refer to specific bacterial species found in healthy hosts that may trigger immune-mediated pathology and/or disease in response to certain genetic or environmental factors. Chow et al., (2011) Curr Op Immunol. Pathobionts of the intestinal microbiota and inflammatory disease. 23: 473-80. Thus, a pathobiont is a pathogen that is mechanistically distinct from an acquired infectious organism. Thus, the term “pathogen” includes both acquired infectious organisms and pathobionts.

As used herein, the term “immunoregulator” refers to an agent or a signaling pathway (or a component thereof) that regulates an immune response. “Regulating,” “modifying” or “modulating” an immune response refers to any alteration of the immune system or in the activity of such cell. Such regulation includes stimulation or suppression of the immune system which may be manifested by an increase or decrease in the number of various cell types, an increase or decrease in the activity of these cells, or any other changes which can occur within the immune system. Both inhibitory and stimulatory immunoregulators have been identified, some of which may have enhanced function or utility as a therapeutic target in a cancer microenvironment.

As used herein, the term “immune evasion” refers to inhibition of a subject's immune system or a component thereof (e.g., endogenous T cell response) by a cancer or tumor cell in order to maximize or allow continued growth or spread of the cancer/tumor.

As used herein, the term “immunotherapy” refers to the treatment or prevention of a disease or condition (e.g., cancer) by a method comprising inducing, enhancing, suppressing or otherwise modifying an immune response.

As used herein, “potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

As used herein, the term “antibody” refers to a whole antibody molecule or a fragment thereof (e.g., fragments such as Fab, Fab′, and F(ab′)₂), it may be a polyclonal or monoclonal antibody, a chimeric antibody, a humanized antibody, a human antibody, etc.

As used herein, “cancer” means all types of cancers. In particular, the cancers can be solid or non-solid cancers. Non-limiting examples of cancers are carcinomas or adenocarcinomas such as breast, prostate, ovary, lung, pancreas or colon cancer, sarcomas, lymphomas, melanomas, leukemias, germ cell cancers and blastomas.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a cancer or enhancing overall survival. Treatment can be of a subject having a cancer or a subject who does not have a cancer (e.g., for prevention or prophylaxis). As used here, the terms “treat,” “treating,” and “treatment” refer to the administration of an effective dose or effective dosage.

The terms “prevent,” “preventing,” and “prevention,” as used herein, refer to partially or completely delaying onset of a cancer; partially or completely delaying onset of one or more symptoms, features, or clinical manifestations of a cancer; partially or completely delaying onset of one or more symptoms, features, or manifestations of a cancer (including, but not limited to, fatigue or reduced exercise capacity); partially or completely delaying progression from a cancer; and/or decreasing the risk of developing pathology associated with a cancer.

### II. General Compositions and Methods of the Disclosure

Provided herein are compositions and methods for treatment and/or prevention and/or reducing risk of a cancer by microbiome manipulation. In particular, the amount, identity, presence, and/or ratio of bacteria in the microbiome (e.g., GI microbiome) in a subject is manipulated to facilitate treatment and/or prevention and/or reducing risk of a cancer. Furthermore, applicants have discovered that the abundance and/or prevalence of certain commensal bacteria in feces can be used to identify fecal donors and/or donations that can improve patient response to a checkpoint inhibitor. Fecal material from such individuals can be used, e.g., in fecal microbiome transplantation or in a processed form derived from such material, for example a preparation enriched in Firmicutes (e.g., Clostridia, Clostridiales, or spore formers), that are in vegetative and/or spore form.

Applicants have identified bacterial species that are useful for increasing the efficacy of cancer treatment, e.g., treatments using checkpoint inhibitors. In some aspects, the effectiveness of an endogenous immune response, immunotherapy, chemotherapeutic, or other treatment (e.g., surgery, radiation, etc.) in the treatment and/or prevention and/or reducing risk of reoccurrence of cancer and/or tumor is dependent upon conditions within the subject (e.g., the tumor microenvironment). In particular, the identity or characteristics (e.g., concentration or level) of the microbiome within a subject can affect the effectiveness of cancer treatments (e.g., generally or specific treatments) and/or the effectiveness of the subject's own response to cancer, e.g., immune response.

In some aspects, the presence or increased level of one or more species of bacteria in a subject facilitates treatment (e.g., immunotherapy, chemotherapy, etc.) and/or the subject's endogenous immune response to cancer and/or tumor cells. In some aspects, the absence and/or decreased level of one or more species of bacteria in a subject discourages cancer/tumor growth, spread, and/or evasion of treatment/immune response. In some aspects, the absence or decreased level of one or more species of bacteria in a subject facilitates treatment (e.g., immunotherapy, chemotherapy, etc.) and/or the subject's endogenous immune response to cancer and/or tumor cells.

In some aspects, the presence of certain microbes (e.g., microbes that facilitate cancer treatment) in a subject creates an environment or microenvironment (e.g., microbiome) that is conducive to the treatment of cancer and/or inhibits cancer/tumor growth. In some aspects, the presence of detrimental microbes (e.g., microbes that facilitate cancer/tumor growth and/or prevent treatment) in a subject creates an environment or microenvironment (e.g., microbiome) that is conducive to the treatment of cancer and/or inhibits cancer/tumor growth. Microbes or their products may act locally at the level of the gut epithelium and the lamina propria to alter immunological tone or immune cell trafficking, or they may act distally by the translocation of microbes or their products into circulation to alter peripheral immune responses, e.g. in blood, liver, spleen, lymph nodes or tumor.

Modulation of microflora levels and/or identity can comprise encouraging or facilitating growth of one or more species of beneficial microbes (e.g., microbes that facilitate cancer treatment), discouraging or inhibiting growth of one or more types of detrimental microbes (e.g., species of bacteria that facilitate cancer/tumor growth and/or prevent treatment), administering one or more types of beneficial microbes (e.g., species of bacteria that facilitate cancer treatment) to the subject, and/or combinations thereof. Aspects within the scope herein are not limited by the mechanisms for introducing one or more microbes (e.g., probiotic administration, fecal transplant, etc.), encouraging growth of beneficial microbes (e.g., administering agents that skew the environment within the subject toward growth conditions for the beneficial microbes), discouraging or inhibiting growth of detrimental microbes (e.g., administering agents that skew the environment within the subject away from growth conditions for the detrimental microbes, administration of antimicrobial(s), etc.), and combinations thereof.

In some aspects, methods are provided for the treatment and/or prevention and/or reducing risk of cancer by the manipulation of the presence, amount, or relative ratio of one or more families, genera, or species of bacteria (e.g., in the gastrointestinal microbiome). In some aspects, the presence, amount, or relative ratio of particular bacteria, fungi, and/or archaea within a subject is altered. For example, in some aspects, the presence, amount, or relative ratio of one or more bacteria from the families Sutterellaceae, Coriobacteriaceae, Akkermansiaceae, Desulfovibrionaceae, Odoribacteraceae, or Rikenellaceae is manipulated.

In some aspects, the presence, amount or relative ratio of a purified or isolated population of bacteria that have 16S rDNA sequence identity of at least 94.5% to 16S rDNA sequences of species belonging to one or more of the families Sutterellaceae, Coriobacteriaceae, Akkermansiaceae, Desulfovibrionaceae, Odoribacteraceae, or Rikenellaceaeare manipulated or adjusted. In some aspects, the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Desulfovibrionaceae, Odoribacteraceae, or Rikenellaceae.

In some aspects, the presence, amount or relative ratio of one or more bacterial species selected from Intestinimonas butyriciproducens and Anaerotruncus colihominis or bacteria having a 16S rDNA sequence having at least 97%, 98%, 98.7% or 99% sequence identity to any one 16S rDNA sequence thereof, are manipulated or adjusted. In some aspects, the presence, amount or relative ratio of one or more bacterial species selected from Murimonas intestini and Faecalicatena orotica or bacteria having a 16S rDNA sequence having at least 97%, 98%, 98.7% or 99% sequence identity to any one 16S rDNA sequence thereof, are manipulated or adjusted.

In some aspects, the presence, amount or relative ratio of one or more bacterial species selected from Anaerotignum lactatifermentans, Anaerotruncus colihominis, Angelakisella sp1, Bacteroides cellulosilyticus, Bacteroides gallinarum, Bacteroides plebeius, Blautia coccoides group, Christensenella minuta, Christensenellaceae NG16_sp1, Clostridium hylemonae, Collinsella bouchesdurhonensis, Coprobacter secundus group, Eisenbergiella massiliensis, Emergencia timonensis, Faecalicatena orotica, Fusicatenibacter sp1, Intestinimonas butyriciproducens, Intestinimonas timonensis, Lachnospiraceae NG17_sp3, Lawsonibacter sp3, Oxalobacter formigenes, Peptoniphilaceae NG4_sp2, Prochlorotrichaceae NG20_sp1, or bacteria having a 16S rDNA sequence having at least 97%, 98%, 98.7% or 99% sequence identity to any one 16S rDNA sequence thereof, are manipulated or adjusted.

In some aspects, the presence, amount or relative ratio of one or more bacterial species selected from Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans or bacteria having a 16S rDNA sequence having at least 97%, 98%, 98.7% or 99% sequence identity to any one 16S rDNA sequence thereof, are manipulated or adjusted.

In some aspects, the presence, amount or relative ratio of one or more bacterial species selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans, or bacteria having 16S rDNA having at least 94.5%, 95%, 97%, 98%, 98.7% or 99% sequence identity to any one of the 16S rDNA thereof, are manipulated or adjusted.

In some aspects, the methods exclude the administration of, the evaluation of, the detection of, or the determination of the amount or relative ratio of one or more bacterial species selected from Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects, the methods exclude the administration of, the evaluation of, the detection of, or the determination of the amount or relative ratio of one or more bacterial species selected from Holdemania massiliensis, Rothia mucilaginosa, Hungatella sp2, Prevotella copri, Campylobacter concisus, [Atopobium] parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus] bromii, [Ruminococcus] gauvreaui, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects, the presence, amount, or relative ratio of one or more bacteria Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof are manipulated. In some aspects, the presence, amount, or relative ratio of one or more bacteria Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof are manipulated. In some aspects, the presence, amount, or relative ratio of one or more bacteria Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof are manipulated. In some aspects, the presence, amount, or relative ratio of one or more bacteria [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, Desulfovibrio desulfuricans, Roseburia intestinalis, or bacteria having 16S rDNA having at least 94.5%, 95%, 97%, 98%, 98.7% or 99% sequence identity to any one of the 16S rDNA thereof are manipulated. In some aspects, the presence, amount, or relative ratio of one or more bacteria [Adlercreutzia] equolifaciens group, [Anaerotruncus]rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof are manipulated.

### III. Therapeutic Compositions

This disclosure relates to a therapeutic composition comprising an effective amount of a purified or isolated population of bacteria that have 16S rDNA sequence identity of at least 94.5% to 16S rDNA sequences of species belonging to one or more of the families Sutterellaceae, Coriobacteriaceae, Akkermansiaceae, Desulfovibrionaceae, Odoribacteraceae, or Rikenellaceae.

In some aspects, the therapeutic composition can comprise an effective amount of a purified or isolated population of bacteria that have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Desulfovibrionaceae, Odoribacteraceae, or Rikenellaceae.

In some aspects, the therapeutic composition can comprise bacteria belonging to two, three, four or more families.

The disclosure also relates to a therapeutic composition comprising an effective amount of a purified or isolated population of bacteria comprising one or more bacteria selected from Intestinimonas butyriciproducens and Anaerotruncus colihominis or bacteria having a 16S rDNA sequence having at least 97%, 98%, 98.7% or 99% sequence identity to any one 16S rDNA sequence thereof. The disclosure also relates to a therapeutic composition comprising an effective amount of a purified or isolated population of bacteria comprising one or more bacteria selected from Murimonas intestini and Faecalicatena orotica or bacteria having a 16S rDNA sequence having at least 97%, 98%, 98.7% or 99% sequence identity to any one 16S rDNA sequence thereof.

The disclosure also relates to a therapeutic composition comprising an effective amount of a purified or isolated population of bacteria comprising one or more bacteria selected from Anaerotignum lactatifermentans, Anaerotruncus colihominis, Angelakisella sp1, Bacteroides cellulosilyticus, Bacteroides gallinarum, Bacteroides plebeius, Blautia coccoides group, Christensenella minuta, Christensenellaceae NG16_sp1, Clostridium hylemonae, Collinsella bouchesdurhonensis, Coprobacter secundus group, Eisenbergiella massiliensis, Emergencia timonensis, Faecalicatena orotica, Fusicatenibacter sp1, Intestinimonas butyriciproducens, Intestinimonas timonensis, Lachnospiraceae NG17_sp3, Lawsonibacter sp3, Oxalobacter formigenes, Peptoniphilaceae NG4_sp2, Prochlorotrichaceae NG20_sp1, or bacteria having a 16S rDNA sequence having at least 97%, 98%, 98.7% or 99% sequence identity to any one 16S rDNA sequence thereof.

The disclosure also relates to a therapeutic composition comprising an effective amount of a purified or isolated population of bacteria comprising one or more bacteria selected from Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans or bacteria having a 16S rDNA sequence having at least 97%, 98%, 98.7% or 99% sequence identity to any one 16S rDNA sequence thereof.

The disclosure also relates to a therapeutic composition comprising an effective amount of a purified or isolated population of bacteria comprising one or more bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans, or bacteria having 16S rDNA having at least 94.5%, 95%, 97%, 98%, 98.7% or 99% sequence identity to any one of the 16S rDNA thereof.

The disclosure also relates to a therapeutic composition comprising an effective amount of a purified or isolated population of bacteria comprising one or more bacteria selected from Akkermansia muciniphila, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

In some aspects, the bacteria in the composition can comprise one or more bacteria selected from Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof. In other aspects, the bacteria in the composition can comprise one or more bacteria selected from Desulfovibrio desulfuricans, Roseburia intestinalis, or bacteria having 16S rDNA having at least 94.5%, 95%, 97%, 98%, 98.7% or 99% sequence identity to any one of the 16S rDNA thereof. In other aspects, the bacteria in the composition can comprise one or more bacteria selected from Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof.

In some aspects, the bacteria in the composition can comprise one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof. In some aspects, the bacteria in the composition can comprise one or more bacteria selected from Akkermansia muciniphila or bacteria having 16S rDNA having at least 94.5%, 95%, 97%, 98%, 98.7% or 99% sequence identity to any one of the 16S rDNA thereof.

In other aspects, the bacteria in the composition can comprise one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

In other aspects, the bacteria in the composition can comprise one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, or bacteria having 16S rDNA having at least 94.5%, 95%, 97%, 98%, 98.7% or 99% sequence identity to any one of the 16S rDNA thereof.

In other aspects, the bacteria in the composition can comprise one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, or bacteria having 16S rDNA having at least 94.5%, 95%, 97%, 98%, 98.7% or 99% sequence identity to any one of the 16S rDNA thereof. In other aspects, the bacteria in the composition can comprise one or more bacteria selected from [Extibacter] muris, Collinsella intestinalis, Collinsella stercoris, Muricomes sp1, Neobitarella massiliensis, Prevotellamassilia sp2, Ruminococcaceae NG33 sp1, or bacteria having 16S rDNA having at least 94.5%, 95%, 97%, 98%, 98.7% or 99% sequence identity to any one of the 16S rDNA thereof.

In other aspects, the bacteria in the composition can comprise one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof. In other aspects, the bacteria in the composition can comprise one or more bacteria selected from [Adlercreutzia] equolifaciens group, [Anaerotruncus] rubiinfantis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

In some aspects, the bacteria in the composition can comprise one or more bacteria selected from bacteria in any of Tables 4-6, 8, or 10-13 associated with responders (R), or bacteria having 16S rDNA having at least 94.5%, 95%, 97%, 98%, 98.7% or 99% sequence identity to any one of the 16S rDNA thereof. In some aspects, the bacteria in the composition can comprise one or more bacteria from Table 17, such as those bacteria associated with a positive spearman.rho value, or bacteria having 16S rDNA having at least 94.5%, 95%, 97%, 98%, 98.7% or 99% sequence identity to any one of the 16S rDNA thereof.

The disclosure also provides a therapeutic composition comprising a purified population of bacteria, wherein the purified or isolated population of bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1066, 1067, 1068, 1069, 1070, 1071, 1072, 1073, 1074, 1075, 1076, 1077, 1078, 1079, 1080, 1081, 1082, 1083, 1084, and 1085.

The disclosure also provides a therapeutic composition comprising a purified population of bacteria, wherein the purified or isolated population of bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1621, 800, 801, 802, 803, 804, 805, 806, 807, 808, 809, and 810.

The disclosure also provides a therapeutic composition comprising a purified population of bacteria, wherein the purified or isolated population of bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1283, 1284, 1285, 1293, 1294, 1295, 1296, 1297, 1298, 1299, 1405, 1406, 1407, 1408, 1409, 1410, 1411, 1412, 1413, 1414, 1415, 1416, 1419, 1420, 1421, 1422, 1423, 1424, 1425, 1426, 1608, 1609, 1610, 1611, 1612, 1613, 1614, 1615, 1010, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, and 1749.

In some aspects, the bacteria in the composition comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1297, 1298, 1299, 1010, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732

In some aspects, the bacteria in the composition comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1293, 1294, 1295, 1296, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects, the bacteria in the composition comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1405, 1406, 1407, 1408, 1409, 1410, 1411, 1412, 1413, 1414, 1415, 1416, 1608, 1609, 1610, 1611, 1612, 1613, 1614, 1615, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1733, 1734, 1735, 1736, 1737, 1738, 1739, 1740, 1741, 1742, 1743, 1744, 1745, 1746, 1747, 1748, and 1749.

The disclosure also provides a therapeutic composition comprising a purified population of bacteria, wherein the purified or isolated population of bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1128, 1129, 1136, 1137, 1138, 1139, 1140, 1141, 1142, 1143, 1144, 1145, 1146, 1147, 1148, 1149, 1150, 1151, 1152, 1153, 1154, 1155, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1275, 1276, 1277, 1278, 1279, 1280, 1281, 1282, 1286, 1287, 1288, 790, 791, 792, 793, 794, 795, 796, 1289, 1290, 1291, 1292, 797, 798, 799, 1300, 1301, 1302, 1303, 1304, 1305, 1306, 1307, 1308, 1309, 1310, 1311, 1312, 1313, 1314, 1315, 1316, 1317, 1318, 1319, 1320, 800, 801, 802, 803, 804, 805, 806, 807, 808, 809, 810, 1350, 1351, 1352, 1353, 1354, 1355, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1403, 1404, 1417, 1418, 1604, 1605, 1606, 1607, 1649, 1650, 1651, 1652, 1653, 172, and 1661.

The disclosure also provides a therapeutic composition comprising a purified population of bacteria, wherein the purified or isolated population of bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772, 773, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1283, 1284, 1285, 1293, 1294, 1295, 1296, 1297, 1298, 1299, 1405, 1406, 1407, 1408, 1409, 1410, 1411, 1412, 1413, 1414, 1415, 1416, 1419, 1420, 1421, 1422, 1423, 1424, 1425, 1426, 1608, 1609, 1610, 1611, 1612, 1613, 1614, 1615, 1010, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749.

In some aspects, the bacteria in the composition comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

In some aspects, the bacteria in the composition comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

In some aspects, the bacteria in the composition comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180, 182, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1297, 1298, 1299, 1010, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects, the bacteria in the composition comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179, 184, 1293, 1294, 1295, 1296, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects, the bacteria in the composition comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183, 186, 1405, 1406, 1407, 1408, 1409, 1410, 1411, 1412, 1413, 1414, 1415, 1416, 1608, 1609, 1610, 1611, 1612, 1613, 1614, 1615, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1733, 1734, 1735, 1736, 1737, 1738, 1739, 1740, 1741, 1742, 1743, 1744, 1745, 1746, 1747, 1748, and 1749.

In other aspects, the therapeutic composition can comprise two, three, four, five, six, seven, eight, nine, ten, eleven, twelve, thirteen, fourteen, fifteen, sixteen, seventeen, eighteen, nineteen, twenty, twenty-five, thirty, or more bacterial species.

In some aspects, the therapeutic composition does not comprise one or more bacteria selected from Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects, the therapeutic composition does not comprise one or more bacteria selected from Holdemania massiliensis, Rothia mucilaginosa, Hungatella sp2, Prevotella copri, Campylobacter concisus, [Atopobium] parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus]bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus]gnavus, Bacteroides dorei group, Blautia sp1, Schaalia sp2, Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects, the therapeutic composition does not comprise one or more bacteria selected from Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects, the therapeutic composition does not comprise Allisonella histaminiformans, Bacteroides faecis, Bacteroides massiliensis, Desulfovibrionaceae NG5_sp1, Gemmiger sp2, Intestinimonas massiliensis, Marvinbryantia sp5, Propionibacterium freudenreichii, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof. In other aspects, the therapeutic composition does not comprise Bifidobacterium animalis, Faecalibacterium prausnitzii, or bacteria having 16S rDNA having at least 94.5%, 95%, 97%, 98%, 98.7% or 99% sequence identity to any one of the 16S rDNA thereof. In other aspects, the therapeutic composition does not comprise Granulicatella adiacens, Lactococcus lactis, Scardovia wiggsiae, or bacteria having 16S rDNA having at least 94.5%, 95%, 97%, 98%, 98.7% or 99% sequence identity to any one of the 16S rDNA thereof.

In some aspects, the therapeutic composition does not comprise one or more bacteria selected from Rothia mucilaginosa, Prevotella copri, Campylobacter concisus, [Atopobium] parvulum, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

In some aspects, the therapeutic composition does not comprise Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof. In some aspects, the therapeutic composition does not comprise Rothia mucilaginosa, Prevotella copri, Campylobacter concisus, or bacteria having 16S rDNA having at least 94.5%, 95%, 97%, 98%, 98.7% or 99% sequence identity to any one of the 16S rDNA thereof.

In some aspects, the therapeutic composition does not comprise Rothia mucilaginosa, [Atopobium] parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof. In some aspects, the therapeutic composition does not comprise Rothia mucilaginosa, [Atopobium] parvulum, Prevotella copri, Campylobacter concisus, or bacteria having 16S rDNA having at least 94.5%, 95%, 97%, 98%, 98.7% or 99% sequence identity to any one of the 16S rDNA thereof.

In some aspects, the therapeutic composition does not comprise Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, or bacteria having 16S rDNA having at least 94.5%, 95%, 97%, 98%, 98.7% or 99% sequence identity to any one of the 16S rDNA thereof. In some aspects, the therapeutic composition does not comprise Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, or bacteria having 16S rDNA having at least 94.5%, 95%, 97%, 98%, 98.7% or 99% sequence identity to any one of the 16S rDNA thereof.

In some aspects, the therapeutic composition does not comprise [Coprococcus] catus, [Lactobacillus] fermentum, [Romboutsia] timonensis, [Ruminococcus] bromii, [Ruminococcus] gauvreaui, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, or bacteria having 16S rDNA having at least 94.5%, 95%, 97%, 98%, 98.7% or 99% sequence identity to any one of the 16S rDNA thereof. In some aspects, the therapeutic composition does not comprise [Coprococcus] catus, [Lactobacillus] fermentum, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, or bacteria having 16S rDNA having at least 94.5%, 95%, 97%, 98%, 98.7% or 99% sequence identity to any one of the 16S rDNA thereof.

In some aspects, the therapeutic composition does not comprise [Atopobium] parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

In some aspects, the therapeutic composition does not comprise one or more bacteria selected from bacteria in any of Tables 4-6, or 9-13 associated with non-responders (R), or bacteria having 16S rDNA having at least 94.5%, 95%, 97%, 98%, 98.7% or 99% sequence identity to any one of the 16S rDNA thereof.

In some aspects, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734, 764, 1011, 1012, 1013, 1014, 1015, 1016, 1017, 774, 1107, 1108, 1109, 1110, 1111, 1112, 1113, 1114, 1115, 1116, 1117, 1118, 1119, 1120, 1121, 1122, 1123, 1124, 1125, 1126, 1127, 1130, 1131, 1132, 1133, 1134, 1135, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 775, 776, 777, 778, 779, 780, 781, 782, 783, 784, 785, 786, 787, 788, 789, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1356, 1357, 1358, 1359, 1360, 1361, 1362, 1363, 1364, 1365, 1366, 1367, 1368, 1395, 1396, 1397, 1398, 1399, 1400, 1401, 1402, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1616, 1617, 1618, 1619, 1620, 1654, 1655, 1656, 1657, 1658, 1659, 1660, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1750, 1751, 1752, 1753, 1754, 1755, and 1756.

In some aspects, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

In some aspects, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

In some aspects, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538, 582, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, o1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349.

In some aspects, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599, 600, 774, 1113, 1114, 1115, 1116, 1117, 1118, 1119, 1120, 1121, 1122, 1123, 1124, 1125, 1126, 1127, 1130, 1131, 1132, 1133, 1134, 1135, 1356, 1357, 1358, 1359, 1360, 1361, 1362, 1395, 1396, 1397, 1398, 1399, 1400, 1401, 1402, 1616, 1617, 1618, 1619, 1620, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1755, and 1756.

In some aspects, the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590, 593, 1363, 1364, 1365, 1366, 1367, 1368, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1750, 1751, 1752, 1753, and 1754.

In some aspects, a therapeutic composition can reduce the rate of tumor growth in an animal model. In some embodiments, a therapeutic composition can reduce the rate of tumor growth in a human subject. In some embodiments, a therapeutic composition can reduce the rate of tumor growth in an in vitro cell culture model. In some embodiments, a therapeutic composition can reduce the rate of tumor growth in an in situ model.

In some aspects, the method of treating a cancer may use any of the therapeutic compositions listed herein, including combinations of genera from therapeutic compositions and/or combinations of species from therapeutic compositions. These methods of treatment, including combination treatment with other anti-cancer agents, are described in further detail below.

In some aspects, the bacteria in the therapeutic compositions may be identified by species, operational taxonomic unit (OTU), whole genome sequence or other methods known in the art for defining different types of bacteria.

Bacterial compositions can comprise two types of bacteria (termed “binary combinations” or “binary pairs”) or greater than two types of bacteria. Bacterial compositions that comprise three types of bacteria are termed “ternary combinations”. For instance, a bacterial composition can comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21, 22, 23, 24, 25, 26, 27, 28, 29 30, 31, 32, 33, 34, 35, 36, 37, 38, 39, or at least 40, at least 50 or greater than 50 types of bacteria, as defined by species or operational taxonomic unit (OTU), or otherwise as provided herein.

In another aspect, the number of types of bacteria present in a bacterial composition is at or below a known value. For example, in such embodiments the bacterial composition comprises 50 or fewer types of bacteria, such as 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 or fewer, or 9 or fewer types of bacteria, 8 or fewer types of bacteria, 7 or fewer types of bacteria, 6 or fewer types of bacteria, 5 or fewer types of bacteria, 4 or fewer types of bacteria, or 3 or fewer types of bacteria. In another aspect, a bacterial composition comprises from 2 to no more than 40, from 2 to no more than 30, from 2 to no more than 20, from 2 to no more than 15, from 2 to no more than 10, or from 2 to no more than 5 types of bacteria.

A bacterial composition useful in a method described herein can be prepared comprising at least one type of isolated bacteria, wherein a first type and a second type are independently chosen from the genera or species listed herein. In another aspect, the first and/or second OTUs can be characterized by one or more of the variable regions of the 16S sequence (V1-V9). These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. (e.g., Brosius et al., Complete nucleotide sequence of a 16S ribosomal RNA gene from Escherichia coli, Proc Nat Acad Sci 75(10):4801-4805 (1978)). In some aspects, at least one of the V1, V2, V3, V4, V5, V6, V7, V8, and V9 regions are used to characterize an OTU. In one aspect, the V1, V2, and V3 regions are used to characterize an OTU. In another aspect, the V3, V4, and V5 regions are used to characterize an OTU. In another embodiment, the V4 region is used to characterize an OTU.

Methods of the disclosure include administration of a combination of therapeutic agents and compositions. The therapy can be administered in any suitable manner known in the art. For example, the therapies may be administered sequentially (at different times) or concurrently (at the same time). In some aspects, the therapies are in a separate composition. In some aspects, the therapies are in the same composition.

Various combinations of the therapies can be employed, for example, one therapy or composition designated “A” and another therapy or composition designated “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

The therapies and compositions of the disclosure can be administered by the same route of administration or by different routes of administration. In some aspects, the therapy is administered intracolonically, intravenously, intramuscularly, subcutaneously, topically, orally, transdermally, intraperitoneally, intraorbitally, by implantation, intrathecally, intraventricularly, or intranasally. In some embodiments, the microbial modulator is administered intravenously, intramuscularly, subcutaneously, topically, orally, transdermally, intraperitoneally, intraorbitally, by implantation, intrathecally, intraventricularly, or intranasally.

In some aspects, the compositions of the disclosure are administered in a therapeutically effective or sufficient amount of each of the at least one isolated or purified population of bacteria or each of the at least two, 3, 4, 5, 6, 7, 8, 9, 10 11, 12, 13, 14, or 15 isolated or purified populations of bacteria of the microbial modulator compositions of the embodiments that is administered to a human will be at least about 1×10³ viable colony forming units (CFU) of bacteria or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ viable CFU (or any derivable range therein). In some aspects, a single dose will contain an amount of bacteria (such as a specific bacteria or species, genus, or family described herein) of at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ viable CFU (or any derivable range therein) of a specified bacteria. In some aspects, a single dose will contain at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ viable CFU (or any derivable range therein) of total bacteria. In specific aspects, the bacteria are provided in spore form or as sporulated bacteria. In particular aspects, the concentration of spores of each isolated or purified population of bacteria, for example of each species, subspecies or strain, is at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ (or any derivable range therein) viable bacterial spores per gram of composition or per administered dose. In some aspects, the composition comprises or the method comprises administration of at least, at most, or exactly 1, 2, 3, 4, 5, 6, 7, 8, 9, 10, 11, 12, 13, 14, 15, 20, 25, 30, 40, or 50 (or any derivable range therein) of different bacterial species, different bacterial genus, or different bacterial family.

In some aspects, the therapeutically effective or sufficient amount of each of the at least one isolated or purified population of bacteria or each of the at least two, 3, 4, 5, 6, 7, 8, 9, 10 11, 12, 13, 14, or 15 isolated or purified populations of bacteria of the microbial modulator compositions of the embodiments that is administered to a human will be at least about 1×10³ cells of bacteria or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ cells (or any derivable range therein). In some aspects, a single dose will contain an amount of bacteria (such as a specific bacteria or species, genus, or family described herein) of at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ cells (or any derivable range therein) of a specified bacteria. In some aspects, a single dose will contain at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ cells (or any derivable range therein) of total bacteria. In specific aspects, the bacteria are provided in spore form or as sporulated bacteria. In particular aspects, the concentration of spores of each isolated or purified population of bacteria, for example of each species, subspecies or strain, is at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ (or any derivable range therein) viable bacterial spores per gram of composition or per administered dose. In some aspects, the composition comprises or the method comprises administration of at least, at most, or exactly 1, 2, 3, 4, 5, 6, 7, 8, 9, 10, 11, 12, 13, 14, 15, 20, 25, 30, 40, or 50 (or any derivable range therein) of different bacterial species, different bacterial genus, or different bacterial family.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose, for example, of CPI, is understood to refer to an amount necessary to achieve a particular effect. In some aspects, it is contemplated that doses in the range from 10 mg/kg to 200 mg/kg can affect the protective capability of these agents. Thus, it is contemplated that the doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some aspects, the therapeutically effective or sufficient amount of a therapeutic composition comprising for example, a CPI, that is administered to a human will be in the range of about 0.01 to about 50 mg/kg of patient body weight whether by one or more administrations. In some aspects, the therapeutic agent used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some aspects, the therapeutic agent is administered at 15 mg/kg. However, other dosage regimens may be useful. In one embodiment, a therapeutic agent described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In some aspects, the effective dose of the therapeutic composition comprising for example, a CPI, is one which can provide a blood level of about 1 μM to 150 μM. In another aspect, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent, for example, a CPI, that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In some aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IV. Methods for Evaluating Bacteria

A. Determining Bacterial Genera and Species

In some aspects, the bacterial genera or species for use in a therapeutic composition is as described in this disclosure.

In some aspects, the bacterial genera or species for use in a therapeutic composition are those genera or species that are found to be prevalent in the microbiome of subjects that respond to an anti-cancer therapy, e.g., subjects who are responders. In some aspects, the genera or species are more prevalent in the microbiome of a responder compared to the microbiome of a subject who does not respond to an anti-cancer therapy, e.g., a non-responder. In other aspects, the genera or species are more prevalent in the microbiome of a responder compared to the microbiome of a healthy subject that does not have a cancer and thus has not been treated with an anti-cancer therapy.

In some aspects, the bacterial genera or species for use in a therapeutic composition are those genera or species that are found to be more abundant in the microbiome of subjects that respond to an anti-cancer therapy, e.g., subjects who are responders. In some aspects, the genera or species are more abundant in the microbiome of a responder compared to the microbiome of a subject who does not respond to an anti-cancer therapy, e.g., a non-responder. In other aspects, the genera or species are more abundant in the microbiome of a responder compared to the microbiome of a healthy subject that does not have a cancer and thus has not been treated with an anti-cancer therapy.

In some aspects, the subject is considered a responder if, following treatment with an anti-cancer therapy, the subject shows a complete response to the therapy, e.g., a complete remission of the cancer. In other aspects, the subject is considered a responder if, following treatment with an anti-cancer therapy, the subject shows a complete response to the therapy or a partial response to the therapy, e.g., a reduction in tumor size or tumor load. In other aspects, the subject is considered a responder if, following treatment with an anti-cancer therapy, the subject shows a complete response to the therapy, a partial response to the therapy, or a stable response to the therapy, e.g. the subject's tumor size or tumor load does not increase.

B. 16S rDNA Sequence Identity

In some embodiments, the 16S rDNA sequence is obtained or determined for a bacterial species to be classified. A percent identity between the query sequence and the compared sequences is determined. If the percent identify of the query sequence is determined to be above a defined threshold, then the bacterial species to be classified is classified as member of the same family of the compared sequences.

In some aspects, the threshold sequence identity is 94.5%. In some aspects, the threshold sequence identity is 98%, 98.7%. In some aspects, the threshold sequence identity is 94.8%. In some aspects, the threshold sequence identity is 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98%, 98.7%, 98.8%, 98.9% 99.0%, 99.1%, 99.2%, 99.3%, 99.4%, 99.5%. 99.6%, 99.7%, 99.8%, 99.9% or 100%. In some aspects, the threshold sequence identify is at least 94.5%, 95%, 97%, 98%, 98.7% or 99%.

C. Methods for Determining 16S rDNA Sequences

Operational taxonomic units (OTUs) can be identified, for example, by sequencing of the 16S rRNA gene, by sequencing of a specific hypervariable region of this gene (i.e., V1, V2, V3, V4, V5, V6, V7, V8, or V9), or by sequencing of any combination of hypervariable regions from this gene (e.g., V1-3 or V3-5). The bacterial 16S rDNA is approximately 1500 nucleotides in length and is used in reconstructing the evolutionary relationships and sequence similarity of one bacterial isolate to another using phylogenetic approaches. 16S rDNA sequences are used for phylogenetic reconstruction as they are in general highly conserved, but contain specific hypervariable regions that harbor sufficient nucleotide diversity to differentiate genera and species of most microbes. Using well known techniques to determine a full 16S rDNA sequence or the sequence of any hypervariable region of the 16S rDNA sequence, genomic DNA is extracted from a bacterial sample, the 16S rDNA (full region or specific hypervariable regions) amplified using polymerase chain reaction (PCR), the PCR products cleaned, and nucleotide sequences delineated to determine the genetic composition of 16S rDNA gene or subdomain of the gene. If full 16S rDNA sequencing is performed, the sequencing method used may be, but is not limited to, Sanger sequencing or a next-generation sequencing method, such as an Illumina (sequencing by synthesis) method using barcoded primers allowing for multiplex reactions. If one or more hypervariable regions are used, such as the V4 region, the sequencing may be, but is not limited to being, performed using the Sanger method or using a next-generation sequencing method, such as an Illumina (sequencing by synthesis) method using barcoded primers allowing for multiplex reactions. In some cases, the 16S rDNA sequence associated with an OTU, species, or strain of bacteria is a composite of multiple 16S rDNA sequences harbored by the OTU, species, or strain.

In some aspects, bacterial species identified as described herein are identified by sequence identity to 16S rDNA sequences as known in the art and described herein.

### V. Methods for Preparing a Bacterial Composition for Administration to a Subject

Methods for producing bacterial compositions are known in the art. For example, a composition can be produced generally via three main processes, combined with one or more methods of mixing. The steps are: organism banking, organism production, and preservation.

For banking, the strains included in the bacterial composition can be, for example isolated directly from a specimen, obtained from a banked stock, optionally cultured on a nutrient agar or in broth that supports growth to generate viable biomass, and the biomass optionally preserved in multiple aliquots in long-term storage.

Stocks of organisms may prepared for storage, e.g., by adding cryoprotectants, lyoprotectants, and/or osmoprotectants.

In general, such methods described for producing bacterial compositions and for banking, production, and preservation, are identified in U.S. Pat. Nos. 10,864,235; 10,064,900; 10,064,901; U.S. Patent Application Publication No. US20160158295A1; US20160243172A1; and published PCT application No. WO2020118054A1, each of which is incorporated by reference herein.

### VI. Immuno-Oncology (Immunotherapy) Drugs that can be Used in Conjunction with the Therapeutic Compositions

In some aspects of the disclosure, the therapeutic composition is an adjunct treatment administered in combination with an immunotherapy drug, generally an immune checkpoint inhibitor (e.g., an antibody, such as a monoclonal antibody). The terms “immune checkpoint inhibitor,” “immune checkpoint blockade,” and “immune checkpoint therapy” are used interchangeably. Examples of such immunotherapy drugs include PD-1 inhibitors (e.g., nivolumab, and pembrolizumab), PD-L1 inhibitors (e.g., atezolizumab, avelumab, and durvalumab), and CTLA-4 inhibitors (e.g., ipilimumab and tremelimumab). In some aspects, more than one checkpoint inhibitor is administered. As is known in the art, dosing of checkpoint inhibitors can be repeated at, for example, 2-3 week intervals, for as long as the patient continues to have a response or stable disease, or as otherwise determined to be appropriate by those of skill in the art.

Examples of cancers that can benefit from treatment with the therapeutic compositions in conjunction with a checkpoint inhibitor, e.g., an inhibitor of PD-1, PD-L1, or CTLA-4, include but are not limited to a metastatic melanoma, a melanoma of the skin, a non-small cell lung cancer, a kidney cancer, a bladder cancer, a head and neck cancer, a Merkel cell skin cancer (Merkel cell carcinoma), and a Hodgkin lymphoma.

In some aspects, the checkpoint inhibitor is selected from an anti-PD-1 antibody, an anti-CTLA-4 antibody, an anti-PD-L1 antibody or combinations thereof.

In some aspects, the immune checkpoint inhibitors are blocking antibodies that bind to PD-1, e.g., nivolumab, cemiplimab (REGN2810), geptanolimab (CBT-501), pacmilimab (CX-072), dostarlimab (TSR-042), sintilimab, tislelizumab, and pembrolizumab; PD-L1, e.g., durvalumab (MEDI4736), avelumab, lodapolimab (LY-3300054), CX-188, and atezolizumab; or CTLA-4, e.g., ipilimumab and tremelimumab. In some aspect, combination of one or more of such antibodies can be used. In some aspects, the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT O11, MPDL3280A, MEDI-4736, MSB-0020718C, AUR-012, LAG-3, OX40 inhibitors, OX40L inhibitors, TIGIT inhibitors, STI-A1010 or combinations thereof.

Tremelimumab, nivolumab, durvalumab and atezolizumab are described, for example, in U.S. Pat. Nos. 6,682,736, 8,008,449, 8,779,108 and 8,217,149, respectively. In some aspects, atezolizumab can be replaced by another immune checkpoint antibody, such as another blocking antibody that binds to CTLA-4, PD-1 (e.g., sintilimab, tislelizumab, pembrolizumab, or an antigen binding portion thereof), PD-L1, or a bispecific blocking antibody that binds to any checkpoint inhibitor. In selecting a different blocking antibody, those of ordinary skill in the art will know the suitable dose and administration schedule from the literature. Suitable examples of anti-CTLA-4 antibodies are those described in U.S. Pat. No. 6,207,156. Other suitable examples of anti-PD-L1 antibodies are those described in U.S. Pat. No. 8,168,179, which particularly concerns treating PD-L1 over-expressing cancers with human anti-PD-L1 antibodies, including chemotherapy combinations; U.S. Pat. No. 9,402,899, which particularly concerns treating tumors with antibodies to PD-L1, including chimeric, humanized and human antibodies; and U.S. Pat. No. 9,439,962, which particularly concerns treating cancers with anti-PD-L1 antibodies and chemotherapy.

In some aspects, the anticancer agent is a combination of a checkpoint inhibitor and a chemotherapy agent.

The term “chemotherapy” refers to various treatment modalities affecting cell proliferation and/or survival. The treatment may include administration of alkylating agents, antimetabolites, anthracyclines, plant alkaloids, topoisomerase inhibitors, and other antitumor agents, including monoclonal antibodies and kinase inhibitors. The term “neoadjuvant chemotherapy” relates to a preoperative therapy regimen consisting of a panel of hormonal, chemotherapeutic and/or antibody agents, which is aimed to shrink the primary tumor, thereby rendering local therapy (surgery or radiotherapy) less destructive or more effective, enabling breast conserving surgery and evaluation of responsiveness of tumor sensitivity towards specific agents in vivo.

Chemotherapeutic drugs can kill proliferating tumor cells, enhancing the necrotic areas created by the overall treatment. The drugs can thus enhance the action of the primary therapeutic agents of the present disclosure.

Chemotherapeutic agents used in cancer treatment can be divided into several groups, depending on their mechanism of action. Some chemotherapeutic agents directly damage DNA and RNA. By disrupting replication of the DNA such chemotherapeutics either completely halt replication, or result in the production of nonsense DNA or RNA. This category includes, for example, cisplatin (PLATINOL®), daunorubicin (CERUBIDINE®), doxorubicin (ADRIAMYCIN®), and etoposide (VEPESID®). Another group of cancer chemotherapeutic agents interferes with the formation of nucleotides or deoxyribonucleotides, so that RNA synthesis and cell replication is blocked. Examples of drugs in this class include methotrexate (ABITREXATE®), mercaptopurine (PURINETHOL®), fluorouracil (ADRUCIL®), and hydroxyurea (HYDREA®). A third class of chemotherapeutic agents affects the synthesis or breakdown of mitotic spindles, and, as a result, interrupts cell division. Examples of drugs in this class include vinblastine (VELBAN®), vincristine (ONCOVIN®) and taxenes, such as, paclitaxel (TAXOL®), and docetaxel (TAXOTERE®).

In some aspects, the anticancer agents include, and methods disclosed herein include treatment with, a taxane derivative, e.g., paclitaxel or docetaxel. In some aspects, the anticancer agents include, and methods disclosed herein include treatment with, an anthracycline derivative, such as, for example, doxorubicin, daunorubicin, and aclacinomycin. In some aspects, the anticancer agents include, and methods disclosed herein include treatment with, a topoisomerase inhibitor, such as, for example, camptothecin, topotecan, irinotecan, 20-S camptothecin, 9-nitro-camptothecin, 9-amino-camptothecin, or water soluble camptothecin analog G1147211. Anticancer agents and treatment with any combination of these and other chemotherapeutic drugs is specifically contemplated.

Patients can receive chemotherapy immediately following surgical removal of a tumor. This approach is commonly referred to as adjuvant chemotherapy. However, chemotherapy can be administered also before surgery, as so-called neoadjuvant chemotherapy.

### VII. Methods of Treating

In general, the therapeutic compositions are administered to a patient diagnosed with a cancer, e.g., melanoma, for example, metastasized melanoma in conjunction with an immunotherapy drug such as checkpoint inhibitor, e.g., an inhibitor of PD-1, PD-L1, or CTLA-4. A therapeutic composition can be administered prior to checkpoint inhibitor (e.g., PD-1/PD-L1 inhibitor or CTLA-4 inhibitor) treatment, for example, at least one week, two weeks, or three weeks in advance of the treatment. In some cases, administration of the therapeutic composition is continued after the initiation of checkpoint inhibitor (e.g., PD-1/PD-L1 or CTLA-4 inhibitor) treatment. The therapeutic compositions may be administered daily, weekly, or monthly to induce and/or maintain an appropriate microbiome in the patient's GI tract.

Prior to initiating administration of a therapeutic composition, the patient may be subject to antibiotic treatment (e.g., with vancomycin, neomycin, rifaximin, or other antibiotic) and/or a bowel cleanse. In some cases, the antibiotic is a non-absorbable or minimally absorbable antibiotic. In some cases, no bowel preparation is performed. Such preparation may increase the speed and or efficacy of engraftment of one or more species in the therapeutic compositions that are associated with an improvement in checkpoint inhibitor (e.g., PD-1/PD-L1 inhibitor) efficacy.

This disclosure also relates to methods of treating or preventing or reducing risk of a cancer in a mammalian subject in need thereof comprising administering to the subject a therapeutic composition comprising an effective amount of a purified or isolated population of bacteria that have 16S rDNA sequence identity of at least 94.5% to 16S rDNA sequences of species belonging to one or more of the families Sutterellaceae, Coriobacteriaceae, Akkermansiaceae, Desulfovibrionaceae, Odoribacteraceae, or Rikenellaceae.

In some aspects, the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Desulfovibrionaceae, Odoribacteraceae, or Rikenellaceae.

In some aspects, the therapeutic composition of the provided methods comprises bacteria belonging to two, three, four or more families.

The disclosure also relates to methods of treating or preventing or reducing risk of a cancer in a mammalian subject in need thereof comprising administering to the subject a therapeutic composition comprising an effective amount of a purified or isolated population of bacteria comprising one or more bacteria selected from Intestinimonas butyriciproducens and Anaerotruncus colihominis, or bacteria having 16S rDNA having at least 94.5%, 95%, 97%, 98%, 98.7% or 99% sequence identity to any one of the 16S rDNA thereof. In some aspects, the 16S rDNA sequence of Intestinimonas butyriciproducens is selected from SEQ ID NOs: 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, and 1394, and the 16S rDNA sequence of Anaerotruncus colihominis is selected from SEQ ID NOs: 1066, 1067, 1068, 1069, 1070, 1071, 1072, 1073, 1074, 1075, 1076, 1077, 1078, 1079, 1080, 1081, 1082, 1083, 1084, and 1085.

The disclosure also relates to methods of treating or preventing or reducing risk of a cancer in a mammalian subject in need thereof comprising administering to the subject a therapeutic composition comprising an effective amount of a purified or isolated population of bacteria comprising one or more bacteria selected from Murimonas intestini and Faecalicatena orotica, or bacteria having 16S rDNA having at least 94.5%, 95%, 97%, 98%, 98.7% or 99% sequence identity to any one of the 16S rDNA thereof. In some aspects, the 16S rDNA sequence of Murimonas intestini is SEQ ID NO: 1621 and the 16S rDNA sequence of Faecalicatena orotica is selected from SEQ ID NOs: 800, 801, 802, 803, 804, 805, 806, 807, 808, 809, and 810.

The disclosure also relates to methods of treating or preventing or reducing risk of a cancer in a mammalian subject in need thereof comprising administering to the subject a therapeutic composition comprising an effective amount of a purified or isolated population of bacteria comprising one or more bacteria selected from Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans, or bacteria having 16S rDNA having at least 94.5%, 95%, 97%, 98%, 98.7% or 99% sequence identity to any one of the 16S rDNA thereof. In some aspects, the 16S rDNA sequence of Anaerostipes hadrus is selected from SEQ ID NOs: 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, and 1057, the 16S rDNA sequence of Christensenella timonensis is selected from SEQ ID NOs: 1283, 1284, and 1285, the 16S rDNA sequence of Desulfovibrio desulfuricans is selected from SEQ ID NOs: 1293, 1294, 1295, and 1296, the 16S rDNA sequence of Dialister invisus is selected from SEQ ID NOs: 1297, 1298, and 1299, the 16S rDNA sequence of Lachnoclostridium edouardi is selected from SEQ ID NOs: 1405, 1406, 1407, 1408, 1409, and 1410, the 16S rDNA sequence of Lachnospira pectinoschiza is selected from SEQ ID NOs: 1411, 1412, 1413, 1414, 1415, and 1416, the 16S rDNA sequence of Lachnospiraceae NG18_sp7 is selected from SEQ ID NOs: 1419, 1420, 1421, 1422, 1423, 1424, 1425, and 1426, the 16S rDNA sequence of Lawsonibacter sp7 is selected from SEQ ID NOs: 1608, 1609, 1610, 1611, 1612, 1613, 1614, and 1615, the 16S rDNA sequence of Merdimonas faecis is SEQ ID NO: 1010, the 16S rDNA sequence of Odoribacter splanchnicus is selected from SEQ ID NOs: 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, and 1648, the 16S rDNA sequence of Roseburia faecis is selected from SEQ ID NOs: 1696, 1697, 1698, 1699, 1700, 1701, 1702, 1703, and 1704, the 16S rDNA sequence of Roseburia intestinalis is selected from SEQ ID NOs: 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732, or the 16S rDNA sequence of Ruthenibacterium lactatiformans is selected from SEQ ID NOs: 1733, 1734, 1735, 1736, 1737, 1738, 1739, 1740, 1741, 1742, 1743, 1744, 1745, 1746, 1747, 1748, and 1749.

The disclosure also relates to methods of treating or preventing or reducing risk of a cancer in a mammalian subject in need thereof comprising administering to the subject a therapeutic composition comprising an effective amount of a purified or isolated population of bacteria comprising one or more bacteria selected from Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof. In some aspects, the 16S rDNA sequence of Anaerostipes hadrus is selected from SEQ ID NOs: 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, and 1057, the 16S rDNA sequence of Dialister invisus is selected from SEQ ID NOs: 1297, 1298, and 1299, the 16S rDNA sequence of Merdimonas faecis is SEQ ID NO: 1010, the 16S rDNA sequence of Roseburia faecis is selected from SEQ ID NOs: 1696, 1697, 1698, 1699, 1700, 1701, 1702, 1703, and 1704, or the 16S rDNA sequence of Roseburia intestinalis is selected from SEQ ID NOs: 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

The disclosure also relates to methods of treating or preventing or reducing risk of a cancer in a mammalian subject in need thereof comprising administering to the subject a therapeutic composition comprising an effective amount of a purified or isolated population of bacteria comprising one or more bacteria selected from Desulfovibrio desulfuricans, Roseburia intestinalis, or bacteria having 16S rDNA having at least 94.5%, 95%, 97%, 98%, 98.7% or 99% sequence identity to any one of the 16S rDNA thereof. In some aspects, the 16S rDNA sequence of Desulfovibrio desulfuricans is selected from SEQ ID NOs: 1293, 1294, 1295, and 1296 or the 16S rDNA sequence of Roseburia intestinalis is selected from SEQ ID NOs: 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

The disclosure also relates to methods of treating or preventing or reducing risk of a cancer in a mammalian subject in need thereof comprising administering to the subject a therapeutic composition comprising an effective amount of a purified or isolated population of bacteria comprising one or more bacteria selected from Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof. In some aspects, the 16S rDNA sequence of Lachnoclostridium edouardi is selected from SEQ ID NOs: 1405, 1406, 1407, 1408, 1409, and 1410, the 16S rDNA sequence of Lachnospira pectinoschiza is selected from SEQ ID NOs: 1411, 1412, 1413, 1414, 1415, and 1416, the 16S rDNA sequence of Lawsonibacter sp7 is selected from SEQ ID NOs: 1608, 1609, 1610, 1611, 1612, 1613, 1614, and 1615, the 16S rDNA sequence of Odoribacter splanchnicus is selected from SEQ ID NOs: 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, and 1648, or the 16S rDNA sequence of Ruthenibacterium lactatiformans is selected from SEQ ID NOs: 1733, 1734, 1735, 1736, 1737, 1738, 1739, 1740, 1741, 1742, 1743, 1744, 1745, 1746, 1747, 1748, and 1749.

The disclosure also relates to methods of treating or preventing or reducing risk of a cancer in a mammalian subject in need thereof comprising administering to the subject a therapeutic composition comprising an effective amount of a purified or isolated population of bacteria comprising one or more bacteria selected from Anaerotignum lactatifermentans, Anaerotruncus colihominis, Angelakisella sp1, Bacteroides cellulosilyticus, Bacteroides gallinarum, Bacteroides plebeius, Blautia coccoides group, Christensenella minuta, Christensenellaceae NG16_sp1, Clostridium hylemonae, Collinsella bouchesdurhonensis, Coprobacter secundus group, Eisenbergiella massiliensis, Emergencia timonensis, Faecalicatena orotica, Fusicatenibacter sp1, Intestinimonas butyriciproducens, Intestinimonas timonensis, Lachnospiraceae NG17_sp3, Lawsonibacter sp3, Oxalobacter formigenes, Peptoniphilaceae NG4_sp2, Prochlorotrichaceae NG20_sp1, or bacteria having 16S rDNA having at least 94.5%, 95%, 97%, 98%, 98.7% or 99% sequence identity to any one of the 16S rDNA thereof. In some aspects, the 16S rDNA sequence of Anaerotignum lactatifermentans is selected from SEQ ID NOs: 1058, 1059, 1060, 1061, 1062, 1063, 1064, and 1065, the 16S rDNA sequence of Anaerotruncus colihominis is selected from SEQ ID NOs: 1066, 1067, 1068, 1069, 1070, 1071, 1072, 1073, 1074, 1075, 1076, 1077, 1078, 1079, 1080, 1081, 1082, 1083, 1084, and 1085, the 16S rDNA sequence of Bacteroides cellulosilyticus is selected from SEQ ID NOs: 1086, 1087, 1088, 1089, 1090, 1091, 1092, 1093, 1094, 1095, 1096, 1097, 1098, 1099, 1100, 1101, 1102, 1103, 1104, 1105, and 1106, the 16S rDNA sequence of Bacteroides gallinarum is selected from SEQ ID NOs: 1128 and 1129, the 16S rDNA sequence of Bacteroides plebeius is selected from SEQ ID NOs: 1136, 1137, 1138, 1139, 1140, 1141, 1142, 1143, 1144, 1145, 1146, 1147, 1148, 1149, 1150, 1151, 1152, 1153, 1154, and 1155, the 16S rDNA sequence of Blautia coccoides group is selected from SEQ ID NOs: 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, and 1272, the 16S rDNA sequence of Christensenella minuta is selected from SEQ ID NOs: 1275, 1276, 1277, 1278, 1279, 1280, 1281, and 1282, the 16S rDNA sequence of Christensenellaceae NG16_sp1 is selected from SEQ ID NOs: 1286, 1287, and 1288, the 16S rDNA sequence of Clostridium hylemonae is selected from SEQ ID NOs: 790, 791, 792, 793, 794, 795, and 796, the 16S rDNA sequence of Collinsella bouchesdurhonensis is selected from SEQ ID NOs: 1289, 1290, 1291, and 1292, the 16S rDNA sequence of Coprobacter secundus group is selected from SEQ ID NOs: 797, 798, and 799, the 16S rDNA sequence of Eisenbergiella massiliensis is selected from SEQ ID NOs: 1300, 1301, 1302, 1303, 1304, 1305, 1306, 1307, and 1308, the 16S rDNA sequence of Emergencia timonensis is selected from SEQ ID NOs: 1309, 1310, 1311, 1312, 1313, 1314, 1315, 1316, 1317, 1318, 1319, and 1320, the 16S rDNA sequence of Faecalicatena orotica is selected from SEQ ID NOs: 800, 801, 802, 803, 804, 805, 806, 807, 808, 809, and 810, the 16S rDNA sequence of Fusicatenibacter sp1 is selected from SEQ ID NOs: 1350, 1351, 1352, 1353, 1354, and 1355, the 16S rDNA sequence of Intestinimonas butyriciproducens is selected from SEQ ID NOs: 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, and 1394, the 16S rDNA sequence of Intestinimonas timonensis is selected from SEQ ID NOs: 1403 and, 1404, the 16S rDNA sequence of Lachnospiraceae NG17_sp3 is selected from SEQ ID NOs: 1417 and 1418, the 16S rDNA sequence of Lawsonibacter sp3 is selected from SEQ ID NOs: 1604, 1605, 1606, and 1607, the 16S rDNA sequence of Oxalobacter formigenes is selected from SEQ ID NOs: 1649, 1650, 1651, 1652, and 1653, the 16S rDNA sequence of Peptoniphilaceae NG4_sp2 is SEQ ID NO: 172, or the 16S rDNA sequence of Prochlorotrichaceae NG20_sp1 is SEQ ID NO: 1661.

This disclosure also relates to methods of treating or preventing or reducing risk of a cancer in a mammalian subject in need thereof comprising administering to the subject a therapeutic composition comprising an effective amount of a purified or isolated population of bacteria comprising one or more bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans, or bacteria having 16S rDNA having at least 94.5%, 95%, 97%, 98%, 98.7% or 99% sequence identity to any one of the 16S rDNA thereof.

This disclosure also relates to methods of treating or preventing or reducing risk of a cancer in a mammalian subject in need thereof comprising administering to the subject a therapeutic composition comprising an effective amount of a purified or isolated population of bacteria comprising one or more bacteria selected from Akkermansia muciniphila, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

In some aspects, the bacteria comprises one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof. In some aspects, the bacteria comprises one or more bacteria selected from Akkermansia muciniphila or bacteria having 16S rDNA having at least 94.5%, 95%, 97%, 98%, 98.7% or 99% sequence identity to any one of the 16S rDNA thereof.

In some aspects, the bacteria comprises one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

In some aspects, the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof. In some aspects, the cancer is a bladder cancer.

In some aspects, the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, Desulfovibrio desulfuricans, Roseburia intestinalis, or bacteria having 16S rDNA having at least 94.5%, 95%, 97%, 98%, 98.7% or 99% sequence identity to any one of the 16S rDNA thereof. In some aspects, the bacteria comprises one or more bacteria selected from [Extibacter] muris, Collinsella intestinalis, Collinsella stercoris, Muricomes sp1, Neobitarella massiliensis, Prevotellamassilia sp2, Ruminococcaceae NG33 sp1, or bacteria having 16S rDNA having at least 94.5%, 95%, 97%, 98%, 98.7% or 99% sequence identity to any one of the 16S rDNA thereof. In some aspects, the cancer is a non-small cell lung cancer.

In some aspects, the bacteria comprises one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof. In some aspects, the bacteria comprises one or more bacteria selected from [Adlercreutzia] equolifaciens group, [Anaerotruncus] rubiinfantis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof. In some aspects, the cancer is a metastatic melanoma.

This disclosure also relates to methods of treating or preventing or reducing risk of a cancer in a mammalian subject in need thereof comprising administering to the subject a therapeutic composition comprising an effective amount of a purified or isolated population of bacteria comprising one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772, 773, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1283, 1284, 1285, 1293, 1294, 1295, 1296, 1297, 1298, 1299, 1405, 1406, 1407, 1408, 1409, 1410, 1411, 1412, 1413, 1414, 1415, 1416, 1419, 1420, 1421, 1422, 1423, 1424, 1425, 1426, 1608, 1609, 1610, 1611, 1612, 1613, 1614, 1615, 1010, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, and 1749.

In some aspects, bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

In some aspects, bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

In some aspects, bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180, 182, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1297, 1298, 1299, 1010, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects, bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179, 184, 1293, 1294, 1295, 1296, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects, bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183, 186, 1405, 1406, 1407, 1408, 1409, 1410, 1411, 1412, 1413, 1414, 1415, 1416, 1608, 1609, 1610, 1611, 1612, 1613, 1614, 1615, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1733, 1734, 1735, 1736, 1737, 1738, 1739, 1740, 1741, 1742, 1743, 1744, 1745, 1746, 1747, 1748, and 1749.

In some aspects, the therapeutic composition of the provided methods comprises two, three, four, five, six, seven, eight, nine, ten, eleven, twelve, thirteen, fourteen, fifteen, sixteen, seventeen, eighteen or more bacterial species.

In some aspects, the therapeutic composition of the provided methods does not comprise one or more bacteria selected from Holdemania massiliensis, Rothia mucilaginosa, Hungatella sp2, Prevotella copri, Campylobacter concisus, [Atopobium] parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects, the therapeutic composition of the provided methods does not comprise one or more bacteria selected from Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects, the therapeutic composition of the provided methods does not comprise one or more bacteria selected from Allisonella histaminiformans, Bacteroides faecis, Bacteroides massiliensis, Desulfovibrionaceae NG5_sp1, Gemmiger sp2, Intestinimonas massiliensis, Marvinbryantia sp5, Propionibacterium freudenreichii, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects, the therapeutic composition of the provided methods does not comprise one or more bacteria selected from Bifidobacterium animalis, Faecalibacterium prausnitzii, or bacteria having 16S rDNA having at least 94.5%, 95%, 97%, 98%, 98.7% or 99% sequence identity to any one of the 16S rDNA thereof.

In some aspects, the therapeutic composition of the provided methods does not comprise one or more bacteria selected from Granulicatella adiacens, Lactococcus lactis, Scardovia wiggsiae, or bacteria having 16S rDNA having at least 94.5%, 95%, 97%, 98%, 98.7% or 99% sequence identity to any one of the 16S rDNA thereof.

In some aspects, the therapeutic composition of the provided methods does not comprise one or more bacteria selected from Holdemania massiliensis, Rothia mucilaginosa, Hungatella sp2, Prevotella copri, Campylobacter concisus, [Atopobium] parvulum, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Ruminococcus] bromii, [Ruminococcus]gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

In some aspects, the therapeutic composition of the provided methods does not comprise Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof. In some aspects, the therapeutic composition of the provided methods does not comprise Rothia mucilaginosa, Prevotella copri, Campylobacter concisus, or bacteria having 16S rDNA having at least 94.5%, 95%, 97%, 98%, 98.7% or 99% sequence identity to any one of the 16S rDNA thereof.

In some aspects, the therapeutic composition of the provided methods does not comprise Rothia mucilaginosa, [Atopobium] parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof. In some aspects, the therapeutic composition of the provided methods does not comprise Rothia mucilaginosa, [Atopobium] parvulum, Prevotella copri, Campylobacter concisus, or bacteria having 16S rDNA having at least 94.5%, 95%, 97%, 98%, 98.7% or 99% sequence identity to any one of the 16S rDNA thereof.

In some aspects, the therapeutic composition of the provided methods does not comprise Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, Bifidobacterium animalis, Faecalibacterium prausnitzii, or bacteria having 16S rDNA having at least 94.5%, 95%, 97%, 98%, 98.7% or 99% sequence identity to any one of the 16S rDNA thereof. In some aspects, the therapeutic composition of the provided methods does not comprise Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, or bacteria having 16S rDNA having at least 94.5%, 95%, 97%, 98%, 98.7% or 99% sequence identity to any one of the 16S rDNA thereof. In some aspects, the cancer is a bladder cancer.

In some aspects, the therapeutic composition of the provided methods does not comprise [Coprococcus] catus, [Lactobacillus] fermentum, [Romboutsia] timonensis, [Ruminococcus] bromii, [Ruminococcus] gauvreaui, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, Allisonella histaminiformans, Bacteroides faecis, Bacteroides massiliensis, Desulfovibrionaceae NG5_sp1, Gemmiger sp2, Intestinimonas massiliensis, Marvinbryantia sp5, Propionibacterium freudenreichii, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof. In some aspects, the therapeutic composition of the provided methods does not comprise [Coprococcus] catus, [Lactobacillus] fermentum, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, or bacteria having 16S rDNA having at least 94.5%, 95%, 97%, 98%, 98.7% or 99% sequence identity to any one of the 16S rDNA thereof. In some aspects, the cancer is a non-small cell lung cancer.

In some aspects, the therapeutic composition of the provided methods does not comprise [Atopobium] parvulum, [Ruminococcus] gnavus, Bacteroides dorei group, Blautia sp1, Schaalia sp2, Granulicatella adiacens, Lactococcus lactis, Scardovia wiggsiae, or bacteria having 16S rDNA having at least 94.5%, 95%, 97%, 98%, 98.7% or 99% sequence identity to any one of the 16S rDNA thereof. In some aspects, the cancer is a metastatic melanoma.

In some aspects, the therapeutic composition of the provided methods does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734, 764, 1011, 1012, 1013, 1014, 1015, 1016, 1017, 774, 1107, 1108, 1109, 1110, 1111, 1112, 1113, 1114, 1115, 1116, 1117, 1118, 1119, 1120, 1121, 1122, 1123, 1124, 1125, 1126, 1127, 1130, 1131, 1132, 1133, 1134, 1135, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 775, 776, 777, 778, 779, 780, 781, 782, 783, 784, 785, 786, 787, 788, 789, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1356, 1357, 1358, 1359, 1360, 1361, 1362, 1363, 1364, 1365, 1366, 1367, 1368, 1395, 1396, 1397, 1398, 1399, 1400, 1401, 1402, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1616, 1617, 1618, 1619, 1620, 1654, 1655, 1656, 1657, 1658, 1659, 1660, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1750, 1751, 1752, 1753, 1754, 1755, and 1756.

In some aspects, the therapeutic composition of the provided methods does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

In some aspects, the therapeutic composition of the provided methods does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

In some aspects, the therapeutic composition of the provided methods does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538, 582, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, o1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349. In some aspects, the cancer is a bladder cancer.

In some aspects, the therapeutic composition of the provided methods does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599, 600, 774, 1113, 1114, 1115, 1116, 1117, 1118, 1119, 1120, 1121, 1122, 1123, 1124, 1125, 1126, 1127, 1130, 1131, 1132, 1133, 1134, 1135, 1356, 1357, 1358, 1359, 1360, 1361, 1362, 1395, 1396, 1397, 1398, 1399, 1400, 1401, 1402, 1616, 1617, 1618, 1619, 1620, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1755, and 1756. In some aspects, the cancer is a non-small cell lung cancer.

In some aspects, the therapeutic composition of the provided methods does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590, 593, 1363, 1364, 1365, 1366, 1367, 1368, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1750, 1751, 1752, 1753, and 1754. In some aspects, the cancer is a metastatic melanoma.

This disclosure further provides methods of treating or preventing or reducing risk of a cancer comprising administering at least one anticancer treatment to a subject determined to have a microbiome sample comprising bacteria have 16S rDNA sequence identity of at least 94.5% to 16S rDNA sequences of species belonging to one or more of the families Sutterellaceae, Coriobacteriaceae, Akkermansiaceae, Desulfovibrionaceae, Odoribacteraceae, or Rikenellaceae.

In some aspects, the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Desulfovibrionaceae, Odoribacteraceae, or Rikenellaceae.

This disclosure further provides methods of treating or preventing or reducing risk of a cancer comprising administering an anticancer treatment to a subject determined to have a microbiome sample comprising bacteria selected from Intestinimonas butyriciproducens and Anaerotruncus colihominis or bacteria having a 16S rDNA sequence having at least 97%, 98%, 98.7% or 99% sequence identity to any one 16S rDNA sequence thereof. In some aspects, the 16S rDNA sequence of Intestinimonas butyriciproducens is selected from SEQ ID NOs: 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, and 1394, and the 16S rDNA sequence of Anaerotruncus colihominis is selected from SEQ ID NOs: 1066, 1067, 1068, 1069, 1070, 1071, 1072, 1073, 1074, 1075, 1076, 1077, 1078, 1079, 1080, 1081, 1082, 1083, 1084, and 1085.

This disclosure further provides methods of treating or preventing or reducing risk of a cancer comprising administering an anticancer treatment to a subject determined to have a microbiome sample comprising bacteria selected from Murimonas intestini and Faecalicatena orotica, or bacteria having a 16S rDNA sequence having at least 97%, 98%, 98.7% or 99% sequence identity to any one 16S rDNA sequence thereof. In some aspects, the 16S rDNA sequence of Murimonas intestini is SEQ ID NO: 1621 and the 16S rDNA sequence of Faecalicatena orotica is selected from SEQ ID NOs: 800, 801, 802, 803, 804, 805, 806, 807, 808, 809, and 810.

This disclosure further provides methods of treating or preventing or reducing risk of a cancer comprising administering an anticancer treatment to a subject determined to have a microbiome sample comprising bacteria selected from Anaerotignum lactatifermentans, Anaerotruncus colihominis, Angelakisella sp1, Bacteroides cellulosilyticus, Bacteroides gallinarum, Bacteroides plebeius, Blautia coccoides group, Christensenella minuta, Christensenellaceae NG16_sp1, Clostridium hylemonae, Collinsella bouchesdurhonensis, Coprobacter secundus group, Eisenbergiella massiliensis, Emergencia timonensis, Faecalicatena orotica, Fusicatenibacter sp1, Intestinimonas butyriciproducens, Intestinimonas timonensis, Lachnospiraceae NG17_sp3, Lawsonibacter sp3, Oxalobacter formigenes, Peptoniphilaceae NG4_sp2, Prochlorotrichaceae NG20_sp1, or bacteria having a 16S rDNA sequence having at least 97%, 98%, 98.7% or 99% sequence identity to any one 16S rDNA sequence thereof. In some aspects, the 16S rDNA sequence of Anaerotignum lactatifermentans is selected from SEQ ID NOs: 1058, 1059, 1060, 1061, 1062, 1063, 1064, and 1065, the 16S rDNA sequence of Anaerotruncus colihominis is selected from SEQ ID NOs: 1066, 1067, 1068, 1069, 1070, 1071, 1072, 1073, 1074, 1075, 1076, 1077, 1078, 1079, 1080, 1081, 1082, 1083, 1084, and 1085, the 16S rDNA sequence of Bacteroides cellulosilyticus is selected from SEQ ID NOs: 1086, 1087, 1088, 1089, 1090, 1091, 1092, 1093, 1094, 1095, 1096, 1097, 1098, 1099, 1100, 1101, 1102, 1103, 1104, 1105, and 1106, the 16S rDNA sequence of Bacteroides gallinarum is selected from SEQ ID NOs: 1128 and 1129, the 16S rDNA sequence of Bacteroides plebeius is selected from SEQ ID NOs: 1136, 1137, 1138, 1139, 1140, 1141, 1142, 1143, 1144, 1145, 1146, 1147, 1148, 1149, 1150, 1151, 1152, 1153, 1154, and 1155, the 16S rDNA sequence of Blautia coccoides group is selected from SEQ ID NOs: 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, and 1272, the 16S rDNA sequence of Christensenella minuta is selected from SEQ ID NOs: 1275, 1276, 1277, 1278, 1279, 1280, 1281, and 1282, the 16S rDNA sequence of Christensenellaceae NG16_sp1 is selected from SEQ ID NOs: 1286, 1287, and 1288, the 16S rDNA sequence of Clostridium hylemonae is selected from SEQ ID NOs: 790, 791, 792, 793, 794, 795, and 796, the 16S rDNA sequence of Collinsella bouchesdurhonensis is selected from SEQ ID NOs: 1289, 1290, 1291, and 1292, the 16S rDNA sequence of Coprobacter secundus group is selected from SEQ ID NOs: 797, 798, and 799, the 16S rDNA sequence of Eisenbergiella massiliensis is selected from SEQ ID NOs: 1300, 1301, 1302, 1303, 1304, 1305, 1306, 1307, and 1308, the 16S rDNA sequence of Emergencia timonensis is selected from SEQ ID NOs: 1309, 1310, 1311, 1312, 1313, 1314, 1315, 1316, 1317, 1318, 1319, and 1320, the 16S rDNA sequence of Faecalicatena orotica is selected from SEQ ID NOs: 800, 801, 802, 803, 804, 805, 806, 807, 808, 809, and 810, the 16S rDNA sequence of Fusicatenibacter sp1 is selected from SEQ ID NOs: 1350, 1351, 1352, 1353, 1354, and 1355, the 16S rDNA sequence of Intestinimonas butyriciproducens is selected from SEQ ID NOs: 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, and 1394, the 16S rDNA sequence of Intestinimonas timonensis is selected from SEQ ID NOs: 1403 and, 1404, the 16S rDNA sequence of Lachnospiraceae NG17_sp3 is selected from SEQ ID NOs: 1417 and 1418, the 16S rDNA sequence of Lawsonibacter sp3 is selected from SEQ ID NOs: 1604, 1605, 1606, and 1607, the 16S rDNA sequence of Oxalobacter formigenes is selected from SEQ ID NOs: 1649, 1650, 1651, 1652, and 1653, the 16S rDNA sequence of Peptoniphilaceae NG4_sp2 is SEQ ID NO: 172, or the 16S rDNA sequence of Prochlorotrichaceae NG20_sp1 is SEQ ID NO: 1661.

This disclosure further provides methods of treating or preventing or reducing risk of a cancer comprising administering an anticancer treatment to a subject determined to have a microbiome sample comprising bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia]timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans, or bacteria having 16S rDNA having at least 94.5%, 95%, 97%, 98%, 98.7% or 99% sequence identity to any one of the 16S rDNA thereof.

This disclosure further provides methods of treating or preventing or reducing risk of a cancer comprising administering an anticancer treatment to a subject determined to have a microbiome sample comprising bacteria selected from Akkermansia muciniphila, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

In some aspects, the bacteria comprises one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof. In some aspects, the bacteria in the composition can comprise one or more bacteria selected from Akkermansia muciniphila or bacteria having 16S rDNA having at least 94.5%, 95%, 97%, 98%, 98.7% or 99% sequence identity to any one of the 16S rDNA thereof.

In some aspects, the bacteria comprises one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

In some aspects, the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof. In some aspects, the cancer is a bladder cancer.

In some aspects, the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, Desulfovibrio desulfuricans, Roseburia intestinalis, or bacteria having 16S rDNA having at least 94.5%, 95%, 97%, 98%, 98.7% or 99% sequence identity to any one of the 16S rDNA thereof. In some aspects, the bacteria comprises one or more bacteria selected from [Extibacter] muris, Collinsella intestinalis, Collinsella stercoris, Muricomes sp1, Neobitarella massiliensis, Prevotellamassilia sp2, Ruminococcaceae NG33 sp1, or bacteria having 16S rDNA having at least 94.5%, 95%, 97%, 98%, 98.7% or 99% sequence identity to any one of the 16S rDNA thereof. In some aspects, the cancer is a non-small cell lung cancer.

In some aspects, the bacteria comprises one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof. In some aspects, the bacteria comprises one or more bacteria selected from [Adlercreutzia] equolifaciens group, [Anaerotruncus] rubiinfantis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof. In some aspects, the cancer is a metastatic melanoma.

This disclosure further provides methods of treating or preventing or reducing risk of a cancer comprising administering an anticancer treatment to a subject determined to have a microbiome sample comprising one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772, 773, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1283, 1284, 1285, 1293, 1294, 1295, 1296, 1297, 1298, 1299, 1405, 1406, 1407, 1408, 1409, 1410, 1411, 1412, 1413, 1414, 1415, 1416, 1419, 1420, 1421, 1422, 1423, 1424, 1425, 1426, 1608, 1609, 1610, 1611, 1612, 1613, 1614, 1615, 1010, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, and 1749.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180, 182, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1297, 1298, 1299, 1010, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732. In some aspects, the cancer is a bladder cancer.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179, 184, 1293, 1294, 1295, 1296, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732. In some aspects, the cancer is a non-small cell lung cancer.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183, 186, 1405, 1406, 1407, 1408, 1409, 1410, 1411, 1412, 1413, 1414, 1415, 1416, 1608, 1609, 1610, 1611, 1612, 1613, 1614, 1615, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1733, 1734, 1735, 1736, 1737, 1738, 1739, 1740, 1741, 1742, 1743, 1744, 1745, 1746, 1747, 1748, and 1749. In some aspects, the cancer is a metastatic melanoma.

In some aspects, the subject is determined to not have a microbiome sample that comprises one or more bacteria selected from Holdemania massiliensis, Rothia mucilaginosa, Hungatella sp2, Prevotella copri, Campylobacter concisus, [Atopobium] parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects, the subject is determined to not have a microbiome sample that comprises one or more bacteria selected from Rothia mucilaginosa, Prevotella copri, Campylobacter concisus, [Atopobium] parvulum, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

In some aspects, the subject is determined to not have a microbiome sample that comprises one or more bacteria selected from Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof. In some aspects, the subject is determined to not have a microbiome sample that comprises one or more bacteria selected from Rothia mucilaginosa, Prevotella copri, Campylobacter concisus, or bacteria having 16S rDNA having at least 94.5%, 95%, 97%, 98%, 98.7% or 99% sequence identity to any one of the 16S rDNA thereof.

In some aspects, the subject is determined to not have a microbiome sample that comprises one or more bacteria selected from Rothia mucilaginosa, [Atopobium] parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof. In some aspects, the subject is determined to not have a microbiome sample that comprises one or more bacteria selected from Rothia mucilaginosa, [Atopobium]parvulum, Prevotella copri, Campylobacter concisus, or bacteria having 16S rDNA having at least 94.5%, 95%, 97%, 98%, 98.7% or 99% sequence identity to any one of the 16S rDNA thereof.

In some aspects, the subject is determined to not have a microbiome sample that comprises one or more bacteria selected from Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, Bifidobacterium animalis, Faecalibacterium prausnitzii, or bacteria having 16S rDNA having at least 94.5%, 95%, 97%, 98%, 98.7% or 99% sequence identity to any one of the 16S rDNA thereof. In some aspects, the subject is determined to not have a microbiome sample that comprises one or more bacteria selected from Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, or bacteria having 16S rDNA having at least 94.5%, 95%, 97%, 98%, 98.7% or 99% sequence identity to any one of the 16S rDNA thereof. In some aspects, the cancer is a bladder cancer.

In some aspects, the subject is determined to not have a microbiome sample that comprises one or more bacteria selected from [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, Allisonella histaminiformans, Bacteroides faecis, Bacteroides massiliensis, Desulfovibrionaceae NG5_sp1, Gemmiger sp2, Intestinimonas massiliensis, Marvinbryantia sp5, Propionibacterium freudenreichii, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof. In some aspects, the subject is determined to not have a microbiome sample that comprises one or more bacteria selected from [Coprococcus] catus, [Lactobacillus] fermentum, [Ruminococcus] bromii, [Ruminococcus]gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, or bacteria having 16S rDNA having at least 94.5%, 95%, 97%, 98%, 98.7% or 99% sequence identity to any one of the 16S rDNA thereof. In some aspects, the cancer is a non-small cell lung cancer.

In some aspects, the subject is determined to not have a microbiome sample that comprises one or more bacteria selected from [Atopobium] parvulum, [Ruminococcus] gnavus, Bacteroides dorei group, Blautia sp1, Schaalia sp2, Granulicatella adiacens, Lactococcus lactis, Scardovia wiggsiae, or bacteria having 16S rDNA having at least 94.5%, 95%, 97%, 98%, 98.7% or 99% sequence identity to any one of the 16S rDNA thereof. In some aspects, the cancer is a metastatic melanoma.

In some aspects, the subject is determined to not have a microbiome sample that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734, 764, 1011, 1012, 1013, 1014, 1015, 1016, 1017, 774, 1107, 1108, 1109, 1110, 1111, 1112, 1113, 1114, 1115, 1116, 1117, 1118, 1119, 1120, 1121, 1122, 1123, 1124, 1125, 1126, 1127, 1130, 1131, 1132, 1133, 1134, 1135, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 775, 776, 777, 778, 779, 780, 781, 782, 783, 784, 785, 786, 787, 788, 789, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1356, 1357, 1358, 1359, 1360, 1361, 1362, 1363, 1364, 1365, 1366, 1367, 1368, 1395, 1396, 1397, 1398, 1399, 1400, 1401, 1402, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1616, 1617, 1618, 1619, 1620, 1654, 1655, 1656, 1657, 1658, 1659, 1660, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1750, 1751, 1752, 1753, 1754, 1755, and 1756.

In some aspects, the subject is determined to not have a microbiome sample that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

In some aspects, the subject is determined to not have a microbiome sample that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

In some aspects, the subject is determined to not have a microbiome sample that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538, 582, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, o1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349. In some aspects, the cancer is a bladder cancer.

In some aspects, the subject is determined to not have a microbiome sample that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599, 600, 774, 1113, 1114, 1115, 1116, 1117, 1118, 1119, 1120, 1121, 1122, 1123, 1124, 1125, 1126, 1127, 1130, 1131, 1132, 1133, 1134, 1135, 1356, 1357, 1358, 1359, 1360, 1361, 1362, 1395, 1396, 1397, 1398, 1399, 1400, 1401, 1402, 1616, 1617, 1618, 1619, 1620, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1755, and 1756. In some aspects, the cancer is a non-small cell lung cancer.

In some aspects, the subject is determined to not have a microbiome sample that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590, 593, 1363, 1364, 1365, 1366, 1367, 1368, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1750, 1751, 1752, 1753, and 1754. In some aspects, the cancer is a metastatic melanoma.

In some aspects, the cancer is selected from a metastatic melanoma, a melanoma of the skin, a non-small cell lung cancer, a kidney cancer, a bladder cancer, a head and neck cancers, a Merkel cell skin cancer (Merkel cell carcinoma), or a Hodgkin lymphoma.

In some aspects, the cancer is a metastatic melanoma. In some aspects, the cancer is a non-small cell lung cancer. In some aspects, the cancer is a bladder cancer.

In some aspects, prior to administration of the population of bacteria the subject is subjected to antibiotic treatment and/or a bowel cleanse.

In some aspects, the subject has previously been treated for the cancer. In some aspects, the subject has been determined to be a non-responder to the previous treatment. In some aspects, the subject has been determined to have a toxic response to the previous treatment.

In some aspects, the previous treatment comprises immune checkpoint blockade monotherapy or immune checkpoint blockade combination therapy.

In some aspects, the cancer is recurrent cancer.

In some aspects, the provided methods further comprising administering at least one anticancer agent to the subject.

In some aspects, the anticancer agent is a checkpoint inhibitor.

In some aspects, the checkpoint inhibitor is selected from an anti-PD-1 antibody, an anti-CTLA-4 antibody, an anti-PD-L1 antibody or combinations thereof.

In some aspects, the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT O11, MPDL3280A, MEDI-4736, MSB-0020718C, AUR-012, LAG-3, OX40 inhibitors, OX40L inhibitors, TIGIT inhibitors, STI-A1010 or combinations thereof.

In some aspects, the anticancer agent comprises a chemotherapy agent.

In some aspects, the anticancer agent is a combination of a checkpoint inhibitor and a chemotherapy agent.

### VIII. Models for Testing

Animal models suitable for testing the efficacy of a microbiome composition for use in immunotherapy are known in the art, for example, as described in Cooper et al. (2014, Cancer Immunol Res 2:643-654) and Gopalakrishnan et al (2018, Science 359(6371):97-103) using the BP cell line, and reviewed in Li et al. (2017, Pharmacol & Therapeutics, dx.doi.org/10.1016/j.pharmthera.2017.02.002). Other useful models include germ-free mouse models (e.g., Matson et al. Science 359:104-108 (2018), Routy et al Science 59(6371):91-97 (2018)).

### IX. Formulations

A microbiome therapeutic composition for use as described herein can be prepared and administered using methods known in the art. In general, compositions are formulated for oral, colonoscopic, or nasogastric delivery although any appropriate method can be used.

A formulation containing a therapeutic composition can contain one or more pharmaceutical excipients suitable for the preparation of such formulations. In some aspects, the formulation is a liquid formulation. In some aspects, a formulation comprising the therapeutic compositions can comprise one or more of surfactants, adjuvants, buffers, antioxidants, tonicity adjusters, thickeners or viscosity modifiers and the like.

In some aspects, treatment includes administering the therapeutic compositions in a formulation that includes a pharmaceutically acceptable carrier. In some aspects, the excipient includes a capsule or other format suitable for providing the therapeutic compositions as an oral dosage form. When an excipient serves as a diluent, it can be a solid, semi-solid, or liquid material, which acts as a vehicle, carrier or medium for the active ingredient. Thus, the formulations can be in the form of tablets, pills, powders, lozenges, sachets, cachets, elixirs, suspensions, emulsions, solutions, syrups, soft or hard capsules, suppositories, or packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, polyethylene glycol, glycerol, and methyl cellulose. The compositions can be formulated so as to provide quick, sustained or delayed release of the active ingredient after administration to the patient by employing procedures known in the art.

In some aspects, the therapeutic composition can be incorporated into a food product. In some aspects the food product is a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In some aspects, the food product is a solid foodstuff. Suitable examples of a solid foodstuff include without limitation a food bar, a snack bar, a cookie, a brownie, a muffin, a cracker, an ice cream bar, a frozen yogurt bar, and the like.

In some aspects, the therapeutic compositions are incorporated into a therapeutic food. In some aspects, the therapeutic food is a ready-to-use food that optionally contains some or all essential macronutrients and micronutrients. In some aspects, the compositions disclosed herein are incorporated into a supplementary food that is designed to be blended into an existing meal. In some aspects, the supplemental food contains some or all essential macronutrients and micronutrients. In some aspects, the bacterial compositions disclosed herein are blended with or added to an existing food to fortify the food's protein nutrition. Examples include food staples (grain, salt, sugar, cooking oil, margarine), beverages (juice, coffee, tea, soda, beer, liquor, sports drinks), snacks, sweets and other foods.

The therapeutic compositions can be formulated in a unit dosage form. In general, a dosage comprises about 1×10² to 1×10⁹ viable colony forming units (CFU). The term “unit dosage forms” refers to physically discrete units suitable as unitary dosages for human subjects and/or other mammals, each unit containing a predetermined quantity of active material calculated to produce the desired therapeutic effect, in association with a suitable pharmaceutical excipient. A dosage may be administered in multiple delivery vehicles, e.g., multiple pills, capsules, foodstuffs or beverages.

The amount and frequency of administering the therapeutic compositions to a patient can vary depending upon the specific composition being administered, the purpose of the administration (such as prophylaxis or therapy), the state of the patient, the manner of administration, and the like. In therapeutic applications, compositions can be administered to a patient already suffering from a disease in an amount sufficient to cure or at least partially arrest or mitigate the symptoms of the disease and its complications. An effective dose can depend on the disease condition being treated as well as by the judgment of the attending clinician depending upon factors such as the severity of the disease, the age, weight and general condition of the patient, and the like.

In some aspects, at least one dose of the therapeutic composition is administered by the attending clinician or a person acting on behalf of the attending clinician. In some aspects, the subject may self-administer some or all of the subsequent doses. In other aspects, all doses of the therapeutic composition are administered by the attending clinician or a person acting on behalf of the attending clinician. In these aspects, prior to the administration of a first dose of the therapeutic composition the attending clinician or a person acting on behalf of the attending clinician may administer an antibiotic treatment and/or a bowel cleanse.

The dosage can refer, for example, to the total number of viable colony forming units (CFUs) of each individual species or strain; or can refer to the total number of microorganisms in the dose. It is understood in the art that determining the number of organisms in a dosage is not exact and can depend on the method used to determine the number of organisms present. If the composition includes spores, for example, the number of spores in a composition may be determined using a dipicolinic acid assay (Fichtel et al, 2007, FEMS Microbiol Ecol, 61:522-32). In some cases, the number of organisms is determined using a culture assay.

Effective doses can be extrapolated from dose-response curves derived from in vitro or animal model test systems.

In some aspects, the purified or isolated population of bacteria is administered at a concentration of at least about 1×10² viable colony forming units.

In some aspects, the purified or isolated population of bacteria is administered at a concentration of at a concentration of about 1×10² to 1×10⁹ viable colony forming units.

In some aspects, a fraction of the purified or isolated population of bacteria comprises a spore-forming bacteria. In some aspects, a fraction of the purified or isolated population of bacteria is in spore form. In some aspects, a fraction of the purified or isolated population of bacteria is in vegetative form.

In some aspects, the composition of the provided methods further comprises a pharmaceutically acceptable excipient. In some aspects, the composition of the provided methods is formulated for delivery to the intestine. In some aspects, the composition of the provided methods is enterically coated. In some aspects, the composition of the provided methods is formulated for oral administration. In some aspects, the composition of the provided methods is formulated into a food or beverage.

### X. Methods of Identifying a Candidate for Immune Checkpoint Therapy in Combination with Microbiome Therapy

This disclosure further relates to methods of identifying a mammalian subject as a candidate for immune checkpoint therapy in combination with microbiome therapy to treat a cancer, the method comprising:
a) obtaining a microbiome sample from the subject,
b) determining the prevalence and/or abundance of the family of bacteria in the microbiome sample, and
c) determining that the subject is a candidate for treatment if the microbiome sample comprises bacteria that have 16S rDNA sequence identity of at least 94.5% to 16S rDNA sequences of species belonging to one or more of the families Sutterellaceae, Coriobacteriaceae, Akkermansiaceae, or Desulfovibrionaceae.

In some aspects, the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Desulfovibrionaceae, Odoribacteraceae, or Rikenellaceae.

This disclosure further relates to methods of identifying a mammalian subject as a candidate for immune checkpoint therapy in combination with microbiome therapy to treat a cancer, the method comprising:
a) obtaining a microbiome sample from the subject,
b) determining the prevalence and/or abundance of the family of bacteria in the microbiome sample, and
c) determining that the subject is a candidate for treatment if the microbiome sample comprises bacteria comprising one or more bacteria selected from Intestinimonas butyriciproducens and Anaerotruncus colihominis, or bacteria having 16S rDNA having at least 94.5%, 95%, 97%, 98%, 98.7% or 99% sequence identity to any one of the 16S rDNA thereof.

This disclosure further relates to methods of identifying a mammalian subject as a candidate for immune checkpoint therapy in combination with microbiome therapy to treat a cancer, the method comprising:
a) obtaining a microbiome sample from the subject,
b) determining the prevalence and/or abundance of the family of bacteria in the microbiome sample, and
c) determining that the subject is a candidate for treatment if the microbiome sample comprises bacteria comprising one or more bacteria selected from Murimonas intestini and Faecalicatena orotica, or bacteria having 16S rDNA having at least 94.5%, 95%, 97%, 98%, 98.7% or 99% sequence identity to any one of the 16S rDNA thereof.

This disclosure further relates to methods of identifying a mammalian subject as a candidate for immune checkpoint therapy in combination with microbiome therapy to treat a cancer, the method comprising:
a) obtaining a microbiome sample from the subject,
b) determining the prevalence and/or abundance of the family of bacteria in the microbiome sample, and
c) determining that the subject is a candidate for treatment if the microbiome sample comprises bacteria comprising one or more bacteria selected from Anaerotignum lactatifermentans, Anaerotruncus colihominis, Angelakisella sp1, Bacteroides cellulosilyticus, Bacteroides gallinarum, Bacteroides plebeius, Blautia coccoides group, Christensenella minuta, Christensenellaceae NG16_sp1, Clostridium hylemonae, Collinsella bouchesdurhonensis, Coprobacter secundus group, Eisenbergiella massiliensis, Emergencia timonensis, Faecalicatena orotica, Fusicatenibacter sp1, Intestinimonas butyriciproducens, Intestinimonas timonensis, Lachnospiraceae NG17_sp3, Lawsonibacter sp3, Oxalobacter formigenes, Peptoniphilaceae NG4_sp2, Prochlorotrichaceae NG20_sp1, or bacteria having 16S rDNA having at least 94.5%, 95%, 97%, 98%, 98.7% or 99% sequence identity to any one of the 16S rDNA thereof.

This disclosure further relates to methods of identifying a mammalian subject as a candidate for immune checkpoint therapy in combination with microbiome therapy to treat a cancer, the method comprising:
a) obtaining a microbiome sample from the subject,
b) determining the prevalence and/or abundance of the family of bacteria in the microbiome sample, and
c) determining that the subject is a candidate for treatment if the microbiome sample comprises bacteria comprising one or more bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans, or bacteria having 16S rDNA having at least 94.5%, 95%, 97%, 98%, 98.7% or 99% sequence identity to any one of the 16S rDNA thereof.

This disclosure further relates to methods of identifying a mammalian subject as a candidate for immune checkpoint therapy in combination with microbiome therapy to treat a cancer, the method comprising:
a) obtaining a microbiome sample from the subject,
b) determining the prevalence and/or abundance of the family of bacteria in the microbiome sample, and
c) determining that the subject is a candidate for treatment if the microbiome sample comprises bacteria comprising one or more bacteria selected from Akkermansia muciniphila, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus]rubiinfantis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

In some aspects, the bacteria comprises one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof. In some aspects, the bacteria comprises one or more bacteria selected from Akkermansia muciniphila or bacteria having 16S rDNA having at least 94.5%, 95%, 97%, 98%, 98.7% or 99% sequence identity to any one of the 16S rDNA thereof.

In some aspects, the bacteria comprises one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

In some aspects, the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof. In some aspects, the cancer is a bladder cancer.

In some aspects, the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, Desulfovibrio desulfuricans, Roseburia intestinalis, or bacteria having 16S rDNA having at least 94.5%, 95%, 97%, 98%, 98.7% or 99% sequence identity to any one of the 16S rDNA thereof. In some aspects, the bacteria comprises one or more bacteria selected from [Extibacter] muris, Collinsella intestinalis, Collinsella stercoris, Muricomes sp1, Neobitarella massiliensis, Prevotellamassilia sp2, Ruminococcaceae NG33 sp1, or bacteria having 16S rDNA having at least 94.5%, 95%, 97%, 98%, 98.7% or 99% sequence identity to any one of the 16S rDNA thereof. In some aspects, the cancer is a non-small cell lung cancer.

In some aspects, the bacteria comprises one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof. In some aspects, the bacteria comprises one or more bacteria selected from [Adlercreutzia] equolifaciens group, [Anaerotruncus] rubiinfantis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof. In some aspects, the cancer is a metastatic melanoma.

This disclosure also relates to methods of identifying a mammalian subject as a candidate for immune checkpoint therapy in combination with microbiome therapy to treat a cancer, the method comprising:
a) obtaining a microbiome sample from the subject,
b) determining the prevalence and/or abundance of the family of bacteria in the microbiome sample, and
c) determining that the subject is a candidate for treatment if the microbiome sample comprises bacteria comprising one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772, 773, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1283, 1284, 1285, 1293, 1294, 1295, 1296, 1297, 1298, 1299, 1405, 1406, 1407, 1408, 1409, 1410, 1411, 1412, 1413, 1414, 1415, 1416, 1419, 1420, 1421, 1422, 1423, 1424, 1425, 1426, 1608, 1609, 1610, 1611, 1612, 1613, 1614, 1615, 1010, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, and 1749.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180, 182, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1297, 1298, 1299, 1010, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179, 184, 1293, 1294, 1295, 1296, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183, 186, 1405, 1406, 1407, 1408, 1409, 1410, 1411, 1412, 1413, 1414, 1415, 1416, 1608, 1609, 1610, 1611, 1612, 1613, 1614, 1615, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1733, 1734, 1735, 1736, 1737, 1738, 1739, 1740, 1741, 1742, 1743, 1744, 1745, 1746, 1747, 1748, and 1749.

In some aspects, determining that the subject is a candidate for treatment further comprises determining if the microbiome sample does not comprise one or more bacteria selected from Holdemania massiliensis, Rothia mucilaginosa, Hungatella sp2, Prevotella copri, Campylobacter concisus, [Atopobium] parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects, determining that the subject is a candidate for treatment further comprises determining if the microbiome sample does not comprise one or more bacteria selected from Rothia mucilaginosa, Prevotella copri, Campylobacter concisus, [Atopobium]parvulum, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Ruminococcus] bromii, [Ruminococcus] gauvreaui, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

In some aspects, determining that the subject is a candidate for treatment further comprises determining if the microbiome sample does not comprise one or more bacteria selected from Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof. In some aspects, determining that the subject is a candidate for treatment further comprises determining if the microbiome sample does not comprise one or more bacteria selected from Rothia mucilaginosa, Prevotella copri, Campylobacter concisus, or bacteria having 16S rDNA having at least 94.5%, 95%, 97%, 98%, 98.7% or 99% sequence identity to any one of the 16S rDNA thereof.

In some aspects, determining that the subject is a candidate for treatment further comprises determining if the microbiome sample does not comprise one or more bacteria selected from Rothia mucilaginosa, [Atopobium] parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof. In some aspects, determining that the subject is a candidate for treatment further comprises determining if the microbiome sample does not comprise one or more bacteria selected from Rothia mucilaginosa, [Atopobium] parvulum, Prevotella copri, Campylobacter concisus, or bacteria having 16S rDNA having at least 94.5%, 95%, 97%, 98%, 98.7% or 99% sequence identity to any one of the 16S rDNA thereof.

In some aspects, determining that the subject is a candidate for treatment further comprises determining if the microbiome sample does not comprise one or more bacteria selected from Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, Bifidobacterium animalis, Faecalibacterium prausnitzii, or bacteria having 16S rDNA having at least 94.5%, 95%, 97%, 98%, 98.7% or 99% sequence identity to any one of the 16S rDNA thereof. In some aspects, determining that the subject is a candidate for treatment further comprises determining if the microbiome sample does not comprise one or more bacteria selected from Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, or bacteria having 16S rDNA having at least 94.5%, 95%, 97%, 98%, 98.7% or 99% sequence identity to any one of the 16S rDNA thereof. In some aspects, the cancer is a bladder cancer.

In some aspects, determining that the subject is a candidate for treatment further comprises determining if the microbiome sample does not comprise one or more bacteria selected from [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, Allisonella histaminiformans, Bacteroides faecis, Bacteroides massiliensis, Desulfovibrionaceae NG5_sp1, Gemmiger sp2, Intestinimonas massiliensis, Marvinbryantia sp5, Propionibacterium freudenreichii, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof. In some aspects, determining that the subject is a candidate for treatment further comprises determining if the microbiome sample does not comprise one or more bacteria selected from [Coprococcus] catus, [Lactobacillus] fermentum, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, or bacteria having 16S rDNA having at least 94.5%, 95%, 97%, 98%, 98.7% or 99% sequence identity to any one of the 16S rDNA thereof. In some aspects, the cancer is a non-small cell lung cancer.

In some aspects, determining that the subject is a candidate for treatment further comprises determining if the microbiome sample does not comprise one or more bacteria selected from [Atopobium] parvulum, [Ruminococcus] gnavus, Bacteroides dorei group, Blautia sp1, Schaalia sp2, Granulicatella adiacens, Lactococcus lactis, Scardovia wiggsiae, or bacteria having 16S rDNA having at least 94.5%, 95%, 97%, 98%, 98.7% or 99% sequence identity to any one of the 16S rDNA thereof. In some aspects, the cancer is a metastatic melanoma.

In some aspects, determining that the subject is a candidate for treatment further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734, 764, 1011, 1012, 1013, 1014, 1015, 1016, 1017, 774, 1107, 1108, 1109, 1110, 1111, 1112, 1113, 1114, 1115, 1116, 1117, 1118, 1119, 1120, 1121, 1122, 1123, 1124, 1125, 1126, 1127, 1130, 1131, 1132, 1133, 1134, 1135, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 775, 776, 777, 778, 779, 780, 781, 782, 783, 784, 785, 786, 787, 788, 789, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1356, 1357, 1358, 1359, 1360, 1361, 1362, 1363, 1364, 1365, 1366, 1367, 1368, 1395, 1396, 1397, 1398, 1399, 1400, 1401, 1402, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1616, 1617, 1618, 1619, 1620, 1654, 1655, 1656, 1657, 1658, 1659, 1660, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1750, 1751, 1752, 1753, 1754, 1755, and 1756.

In some aspects, determining that the subject is a candidate for treatment further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

In some aspects, determining that the subject is a candidate for treatment further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

In some aspects, determining that the subject is a candidate for treatment further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538, 582, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, o1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349. In some aspects, the cancer is a bladder cancer.

In some aspects, determining that the subject is a candidate for treatment further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599, 600, 774, 1113, 1114, 1115, 1116, 1117, 1118, 1119, 1120, 1121, 1122, 1123, 1124, 1125, 1126, 1127, 1130, 1131, 1132, 1133, 1134, 1135, 1356, 1357, 1358, 1359, 1360, 1361, 1362, 1395, 1396, 1397, 1398, 1399, 1400, 1401, 1402, 1616, 1617, 1618, 1619, 1620, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1755, and 1756. In some aspects, the cancer is a non-small cell lung cancer.

In some aspects, determining that the subject is a candidate for treatment further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590, 593, 1363, 1364, 1365, 1366, 1367, 1368, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1750, 1751, 1752, 1753, and 1754. In some aspects, the cancer is a metastatic melanoma.

In some aspects, the subject is determined to be a candidate for immune checkpoint inhibitor therapy.

In some aspects, the immune checkpoint therapy comprises immune checkpoint blockade monotherapy or immune checkpoint blockade combination therapy.

In some aspects, the treatment further comprises chemotherapy.

In some aspects, the mammalian subject is a human.

In some aspects, the cancer is selected from a metastatic melanoma, a melanoma of the skin, a non-small cell lung cancer, a kidney cancer, a bladder cancer, a head and neck cancers, a Merkel cell skin cancer (Merkel cell carcinoma), or a Hodgkin lymphoma.

In some aspects, the cancer is a metastatic melanoma. In some aspects, the cancer is a non-small cell lung cancer. In some aspects, the cancer is a bladder cancer.

### XI. Methods of Identifying FMT Donors

This disclosure also relates to methods of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises bacteria that have 16S rDNA sequence identity of at least 94.5% to 16S rDNA sequences of species belonging to one or more of the families Sutterellaceae, Coriobacteriaceae, Akkermansiaceae, or Desulfovibrionaceae.

In some aspects, the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Desulfovibrionaceae, Odoribacteraceae, or Rikenellaceae.

This disclosure also relates to methods of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises bacteria comprising one or more bacteria selected from Intestinimonas butyriciproducens and Anaerotruncus colihominis or bacteria having 16S rDNA having at least 94.5%, 95%, 97%, 98%, 98.7% or 99% sequence identity to any one of the 16S rDNA thereof.

This disclosure also relates to methods of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises bacteria comprising one or more bacteria selected from Murimonas intestini and Faecalicatena orotica, or bacteria having 16S rDNA having at least 94.5%, 95%, 97%, 98%, 98.7% or 99% sequence identity to any one of the 16S rDNA thereof.

This disclosure also relates to methods of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises bacteria comprising one or more bacteria selected from Anaerotignum lactatifermentans, Anaerotruncus colihominis, Angelakisella sp1, Bacteroides cellulosilyticus, Bacteroides gallinarum, Bacteroides plebeius, Blautia coccoides group, Christensenella minuta, Christensenellaceae NG16_sp1, Clostridium hylemonae, Collinsella bouchesdurhonensis, Coprobacter secundus group, Eisenbergiella massiliensis, Emergencia timonensis, Faecalicatena orotica, Fusicatenibacter sp1, Intestinimonas butyriciproducens, Intestinimonas timonensis, Lachnospiraceae NG17_sp3, Lawsonibacter sp3, Oxalobacter formigenes, Peptoniphilaceae NG4_sp2, Prochlorotrichaceae NG20_sp1, or bacteria having 16S rDNA having at least 94.5%, 95%, 97%, 98%, 98.7% or 99% sequence identity to any one of the 16S rDNA thereof.

This disclosure also relates to methods of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises bacteria comprising one or more bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans, or bacteria having 16S rDNA having at least 94.5%, 95%, 97%, 98%, 98.7% or 99% sequence identity to any one of the 16S rDNA thereof.

This disclosure also relates to methods of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises bacteria comprising one or more bacteria selected from Akkermansia muciniphila, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus]rubiinfantis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

In some aspects, the bacteria comprises one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof. In some aspects, the bacteria comprises one or more bacteria selected from Akkermansia muciniphila or bacteria having 16S rDNA having at least 94.5%, 95%, 97%, 98%, 98.7% or 99% sequence identity to any one of the 16S rDNA thereof.

In some aspects, the bacteria comprises one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

In some aspects, the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof.

In some aspects, the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, Desulfovibrio desulfuricans, Roseburia intestinalis, or bacteria having 16S rDNA having at least 94.5%, 95%, 97%, 98%, 98.7% or 99% sequence identity to any one of the 16S rDNA thereof. In some aspects, the bacteria comprises one or more bacteria selected from [Extibacter] muris, Collinsella intestinalis, Collinsella stercoris, Muricomes sp1, Neobitarella massiliensis, Prevotellamassilia sp2, Ruminococcaceae NG33 sp1, Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof.

In some aspects, the bacteria comprises one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof. In some aspects, the bacteria comprises one or more bacteria selected from [Adlercreutzia] equolifaciens group, [Anaerotruncus] rubiinfantis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

This disclosure also relates to methods of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises bacteria comprising one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772, 773, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1283, 1284, 1285, 1293, 1294, 1295, 1296, 1297, 1298, 1299, 1405, 1406, 1407, 1408, 1409, 1410, 1411, 1412, 1413, 1414, 1415, 1416, 1419, 1420, 1421, 1422, 1423, 1424, 1425, 1426, 1608, 1609, 1610, 1611, 1612, 1613, 1614, 1615, 1010, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, and 1749.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180, 182, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1297, 1298, 1299, 1010, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179, 184, 1293, 1294, 1295, 1296, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

In some aspects, the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183, 186, 1405, 1406, 1407, 1408, 1409, 1410, 1411, 1412, 1413, 1414, 1415, 1416, 1608, 1609, 1610, 1611, 1612, 1613, 1614, 1615, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1733, 1734, 1735, 1736, 1737, 1738, 1739, 1740, 1741, 1742, 1743, 1744, 1745, 1746, 1747, 1748, and 1749.

In some aspects, determining that the donor's feces is useful for fecal matter transfer further comprises determining if the microbiome sample does not comprise one or more bacteria selected from Holdemania massiliensis, Rothia mucilaginosa, Hungatella sp2, Prevotella copri, Campylobacter concisus, [Atopobium] parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus]bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus]gnavus, Bacteroides dorei group, Blautia sp1, Schaalia sp2, Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

In some aspects, determining that the donor's feces is useful for fecal matter transfer further comprises determining if the microbiome sample does not comprise one or more bacteria selected from Rothia mucilaginosa, Prevotella copri, Campylobacter concisus, [Atopobium] parvulum, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

In some aspects, determining that the donor's feces is useful for fecal matter transfer further comprises determining if the microbiome sample does not comprise one or more bacteria selected from Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof. In some aspects, determining that the donor's feces is useful for fecal matter transfer further comprises determining if the microbiome sample does not comprise one or more bacteria selected from Rothia mucilaginosa, Prevotella copri, Campylobacter concisus, or bacteria having 16S rDNA having at least 94.5%, 95%, 97%, 98%, 98.7% or 99% sequence identity to any one of the 16S rDNA thereof.

In some aspects, determining that the donor's feces is useful for fecal matter transfer further comprises determining if the microbiome sample does not comprise one or more bacteria selected from Rothia mucilaginosa, [Atopobium] parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof. In some aspects, determining that the donor's feces is useful for fecal matter transfer further comprises determining if the microbiome sample does not comprise one or more bacteria selected from Rothia mucilaginosa, [Atopobium] parvulum, Prevotella copri, Campylobacter concisus, or bacteria having 16S rDNA having at least 94.5%, 95%, 97%, 98%, 98.7% or 99% sequence identity to any one of the 16S rDNA thereof.

In some aspects, determining that the donor's feces is useful for fecal matter transfer further comprises determining if the microbiome sample does not comprise one or more bacteria selected from Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, Bifidobacterium animalis, Faecalibacterium prausnitzii, or bacteria having 16S rDNA having at least 94.5%, 95%, 97%, 98%, 98.7% or 99% sequence identity to any one of the 16S rDNA thereof. In some aspects, determining that the donor's feces is useful for fecal matter transfer further comprises determining if the microbiome sample does not comprise one or more bacteria selected from Rothia mucilaginosa, [Atopobium]parvulum, Prevotella copri, Campylobacter concisus, or bacteria having 16S rDNA having at least 94.5%, 95%, 97%, 98%, 98.7% or 99% sequence identity to any one of the 16S rDNA thereof. In some aspects, the cancer is a bladder cancer.

In some aspects, determining that the donor's feces is useful for fecal matter transfer further comprises determining if the microbiome sample does not comprise one or more bacteria selected from [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, Allisonella histaminiformans, Bacteroides faecis, Bacteroides massiliensis, Desulfovibrionaceae NG5_sp1, Gemmiger sp2, Intestinimonas massiliensis, Marvinbryantia sp5, Propionibacterium freudenreichii, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof. In some aspects, determining that the donor's feces is useful for fecal matter transfer further comprises determining if the microbiome sample does not comprise one or more bacteria selected from [Coprococcus] catus, [Lactobacillus] fermentum, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, or bacteria having 16S rDNA having at least 94.5%, 95%, 97%, 98%, 98.7% or 99% sequence identity to any one of the 16S rDNA thereof. In some aspects, the cancer is a non-small cell lung cancer.

In some aspects, determining that the donor's feces is useful for fecal matter transfer further comprises determining if the microbiome sample does not comprise one or more bacteria selected from [Atopobium] parvulum, [Ruminococcus] gnavus, Bacteroides dorei group, Blautia sp1, Schaalia sp2, Granulicatella adiacens, Lactococcus lactis, Scardovia wiggsiae, or bacteria having 16S rDNA having at least 94.5%, 95%, 97%, 98%, 98.7% or 99% sequence identity to any one of the 16S rDNA thereof. In some aspects, the cancer is a metastatic melanoma.

In some aspects, determining that the donor's feces is useful for fecal matter transfer further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734, 764, 1011, 1012, 1013, 1014, 1015, 1016, 1017, 774, 1107, 1108, 1109, 1110, 1111, 1112, 1113, 1114, 1115, 1116, 1117, 1118, 1119, 1120, 1121, 1122, 1123, 1124, 1125, 1126, 1127, 1130, 1131, 1132, 1133, 1134, 1135, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 775, 776, 777, 778, 779, 780, 781, 782, 783, 784, 785, 786, 787, 788, 789, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1356, 1357, 1358, 1359, 1360, 1361, 1362, 1363, 1364, 1365, 1366, 1367, 1368, 1395, 1396, 1397, 1398, 1399, 1400, 1401, 1402, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1616, 1617, 1618, 1619, 1620, 1654, 1655, 1656, 1657, 1658, 1659, 1660, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1750, 1751, 1752, 1753, 1754, 1755, and 1756.

In some aspects, determining that the donor's feces is useful for fecal matter transfer further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

In some aspects, determining that the donor's feces is useful for fecal matter transfer further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

In some aspects, determining that the donor's feces is useful for fecal matter transfer further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538, 582, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, o1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349.

In some aspects, determining that the donor's feces is useful for fecal matter transfer further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599, 600, 774, 1113, 1114, 1115, 1116, 1117, 1118, 1119, 1120, 1121, 1122, 1123, 1124, 1125, 1126, 1127, 1130, 1131, 1132, 1133, 1134, 1135, 1356, 1357, 1358, 1359, 1360, 1361, 1362, 1395, 1396, 1397, 1398, 1399, 1400, 1401, 1402, 1616, 1617, 1618, 1619, 1620, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1755, and 1756.

In some aspects, determining that the donor's feces is useful for fecal matter transfer further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590, 593, 1363, 1364, 1365, 1366, 1367, 1368, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1750, 1751, 1752, 1753, and 1754.

This disclosure also provides a therapeutic composition derived from fecal matter from a donor identified using the methods disclosed herein. Such compositions or methods include the aspects discussed throughout the application.

### XII. Embodiments

The present disclosure is also described in the following embodiments:

Embodiment 1. A therapeutic composition comprising an effective amount of a purified or isolated population of bacteria that have 16S rDNA sequence identity of at least 94.5% to 16S rDNA sequences of species belonging to one or more of the families Sutterellaceae, Coriobacteriaceae, Akkermansiaceae, Campylobacteraceae, or Desulfovibrionaceae.

Embodiment 2. The therapeutic composition of embodiment 1, wherein the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Campylobacteraceae, or Desulfovibrionaceae.

Embodiment 3. The therapeutic composition of embodiment 1 or 2, wherein the therapeutic composition comprises bacteria belonging to two, three, four or more families.

Embodiment 4. A therapeutic composition comprising an effective amount of a purified or isolated population of bacteria comprising one or more bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

Embodiment 5. The therapeutic composition of embodiment 4, wherein the bacteria comprises one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof.

Embodiment 6. The therapeutic composition of embodiment 4, wherein the bacteria comprises one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

Embodiment 7. The therapeutic composition of embodiment 4, wherein the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, or bacteria having 16S rDNA having at least 94.5%, 95%, 97%, 98%, 98.7% or 99% sequence identity to any one of the 16S rDNA thereof.

Embodiment 8. The therapeutic composition of embodiment 4, wherein the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, or bacteria having 16S rDNA having at least 94.5%, 95%, 97%, 98%, 98.7% or 99% sequence identity to any one of the 16S rDNA thereof.

Embodiment 9. The therapeutic composition of embodiment 4, wherein the bacteria comprises one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

Embodiment 10. A therapeutic composition comprising a purified population of bacteria, wherein the purified or isolated population of bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772 and 773.

Embodiment 11. The therapeutic composition of embodiment 10, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

Embodiment 12. The therapeutic composition of embodiment 10, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

Embodiment 13. The therapeutic composition of embodiment 10, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180 and 182.

Embodiment 14. The therapeutic composition of embodiment 10, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179 and 184.

Embodiment 15. The therapeutic composition of embodiment 10, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183 and 186.

Embodiment 16. The therapeutic composition of any of embodiments 4-15, wherein the therapeutic composition comprises two, three, four, five, six, seven, eight, nine, ten, eleven, twelve, thirteen, fourteen, fifteen, sixteen, seventeen, eighteen or more bacterial species.

Embodiment 17. The therapeutic composition of any of embodiments 1-16, wherein the therapeutic composition does not comprise one or more bacteria selected from Holdemania massiliensis, Rothia mucilaginosa, Hungatella sp2, Prevotella copri, Campylobacter concisus, [Atopobium] parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus]gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

Embodiment 18. The therapeutic composition of any of embodiments 1-16, wherein the therapeutic composition does not comprise Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof.

Embodiment 19. The therapeutic composition of any of embodiments 1-16, wherein the therapeutic composition does not comprise Rothia mucilaginosa, [Atopobium] parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof.

Embodiment 20. The therapeutic composition of any of embodiments 1-16, wherein the therapeutic composition does not comprise Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, or bacteria having 16S rDNA having at least 94.5%, 95%, 97%, 98%, 98.7% or 99% sequence identity to any one of the 16S rDNA thereof.

Embodiment 21. The therapeutic composition of any of embodiments 1-16, wherein the therapeutic composition does not comprise [Coprococcus] catus, [Lactobacillus]fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, or bacteria having 16S rDNA having at least 94.5%, 95%, 97%, 98%, 98.7% or 99% sequence identity to any one of the 16S rDNA thereof.

Embodiment 22. The therapeutic composition of any of embodiments 1-16, wherein the therapeutic composition does not comprise [Atopobium] parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

Embodiment 23. The therapeutic composition of any of embodiments 1-16, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

Embodiment 24. The therapeutic composition of any of embodiments 1-16, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

Embodiment 25. The therapeutic composition of any of embodiments 1-16, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

Embodiment 26. The therapeutic composition of any of embodiments 1-16, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538 and 582.

Embodiment 27. The therapeutic composition of any of embodiments 1-16, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599 and 600.

Embodiment 28. The therapeutic composition of any of embodiments 1-16, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590 and 593.

Embodiment 29. The therapeutic composition of any of embodiments 1-28, further comprising at least one anticancer agent.

Embodiment 30. The therapeutic composition of embodiment 29, wherein the anticancer agent is a checkpoint inhibitor.

Embodiment 31. The therapeutic composition of embodiment 30, wherein the checkpoint inhibitor is selected from an anti-PD-1 antibody, an anti-CTLA-4 antibody, an anti-PD-L1 antibody or combinations thereof.

Embodiment 32. The therapeutic composition of embodiment 30, wherein the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT O11, MPDL3280A, MEDI-4736, MSB-0020718C, AUR-012, LAG-3, OX40 inhibitors, OX40L inhibitors, TIGIT inhibitors or STI-A1010, or combinations thereof.

Embodiment 33. The therapeutic composition of embodiment 29, wherein the anticancer agent is a chemotherapy agent.

Embodiment 34. The therapeutic composition of any of embodiments 29-33, wherein the anticancer agent is a combination of a checkpoint inhibitor and a chemotherapy agent.

Embodiment 35. The therapeutic composition of any of embodiments 1-34, wherein each of the purified or isolated populations of bacteria is present in the composition at a concentration of at least about 1×10² viable colony forming units (CFU).

Embodiment 36. The therapeutic composition of any of embodiments 1-35, wherein each purified or isolated population of bacteria is present in the composition at a concentration of about 1×10² to 1×10⁹ viable CFU.

Embodiment 37. The therapeutic composition of any of embodiments 1-36, wherein a fraction of the purified or isolated population of bacteria comprises a spore-forming bacteria.

Embodiment 38. The therapeutic composition of any of embodiments 1-36, wherein a fraction of the purified or isolated population of bacteria is in spore form.

Embodiment 39. The therapeutic composition of any of embodiments 1-36, wherein a fraction of the purified or isolated population of bacteria is in vegetative form.

Embodiment 40. The therapeutic composition of any of embodiments 1-39, wherein the composition further comprises a pharmaceutically acceptable excipient.

Embodiment 41. The therapeutic composition of any of embodiments 1-40, wherein the composition is formulated for delivery to the intestine.

Embodiment 42. The therapeutic composition of any of embodiments 1-41, wherein the composition is enterically coated.

Embodiment 43. The therapeutic composition of any of embodiments 1-42, wherein the composition is formulated for oral administration.

Embodiment 44. The therapeutic composition of any of embodiments 1-39, wherein the composition is formulated into a food or beverage.

Embodiment 45. The therapeutic composition of any of embodiments 1-44, wherein the composition can reduce the rate of tumor growth in an animal model or in a mammalian subject.

Embodiment 46. The therapeutic composition of any of embodiments 1-44, wherein the composition can reduce the rate of tumor growth in an animal model or in a mammalian subject when administered concomitantly with, prior to, or subsequent to the administration of at least one anticancer agent.

Embodiment 47. The therapeutic composition of any of embodiments 1-44, wherein the composition can increase the number of active CD8 T cells in a tumor of an animal model or of a mammalian subject when administered concomitantly with, prior to, or subsequent to the administration of at least one anticancer agent.

Embodiment 48. The therapeutic composition of any one of embodiments 1-47, wherein the composition is formulated for multiple administrations.

Embodiment 49. The therapeutic composition of any one of embodiments 1-48, wherein each of the populations of bacteria is present in the composition at a concentration of at least 1×10³ viable CFU.

Embodiment 50. A method of treating or preventing or reducing risk of a cancer in a mammalian subject in need thereof comprising administering to the subject a therapeutic composition comprising an effective amount of a purified or isolated population of bacteria that have 16S rDNA sequence identity of at least 94.5% to 16S rDNA sequences of species belonging to one or more of the families Sutterellaceae, Coriobacteriaceae, Akkermansiaceae, Campylobacteraceae, or Desulfovibrionaceae.

Embodiment 51. The method of embodiment 50, wherein the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Campylobacteraceae, or Desulfovibrionaceae.

Embodiment 52. The method of embodiment 50 or 51, wherein the therapeutic composition comprises bacteria belonging to two, three, four or more families.

Embodiment 53. A method of treating or preventing or reducing risk of a cancer in a mammalian subject in need thereof comprising administering to the subject a therapeutic composition comprising an effective amount of a purified or isolated population of bacteria comprising one or more bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

Embodiment 54. The method of embodiment 53, wherein the bacteria comprises one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof.

Embodiment 55. The method of embodiment 53, wherein the bacteria comprises one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

Embodiment 56. The method of embodiment 53, wherein the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, or bacteria having 16S rDNA having at least 94.5%, 95%, 97%, 98%, 98.7% or 99% sequence identity to any one of the 16S rDNA thereof.

Embodiment 57. The method of embodiment 56, wherein the cancer is a bladder cancer.

Embodiment 58. The method of embodiment 53, wherein the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, or bacteria having 16S rDNA having at least 94.5%, 95%, 97%, 98%, 98.7% or 99% sequence identity to any one of the 16S rDNA thereof.

Embodiment 59. The method of embodiment 58, wherein the cancer is a non-small cell lung cancer.

Embodiment 60. The method of embodiment 53, wherein the bacteria comprises one or more bacteria selected from [Adlercreutzia] equolifaciens group, [Anaerotruncus]rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

Embodiment 61. The method of embodiment 60, wherein the cancer is a metastatic melanoma.

Embodiment 62. A method of treating or preventing or reducing risk of a cancer in a mammalian subject in need thereof comprising administering to the subject a therapeutic composition comprising an effective amount of a purified or isolated population of bacteria comprising one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772 and 773.

Embodiment 63. The method of embodiment 62, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

Embodiment 64. The method of embodiment 62, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

Embodiment 65. The method of embodiment 62, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180 and 182.

Embodiment 66. The method of embodiment 65, wherein the cancer is a bladder cancer.

Embodiment 67. The method of embodiment 62, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179 and 184.

Embodiment 68. The method of embodiment 67, wherein the cancer is a non-small cell lung cancer.

Embodiment 69. The method of embodiment 62, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183 and 186.

Embodiment 70. The method of embodiment 69, wherein the cancer is a metastatic melanoma.

Embodiment 71. The method of any of embodiment 53-70, wherein the therapeutic composition comprises bacteria two, three, four, five, six, seven, eight, nine, ten, eleven, twelve, thirteen, fourteen, fifteen, sixteen, seventeen, eighteen or more bacterial species.

Embodiment 72. The method of any of embodiment 50-71, wherein the therapeutic composition does not comprise one or more bacteria selected from Holdemania massiliensis, Rothia mucilaginosa, Hungatella sp2, Prevotella copri, Campylobacter concisus, [Atopobium] parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

Embodiment 73. The method of any of embodiment 50-71, wherein the therapeutic composition does not comprise Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof.

Embodiment 74. The method of any of embodiment 50-71, wherein the therapeutic composition does not comprise Rothia mucilaginosa, [Atopobium] parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof.

Embodiment 75. The method of any of embodiment 50-71, wherein the therapeutic composition does not comprise Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, or bacteria having 16S rDNA having at least 94.5%, 95%, 97%, 98%, 98.7% or 99% sequence identity to any one of the 16S rDNA thereof.

Embodiment 76. The method of embodiment 75, wherein the cancer is a bladder cancer.

Embodiment 77. The method of any of embodiments 50-71, wherein the therapeutic composition does not comprise [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, or bacteria having 16S rDNA having at least 94.5%, 95%, 97%, 98%, 98.7% or 99% sequence identity to any one of the 16S rDNA thereof.

Embodiment 78. The method of embodiment 77, wherein the cancer is a non-small cell lung cancer.

Embodiment 79. The method of any of embodiments 50-71, wherein the therapeutic composition does not comprise [Atopobium] parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

Embodiment 80. The method of embodiment 79, wherein the cancer is a metastatic melanoma.

Embodiment 81. The method of any of embodiment 50-71, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

Embodiment 82. The method of any of embodiment 50-71, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

Embodiment 83. The method of any of embodiment 50-71, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

Embodiment 84. The method of any of embodiment 50-71, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538 and 582.

Embodiment 85. The method of embodiment 84, wherein the cancer is a bladder cancer.

Embodiment 86. The method of any of embodiment 50-71, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599 and 600.

Embodiment 87. The method of embodiment 86, wherein the cancer is non-small cell lung cancer.

Embodiment 88. The method of any of embodiment 50-71, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590 and 593.

Embodiment 89. The method of embodiment 88, wherein the cancer is metastatic melanoma.

Embodiment 90. The method of any one of embodiments 50-89, further comprising administering at least one anticancer agent to the subject.

Embodiment 91. The method of embodiment 90, wherein the anticancer agent is a checkpoint inhibitor.

Embodiment 92. The method of embodiment 91, wherein the checkpoint inhibitor is selected from an anti-PD-1 antibody, an anti-CTLA-4 antibody, an anti-PD-L1 antibody or combinations thereof.

Embodiment 93. The method of embodiment 91, wherein the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT O11, MPDL3280A, MEDI-4736, MSB-0020718C, AUR-012, LAG-3, OX40 inhibitors, OX40L inhibitors, TIGIT inhibitors, STI-A1010 or combinations thereof.

Embodiment 94. The method of embodiment 90, wherein the anticancer agent comprises a chemotherapy agent.

Embodiment 95. The method of any of embodiments 90-94, wherein the anticancer agent is a combination of a checkpoint inhibitor and a chemotherapy agent.

Embodiment 96. The method of any of embodiments 50-95, wherein the purified or isolated population of bacteria is administered at a concentration of at least about 1×10² viable colony forming units.

Embodiment 97. The method of any of embodiments 50-96, wherein the purified or isolated population of bacteria is administered at a concentration of at a concentration of about 1×10² to 1×10⁹ viable colony forming units.

Embodiment 98. The method of any of embodiments 50-97, wherein a fraction of the purified or isolated population of bacteria comprises a spore-forming bacteria.

Embodiment 99. The method of any of embodiments 50-97, wherein a fraction of the purified or isolated population of bacteria is in spore form.

Embodiment 100. The method of any of embodiments 50-97, wherein a fraction of the purified or isolated population of bacteria is in vegetative form.

Embodiment 101. The method of any of embodiments 50-100, wherein the composition further comprises a pharmaceutically acceptable excipient.

Embodiment 102. The method of any of embodiments 50-101, wherein the composition is formulated for delivery to the intestine.

Embodiment 103. The method of any of embodiments 50-102, wherein the composition is enterically coated.

Embodiment 104. The method of any of embodiments 50-103, wherein the composition is formulated for oral administration.

Embodiment 105. The method of any of embodiments 50-104, wherein the composition is formulated into a food or beverage.

Embodiment 106. The method of any of embodiments 50-105, wherein the mammalian subject is a human.

Embodiment 107. The method of any of embodiments 50-106, wherein the cancer is selected from a metastatic melanoma, melanoma of the skin, non-small cell lung cancer, a kidney cancer, a bladder cancer, a head and neck cancer, a Merkel cell skin cancer (Merkel cell carcinoma), or a Hodgkin lymphoma.

Embodiment 108. The method of embodiment 107, wherein the cancer is a metastatic melanoma.

Embodiment 109. The method of embodiment 107, wherein the cancer is a non-small cell lung cancer.

Embodiment 110. The method of embodiment 107, wherein the cancer is a bladder cancer.

Embodiment 111. The method of any of embodiments 50-110, wherein prior to administration of the population of bacteria the subject is subjected to antibiotic treatment and/or a bowel cleanse.

Embodiment 112. The method of any one of embodiments 50-111, wherein the subject has previously been treated for the cancer.

Embodiment 113. The method of embodiment 112, wherein the subject has been determined to be a non-responder to the previous treatment.

Embodiment 114. The method of embodiment 112 or 113, wherein the subject has been determined to have a toxic response to the previous treatment.

Embodiment 115. The method of any one of embodiments 112-114, wherein the previous treatment comprises immune checkpoint blockade monotherapy or immune checkpoint blockade combination therapy.

Embodiment 116. The method of any one of embodiments 50-115, wherein the cancer is recurrent cancer.

Embodiment 117. A method of identifying a mammalian subject as a candidate for immune checkpoint therapy in combination with adjuvant microbiome therapy to treat a cancer, the method comprising:
a) obtaining a microbiome sample from the subject,
b) determining the prevalence and/or abundance of the family of bacteria in the microbiome sample, and
c) determining that the subject is a candidate for treatment if the microbiome sample comprises bacteria that have 16S rDNA sequence identity of at least 94.5% to 16S rDNA sequences of species belonging to one or more of the families Sutterellaceae, Coriobacteriaceae, Akkermansiaceae, Campylobacteraceae, or Desulfovibrionaceae.

Embodiment 118. The method of embodiment 117, wherein the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Campylobacteraceae, or Desulfovibrionaceae.

Embodiment 119. A method of identifying a mammalian subject as a candidate for immune checkpoint therapy in combination with adjuvant microbiome therapy to treat a cancer, the method comprising:
a) obtaining a microbiome sample from the subject,
b) determining the prevalence and/or abundance of the family of bacteria in the microbiome sample, and
c) determining that the subject is a candidate for treatment if the microbiome sample comprises bacteria comprising one or more bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

Embodiment 120. The method of embodiment 119, wherein the bacteria comprises one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof.

Embodiment 121. The method of embodiment 119, wherein the bacteria comprises one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

Embodiment 122. The method of embodiment 119, wherein the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, or bacteria having 16S rDNA having at least 94.5%, 95%, 97%, 98%, 98.7% or 99% sequence identity to any one of the 16S rDNA thereof.

Embodiment 123. The method of embodiment 122, wherein the cancer is a bladder cancer.

Embodiment 124. The method of embodiment 119, wherein the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, or bacteria having 16S rDNA having at least 94.5%, 95%, 97%, 98%, 98.7% or 99% sequence identity to any one of the 16S rDNA thereof.

Embodiment 125. The method of embodiment 124, wherein the cancer is a non-small cell lung cancer.

Embodiment 126. The method of embodiment 119, wherein the bacteria comprises one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

Embodiment 127. The method of embodiment 126, wherein the cancer is a metastatic melanoma.

Embodiment 128. A method of identifying a mammalian subject as a candidate for immune checkpoint therapy in combination with adjuvant microbiome therapy to treat a cancer, the method comprising:
a) obtaining a microbiome sample from the subject,
b) determining the prevalence and/or abundance of the family of bacteria in the microbiome sample, and
c) determining that the subject is a candidate for treatment if the microbiome sample comprises bacteria comprising one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772 and 773.

Embodiment 129. The method of embodiment 128, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

Embodiment 130. The method of embodiment 128, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

Embodiment 131. The method of embodiment 128, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180 and 182.

Embodiment 132. The method of embodiment 131, wherein the cancer is a bladder cancer.

Embodiment 133. The method of embodiment 128, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179 and 184.

Embodiment 134. The method of embodiment 133, wherein the cancer is a non-small cell lung cancer.

Embodiment 135. The method of embodiment 128, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183 and 186.

Embodiment 136. The method of embodiment 135, wherein the cancer is a metastatic melanoma.

Embodiment 137. The method of any of embodiments 117-136, wherein determining that the subject is a candidate for treatment further comprises determining if the microbiome sample does not comprise one or more bacteria selected from Holdemania massiliensis, Rothia mucilaginosa, Hungatella sp2, Prevotella copri, Campylobacter concisus, [Atopobium] parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

Embodiment 138. The method of any of embodiments 117-136, wherein determining that the subject is a candidate for treatment further comprises determining if the microbiome sample does not comprise one or more bacteria selected from Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof.

Embodiment 139. The method of any of embodiments 117-136, wherein determining that the subject is a candidate for treatment further comprises determining if the microbiome sample does not comprise one or more bacteria selected from Rothia mucilaginosa, [Atopobium] parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof.

Embodiment 140. The method of any of embodiments 117-136, wherein determining that the subject is a candidate for treatment further comprises determining if the microbiome sample does not comprise one or more bacteria selected from Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, or bacteria having 16S rDNA having at least 94.5%, 95%, 97%, 98%, 98.7% or 99% sequence identity to any one of the 16S rDNA thereof.

Embodiment 141. The method of embodiment 140, wherein the cancer is a bladder cancer.

Embodiment 142. The method of any of embodiments 117-136, wherein determining that the subject is a candidate for treatment further comprises determining if the microbiome sample does not comprise one or more bacteria selected from [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, or bacteria having 16S rDNA having at least 94.5%, 95%, 97%, 98%, 98.7% or 99% sequence identity to any one of the 16S rDNA thereof.

Embodiment 143. The method of embodiment 142, wherein the cancer is a non-small cell lung cancer.

Embodiment 144. The method of any of embodiments 117-136, wherein determining that the subject is a candidate for treatment further comprises determining if the microbiome sample does not comprise one or more bacteria selected from [Atopobium] parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

Embodiment 145. The method of embodiment 144, wherein the cancer is a metastatic melanoma.

Embodiment 146. The method of any of embodiments 117-136, wherein determining that the subject is a candidate for treatment further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

Embodiment 147. The method of any of embodiments 117-136, wherein determining that the subject is a candidate for treatment further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

Embodiment 148. The method of any of embodiments 117-136, wherein determining that the subject is a candidate for treatment further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

Embodiment 149. The method of any of embodiments 117-136, wherein determining that the subject is a candidate for treatment further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538 and 582.

Embodiment 150. The method of embodiment 149, wherein the cancer is a bladder cancer.

Embodiment 151. The method of any of embodiments 117-136, wherein determining that the subject is a candidate for treatment further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599 and 600.

Embodiment 152. The method of embodiment 151, wherein the cancer is a non-small cell lung cancer.

Embodiment 153. The method of any of embodiments 117-136, wherein determining that the subject is a candidate for treatment further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590 and 593.

Embodiment 154. The method of embodiment 153, wherein the cancer is a metastatic melanoma.

Embodiment 155. The method of any of embodiments 117-154, wherein the subject is determined to be a candidate for immune checkpoint inhibitor therapy.

Embodiment 156. The method of any one of embodiments 117-155, wherein the immune checkpoint therapy comprises immune checkpoint blockade monotherapy or immune checkpoint blockade combination therapy.

Embodiment 157. The method of any of embodiments 117-154, wherein the treatment further comprises a chemotherapy.

Embodiment 158. The method of any of embodiments 117-157, wherein the mammalian subject is a human.

Embodiment 159. The method of any of embodiments 117-158, wherein the cancer is selected from a metastatic melanoma, a melanoma of the skin, a non-small cell lung cancer, a kidney cancer, a bladder cancer, a head and neck cancer, a Merkel cell skin cancer (Merkel cell carcinoma), or a Hodgkin lymphoma.

Embodiment 160. The method of embodiment 159, wherein the cancer is a metastatic melanoma.

Embodiment 161. The method of embodiment 159, wherein the cancer is a non-small cell lung cancer.

Embodiment 162. The method of embodiment 159, wherein the cancer is a bladder cancer.

Embodiment 163. A method of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises bacteria that have 16S rDNA sequence identity of at least 94.5% to 16S rDNA sequences of species belonging to one or more of the families Sutterellaceae, Coriobacteriaceae, Akkermansiaceae, Campylobacteraceae, or Desulfovibrionaceae.

Embodiment 164. The method of embodiment 163, wherein the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Campylobacteraceae, or Desulfovibrionaceae.

Embodiment 165. A method of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises bacteria comprising one or more bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

Embodiment 166. The method of embodiment 165, wherein the bacteria comprises one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof.

Embodiment 167. The method of embodiment 165, wherein the bacteria comprises one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

Embodiment 168. The method of embodiment 165, wherein the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, or bacteria having 16S rDNA having at least 94.5%, 95%, 97%, 98%, 98.7% or 99% sequence identity to any one of the 16S rDNA thereof.

Embodiment 169. The method of embodiment 165, wherein the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, or bacteria having 16S rDNA having at least 94.5%, 95%, 97%, 98%, 98.7% or 99% sequence identity to any one of the 16S rDNA thereof.

Embodiment 170. The method of embodiment 165, wherein the bacteria comprises one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

Embodiment 171. A method of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises bacteria comprising one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772 and 773.

Embodiment 172. The method of embodiment 171, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

Embodiment 173. The method of embodiment 171, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

Embodiment 174. The method of embodiment 171, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180 and 182.

Embodiment 175. The method of embodiment 171, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179 and 184.

Embodiment 176. The method of embodiment 171, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183 and 186.

Embodiment 177. The method of any one of embodiments 163-176, wherein determining that the donor's feces is useful for fecal matter transfer further comprises determining if the microbiome sample does not comprise one or more bacteria selected from Holdemania massiliensis, Rothia mucilaginosa, Hungatella sp2, Prevotella copri, Campylobacter concisus, [Atopobium] parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus]gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

Embodiment 178. The method of any one of embodiments 163-176, wherein determining that the donor's feces is useful for fecal matter transfer further comprises determining if the microbiome sample does not comprise one or more bacteria selected from Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof.

Embodiment 179. The method of any one of embodiments 163-176, wherein determining that the donor's feces is useful for fecal matter transfer further comprises determining if the microbiome sample does not comprise one or more bacteria selected from Rothia mucilaginosa, [Atopobium] parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof.

Embodiment 180. The method of any one of embodiments 163-176, wherein determining that the donor's feces is useful for fecal matter transfer further comprises determining if the microbiome sample does not comprise one or more bacteria selected from Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, or bacteria having 16S rDNA having at least 94.5%, 95%, 97%, 98%, 98.7% or 99% sequence identity to any one of the 16S rDNA thereof.

Embodiment 181. The method of any one of embodiments 163-176, wherein determining that the donor's feces is useful for fecal matter transfer further comprises determining if the microbiome sample does not comprise one or more bacteria selected from [Coprococcus] catus, [Lactobacillus] fermentum, [Romboutsia] timonensis, [Ruminococcus]bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, or bacteria having 16S rDNA having at least 94.5%, 95%, 97%, 98%, 98.7% or 99% sequence identity to any one of the 16S rDNA thereof.

Embodiment 182. The method of any one of embodiments 163-176, wherein determining that the donor's feces is useful for fecal matter transfer further comprises determining if the microbiome sample does not comprise one or more bacteria selected from [Atopobium] parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

Embodiment 183. The method of any one of embodiments 163-176, wherein determining that the donor's feces is useful for fecal matter transfer further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

Embodiment 184. The method of any of embodiments 163-176, wherein determining that the donor's feces is useful for fecal matter transfer further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

Embodiment 185. The method of any of embodiments 163-176, wherein determining that the donor's feces is useful for fecal matter transfer further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

Embodiment 186. The method of any of embodiments 163-176, wherein determining that the donor's feces is useful for fecal matter transfer further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538 and 582.

Embodiment 187. The method of any of embodiments 163-176, wherein determining that the donor's feces is useful for fecal matter transfer further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599 and 600.

Embodiment 188. The method of any of embodiments 163-176, wherein determining that the donor's feces is useful for fecal matter transfer further comprises determining if the microbiome sample does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590 and 593.

Embodiment 189. A therapeutic composition derived from fecal matter from a donor identified using the method of any one of embodiments 163-188.

Embodiment 190. The therapeutic composition of embodiment 189, further comprising a pharmaceutically acceptable excipient.

Embodiment 191. The therapeutic composition of embodiment 189, wherein the therapeutic composition comprises bacteria that are in vegetative and/or spore form.

Embodiment 192. The therapeutic composition of embodiment 189, wherein the therapeutic composition further comprises a checkpoint inhibitor.

Embodiment 193. The therapeutic composition of embodiment 192, wherein the checkpoint inhibitor is selected from anti-PD-1 antibody, an anti-CTLA-4 antibody, an anti-PD-L1 antibody or combinations thereof.

Embodiment 194. The therapeutic composition of embodiment 192, wherein the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT O11, MPDL3280A, MEDI-4736, MSB-0020718C, AUR-012, LAG-3, OX40 inhibitors, OX40L inhibitors, TIGIT inhibitors, STI-A1010 or combinations thereof.

Embodiment 195. A method of treating or preventing or reducing risk of a cancer in a mammalian subject comprising administering to the subject a therapeutic composition of any one of embodiments 189-194.

Embodiment 196. A method of treating or preventing or reducing risk of a cancer comprising administering at least one anticancer treatment to a subject determined to have a microbiome sample comprising bacteria have 16S rDNA sequence identity of at least 94.5% to 16S rDNA sequences of species belonging to one or more of the families Sutterellaceae, Coriobacteriaceae, Akkermansiaceae, Campylobacteraceae, or Desulfovibrionaceae.

Embodiment 197. The method of embodiment 196, wherein the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Campylobacteraceae, or Desulfovibrionaceae.

Embodiment 198. A method of treating or preventing or reducing risk of a cancer comprising administering an anticancer treatment to a subject determined to have a microbiome sample comprising bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus]rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

Embodiment 199. The method of embodiment 198, wherein the bacteria comprises one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof.

Embodiment 200. The method of embodiment 198, wherein the bacteria comprises one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

Embodiment 201. The method of embodiment 198, wherein the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, or bacteria having 16S rDNA having at least 94.5%, 95%, 97%, 98%, 98.7% or 99% sequence identity to any one of the 16S rDNA thereof.

Embodiment 202. The method of embodiment 201, wherein the cancer is a bladder cancer.

Embodiment 203. The method of embodiment 198, wherein the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, or bacteria having 16S rDNA having at least 94.5%, 95%, 97%, 98%, 98.7% or 99% sequence identity to any one of the 16S rDNA thereof.

Embodiment 204. The method of embodiment 203, wherein the cancer is a non-small cell lung cancer.

Embodiment 205. The method of embodiment 198, wherein the bacteria comprises one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

Embodiment 206. The method of embodiment 205, wherein the cancer is a metastatic melanoma.

Embodiment 207. A method of treating or preventing or reducing risk of a cancer comprising administering an anticancer treatment to a subject determined to have a microbiome sample comprising one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772 and 773.

Embodiment 208. The method of embodiment 207, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

Embodiment 209. The method of embodiment 207, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

Embodiment 210. The method of embodiment 207, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180 and 182.

Embodiment 211. The method of embodiment 210, wherein the cancer is a bladder cancer.

Embodiment 212. The method of embodiment 207, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179 and 184.

Embodiment 213. The method of embodiment 212, wherein the cancer is a non-small cell lung cancer.

Embodiment 214. The method of embodiment 207, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183 and 186.

Embodiment 215. The method of embodiment 214, wherein the cancer is a metastatic melanoma.

Embodiment 216. The method of any of embodiments 196-215, wherein the subject is determined to not have a microbiome sample that comprises one or more bacteria selected from Holdemania massiliensis, Rothia mucilaginosa, Hungatella sp2, Prevotella copri, Campylobacter concisus, [Atopobium] parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus]gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

Embodiment 217. The method of any of embodiments 196-215, wherein the subject is determined to not have a microbiome sample that comprises Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof.

Embodiment 218. The method of any of embodiments 196-215, wherein the subject is determined to not have a microbiome sample that comprises Rothia mucilaginosa, [Atopobium] parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof.

Embodiment 219. The method of any of embodiments 196-215, wherein the subject is determined to not have a microbiome sample that comprises Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, or bacteria having 16S rDNA having at least 94.5%, 95%, 97%, 98%, 98.7% or 99% sequence identity to any one of the 16S rDNA thereof.

Embodiment 220. The method of embodiment 219, wherein the cancer is a bladder cancer.

Embodiment 221. The method of any of embodiments 196-215, wherein the subject is determined to not have a microbiome sample that comprises [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, or bacteria having 16S rDNA having at least 94.5%, 95%, 97%, 98%, 98.7% or 99% sequence identity to any one of the 16S rDNA thereof.

Embodiment 222. The method of embodiment 221, wherein the cancer is a non-small cell lung cancer.

Embodiment 223. The method of any of embodiments 196-215, wherein the subject is determined to not have a microbiome sample that comprises [Atopobium]parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

Embodiment 224. The method of embodiment 223, wherein the cancer is a metastatic melanoma.

Embodiment 225. The method of any of embodiments 196-215, wherein the subject is determined to not have a microbiome sample that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

Embodiment 226. The method of any of embodiments 196-215, wherein the subject is determined to not have a microbiome sample that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

Embodiment 227. The method of any of embodiments 196-215, wherein the subject is determined to not have a microbiome sample that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

Embodiment 228. The method of any of embodiments 196-215, wherein the subject is determined to not have a microbiome sample that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538 and 582.

Embodiment 229. The method of embodiment 228, wherein the cancer is a bladder cancer.

Embodiment 230. The method of any of embodiments 196-215, wherein the subject is determined to not have a microbiome sample that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599 and 600.

Embodiment 231. The method of embodiment 230, wherein the cancer is a non-small cell lung cancer.

Embodiment 232. The method of any of embodiments 196-215, wherein the subject is determined to not have a microbiome sample that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590 and 593.

Embodiment 233. The method of embodiment 232, wherein the cancer is a metastatic melanoma.

Embodiment 234. A method comprising evaluating a microbiome profile in a sample from a subject for bacteria that have 16S rDNA sequence identity of at least 94.5% to 16S rDNA sequences of species belonging to one or more of the families Sutterellaceae, Coriobacteriaceae, Akkermansiaceae, Campylobacteraceae, or Desulfovibrionaceae.

Embodiment 235. The method of embodiment 234, wherein the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Campylobacteraceae, or Desulfovibrionaceae.

Embodiment 236. A method comprising evaluating a microbiome profile in a sample from a subject for bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter]muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

Embodiment 237. The method of embodiment 236, wherein the bacteria comprises one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof.

Embodiment 238. The method of embodiment 236, wherein the bacteria comprises one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

Embodiment 239. The method of embodiment 236, wherein the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, or bacteria having 16S rDNA having at least 94.5%, 95%, 97%, 98%, 98.7% or 99% sequence identity to any one of the 16S rDNA thereof.

Embodiment 240. The method of embodiment 236, wherein the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, or bacteria having 16S rDNA having at least 94.5%, 95%, 97%, 98%, 98.7% or 99% sequence identity to any one of the 16S rDNA thereof.

Embodiment 241. The method of embodiment 236, wherein the bacteria comprises one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, or bacteria having 16S rDNA having at least 94.5%, 95%, 97%, 98%, 98.7% or 99% sequence identity to any one of the 16S rDNA thereof.

Embodiment 242. A method comprising evaluating a microbiome profile in a sample from a subject for bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772 and 773.

Embodiment 243. The method of embodiment 242, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

Embodiment 244. The method of embodiment 242, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

Embodiment 245. The method of embodiment 242, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180 and 182.

Embodiment 246. The method of embodiment 242, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179 and 184.

Embodiment 247. The method of embodiment 242, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183 and 186.

Embodiment 248. The method of any of embodiments 234-247, further comprising evaluating the microbiome profile for bacteria that comprises one or more bacteria selected from Holdemania massiliensis, Rothia mucilaginosa, Hungatella sp2, Prevotella copri, Campylobacter concisus, [Atopobium] parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus]bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium]parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

Embodiment 249. The method of any of embodiments 234-247, further comprising evaluating the microbiome profile for bacteria that comprises Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof.

Embodiment 250. The method of any of embodiments 234-247, further comprising evaluating the microbiome profile for bacteria that comprises Rothia mucilaginosa, [Atopobium] parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof.

Embodiment 251. The method of any of embodiments 234-247, further comprising evaluating the microbiome profile for bacteria that comprises Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, or bacteria having 16S rDNA having at least 94.5%, 95%, 97%, 98%, 98.7% or 99% sequence identity to any one of the 16S rDNA thereof.

Embodiment 252. The method of any of embodiments 234-247, further comprising evaluating the microbiome profile for bacteria that comprises [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, or bacteria having 16S rDNA having at least 94.5%, 95%, 97%, 98%, 98.7% or 99% sequence identity to any one of the 16S rDNA thereof.

Embodiment 253. The method of any of embodiments 234-247, further comprising evaluating the microbiome profile for bacteria that comprises [Atopobium]parvulum, [Ruminococcus] gnavus, Bacteroides dorei group, Blautia sp1, Schaalia sp2, or bacteria having 16S rDNA having at least 94.5%, 95%, 97%, 98%, 98.7% or 99% sequence identity to any one of the 16S rDNA thereof.

Embodiment 254. The method of any of embodiments 234-247, further comprising evaluating the microbiome profile for bacteria that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

Embodiment 255. The method of any of embodiments 234-247, further comprising evaluating the microbiome profile for bacteria that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

Embodiment 256. The method of any of embodiments 234-247, further comprising evaluating the microbiome profile for bacteria that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

Embodiment 257. The method of any of embodiments 234-247, further comprising evaluating the microbiome profile for bacteria that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538 and 582.

Embodiment 258. The method of any of embodiments 234-247, further comprising evaluating the microbiome profile for bacteria that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599 and 600.

Embodiment 259. The method of any of embodiments 234-247, further comprising evaluating the microbiome profile for bacteria that comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590 and 593.

Embodiment 260. The method of any of embodiments 234-259, wherein the method further comprises comparing the microbiome profile to a control microbiome.

Embodiment 261. The method of embodiment 260, wherein the control microbiome comprises a microbiome sample from a subject determined to be a responder to an anticancer treatment.

Embodiment 262. The method of embodiment 261, wherein the comparing comprises comparison of the bacteria from any of embodiments 234-247 to the control microbiome from a subject determined to be a responder to an anticancer treatment.

Embodiment 263. The method of embodiment 260, wherein the control microbiome comprises a microbiome sample from a subject determined to be a non-responder to an anticancer treatment.

Embodiment 264. The method of embodiment 263, wherein the comparing comprises comparison of the bacteria from any of embodiments 248-259 to the control microbiome from a subject determined to be a non-responder to an anticancer treatment.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: Characterization of Microbiomes Associated with Immunotherapy

Gut microbiome samples were obtained from patients with metastatic melanoma, lung cancer, urothelial cancer, or renal cell carcinoma immediately before CPI therapy was initiated and also while on treatment. The samples were sequenced using whole metagenome sequencing. Clinical characteristics were obtained from patient medical records; clinical response to CPI was defined as patients obtaining a partial or complete response or remaining on therapy for more than 6 months.

97 patients were enrolled and provided samples in this prospective study. The cohorts consisted of patients with metastatic lung cancer (n=44), urothelial cancer (n=25), renal cell carcinoma (RCC) (n=10), or melanoma (n=18). Patients received anti-PD1/anti-PDL1 monotherapy (n=54), anti-PD1+anti-CTLA4 combination therapy (n=17), or a combination of anti-PD1 and chemotherapy (n=26). Clinical response was observed in patients, including partial or complete response (45%) and on treatment for more than 6 months (55%, with 31% on treatment for more than 1 year). The patient characteristics are recorded in Table 1. Table 1 shows the number of patients providing microbiome samples and their therapy outcomes, i.e., responders (R) and non-responders (NR), and specific cancer types, i.e., bladder cancer, lung cancer, melanoma, and renal cell carcinoma (RCC).

TABLE 1
Number of patients providing microbiome
samples and their therapy outcomes

| Cancer Type | Responders (R) | Non-Responders (NR) |
|---|---|---|
| Lung | 22 | 22 |
| Bladder | 13 | 12 |
| Renal Cell | 8 | 2 |
| Carcinoma (RCC) | | |
| Melanoma | 11 | 7 |

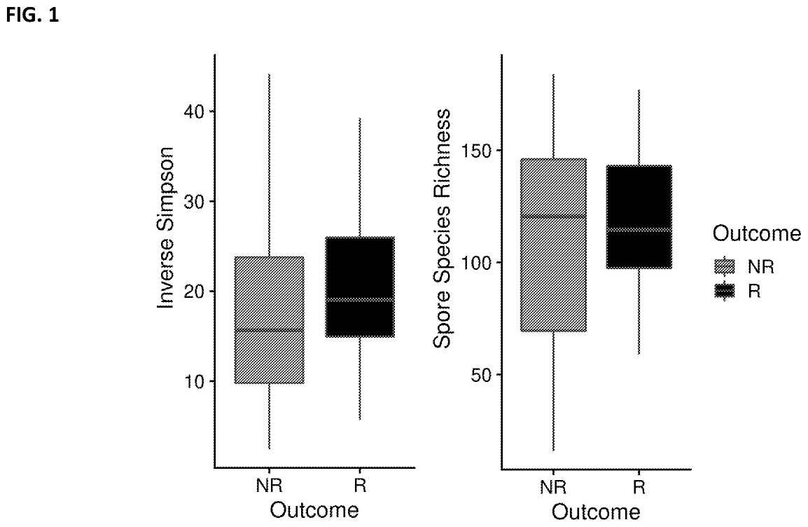

Whole metagenomics sequencing (WMS) raw data were obtained and analyzed as described herein. The WMS sequences were generated using fecal microbiome samples from patients of various types of cancers who were classified as responders or non-responders to immunothepraties. The raw data sets were pre-processed following the guidelines set by the Human Microbiome Project. The pre-processing analysis was used to perform error analysis and removal of low-quality sequences and other undesirable data, such as sequences present from PCR amplification steps. Species-level taxonomic profiles of each WMS sample were obtained using MetaPhlAn 2 with an expanded, proprietary reference database. Briefly, the software tool is used to align each sample to a curated reference database of marker genes, each of which is unique to a bacterial species. The reference database contains more than one million marker genes, representing more than thirteen thousand bacterial species. The alpha diversity of microbiomes, e.g., inversed Simpson index and spore species richness, of WMS samples from responders (R) and non-responders (NR) is shown in FIG. 1.

Abundance data were obtained after profiling WMS data. For a given sample, the sum of the abundances of all species sums to 100. Prevalence data are discretized so that species are analyzed only as being either present or absent. This is a population-wide data type, meaning that it can only be assessed for a set of samples and not individually for any given sample. For example, the prevalence of a species that appears in 4 out of 10 responders is 40%.

Microbiome community analysis, i.e., Bray-Curtis, Jaccard, and weighted and unweighted Unifrac, was conducted to characterize these microbiome samples and to identify microbiome signatures associated with responses in the immunotherapies across various cancer types.

Mann-Whitney U tests were conducted on continuous or integer-based data (e.g., relative abundance, species diversity), while Fisher's exact tests were conducted on categorical data (prevalence data). Wilcoxon rank-sum tests were conducted to compare the axis values between responders and non-responders of weighted UniFrac or unweighted UniFrac analysis.

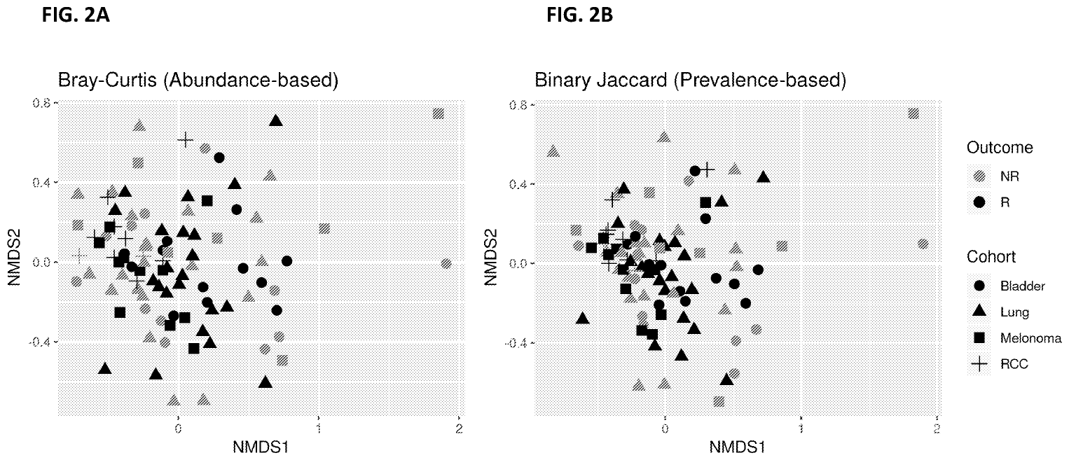

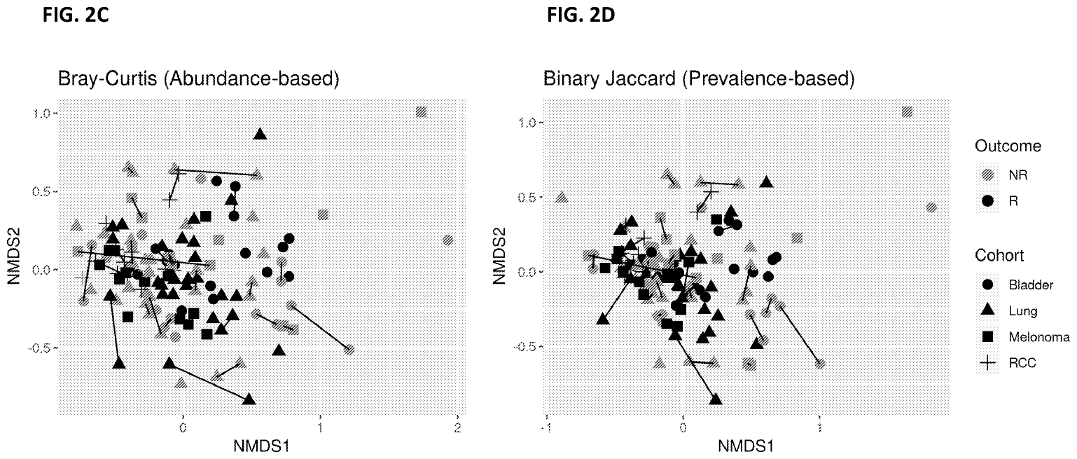

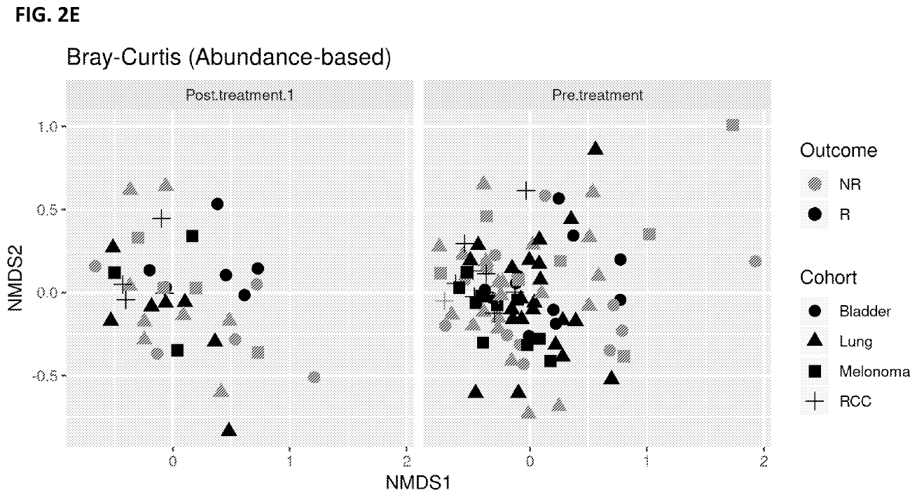

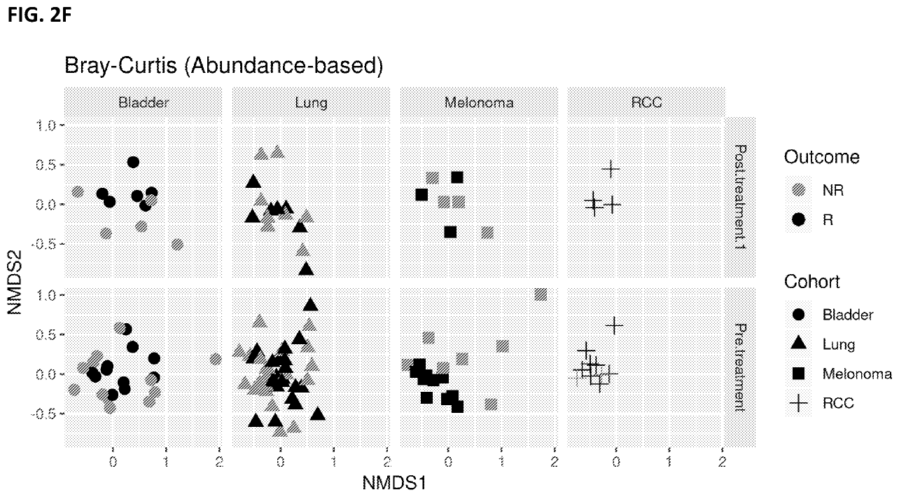

Combining all cancer types, the microbiomes of responder and non-responder groups did not differ significantly by beta diversity. According to NMDS plots of the abundance-based Bray-Curtis dissimilarity index (FIGS. 2A and 2C) or the prevalence-based binary Jaccard distance (FIGS. 2B and 2D), the microbiomes did not cluster by specific cancer types or outcomes. The samples from each patient (linked by solid lines in FIGS. 2C and 2D) or as a whole (FIG. 2E) at different time points (i.e., pre-treatment and post treatment) did not show consistent trends on the NMDS plots of these dissimilarity index. As shown in FIG. 2F, these microbiomes show some clustering of responders and nonresponders by the specific cancer types.

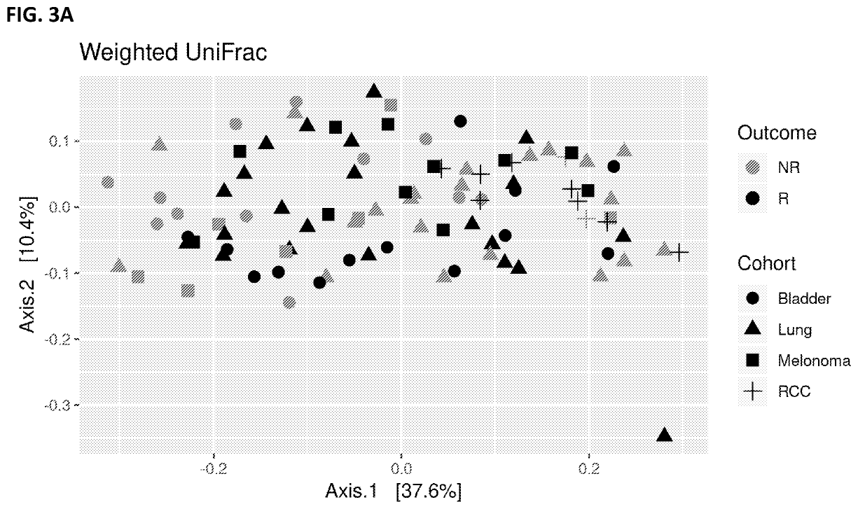

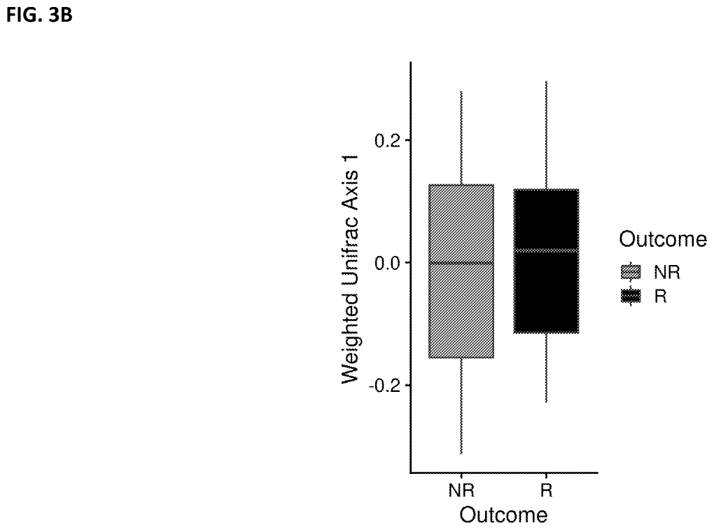

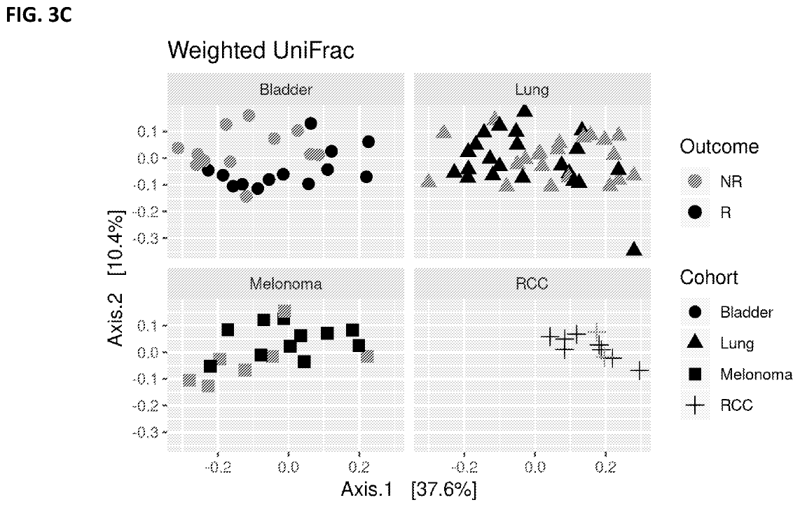

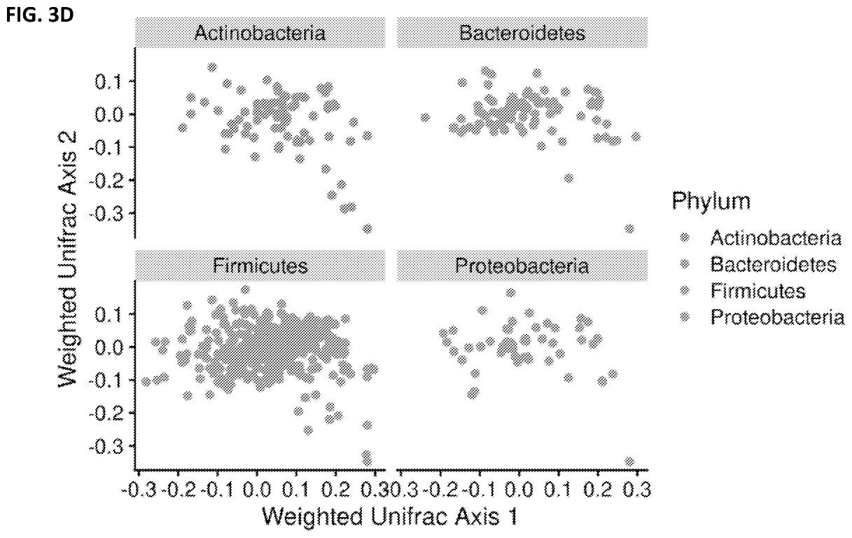

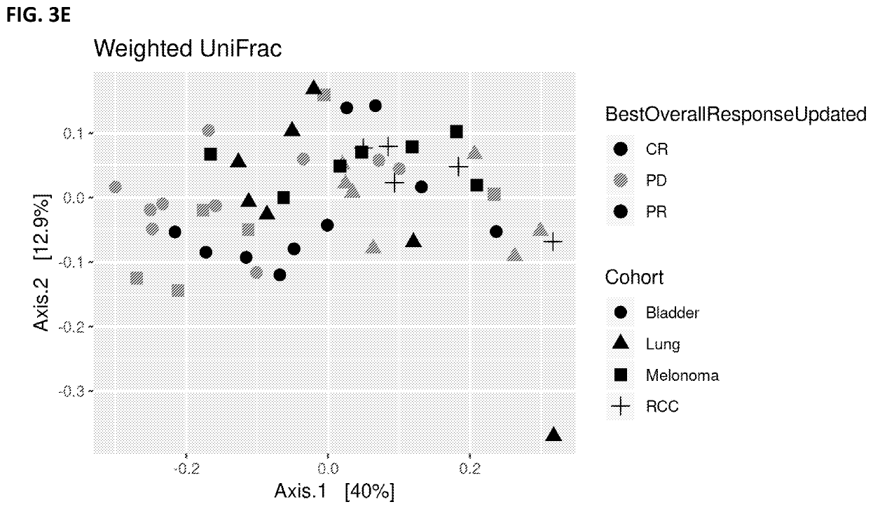

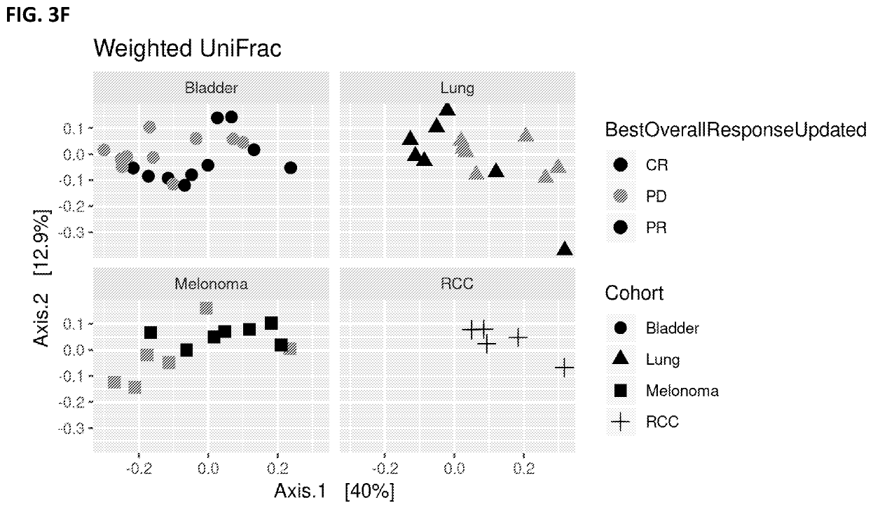

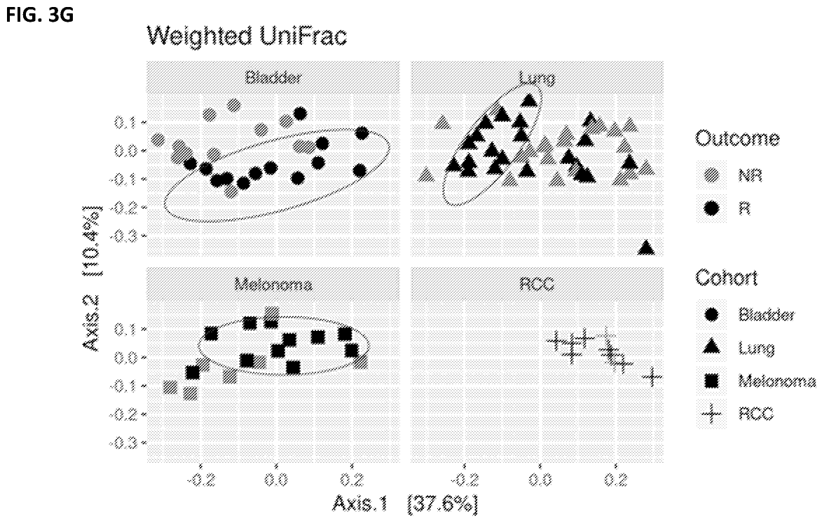

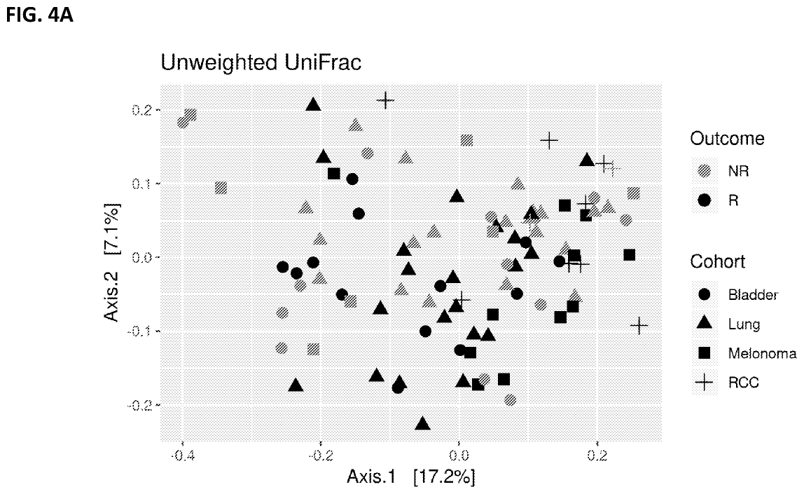

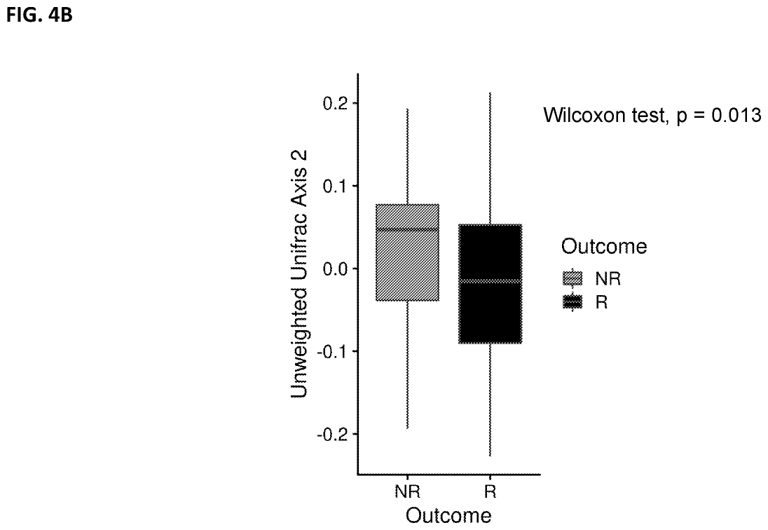

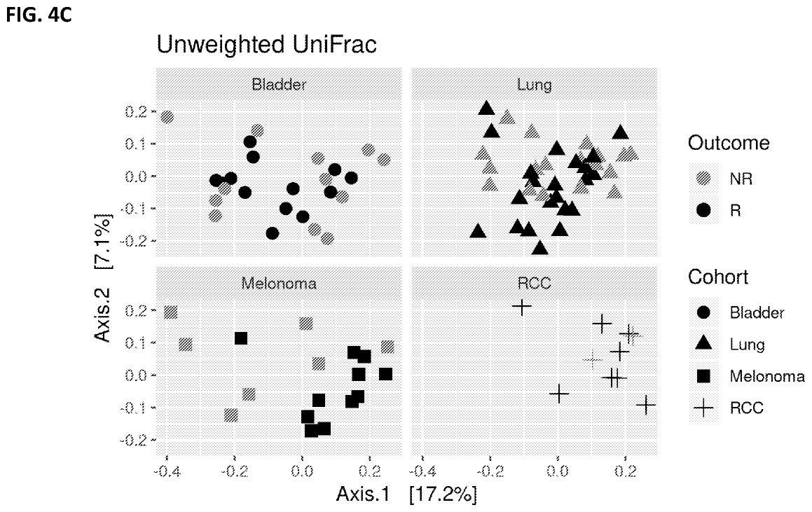

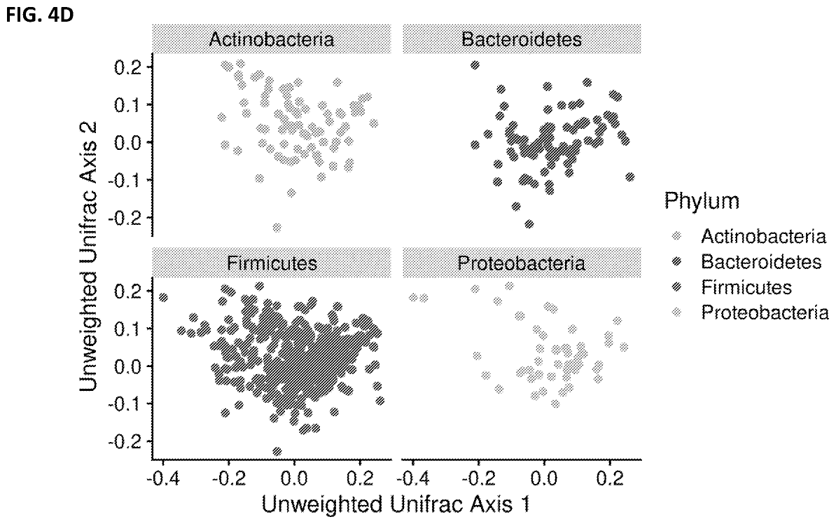

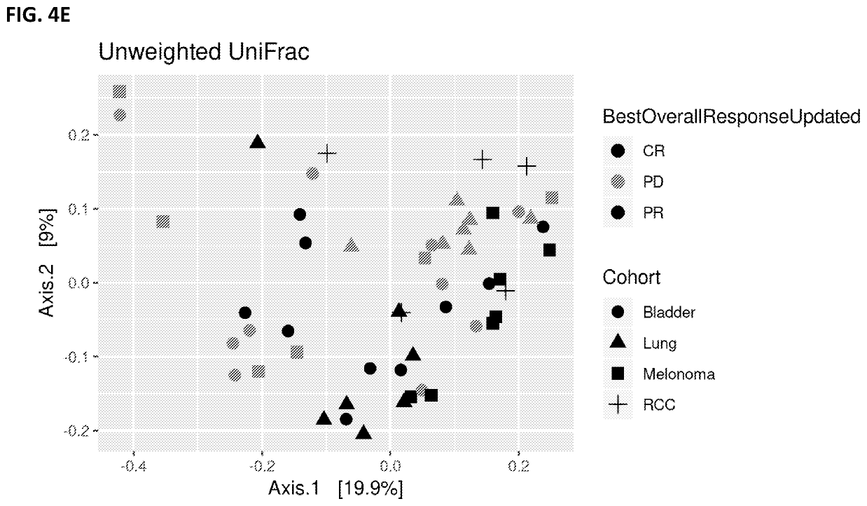

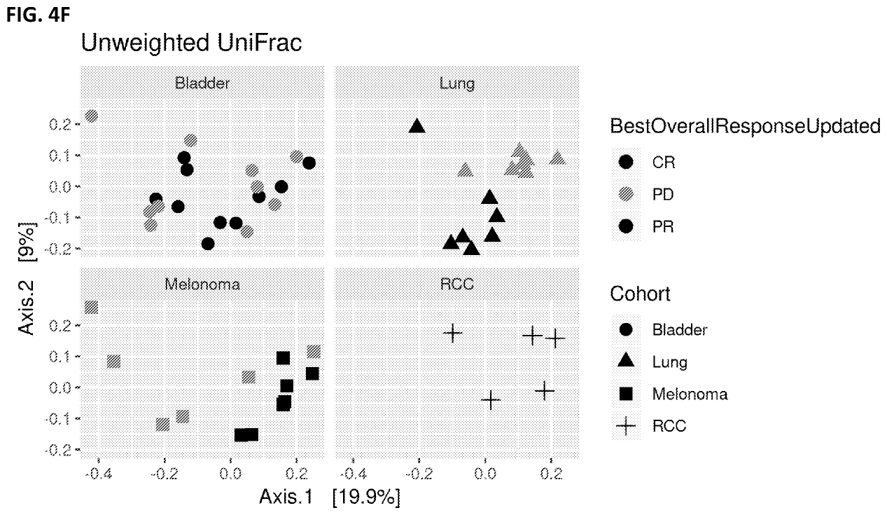

Weighted (FIGS. 3A-3G) and unweighted (FIGS. 4A-4H) Unifrac distance analysis were further conducted. In the weighted Unifrac distance analysis, Axis 1 explained 37.6% of the variance (FIG. 3A) but was not correlated with the R and NR outcomes (Wilcoxon test, p˜0.49, FIG. 3B). Table 2 shows the species having the lowest Axis 1 values.

TABLE 2
Species with lowest axis 1 values

| Species | Axis 1 value |
|---|---|
| Clostridioides_difficile | −0.28 |
| Fusobacterium_ulcerans | −0.26 |
| Anaeromassilibacillus_senegalensis | −0.26 |
| Lactococcus_piscium | −0.25 |
| [Enterococcus]_dispar | −0.24 |
| Prevotella_buccae | −0.24 |
| Streptococcus_infantarius_group | −0.23 |
| Fournierella_massiliensis | −0.23 |

However, the NMDS plot of the weighted UniFrac distance faceted by specific cancer types, i.e., bladder cancer, lung cancer, melanoma, and RCC showed distinct clustering of R and NR outcomes within individual cancer types (FIG. 3C). FIG. 3D depicts the NMDS plots of the weighted UniFrac index among several major phyla, i.e., Actinobacteria, Bacteroidetes, Firmicutes, and Proteobacteria. FIGS. 3E and 3F show the NDMS plots of the weighted UniFrac index by the best overall responses categories, i.e., 1) complete response, 2) progressive disease, and 3) partial response, overall (FIG. 3E) or faceted by specific cancer types (FIG. 3F). FIG. 3G demonstrated the supervised clustering to identify the top species with the R or NR outcomes based on the clustering of individual cancer types.

In the unweighted Unifrac distance analysis, Axis 2 explained 7.1% of the variance (FIG. 4A) and was significantly correlated with the R and NR outcomes (Wilcoxon test, p˜0.013, FIG. 4B). Table 3 shows the species having the lowest Axis 2 values.

TABLE 3
Species with lowest axis 2 values

| Species | Axis 2 value |
|---|---|
| [Enorma]_phocaeensis | −0.23 |
| Flavonifractor_sp1 | −0.23 |
| Lachnospiraceae_NG17_sp4 | −0.23 |
| Parabacteroides_timonensis | −0.22 |
| [Enterococcus]_avium | −0.17 |
| Prevotella_salivae | −0.17 |
| Lactobacillus_algidus | −0.17 |
| Clostridium_carnis_group | −0.17 |

The NMDS plot of the unweighted UniFrac distance faceted by specific cancer types, i.e., bladder cancer, lung cancer, melanoma, and RCC, showed clusters along Axis 2 (FIG. 4C). FIG. 4D depicts the NMDS plots of the unweighted UniFrac index among several major phyla, i.e., Actinobacteria, Bacteroidetes, Firmicutes, and Proteobacteria. FIGS. 4E and 4F show the NDMS plots of the unweighted UniFrac index by the best overall responses categories, i.e., 1) complete response, 2) progressive disease, and 3) partial response, overall (FIG. 4E) or faceted by specific cancer types (FIG. 4F).

Enrichment analysis revealed that across all cancer types, excluding RCC samples, families Sutterellaceae, Coriobacteriaceae, Akkermansiaceae, or Desulfovibrionaceae were significantly correlated with response (Table 4). Tables 4, 5 and 6 show the overall enrichment analysis results of the microbiome samples. Table 4 shows the top families associated with responders (R) and non-responder (NR) (i.e. Campylobacteraceae). Tables 5 and 6 show the top species associated with responders (R) and non-responders (NR) according to abundance (Table 5) and prevalence (Table 6), respectively.

TABLE 4
Top families associated with responders
(R) and non-responders (NR)

| Family | Unadj P-value | Mean % R | Mean % NR |
|---|---|---|---|
| Sutterellaceae | 0.0027 | 1.1 | 0.61 |
| Coriobacteriaceae | 0.0086 | 2.0 | 1.2 |
| Akkermansiaceae | 0.0099 | 1.9 | 0.60 |
| Campylobacteraceae | 0.013 | 0.00025 | 0.0023 |
| Desulfovibrionaceae | 0.027 | 0.51 | 0.40 |

TABLE 5
Top species associated with responders(R) and
non-responders (NR) according to abundance

| Top 5 enriched in R | | Top 5 enriched in NR | |
|---|---|---|---|
| Species | Unadj P-value | Species | Unadj P-value |
| Dysosmobacter_welbionis | 0.0061 | Holdemania_massiliensis | 0.0037 |
| Anaerobutyricum_hallii | 0.0069 | Rothia_mucilaginosa | 0.0042 |
| Blautia_caecimuris | 0.0093 | Hungatella_sp2 | 0.012 |
| Akkermansia_muciniphila | 0.010 | Prevotella_copri | 0.015 |
| Holdemania_filiformis | 0.012 | Campylobacter_concisus | 0.015 |

TABLE 6
Top species associated with responders (R) and
non-responders (NR) according to prevalence

| Top 5 enriched in R | | Top 5 enriched in NR | |
|---|---|---|---|
| Species | Unadj P-value | Species | Unadj P-value |
| Acetatifactor_sp1 | 0.0031 | Rothia_mucilaginosa | 0.0045 |
| Blautia_wexlerae | 0.0035 | [Atopobium]_parvulum | 0.0090 |
| Ruminococcaceae_NG14_sp1 | 0.0070 | Holdemania_massiliensis | 0.0093 |
| Hungatella_sp2 | 0.0089 | Prevotella_copri | 0.017 |
| Holdemania_filiformis | 0.019 | Campylobacter_concisus | 0.022 |

According to abundance-based analysis, Akkermansia muciniphila, Dysosmobacter welbionis, Anaerobutyricum hallii, Blautia caecimuris, and Holdemania filiformis were the top species enriched in the R group samples while Holdemania massiliensis, Rothia mucilaginosa, Hungatella sp2, Prevotella copri, and Campylobacter concisus were the top species enriched in the NR group samples. According to prevalence-based analysis, Acetatifactor sp1, Blautia wexlerae, Ruminococcaceae NG14 sp1, Hungatella sp2, and Holdemania filiformis were the top species enriched in the R group samples while Rothia mucilaginosa, [Atopobium]parvulum, Holdemania massiliensis, Prevotella copri, and Campylobacter concisus were the top species enriched in the NR group samples.

Tables 7, 8 and 9 show the enrichment analysis results of the microbiome samples by specific cancer types. Table 7 shows the result of the permutational multivariate analysis of variance (Permanova). Tables 8 and 9 show the top species associated with R (Table 8) and NR (Table 9) from supervised clustering by specific cancer types, i.e., urothelial (i.e., bladder), lung cancer (i.e., non-small cell lung cancer, NSCLC), and melanoma. As shown in Table 7, PERMANOVA test based on the Bray-Curtis dissimilarity index showed that the interaction of cancer types and outcomes (i.e., R or NR) explained 4.2% of the microbiome compositions with significance (p value˜0.023) but not the individual factors of cancer types or treatment outcomes.

Using supervised clustering demonstrated in FIG. 3H, the top species of each specific cancer type associated with response and non-response were identified, as shown in Tables 8 and 9.

TABLE 7
Permutational multivariate analysis of variance

| Variable | % Variance Explained | P-value |
|---|---|---|
| Cancer Type | 3.89 | 0.052 |
| Outcome | 1.29 | 0.16 |
| Cancer Type * Outcome | 4.152 | 0.023 |
| Total | 9.33 | |

TABLE 8
Top species associated with R from supervised
clustering by specific cancer types

| Species | Cancer Type |
|---|---|
| Bifidobacterium_catenulatum_group | Urothelial |
| Blautia_wexlerae | Urothelial |
| Hungatella_sp1 | Urothelial |
| Intestinibacter_bartlettii | Urothelial |
| Lachnospiraceae_NG31_sp1 | Urothelial |
| Lactonifactor_longoviformis | Urothelial |
| Leuconostoc_gelidum | Urothelial |
| Sutterella_wadsworthensis | Urothelial |
| [Extibacter]_muris | NSCLC |
| Alistipes_onderdonkii | NSCLC |
| Collinsella_intestinalis | NSCLC |
| Collinsella_stercoris | NSCLC |
| Muricomes_sp1 | NSCLC |
| Neobitarella_massiliensis | NSCLC |
| Prevotellamassilia_sp2 | NSCLC |
| Ruminococcaceae_NG33_sp1 | NSCLC |
| [Adlercreutzia]_equolifaciens_group | Melanoma |
| [Anaerotruncus]_rubiinfantis | Melanoma |
| [Romboutsia]_timonensis | Melanoma |
| Alistipes_finegoldii | Melanoma |
| Alistipes_indistinctus | Melanoma |
| Alistipes_obesi | Melanoma |
| Alistipes_putredinis | Melanoma |
| Alistipes_shahii | Melanoma |
| Anaerofustis_stercorihominis | Melanoma |
| Anaeromassilibacillus_sp3 | Melanoma |
| Barnesiella_intestinihominis | Melanoma |
| Bilophila_wadsworthia | Melanoma |
| Blautia_faecis | Melanoma |
| Coriobacteriaceae_NG5_sp1 | Melanoma |
| Frisingicoccus_sp3 | Melanoma |
| Gordonibacter_pamelaeae | Melanoma |
| Harryflintia_acetispora | Melanoma |
| Lachnospiraceae_NG29_sp1 | Melanoma |
| Marvinbryantia_sp8 | Melanoma |
| Massilimaliae_massiliensis | Melanoma |
| Massilimaliae_timonensis | Melanoma |
| Neglecta_timonensis | Melanoma |
| Parabacteroides_merdae | Melanoma |
| Peptoniphilaceae_NG4_sp1 | Melanoma |
| Peptoniphilaceae_NG4_sp3 | Melanoma |
| Pseudoflavonifractor_capillosus | Melanoma |
| Ruminococcaceae_NG12_sp1 | Melanoma |
| Ruminococcaceae_NG15_sp3 | Melanoma |
| Ruminococcaceae_NG17_sp3 | Melanoma |
| Ruthenibacterium_sp1 | Melanoma |

TABLE 9
Top species associated with NR from supervised
clustering by specific cancer types

| Species | Cancer Type |
|---|---|
| Alistipes_onderdonkii | Urothelial |
| Angelakisella_massiliensis | Urothelial |
| Christensenellaceae_NG5_sp2 | Urothelial |
| Cuneatibacter_caecimuris | Urothelial |
| Desulfovibrio_fairfieldensis | Urothelial |
| Ruminococcaceae_NG13_sp1 | Urothelial |
| Ruminococcaceae_NG16_sp1 | Urothelial |
| [Coprococcus]_catus | NSCLC |
| [Lactobacillus]_fermentum | NSCLC |
| [Romboutsia]_timonensis | NSCLC |
| [Ruminococcus]_bromii | NSCLC |
| [Ruminococcus]_gauvreauii | NSCLC |
| Actinomyces_graevenitzii | NSCLC |
| Agathobaculum_butyriciproducens_group | NSCLC |
| Bacteroides_caccae | NSCLC |
| Bacteroides_intestinalis | NSCLC |
| Bittarella_massiliensis | NSCLC |
| Blautia_luti_group | NSCLC |
| Clostridium_saudiense | NSCLC |
| Corynebacterium_durum | NSCLC |
| Fusicatenibacter_sp2 | NSCLC |
| Lachnospiraceae_NG30_sp3 | NSCLC |
| Massilimaliae_sp1 | NSCLC |
| Monoglobus_pectinilyticus | NSCLC |
| Rothia_dentocariosa | NSCLC |
| Rothia_mucilaginosa | NSCLC |
| Ruminococcaceae_NG17_sp1 | NSCLC |
| Schaalia_odontolytica | NSCLC |
| Streptococcus_australis | NSCLC |
| Streptococcus_gordonii | NSCLC |
| Streptococcus_infantis_group | NSCLC |
| Streptococcus_parasanguinis | NSCLC |
| Streptococcus_salivarius_group | NSCLC |
| [Atopobium]_parvulum | Melanoma |
| [Ruminococcus]_gnavus | Melanoma |
| Bacteroides_dorei_group | Melanoma |
| Blautia_sp1 | Melanoma |
| Schaalia_sp2 | Melanoma |

The variance in the composition of pretreatment microbiome samples did not explain response alone (R vs NR, PERMANOVA based on the weighted UniFrac distance, p=0.273), a significant portion of the variance in microbiome composition was explained by the interaction of cancer type and outcome (PERMANOVA, p=0.014), suggesting a cancer-specific microbiome relationship.

### Example 2: In Vivo Tumor Models with Non-Responder Fecal Matter Transplants

A subset of the microbiome samples identified in this NR cluster were evaluated using in vivo preclinical models. Colonization of germ-free mice with stool from NR patients elicited a nonresponsive result in syngeneic tumors treated with CPI in contrast to stool from healthy subjects, which confers anti-tumor response.

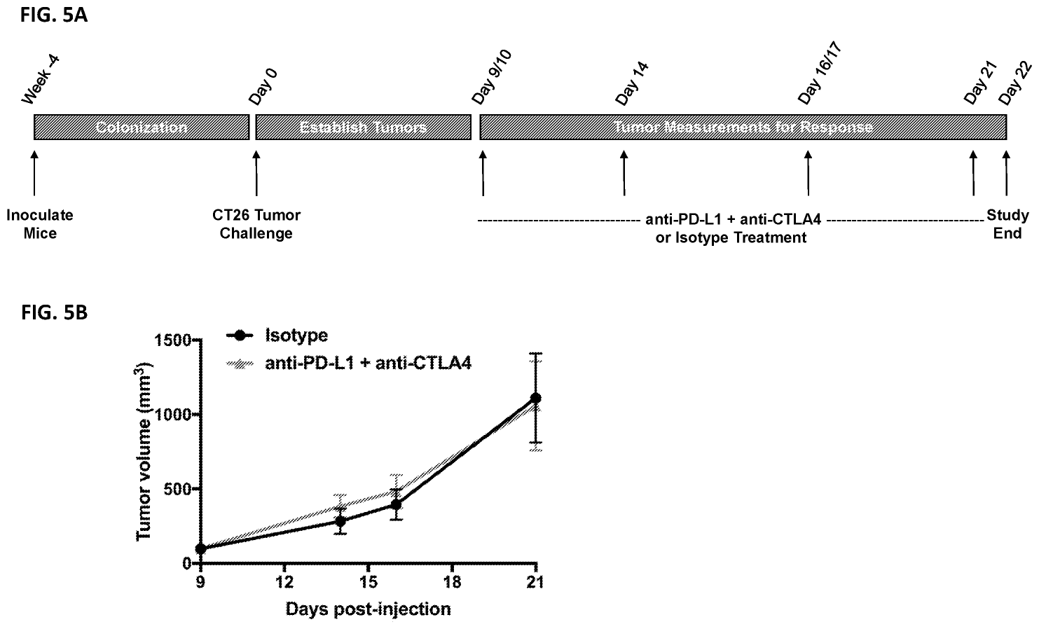

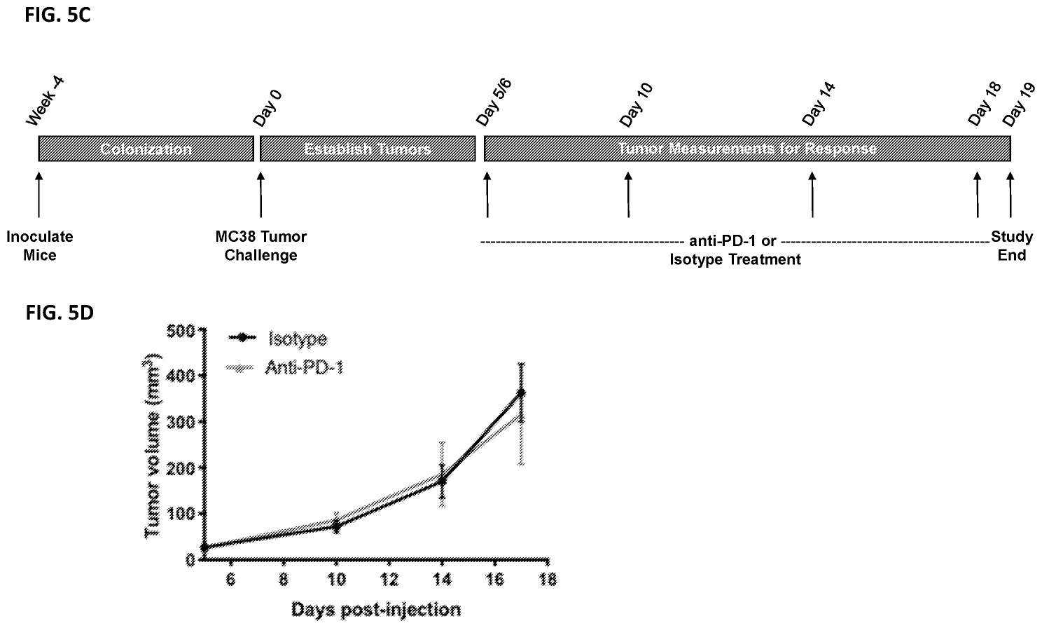

As shown in FIG. 5A, germ-free mice models were inoculated at week −4 with the stool samples from a lung cancer patient who was non-responsive to checkpoint therapy and anti-PD-L1+anti-CTLA4 combination therapy. At Day 0, the mice models were challenged with CT26 tumors. After 9-10 days of tumor establishment, anti-PD-L1+anti-CTLA4 combination therapy or isotype treatment was administered at Day 9/10, Day 14, Day 16/17, and Day 21 before the mice models were sacrificed at Day 22 for analysis. The analysis results, as shown in FIG. 5B, suggests the lack of efficacy in mice colonized with stool from a lung cancer patient who was non-responsive to checkpoint therapy and anti-PD-L1+anti-CTLA4 combination therapy in treating CT26 tumors in an animal model.

Similar tests were conducted in MC38 tumor models. Briefly, as shown in FIG. 5C, germ-free mice models were inoculated at week −4 with the stool samples from a lung cancer patient who was non-responsive to checkpoint therapy and anti-PD-L1 monotherapy. At Day 0, the mice models were challenged with MC38 tumors. After 5-6 days of tumor establishment, anti-PD-L1 therapy or isotype treatment was administered at Day 5/6, Day 10, Day 14, and Day 18 before the mice models were sacrificed at Day 19 for analysis. The analysis results, as shown in FIG. 5D, suggests the lack of efficacy in mice colonized with stool from a lung cancer patient who was non-responsive to checkpoint therapy and anti-PD-L1 monotherapy in treating MC38 tumors in an animal model.

Conclusions: Analysis of the gut microbiome composition from patients with metastatic lung cancer, urothelial cancer, renal cell carcinoma, and metastatic melanoma identified a cancer-specific signature of R and NR to CPI. Across three cancer types, a consistent signature of NR was identified and corroborated experimentally in preclinical animal models.

### Example 3: Bacterial Taxonomic Correlates with Outcome

Gut microbiome samples were obtained from patients with metastatic melanoma, lung, urothelial, or renal cell cancer immediately before CPI therapy was initiated (as described in example 1). Clinical characteristics were obtained from patient medical records; clinical response to CPI was defined as patients obtaining a partial or complete response or remaining on therapy for more than 6 months.

Shotgun whole metagenomic sequencing of patient stool samples were analyzed via Metaphlan to produce relative abundance of different taxonomic groupings including species, genera, and families. Differential abundance of these taxa between responders and non-responders was tested via non-parametric Wilcoxon ranksum tests and parametric t-tests. There were 112 samples included in this analysis (50NR, 62R). Many taxa were nominally associated with outcome at p<0.05.

The results of Wilcoxon rank sum non-parametric test for association between quantitative abundance of a taxon against outcome (responder vs non-responder to ICI therapy) are shown in Table 10. This analysis was also performed for individual cancer types: urothelial, lung, and melanoma (see Tables 11-13).

Thus, the quantitative abundances of several bacterial species are associated with patient response or non-response to CPI when considering all cancer types jointly or when considering urothelial, lung, and melanoma cancer types separately. Differences in taxonomic association among individual cancer types is in agreement with analysis in Example 1 where there is a significant interaction between cancer type and response in regard to microbiome composition. This indicates the relative abundance of bacterial species may be important to response to ICI in different cancer indications, with certain taxa potentially promoting response and certain taxa potentially preventing response.

TABLE 10
Direct associations between taxa abundance with outcome

| Taxa | Taxa Level | wc.pval | Mean.NR | Mean.R |
|---|---|---|---|---|
| Sutterellaceae | Family | 0.000665 | 0.568621761 | 1.041308053 |
| Roseburia_intestinalis | Species | 0.001784 | 0.177107026 | 0.637295571 |
| Hungatella_sp1 | Species | 0.003606 | 0.089281375 | 0.239019008 |
| Lactobacillus | Genus | 0.003671 | 0.098630968 | 0.034492386 |
| Coriobacteriaceae_NG1 | Genus | 0.005854 | 0.018613172 | 0.014840513 |
| Blautia_wexlerae | Species | 0.006317 | 1.346078627 | 2.062722529 |
| [Atopobium]_parvulum | Species | 0.006819 | 0.002140907 | 0.00268814 |
| Ruminococcaceae_NG13_sp1 | Species | 0.008767 | 0.640427595 | 0.133612395 |
| Lactobacilluseae | Family | 0.011059 | 0.214612881 | 0.054915972 |
| Negativibacillus_sp1 | Species | 0.01185 | 0.003491553 | 0.040363482 |
| Lachnospiraceae | Family | 0.012143 | 18.96092172 | 23.41022483 |
| Christensenella_timonensis | Species | 0.013255 | 0.000829049 | 5.10474E−05 |
| Lachnospiraceae_NG18_sp7 | Species | 0.01415 | 0.000837434 | 0.040002837 |
| Rothia_mucilaginosa | Species | 0.01546 | 0.007089752 | 0.005687923 |
| [Lactobacillus]_paracasei | Species | 0.017934 | 0.037207492 | 0.01007938 |
| Propionibacterium_freudenreichii | Species | 0.021432 | 0.004878945 | 0.000352685 |
| Prevotella_buccalis | Species | 0.024029 | 0.000887426 | 6.69363E−05 |
| Agathobaculum | Genus | 0.024411 | 0.147184416 | 0.219885696 |
| Holdemania_massiliensis | Species | 0.025973 | 0.013800455 | 0.00301813 |
| Ruminococcaceae_NG18_sp7 | Species | 0.02608 | 0.075158206 | 0.007105781 |
| Lactobacillus_delbrueckii | Species | 0.026893 | 0.041643503 | 0.000775484 |
| Scardovia | Genus | 0.027061 | 0.000923478 | 0.000140386 |
| Actinomycetaceae_NG7_sp1 | Species | 0.028012 | 0.000123021 | 8.77419E−05 |
| Actinomycetaceae_NG7 | Genus | 0.028012 | 0.000123021 | 8.77419E−05 |
| Agathobaculum_butyriciproducens_group | Species | 0.029575 | 0.124675699 | 0.19633037 |
| Lachnospiraceae_NG17 | Genus | 0.030953 | 0.104924489 | 0.180521288 |
| Campylobacter | Genus | 0.037792 | 0.002252076 | 0.000666115 |
| Campylobacteraceae | Family | 0.037792 | 0.002252076 | 0.000666115 |
| Neobitarella_massiliensis | Species | 0.041278 | 0.000990089 | 0.001722737 |
| Neobitarella | Genus | 0.041278 | 0.000990089 | 0.001722737 |
| Bacteroides_coprocola | Species | 0.041904 | 0.583587343 | 0.088604183 |
| [Clostridium]_asparagiforme_group | Species | 0.041962 | 0.108863981 | 0.064622461 |
| Anaerobutyricum | Genus | 0.04213 | 0.490307117 | 0.806618606 |
| Ruminococcaceae_NG11 | Genus | 0.042909 | 0.251108242 | 0.437416925 |
| [Lactobacillus]_reuteri | Species | 0.043639 | 0.006739489 | 0.00308371 |
| Rothia | Genus | 0.044094 | 0.00794458 | 0.006682386 |
| Micrococcaceae | Family | 0.044094 | 0.00794458 | 0.006682386 |
| Roseburia | Genus | 0.044978 | 3.443646555 | 4.463988005 |
| Negativibacillus | Genus | 0.045504 | 0.118568768 | 0.119489079 |
| Lachnospiraceae_NG31_sp1 | Species | 0.046349 | 0.005526622 | 0.041640896 |
| Lachnospiraceae_NG31 | Genus | 0.046349 | 0.005526622 | 0.041640896 |
| Propionibacterium | Genus | 0.047427 | 0.004988621 | 0.000384194 |
| Propionibacteriaceae | Family | 0.047427 | 0.004992445 | 0.000384194 |
| Lactobacilluseae_NG5 | Genus | 0.048174 | 0.115981913 | 0.020423586 |
| Dialister_invisus | Species | 0.049692 | 0.103947311 | 0.374285534 |

| Taxa | mean.diff | mean.log2ratio | wc.qval | wc.qval.global |
|---|---|---|---|---|
| Sutterellaceae | 0.472686 | 0.872855711 | 0.027937816 | 0.422393175 |
| Roseburia_intestinalis | 0.460189 | 1.847341186 | 0.65162402 | 0.566425524 |
| Hungatella_sp1 | 0.149738 | 1.420694202 | 0.65162402 | 0.582807457 |
| Lactobacillus | −0.06414 | −1.515762758 | 0.541449849 | 0.582807457 |
| Coriobacteriaceae_NG1 | −0.00377 | −0.326782996 | 0.541449849 | 0.608501842 |
| Blautia_wexlerae | 0.716644 | 0.615787085 | 0.65162402 | 0.608501842 |
| [Atopobium]_parvulum | 0.000547 | 0.328385876 | 0.65162402 | 0.608501842 |
| Ruminococcaceae_NG13_sp1 | −0.50682 | −2.260981625 | 0.65162402 | 0.608501842 |
| Lactobacilluseae | −0.1597 | −1.966438948 | 0.170000424 | 0.608501842 |
| Negativibacillus_sp1 | 0.036872 | 3.531109863 | 0.65162402 | 0.608501842 |
| Lachnospiraceae | 4.449303 | 0.304109693 | 0.170000424 | 0.608501842 |
| Christensenella_timonensis | −0.00078 | −4.021549189 | 0.65162402 | 0.608501842 |
| Lachnospiraceae_NG18_sp7 | 0.039165 | 5.577983565 | 0.65162402 | 0.608501842 |
| Rothia_mucilaginosa | −0.0014 | −0.317833019 | 0.65162402 | 0.608501842 |
| [Lactobacillus]_paracasei | −0.02713 | −1.884186257 | 0.65162402 | 0.608501842 |
| Propionibacterium_freudenreichii | −0.00453 | −3.790117001 | 0.65162402 | 0.608501842 |
| Prevotella_buccalis | −0.00082 | −3.728766071 | 0.65162402 | 0.608501842 |
| Agathobaculum | 0.072701 | 0.579128831 | 0.55701311 | 0.608501842 |
| Holdemania_massiliensis | −0.01078 | −2.192989021 | 0.65162402 | 0.608501842 |
| Ruminococcaceae_NG18_sp7 | −0.06805 | −3.402865452 | 0.65162402 | 0.608501842 |
| Lactobacillus_delbrueckii | −0.04087 | −5.746850736 | 0.65162402 | 0.608501842 |
| Scardovia | −0.00078 | −2.717674556 | 0.55701311 | 0.608501842 |
| Actinomycetaceae_NG7_sp1 | −3.5E−05 | −0.487561673 | 0.65162402 | 0.608501842 |
| Actinomycetaceae_NG7 | −3.5E−05 | −0.487561673 | 0.55701311 | 0.608501842 |
| Agathobaculum_butyriciproducens_group | 0.071655 | 0.655103071 | 0.65162402 | 0.608501842 |
| Lachnospiraceae_NG17 | 0.075597 | 0.782817544 | 0.55701311 | 0.608501842 |
| Campylobacter | −0.00159 | −1.757412229 | 0.55701311 | 0.608501842 |
| Campylobacteraceae | −0.00159 | −1.757412229 | 0.331988138 | 0.608501842 |
| Neobitarella_massiliensis | 0.000733 | 0.79907325 | 0.65162402 | 0.608501842 |
| Neobitarella | 0.000733 | 0.79907325 | 0.55701311 | 0.608501842 |
| Bacteroides_coprocola | −0.49498 | −2.719501872 | 0.65162402 | 0.608501842 |
| [Clostridium]_asparagiforme_group | −0.04424 | −0.752419114 | 0.65162402 | 0.608501842 |
| Anaerobutyricum | 0.316311 | 0.718200981 | 0.55701311 | 0.608501842 |
| Ruminococcaceae_NG11 | 0.186309 | 0.80069966 | 0.55701311 | 0.608501842 |
| [Lactobacillus]_reuteri | −0.00366 | −1.127972137 | 0.65162402 | 0.608501842 |
| Rothia | −0.00126 | −0.249607606 | 0.55701311 | 0.608501842 |
| Micrococcaceae | −0.00126 | −0.249607606 | 0.331988138 | 0.608501842 |
| Roseburia | 1.020341 | 0.374396074 | 0.55701311 | 0.608501842 |
| Negativibacillus | 0.00092 | 0.011154719 | 0.55701311 | 0.608501842 |
| Lachnospiraceae_NG31_sp1 | 0.036114 | 2.913531193 | 0.65162402 | 0.608501842 |
| Lachnospiraceae_NG31 | 0.036114 | 2.913531193 | 0.55701311 | 0.608501842 |
| Propionibacterium | −0.0046 | −3.698735485 | 0.55701311 | 0.608501842 |
| Propionibacteriaceae | −0.00461 | −3.699841208 | 0.331988138 | 0.608501842 |
| Lactobacilluseae_NG5 | −0.09556 | −2.505591732 | 0.55701311 | 0.608501842 |
| Dialister_invisus | 0.270338 | 1.848286856 | 0.65162402 | 0.608501842 |

Taxa (name), Taxa Level (species, genus, or family level taxonomic unit), wc.pval (raw p-value from wilcoxon test), Mean.NR (mean abundance of non-responders), mean.R (mean abundance of responders), mean.diff (mean of responders minus mean of non-responders, positive is higher in responder), mean.log2ratio (log2 fold-change of mean responder to mean non-responder, positive is higher in responder), wc.qval (FDR corrected q-value, Benjamini-Hochberg procedure, for analysis WITHIN taxon level), wc.qval.global (FDR corrected q-value, Benjamini-Hochberg procedure, for analysis considering ALL taxon levels)

TABLE 11
Direct associations between taxa abundance with outcome for urothelial cancer

| | Taxa | | | | | | | |
|---|---|---|---|---|---|---|---|---|
| Taxa | Level | wc.pval | Mean.NR | Mean.R | mean.diff | mean.log2ratio | wc.qval | wc.qval.global |
| Lachnospiraceae | Family | 0.000123 | 15.10219 | 27.63152 | 12.52933 | 0.87155735 | 0.005161 | 0.082204496 |
| Ruminococcaceae_NG11 | Genus | 0.000952 | 0.020834 | 0.67852 | 0.657686 | 5.025386625 | 0.093183 | 0.213127352 |
| Anaerostipes | Genus | 0.000956 | 0.42348 | 1.429287 | 1.005808 | 1.754931573 | 0.093183 | 0.213127352 |
| Roseburia | Genus | 0.001859 | 1.882095 | 4.483526 | 2.601431 | 1.252294483 | 0.120848 | 0.310950189 |
| Anaerostipes_hadrus | Species | 0.004781 | 0.372776 | 1.389347 | 1.016571 | 1.898026145 | 0.748495 | 0.639652174 |
| Prevotellaceae | Family | 0.006194 | 1.877201 | 0.478379 | −1.39882 | −1.97235643 | 0.130065 | 0.684175153 |
| Blautia_wexlerae | Species | 0.007159 | 0.666925 | 2.973063 | 2.306138 | 2.156353877 | 0.748495 | 0.684175153 |
| Intestinibacter_bartlettii | Species | 0.009799 | 0.116416 | 0.123506 | 0.00709 | 0.085289281 | 0.748495 | 0.76522366 |
| Faecalibacterium_prausnitzii | Species | 0.011493 | 0.84697 | 0.073341 | −0.77363 | −3.529622751 | 0.748495 | 0.76522366 |
| Ruminococcaceae_NG11_sp5 | Species | 0.012999 | 0.020323 | 0.436265 | 0.415942 | 4.424034047 | 0.748495 | 0.76522366 |
| Roseburia_faecis | Species | 0.02043 | 0.649866 | 1.662439 | 1.012572 | 1.355085896 | 0.748495 | 0.76522366 |
| Prevotella | Genus | 0.02324 | 1.24957 | 0.362958 | −0.88661 | −1.783557357 | 0.775672 | 0.76522366 |
| Roseburia_intestinalis | Species | 0.027748 | 0.14802 | 0.821333 | 0.673313 | 2.472174617 | 0.748495 | 0.76522366 |
| Lachnospiraceae_NG13 | Genus | 0.03095 | 0.451904 | 0.818482 | 0.366578 | 0.856935078 | 0.775672 | 0.76522366 |
| [Merdimonas]_faecis | Species | 0.031222 | 0 | 0.016375 | 0.016375 | | 0.748495 | 0.76522366 |
| Bifidobacterium_animalis | Species | 0.031508 | 0.018062 | 0 | −0.01806 | | 0.748495 | 0.76522366 |
| Ruminococcaceae_NG13_sp1 | Species | 0.031508 | 0.983704 | 0 | −0.9837 | | 0.748495 | 0.76522366 |
| [Ruminococcus]_gnavus | Species | 0.035404 | 0.274483 | 1.46711 | 1.192627 | 2.418189612 | 0.748495 | 0.76522366 |
| Dialister_invisus | Species | 0.044415 | 0.076548 | 0.466525 | 0.389977 | 2.607512134 | 0.748495 | 0.76522366 |
| Leuconostoc_gelidum | Species | 0.046748 | 2.67E−05 | 0.001726 | 0.001699 | 6.016176563 | 0.748495 | 0.76522366 |
| Alistipes_finegoldii | Species | 0.048256 | 0.910167 | 0.162864 | −0.7473 | −2.482466479 | 0.748495 | 0.76522366 |

TABLE 12
Direct associations between taxa abundance with outcome for lung cancer

| | Taxa | | | | | | | |
|---|---|---|---|---|---|---|---|---|
| Taxa | Level | wc.pval | Mean.NR | Mean.R | mean.diff | mean.log2ratio | wc.qval | wc.qval.global |
| Holdemania_massiliensis | Species | 0.00203 | 0.020713 | 0.002395 | −0.01832 | −3.11233 | 0.675926 | 0.70402 |
| Lactobacillus | Genus | 0.005552 | 0.177023 | 0.062739 | −0.11428 | −1.49651 | 0.642538 | 0.70402 |
| Propionibacterium_freudenreichii | Species | 0.00603 | 0.011005 | 8.26E−06 | −0.011 | −10.3795 | 0.675926 | 0.70402 |
| Bacteroides_intestinalis | Species | 0.010737 | 0.063218 | 0.004166 | −0.05905 | −3.92371 | 0.675926 | 0.70402 |
| Thermodesulfobiaceae_NG1_sp1 | Species | 0.01096 | 0.653097 | 0.056735 | −0.59636 | −3.52499 | 0.675926 | 0.70402 |
| Thermodesulfobiaceae_NG1 | Genus | 0.01096 | 0.653097 | 0.056735 | −0.59636 | −3.52499 | 0.642538 | 0.70402 |
| Thermodesulfobiaceae | Family | 0.01096 | 0.653097 | 0.056735 | −0.59636 | −3.52499 | 0.232311 | 0.70402 |
| Gemmiger_sp2 | Species | 0.013732 | 1.256942 | 0.437732 | −0.81921 | −1.5218 | 0.675926 | 0.70402 |
| Coprococcus | Genus | 0.015609 | 0.834732 | 0.005031 | −0.8297 | −7.3742 | 0.642538 | 0.70402 |
| Sutterellaceae | Family | 0.0158 | 0.579347 | 1.267489 | 0.688142 | 1.129473 | 0.232311 | 0.70402 |
| Parabacteroides_merdae | Species | 0.016637 | 1.132578 | 0.500235 | −0.63234 | −1.17893 | 0.675926 | 0.70402 |
| Coriobacteriaceae_NG1 | Genus | 0.016801 | 0.037141 | 0.00645 | −0.03069 | −2.52562 | 0.642538 | 0.70402 |
| Propionibacterium | Genus | 0.016998 | 0.011109 | 4.54E−05 | −0.01106 | −7.93598 | 0.642538 | 0.70402 |
| Propionibacteriaceae | Family | 0.016998 | 0.011118 | 4.54E−05 | −0.01107 | −7.93711 | 0.232311 | 0.70402 |
| Intestinimonas_massiliensis | Species | 0.02133 | 0.072559 | 0.009274 | −0.06329 | −2.96785 | 0.675926 | 0.70402 |
| Alistipes_shahii | Species | 0.021461 | 0.864728 | 0.367111 | −0.49762 | −1.23603 | 0.675926 | 0.70402 |
| Gemmiger_Subdoligranulum | Genus | 0.025745 | 1.76899 | 0.652741 | −1.11625 | −1.43834 | 0.730523 | 0.70402 |
| Marvinbryantia_sp5 | Species | 0.026562 | 0.029754 | 0.008017 | −0.02174 | −1.89197 | 0.675926 | 0.70402 |
| [Anaerotruncus]_rubiinfantis | Species | 0.027056 | 0.011209 | 0.003505 | −0.0077 | −1.6771 | 0.675926 | 0.70402 |
| Marasmitruncus | Genus | 0.027056 | 0.011209 | 0.003505 | −0.0077 | −1.6771 | 0.730523 | 0.70402 |
| Bacteroides_massiliensis | Species | 0.035938 | 1.531609 | 0.688603 | −0.84301 | −1.1533 | 0.675926 | 0.70402 |
| Desulfovibrionaceae_NG5_sp1 | Species | 0.036386 | 0.084696 | 0 | −0.0847 | | 0.675926 | 0.70402 |
| Desulfovibrionaceae_NG5 | Genus | 0.036386 | 0.084696 | 0 | −0.0847 | | 0.795987 | 0.70402 |
| Streptococcus_parasanguinis | Species | 0.036858 | 0.219655 | 0.28578 | 0.066125 | 0.379664 | 0.675926 | 0.70402 |
| Blautia_caecimuris | Species | 0.043334 | 0.187781 | 0.660192 | 0.472411 | 1.813833 | 0.675926 | 0.70402 |
| Bacteroides_faecis | Species | 0.044183 | 0.667635 | 0.0416 | −0.62603 | −4.0044 | 0.675926 | 0.70402 |
| Roseburia_intestinalis | Species | 0.044476 | 0.117159 | 0.72297 | 0.605812 | 2.625472 | 0.675926 | 0.70402 |
| Desulfovibrio_desulfuricans | Species | 0.045435 | 0 | 0.021986 | 0.021986 | | 0.675926 | 0.70402 |
| [Allisonella]_histaminiformans | Species | 0.045759 | 0.013993 | 0.001585 | −0.01241 | −3.14237 | 0.675926 | 0.70402 |
| Lactobacillaceae | Family | 0.047158 | 0.380163 | 0.091366 | −0.2888 | −2.0569 | 0.350657 | 0.70402 |
| [Ruminococcus]_gauvreauii | Species | 0.047434 | 0.000436 | 1.91E−05 | −0.00042 | −4.50876 | 0.675926 | 0.70402 |
| Prevotella_copri | Species | 0.049807 | 1.185374 | 1.391739 | 0.206366 | 0.231547 | 0.675926 | 0.70402 |
| Slackia_isoflavoniconvertens | Species | 0.049807 | 0.058124 | 0.01394 | −0.04418 | −2.05985 | 0.675926 | 0.70402 |

TABLE 13
Direct associations between taxa abundance with outcome for melanoma

| | Taxa | | | | | | | |
|---|---|---|---|---|---|---|---|---|
| Taxa | Level | wc.pval | Mean.NR | Mean.R | mean.diff | mean.log2ratio | wc.qval | wc.qval.global |
| Gordonibacter_pamelaeae | Species | 0.003238 | 0 | 0.015457 | 0.015457 | | 0.615778 | 0.620905 |
| Lactococcus | Genus | 0.004085 | 0.09566 | 0.0004 | −0.09526 | −7.90327 | 0.458511 | 0.620905 |
| Lactococcus_lactis | Species | 0.005191 | 0.062695 | 0.0004 | −0.0623 | −7.29369 | 0.615778 | 0.620905 |
| Coriobacteriaceae_NG5_sp1 | Species | 0.00656 | 0 | 0.173427 | 0.173427 | | 0.615778 | 0.620905 |
| Odoribacteraceae | Family | 0.006702 | 0.201887 | 0.834257 | 0.63237 | 2.046943 | 0.272832 | 0.620905 |
| Odoribacter_splanchnicus | Species | 0.010752 | 0.162764 | 0.574776 | 0.412012 | 1.820215 | 0.615778 | 0.620905 |
| Parabacteroides_merdae | Species | 0.010752 | 0.190125 | 0.993755 | 0.803631 | 2.385944 | 0.615778 | 0.620905 |
| Odoribacter | Genus | 0.010752 | 0.162764 | 0.574776 | 0.412012 | 1.820215 | 0.458511 | 0.620905 |
| [Adlercreutzia]_equolifaciens_group | Species | 0.012231 | 0.032452 | 0.118215 | 0.085763 | 1.865032 | 0.615778 | 0.620905 |
| Adlercreutzia_Asaccharobacter | Genus | 0.012231 | 0.032452 | 0.118215 | 0.085763 | 1.865032 | 0.458511 | 0.620905 |
| Ruminococcaceae_NG15_sp3 | Species | 0.012629 | 0 | 0.198694 | 0.198694 | | 0.615778 | 0.620905 |
| Coriobacteriaceae | Family | 0.012847 | 0.519882 | 1.221923 | 0.702042 | 1.232898 | 0.272832 | 0.620905 |
| Ruminococcaceae_NG12_sp1 | Species | 0.013906 | 2.13E−05 | 0.005941 | 0.005919 | 8.126983 | 0.615778 | 0.620905 |
| Ruminococcaceae_NG12 | Genus | 0.013906 | 2.13E−05 | 0.005941 | 0.005919 | 8.126983 | 0.458511 | 0.620905 |
| Alistipes_putredinis | Species | 0.014737 | 0.479456 | 2.780926 | 2.30147 | 2.536095 | 0.615778 | 0.620905 |
| Barnesiella_intestinihominis | Species | 0.017073 | 0.14669 | 1.182584 | 1.035894 | 3.011099 | 0.615778 | 0.620905 |
| Barnesiella | Genus | 0.017073 | 0.14669 | 1.182584 | 1.035894 | 3.011099 | 0.458511 | 0.620905 |
| [Anaerotruncus]_rubiinfantis | Species | 0.017391 | 0.001803 | 0.011508 | 0.009705 | 2.674266 | 0.615778 | 0.620905 |
| Marasmitruncus | Genus | 0.017391 | 0.001803 | 0.011508 | 0.009705 | 2.674266 | 0.458511 | 0.620905 |
| Bilophila_wadsworthia | Species | 0.019086 | 0.07381 | 0.354725 | 0.280916 | 2.264817 | 0.615778 | 0.620905 |
| Bilophila | Genus | 0.019086 | 0.07381 | 0.354725 | 0.280916 | 2.264817 | 0.458511 | 0.620905 |
| Desulfovibrionaceae | Family | 0.021852 | 0.183389 | 0.517787 | 0.334398 | 1.497454 | 0.272832 | 0.620905 |
| Granulicatella_adiacens | Species | 0.02322 | 0.002723 | 0.000595 | −0.00213 | −2.19528 | 0.615778 | 0.620905 |
| Granulicatella | Genus | 0.02322 | 0.002723 | 0.000595 | −0.00213 | −2.19528 | 0.458511 | 0.620905 |
| Lachnoclostridium | Genus | 0.02322 | 0 | 0.00747 | 0.00747 | | 0.458511 | 0.620905 |
| Ruminococcaceae_NG11 | Genus | 0.02322 | 0 | 0.79652 | 0.79652 | | 0.458511 | 0.620905 |
| Aerococcaceae | Family | 0.02322 | 0.002723 | 0.000595 | −0.00213 | −2.19528 | 0.272832 | 0.620905 |
| Ruthenibacterium | Genus | 0.024017 | 0.138317 | 0.482226 | 0.343908 | 1.801725 | 0.458511 | 0.620905 |
| Massilimaliae_massiliensis | Species | 0.028814 | 0.000238 | 0.002857 | 0.002619 | 3.582739 | 0.615778 | 0.620905 |
| Coriobacteriaceae_NG5 | Genus | 0.028814 | 0.021528 | 0.177794 | 0.156266 | 3.0459 | 0.504248 | 0.620905 |
| Lachnospira_pectinoschiza | Species | 0.031082 | 0.045962 | 0.418858 | 0.372896 | 3.187942 | 0.615778 | 0.620905 |
| Catabacter_Christensenella | Genus | 0.031436 | 0.00033 | 0.008129 | 0.007799 | 4.620971 | 0.507814 | 0.620905 |
| Ruthenibacterium_lactatiformans | Species | 0.031511 | 0.130265 | 0.440734 | 0.310469 | 1.758454 | 0.615778 | 0.620905 |
| Ruthenibacterium_sp1 | Species | 0.035503 | 0.008052 | 0.041491 | 0.033439 | 2.365393 | 0.615778 | 0.620905 |
| Alistipes_finegoldii | Species | 0.037039 | 0.059595 | 1.12569 | 1.066095 | 4.239474 | 0.615778 | 0.620905 |
| Massilimaliae_timonensis | Species | 0.037039 | 0.000458 | 0.004091 | 0.003633 | 3.159679 | 0.615778 | 0.620905 |
| Anaerofustis_stercorihominis | Species | 0.040999 | 0 | 0.00191 | 0.00191 | | 0.615778 | 0.620905 |
| Lachnoclostridium_edouardi | Species | 0.040999 | 0 | 0.002753 | 0.002753 | | 0.615778 | 0.620905 |
| Scardovia_wiggsiae | Species | 0.040999 | 0.000683 | 2.57E−05 | −0.00066 | −4.7301 | 0.615778 | 0.620905 |
| Anaerofustis | Genus | 0.040999 | 0 | 0.00191 | 0.00191 | | 0.51685 | 0.620905 |
| Scardovia | Genus | 0.040999 | 0.000683 | 2.57E−05 | −0.00066 | −4.7301 | 0.51685 | 0.620905 |
| Lachnospiraceae_NG23 | Genus | 0.04256 | 0.003895 | 0.013988 | 0.010093 | 1.844422 | 0.51685 | 0.620905 |
| Lawsonibacter_sp7 | Species | 0.043829 | 0.025363 | 0.06054 | 0.035177 | 1.255141 | 0.615778 | 0.620905 |
| Rikenellaceae | Family | 0.044008 | 4.778801 | 9.439906 | 4.661105 | 0.982124 | 0.353029 | 0.620905 |
| Rothia_mucilaginosa | Species | 0.045683 | 0.002854 | 0.003089 | 0.000234 | 0.113785 | 0.615778 | 0.620905 |

Tables 11-13. Taxa (name), Taxa Level (species, genus, or family level taxonomic unit), wc.pval (raw p-value from wilcoxon test), Mean.NR (mean abundance of non-responders), mean.R (mean abundance of responders), mean.diff (mean of responders minus mean of non-responders, positive is higher in responder), mean.log 2ratio (log 2 fold-change of mean responder to mean non-responder, positive is higher in responder), wc.qval (FDR corrected q-value, Benjamini-Hochberg procedure, for analysis WITHIN taxon level), wc.qval.global (FDR corrected q-value, Benjamini-Hochberg procedure, for analysis considering ALL taxon levels

### Example 4: Stool Metabolite Concentration Correlates with Outcome

Correlation between stool metabolite levels and treatment outcome (i.e., responder and non-responder) was determined. Briefly, stool metabolite levels were measured by Metabolonn Inc. and returned as log-transformed concentrations in the stool. Targeted analysis of short and medium chain fatty acids was performed separately from global untargeted metabolomics. Differential abundance of these metabolites between responders and non-responders was tested via non-parametric Wilcoxon ranksum tests and parametric t-tests. Super-pathway and sub-pathway enrichment analysis was performed in the global analysis based on Wilcoxon ranksum test outcomes. There were 64 patient samples included in the short chain and medium chain fatty acid (SCFA/MCFA) analysis (30NR, 34R) and 65 patient samples included in the global metabolite analysis (30NR, 35R) with 63 samples analyzed with both methods.

As shown in Table 14, the SCFA metabolites acetic acid, propionic acid, and butyric acid are higher in responders compared with non-responders as is total SCFA/MCFA concentration (see Table 14). SCFA and MCFA are products of metabolism by specific bacterial taxa, and these functions are enriched or repressed by additional members of the microbial ecology, thus the data indicate that patients with a microbial ecology/microbiome conducive to SCFA/MCFA production are more likely to respond to CPI. The concentration of propionic acid and butyric acid are shown to directly affect expression of several CD8+ T-cell activation markers in Example 5 supporting a direct role for these metabolites in T-cell function.

TABLE 14
SCFA/MCFA metabolites compared to outcome

| Metabolite | NR.Mean | R.Mean | R-NR | wc.pval | wc.qval | ttest.p | ttest.qval |
|---|---|---|---|---|---|---|---|
| Propionic acid | 2.986434326 | 3.134712182 | 0.148277857 | 0.023506889 | 0.070521 | 0.019826 | 0.089823 |
| Acetic.acid | 3.34317748 | 3.500896124 | 0.157718644 | 0.01560315 | 0.070521 | 0.019961 | 0.089823 |
| Total.FA | 3.702817864 | 3.830940821 | 0.128122957 | 0.021856856 | 0.070521 | 0.03578 | 0.107341 |
| Butyric.acid | 2.897343529 | 3.050304222 | 0.152960693 | 0.096807223 | 0.197752 | 0.09862 | 0.221895 |

Metabolite (metabolite measured), NR.Mean (mean of log transformed metabolite concentration in non-responders), R.Mean (mean of log transformed metabolite concentration in responders), R-NR (difference in the mean of the logs, >0 means higher in responders vs non-responders), wc.pval (Wilcoxon rank sum test p-value), wc.qval (Wilcoxon rank sum test q-value FDR corrected), ttest.p (t-test p-value), ttest.qval (t-test q-value FDR corrected)

Global metabolite analysis identified 40 metabolites in stool samples with significantly different quantitative abundance between CPI responders and non-responders (nominal t-test p-value <0.05). (See Table 15.) Thus, production or depletion of these metabolites by bacterial metabolism may influence response to CPI.

TABLE 15
Global metabolite analysis compared to outcome

| Metabolite | SUPER_PATHWAY | SUB_PATHWAY | NR.Mean | R.Mean |
|---|---|---|---|---|
| X-23587 | | | −1.357779211 | 0.010813212 |
| N-methylalanine | Amino Acid | Alanine and Aspartate | −0.446051099 | 0.101791067 |
| | | Metabolism | | |
| erucate (22:1n9) | Lipid | Long Chain | −0.447037897 | 0.466569868 |
| | | Monounsaturated Fatty | | |
| | | Acid | | |
| dodecenedioate | Lipid | Fatty Acid, | −0.969798398 | −0.402784278 |
| (C12:1-DC)* | | Dicarboxylate | | |
| N- | Amino Acid | Polyamine Metabolism | −1.372474465 | −0.593451745 |
| carbamoylputrescine | | | | |
| 7,12- | Lipid | Secondary Bile Acid | −1.291532846 | −0.76525094 |
| diketolithocholate | | Metabolism | | |
| butyrylputrescine/ | Amino Acid | Polyamine Metabolism | −2.026417264 | −0.681485873 |
| isobutyrylputrescine | | | | |
| glycerophosphoserine* | Lipid | Phospholipid | −0.947240281 | −0.342436038 |
| | | Metabolism | | |
| 2S,3R- | Lipid | Fatty Acid, Dihydroxy | −0.615621454 | −0.21320387 |
| dihydroxybutyrate | | | | |
| N-acetylglucosamine/ | Carbohydrate | Aminosugar | −0.812586315 | −0.287797707 |
| N-acetylgalactosamine | | Metabolism | | |
| X-23450 | | | −0.69346716 | −1.305405299 |
| X-23469 | | | −0.986807881 | −0.392973147 |
| X-25045 | | | −0.777862683 | −0.587836558 |
| tyramine O-sulfate | Amino Acid | Tyrosine Metabolism | −1.0639837 | 0.130294714 |
| N-acetylneuraminate | Carbohydrate | Aminosugar | −0.886467473 | −0.236515121 |
| | | Metabolism | | |
| X-24412 | | | −0.903421829 | −0.252711376 |
| mannose | Carbohydrate | Fructose, Mannose and | −2.268406944 | −1.777925664 |
| | | Galactose Metabolism | | |
| malate | Energy | TCA Cycle | −0.835941808 | −0.384672267 |
| 4-ureidobutyrate | Nucleotide | Pyrimidine Metabolism, | −1.250209283 | −0.709009454 |
| | | Uracil containing | | |
| 2-oleoylglycerol | Lipid | Monoacylglycerol | −1.370105786 | −0.450503754 |
| (18:1) | | | | |
| X-23277 | | | −0.731843137 | −0.276769516 |
| X-25323 | | | −1.314292547 | −1.088912135 |
| 1-palmitoyl-2- | Lipid | Galactosyl | −1.058334327 | −0.673322002 |
| linoleoyl- | | Glycerolipids | | |
| galactosylglycerol | | | | |
| (16:0/18:2)* | | | | |
| adrenate (22:4n6) | Lipid | Long Chain | −1.308424242 | −0.672961052 |
| | | Polyunsaturated Fatty | | |
| | | Acid (n3 and n6) | | |
| benzoate | Xenobiotics | Benzoate Metabolism | −0.019319211 | −0.516787092 |
| 1-myristoylglycerol | Lipid | Monoacylglycerol | −1.40483116 | −0.936728807 |
| (14:0) | | | | |
| X-25009 | | | −1.48324015 | −0.964363009 |
| 1-stearoyl-GPE (18:0) | Lipid | Lysophospholipid | −0.839502564 | −0.210275851 |
| 1-oleoyl-GPE (18:1) | Lipid | Lysophospholipid | −0.571445315 | −0.043112194 |
| X-25949 | | | −1.952160224 | −1.078349158 |
| 1-palmitoyl- | Lipid | Galactosyl | −1.267933374 | −0.57495125 |
| galactosylglycerol | | Glycerolipids | | |
| (16:0)* | | | | |
| rosuvastatin lactone | Xenobiotics | Drug - Metabolic | −1.610503507 | −1.938403416 |
| X-23671 | | | −1.328559631 | −1.55343223 |
| N- | Xenobiotics | Food Component/Plant | −1.251749075 | −0.310117083 |
| carboxymethylalanine | | | | |
| N-carbamoylalanine | Amino Acid | Alanine and Aspartate | −0.551843628 | −0.118054493 |
| | | Metabolism | | |
| N-acetylisoleucine | Amino Acid | Leucine, Isoleucine and | −0.970972019 | −0.377608305 |
| | | Valine Metabolism | | |
| dihomo-linoleate | Lipid | Long Chain | −0.267484754 | 0.271754731 |
| (20:2n6) | | Polyunsaturated Fatty | | |
| | | Acid (n3 and n6) | | |
| rosmarinate | Xenobiotics | Food Component/Plant | −0.803281844 | −1.315609948 |
| X-23173 | | | −0.696242073 | −0.10335303 |
| hypoxanthine | Nucleotide | Purine Metabolism, | −0.504907897 | −0.168548527 |
| | | (Hypo)Xanthine/Inosine | | |
| | | containing | | |

| Metabolite | R-NR | wc.pval | wc.qval | ttest.pval | ttest.qva |
|---|---|---|---|---|---|
| X-23587 | 1.368592 | 0.004264 | 0.956851 | 0.00398 | 0.967745 |
| N-methylalanine | 0.547842 | 0.008032 | 0.956851 | 0.005081 | 0.967745 |
| erucate (22:1n9) | 0.913608 | 0.004463 | 0.956851 | 0.00699 | 0.967745 |
| dodecenedioate | 0.567014 | 0.003635 | 0.956851 | 0.007841 | 0.967745 |
| (C12:1-DC)* | | | | | |
| N- | 0.779023 | 0.011497 | 0.956851 | 0.008522 | 0.967745 |
| carbamoylputrescine | | | | | |
| 7,12- | 0.526282 | 0.013224 | 0.956851 | 0.011343 | 0.967745 |
| diketolithocholate | | | | | |
| butyrylputrescine/ | 1.344931 | 0.021995 | 0.956851 | 0.012954 | 0.967745 |
| isobutyrylputrescine | | | | | |
| glycerophosphoserine* | 0.604804 | 0.012846 | 0.956851 | 0.01338 | 0.967745 |
| 2S,3R- | 0.402418 | 0.01156 | 0.956851 | 0.014329 | 0.967745 |
| dihydroxybutyrate | | | | | |
| N-acetylglucosamine/ | 0.524789 | 0.012425 | 0.956851 | 0.016089 | 0.967745 |
| N-acetylgalactosamine | | | | | |
| X-23450 | −0.61194 | 0.015992 | 0.956851 | 0.019009 | 0.967745 |
| X-23469 | 0.593835 | 0.014572 | 0.956851 | 0.019178 | 0.967745 |
| X-25045 | 0.190026 | 0.030114 | 0.956851 | 0.021375 | 0.967745 |
| tyramine O-sulfate | 1.194278 | 0.019903 | 0.956851 | 0.021963 | 0.967745 |
| N-acetylneuraminate | 0.649952 | 0.038866 | 0.956851 | 0.024754 | 0.967745 |
| X-24412 | 0.65071 | 0.037383 | 0.956851 | 0.025778 | 0.967745 |
| mannose | 0.490481 | 0.071121 | 0.956851 | 0.027718 | 0.967745 |
| malate | 0.45127 | 0.028735 | 0.956851 | 0.028546 | 0.967745 |
| 4-ureidobutyrate | 0.5412 | 0.029189 | 0.956851 | 0.028861 | 0.967745 |
| 2-oleoylglycerol | 0.919602 | 0.037709 | 0.956851 | 0.029036 | 0.967745 |
| (18:1) | | | | | |
| X-23277 | 0.455074 | 0.028777 | 0.956851 | 0.029801 | 0.967745 |
| X-25323 | 0.22538 | 0.0547 | 0.956851 | 0.030324 | 0.967745 |
| 1-palmitoyl-2- | 0.385012 | 0.130745 | 0.956851 | 0.036118 | 0.967745 |
| linoleoyl- | | | | | |
| galactosylglycerol | | | | | |
| (16:0/18:2)* | | | | | |
| adrenate (22:4n6) | 0.635463 | 0.273176 | 0.978644 | 0.037122 | 0.967745 |
| benzoate | −0.49747 | 0.032177 | 0.956851 | 0.03994 | 0.967745 |
| 1-myristoylglycerol | 0.468102 | 0.048228 | 0.956851 | 0.040198 | 0.967745 |
| (14:0) | | | | | |
| X-25009 | 0.518877 | 0.041338 | 0.956851 | 0.041548 | 0.967745 |
| 1-stearoyl-GPE (18:0) | 0.629227 | 0.055389 | 0.956851 | 0.042814 | 0.967745 |
| 1-oleoyl-GPE (18:1) | 0.528333 | 0.031281 | 0.956851 | 0.04346 | 0.967745 |
| X-25949 | 0.873811 | 0.041483 | 0.956851 | 0.043735 | 0.967745 |
| 1-palmitoyl- | 0.692982 | 0.059702 | 0.956851 | 0.043756 | 0.967745 |
| galactosylglycerol | | | | | |
| (16:0)* | | | | | |
| rosuvastatin lactone | −0.3279 | 0.014991 | 0.956851 | 0.04465 | 0.967745 |
| X-23671 | −0.22487 | 0.028125 | 0.956851 | 0.045691 | 0.967745 |
| N- | 0.941632 | 0.083367 | 0.956851 | 0.045692 | 0.967745 |
| carboxymethylalanine | | | | | |
| N-carbamoylalanine | 0.433789 | 0.016679 | 0.956851 | 0.046801 | 0.967745 |
| N-acetylisoleucine | 0.593364 | 0.05516 | 0.956851 | 0.04687 | 0.967745 |
| dihomo-linoleate | 0.539239 | 0.041483 | 0.956851 | 0.047064 | 0.967745 |
| (20:2n6) | | | | | |
| rosmarinate | −0.51233 | 0.006176 | 0.956851 | 0.047639 | 0.967745 |
| X-23173 | 0.592889 | 0.027143 | 0.956851 | 0.04875 | 0.967745 |
| hypoxanthine | 0.336359 | 0.064211 | 0.956851 | 0.049438 | 0.967745 |

Metabolite (Metabolite measured), SUPER_PATHWAY (metabolite class as determined by Metabolon), SUB_PATHWAY (Metabolite sub-class as determined by Metabolon), NR.Mean (Mean of log transformed metabolite concentration in non-responders), R.Mean (Mean of log transformed metabolite concentration in responders), R-NR (difference in the mean of the logs, >0 means higher in responders vs non-responders), wc.pval (Wilcoxon rank sum test p-value), wc.qval (Wilcoxon rank sum test q-value FDR corrected), ttest.pval (t-test p-value), ttest.qval (t-test q-value FDR corrected)

### Example 5: FMT Supernatant and Single Species CD8 Gene Expression Assay Correlates with Outcome

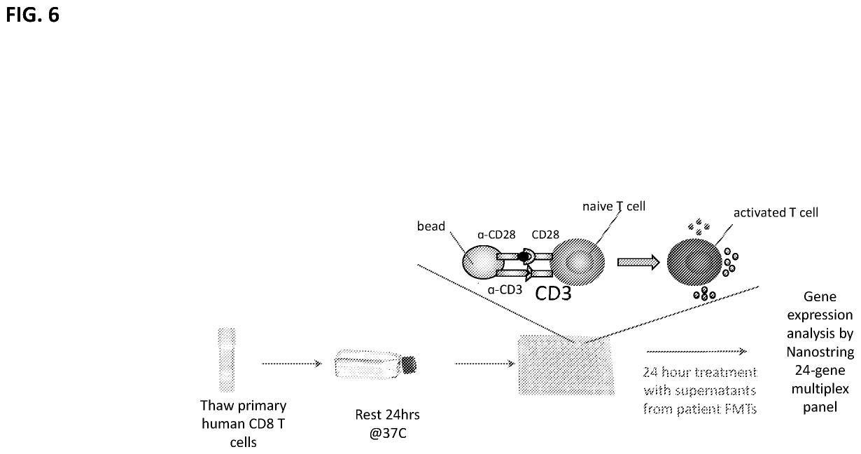

The effects of cancer patient FMTs on CD8 T cell activation and cytotoxicity using a human CD8 T cell activation assay were assessed. The in vitro assay mimics in vivo T cell activation from antigen-presenting cells by utilizing the two activation signals CD3 and CD28, bound to a three-dimensional bead mimicking antigen-presenting cells (see FIG. 6).

Patient FMTs from stool samples were prepared in vitro and cultured. FMTs were prepared under anaerobic conditions from patient stool samples for a final concentration of 10% FMT in (0.9% saline+15% glycerol), aliquoted and stored at −80° C. Then, Supernatants were generated and used in a CD8 assay. To generate the supernatants, fecal culture medium 4 (FCM4) media was pre-reduced overnight in the anaerobic chamber. FCM4 is composed of complex carbohydrates and mucin in addition to other nutrients that support growth of numerous phylogenetically diverse anaerobic species found in the colon. One vial of FMT material was thawed at RT and it was the spun at 10,000 g for 10 mins. The FMT vial was brought inside the anaerobic chamber and the supernatant was discarded, the volume of the supernatant was removed and recorded, and the same amount of pre-reduced PBS was added to the vial and mixed. Samples were inoculated at a target titer of 5×10⁶-1×10⁷ in a 2 mL deep well plates. The plates were covered with an Aeraseal membrane and incubated at 37° C. anaerobically for 7 days. The plates were centrifuged at 4000 rpm for 10 minutes, supernatants were passed through filter plates, aliquoted and stored at −80° C. for use in in vitro assays.

Briefly, the human primary CD8 T cells were thawed at 37° C. for 24 hr and activated by CD3 and CD28 at 37° C. for 2 days. The cells were treated at 37° C. for 24 hr with bacterial supernatants from different cancer (i.e., bladder, melanoma, lung and breast cancer) patient FMTs or negative control bacterial media. The cells were analyzed by flow cytometry for viability and proliferation. At the end of the 24 hr treatment with bacterial supernatants, the cells were lysed and the cell lysates were used in a gene expression analysis using multiplexed molecular barcode (available from NanoString Technologies) or multiplex panels.

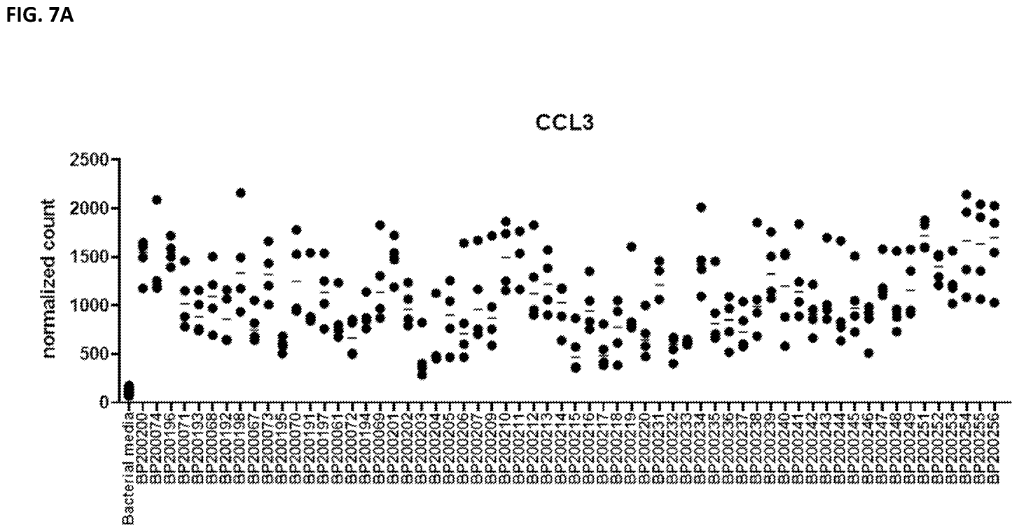

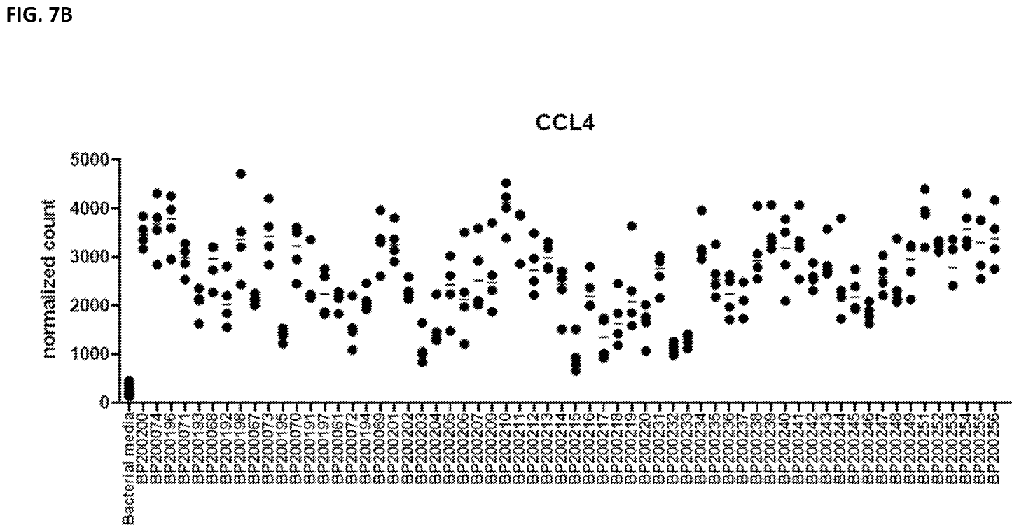

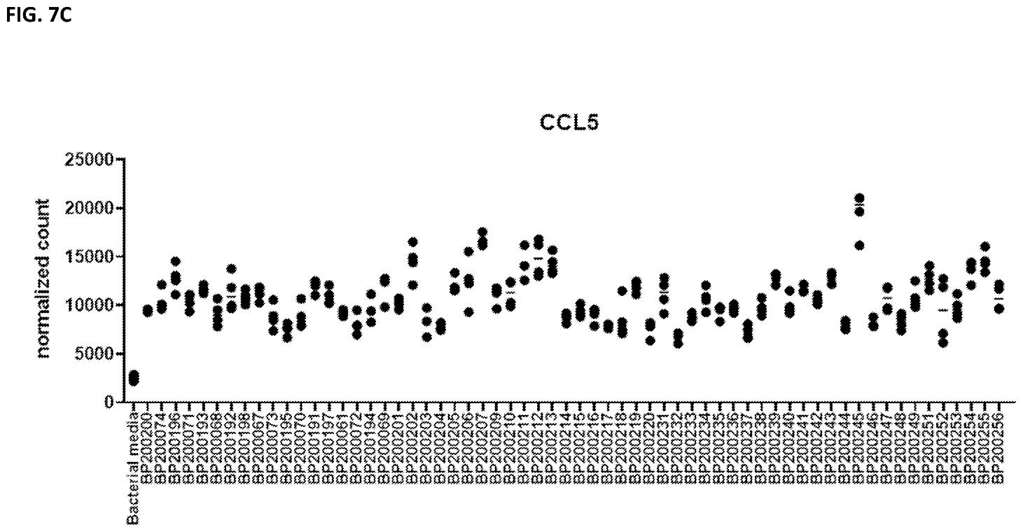

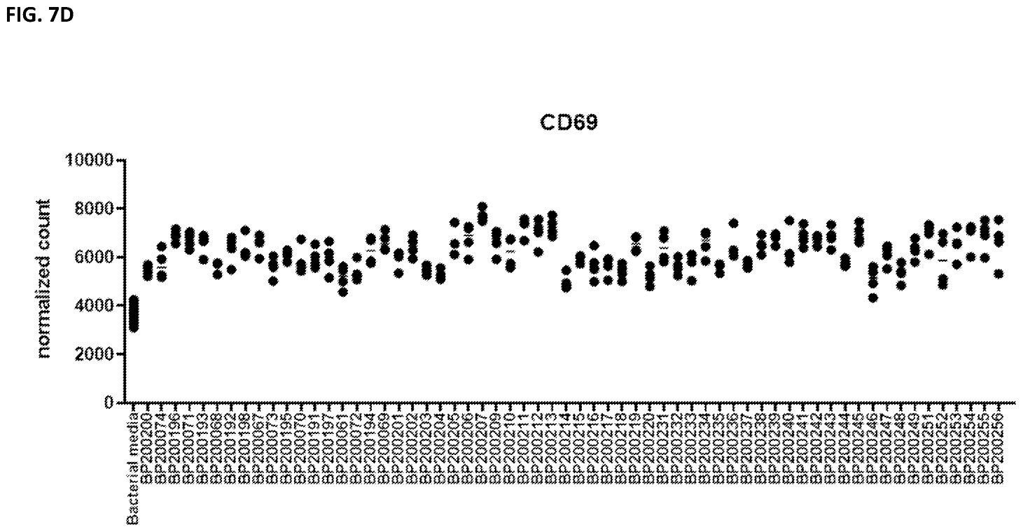

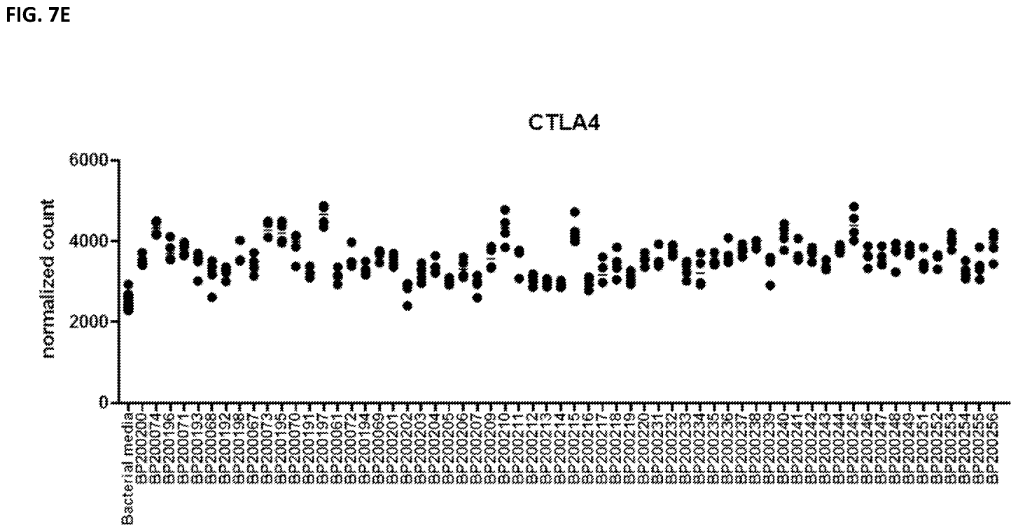

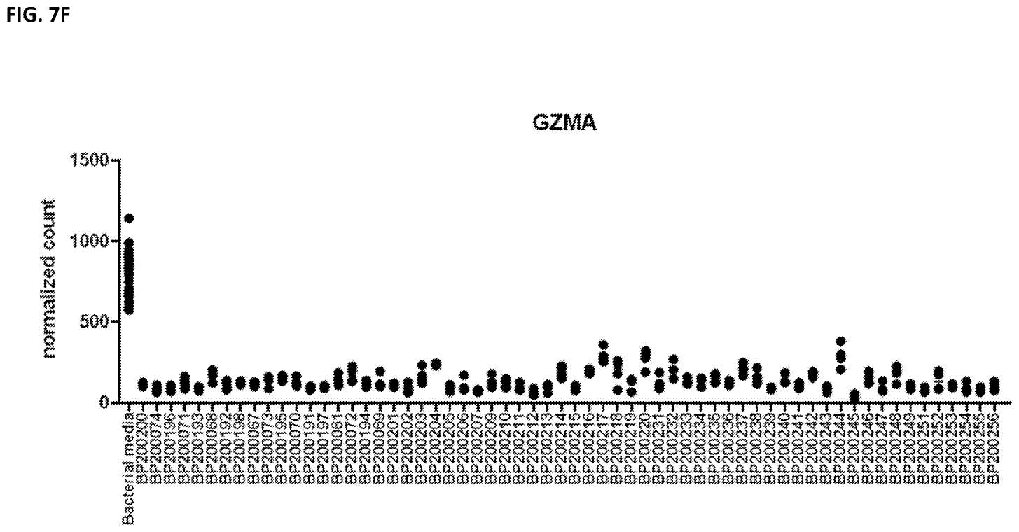

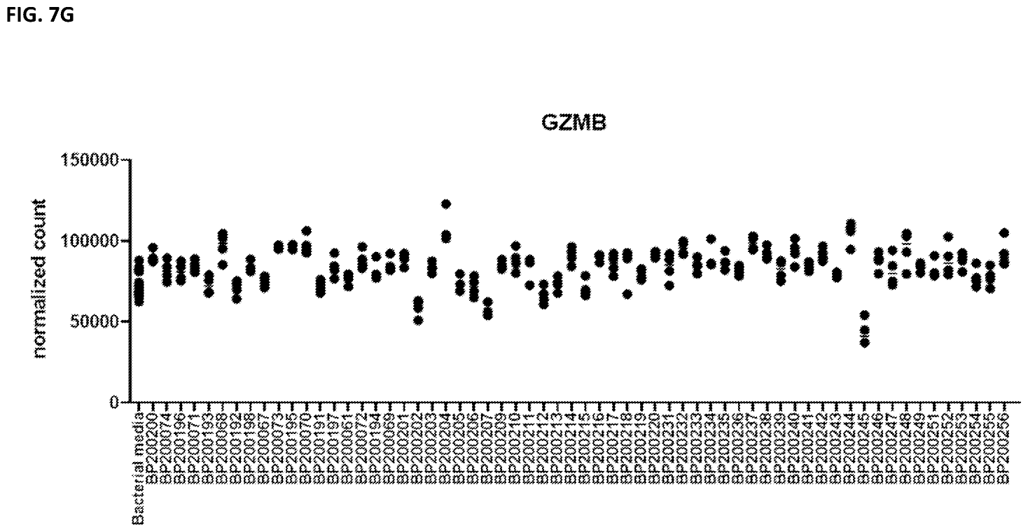

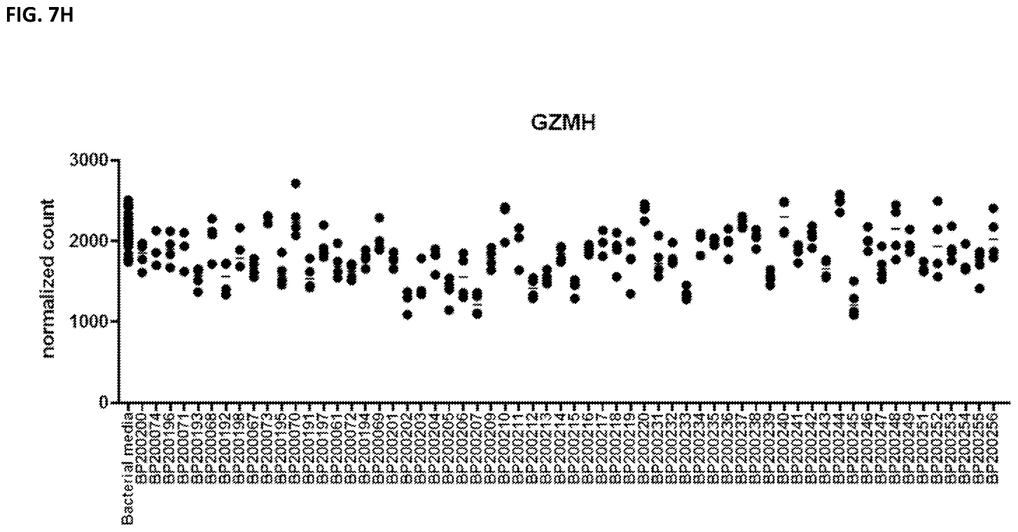

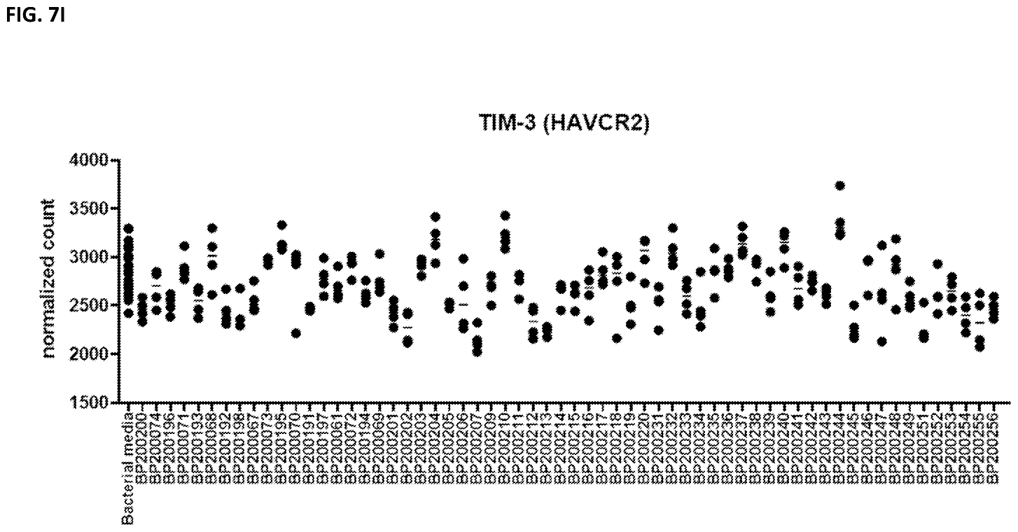

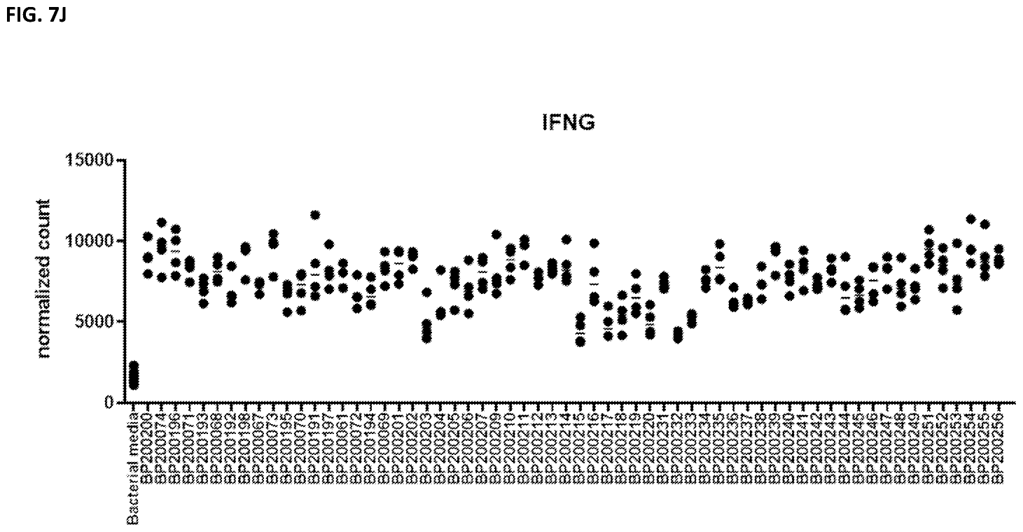

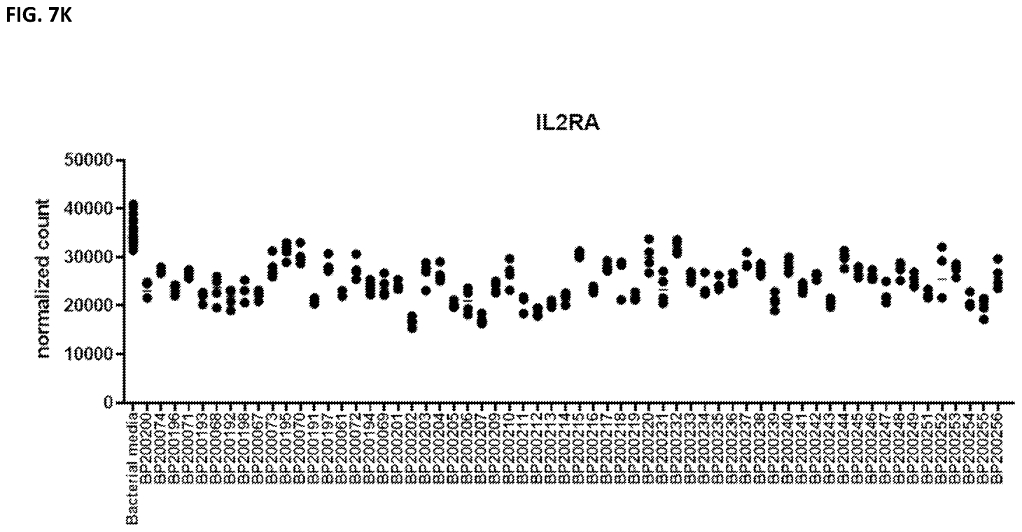

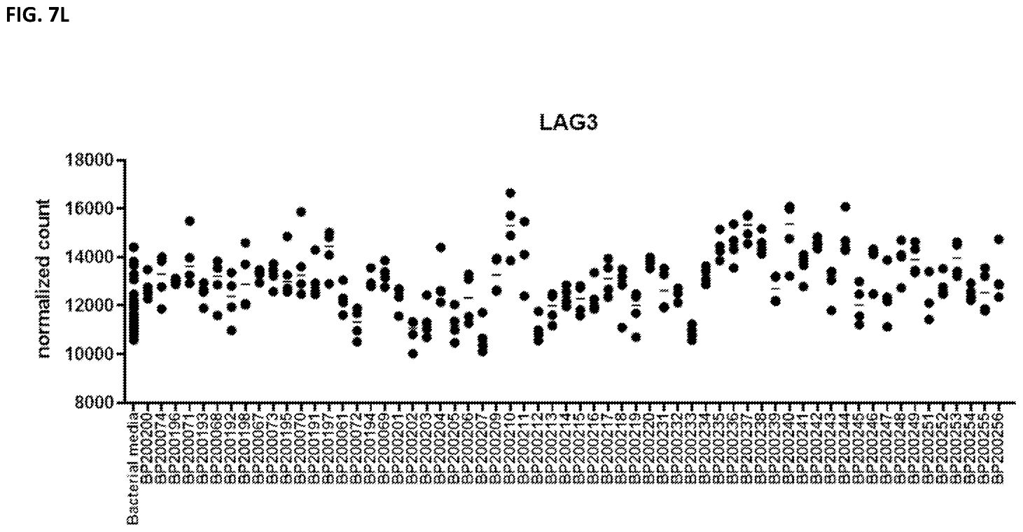

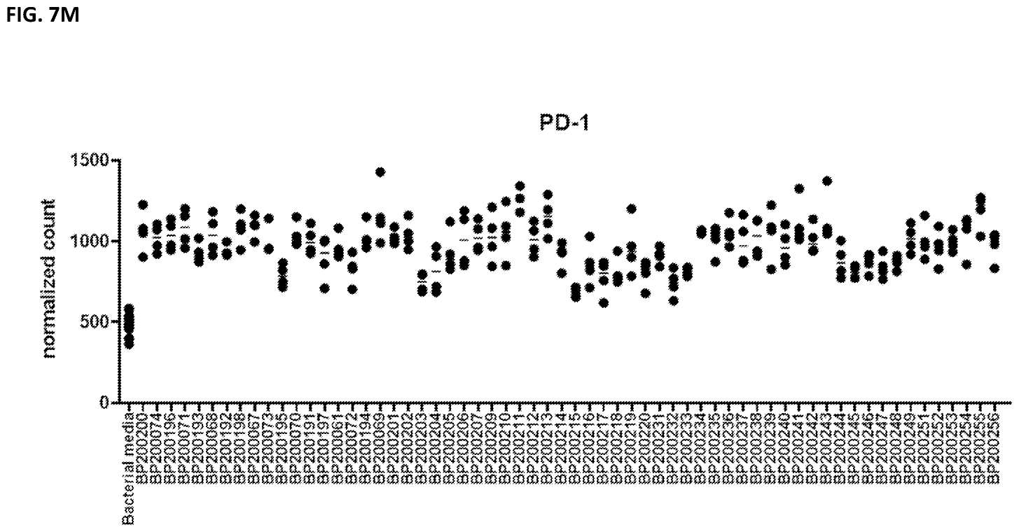

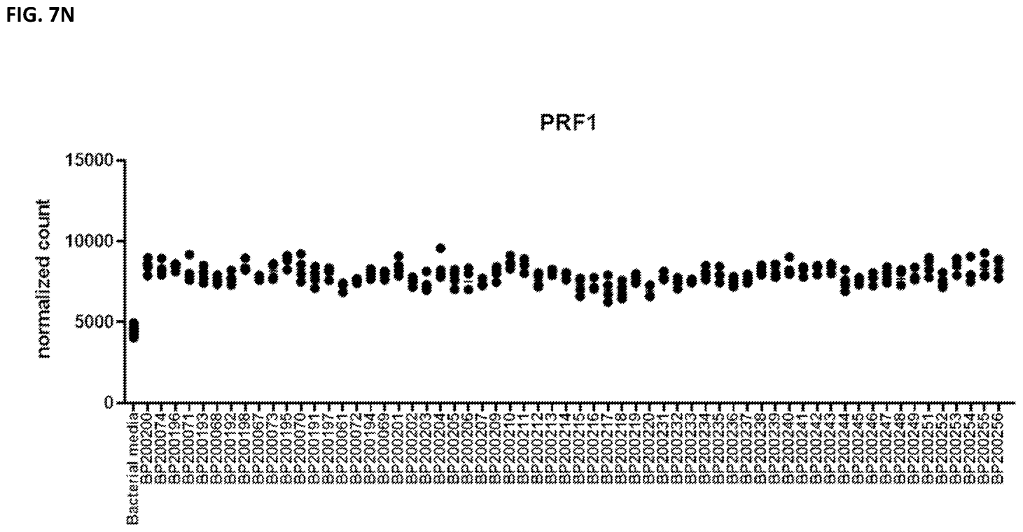

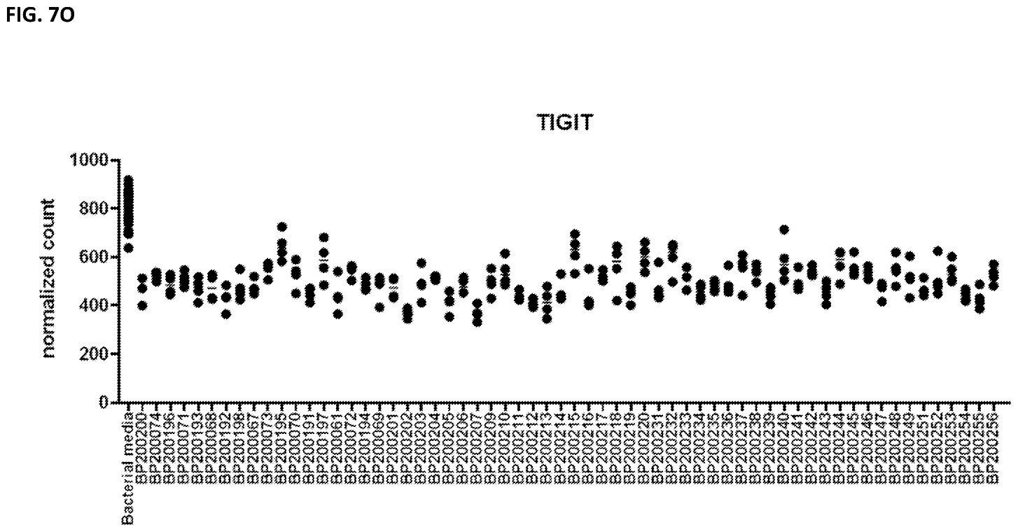

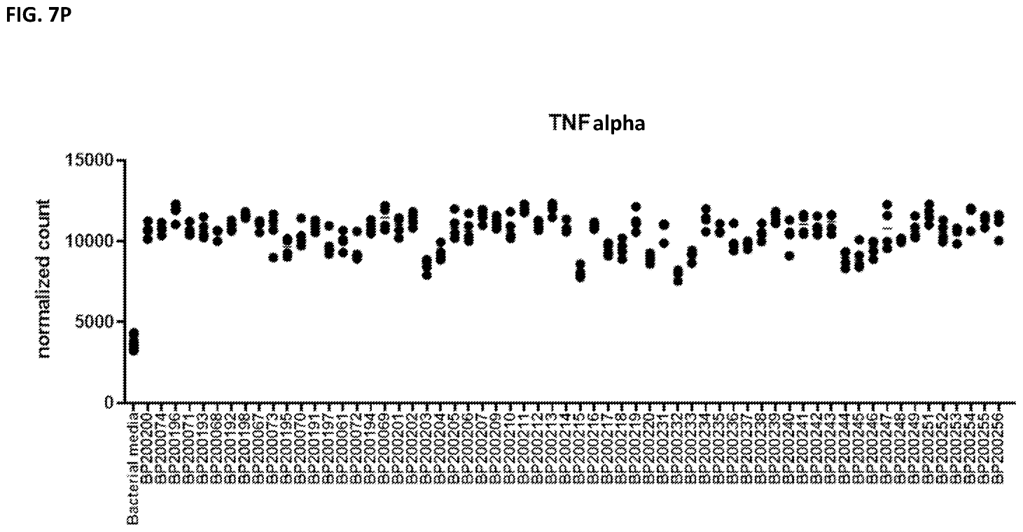

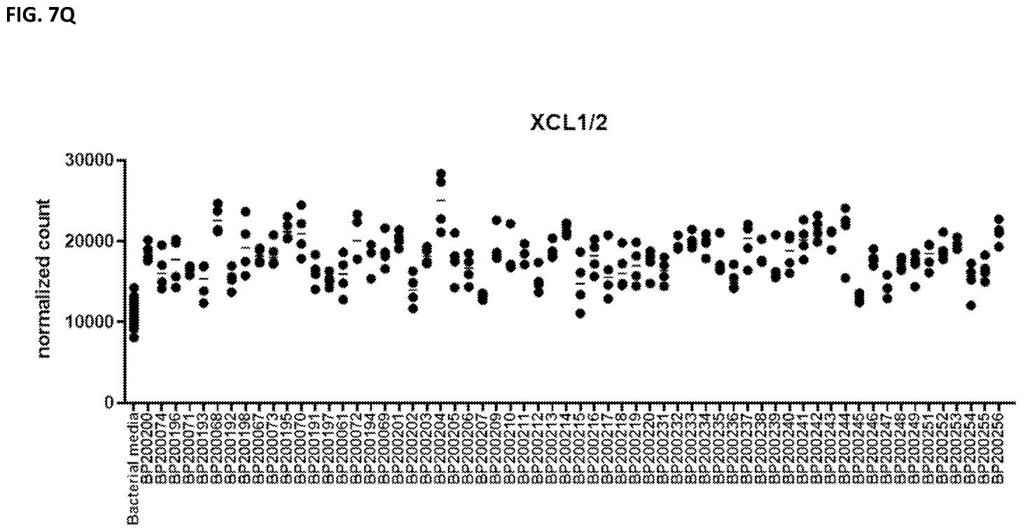

Treatment of activated CD8 T cells with these FMT supernatants significantly modulated the expression of many genes involved in T cell activation, cytotoxicity, exhaustion and effector function (see FIGS. 7A-Q; CCL3, CCL4, CCL5, CD69, CTLA4, GZMA, GZMB, GZMH, TIM-3 (HAVCR2), IFNG, IL2RA, LAG3, PD-1, PRF1, TIGIT, TNFα, XCL1/2). Cancer patient FMTs modulate T cell activation and effector function to variable degrees.

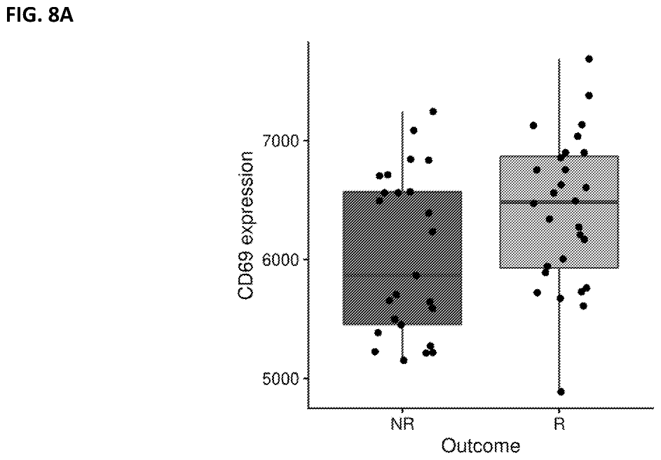

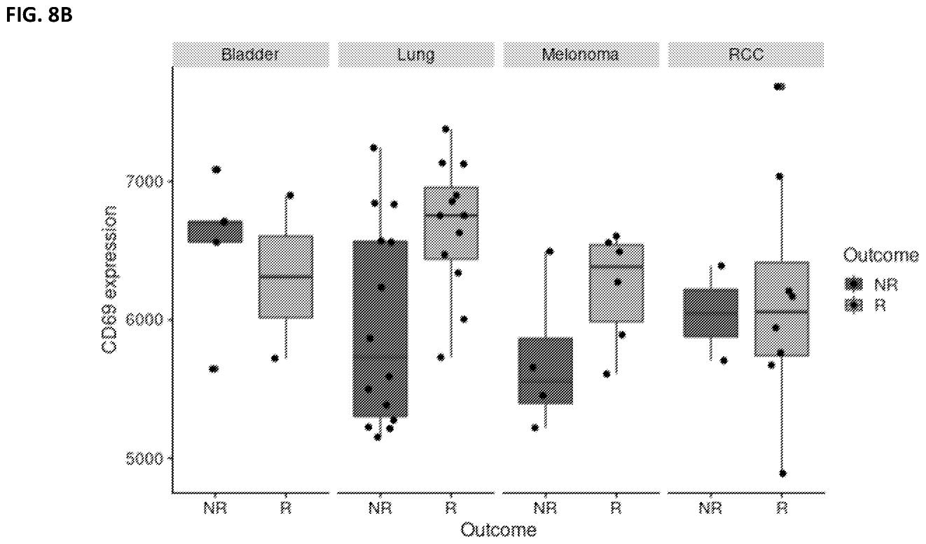

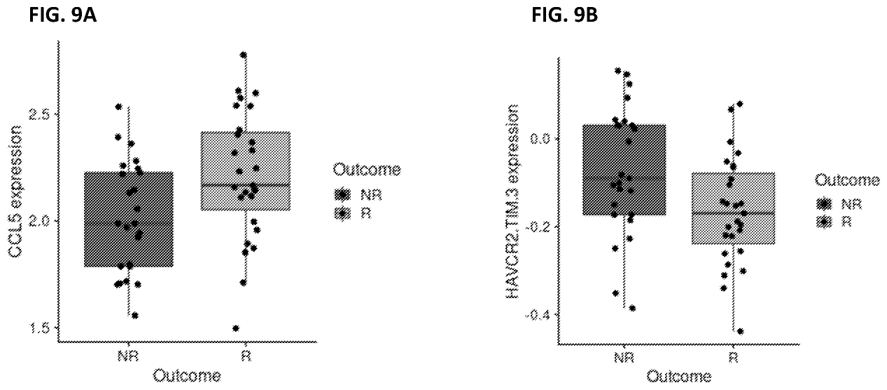

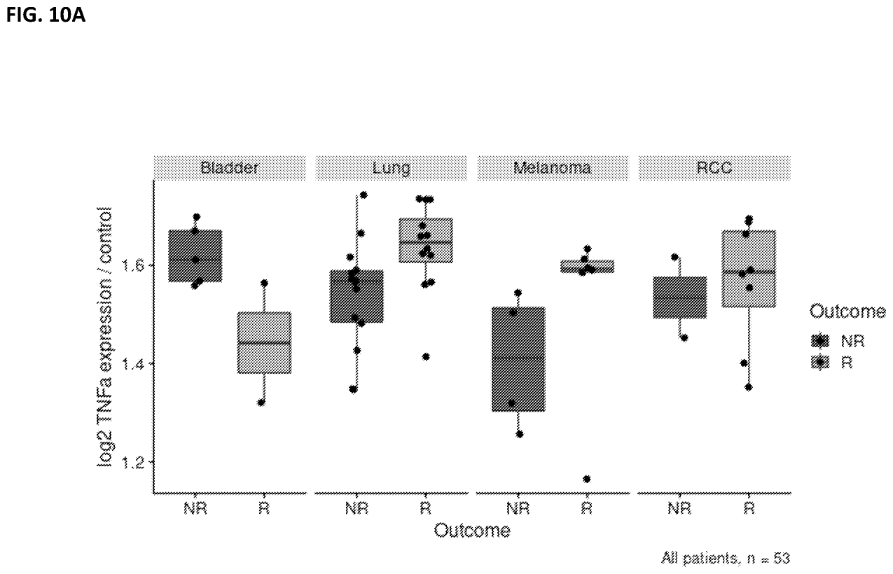

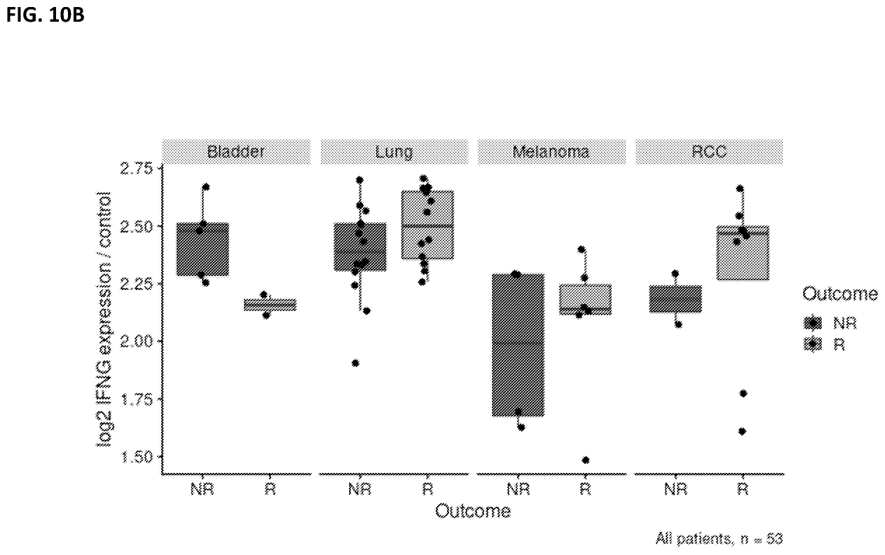

The results of the in vitro CD8 gene expression assay were correlated with clinical response. CD8 expression profiles were converted to log-2 fold change compared to media control. Differential expression of these genes between responders and non-responders was tested via non-parametric Wilcoxon ranksum tests and parametric t-tests. There were 53 samples included in this analysis (25NR and 28R). Several individual CD8 expression profiles were positively and negatively associated with responder status. Responder FMTs induce significantly higher expression of the CD8 activation marker CD69 (FIG. 8A), an effect that is driven by the lung, melanoma, and renal cell carcinoma (RCC) cancer groups (FIG. 8B). T/NK cell recruiting and activating chemokine CCL5 is correlated with outcome, whereas inhibitory receptor TIM3 is inversely correlated with outcome. That is, responder samples induce significantly higher expression of CCL5 (FIG. 9A), whereas responder FMT samples suppress the expression of TIM-3 (FIG. 9B). In certain cancer types responder FMTs induce higher expression of the CD8 effector cytokines TNFalpha (FIG. 10A) and IFNgamma (FIG. 10B), suggesting a cancer-specific microbiome effect on CD8 T cell effector function.

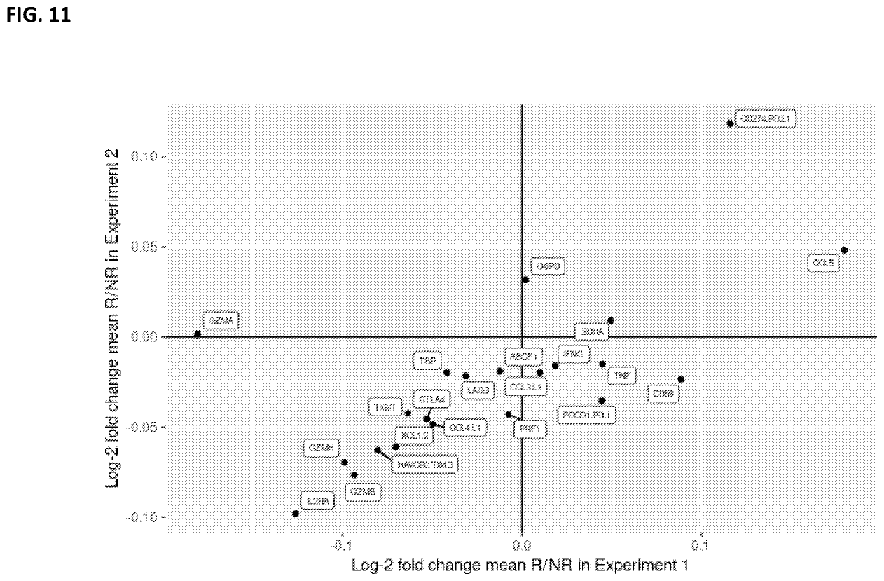

The CD8 gene expression assay described above was then run with different methods to prepare the FMT sample. Briefly, input titer of bacterial cells was normalized via dilution, bile-acids were added to outgrowth of the FMT, and a new donor was used to isolate CD8+ T-cells. Individual gene level analysis showed greater variation within responder and non-responder categories such that the univariate associations were no longer significant. However the overall magnitude of the mean differences between the two experiments was largely consistent and significantly correlated (R1 vs R2 Pearson correlation r²=0.43, p=0.0006; FIG. 11). The results of the experiment 1 (R1) and the experiment 2 (R2) are summarized in Table 16.

TABLE 16
CD8 gene-level log2 fold changes in responders vs. non-
responders for experiment 1 (R1) and experiment 2 (R2)

| | R1 t-test | R1 Log2 Fold | R2 t-test | R2 Log2 Fold |
|---|---|---|---|---|
| Gene | p-value | Change | p-value | Change |
| SDHA | 0.018262 | 0.049461543 | 0.55619 | 0.009176165 |
| IL2RA | 0.022572 | −0.126060156 | 0.16881 | −0.098000926 |
| TBP | 0.025021 | −0.041780292 | 0.391801 | −0.019734451 |
| CCL5 | 0.025883 | 0.179741705 | 0.572173 | 0.048050663 |
| CD69 | 0.040606 | 0.08851757 | 0.629959 | −0.023557994 |
| HAVCR2.TIM.3 | 0.046484 | −0.080191956 | 0.33861 | −0.063010487 |
| GZMB | 0.049188 | −0.093354338 | 0.205639 | −0.076579714 |
| IL24 | 0.064497 | 0.262238142 | | |
| CD274.PD.L1 | 0.076417 | 0.116054843 | 0.155815 | 0.118383498 |
| GZMH | 0.114346 | −0.09881079 | 0.324135 | −0.06963858 |
| TIGIT | 0.162259 | −0.063503346 | 0.468535 | −0.042340923 |
| XCL1.2 | 0.1656 | −0.070313442 | 0.221975 | −0.061251938 |
| TNF | 0.207122 | 0.044960334 | 0.664535 | −0.015020493 |
| CTLA4 | 0.209005 | −0.052946268 | 0.272593 | −0.045552829 |
| GZMA | 0.225628 | −0.180635199 | 0.991461 | 0.001321844 |
| ABCF1 | 0.280387 | −0.0122174 | 0.113128 | −0.019072405 |
| PDCD1.PD.1 | 0.337851 | 0.044400947 | 0.457329 | −0.035486896 |
| LAG3 | 0.370951 | −0.031286516 | 0.589852 | −0.021682625 |
| CCL4.L1 | 0.707986 | −0.049602514 | 0.587183 | −0.048436733 |
| PRF1 | 0.73927 | −0.007366742 | 0.219371 | −0.043148937 |
| IFNG | 0.816098 | 0.018540369 | 0.79846 | −0.01603919 |
| G6PD | 0.908788 | 0.002094847 | 0.200437 | 0.031731607 |
| CCL3.L1 | 0.941898 | 0.0100926 | 0.843514 | −0.019698694 |

Spearman correlation of taxa abundance with CD8 expression profile across MSK patients (n = 53 patients).

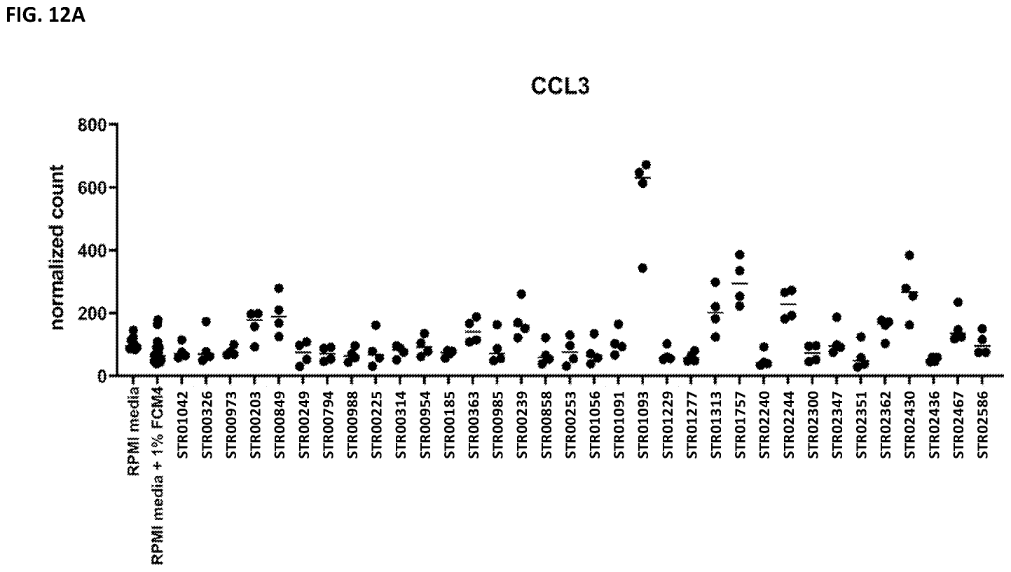

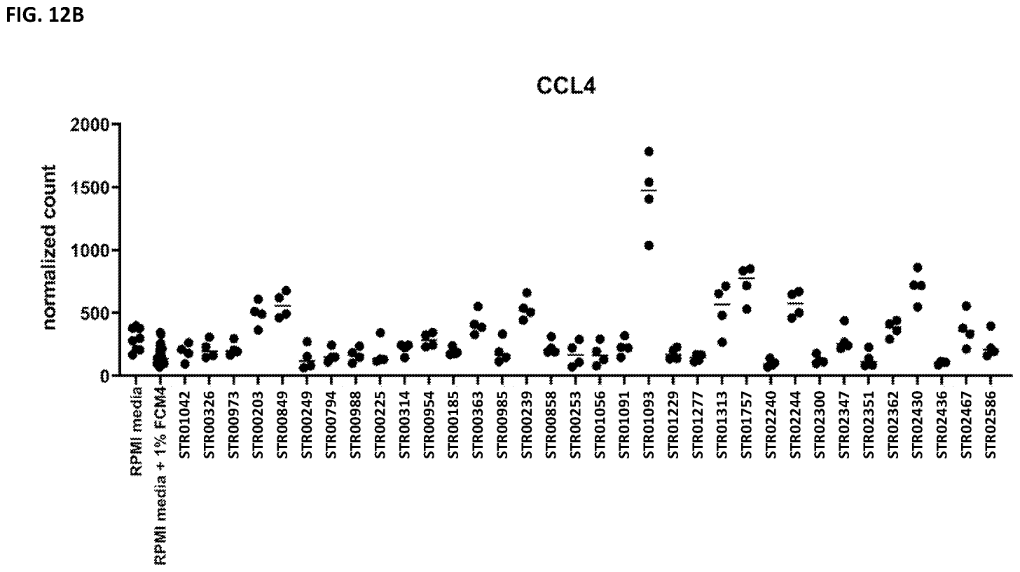

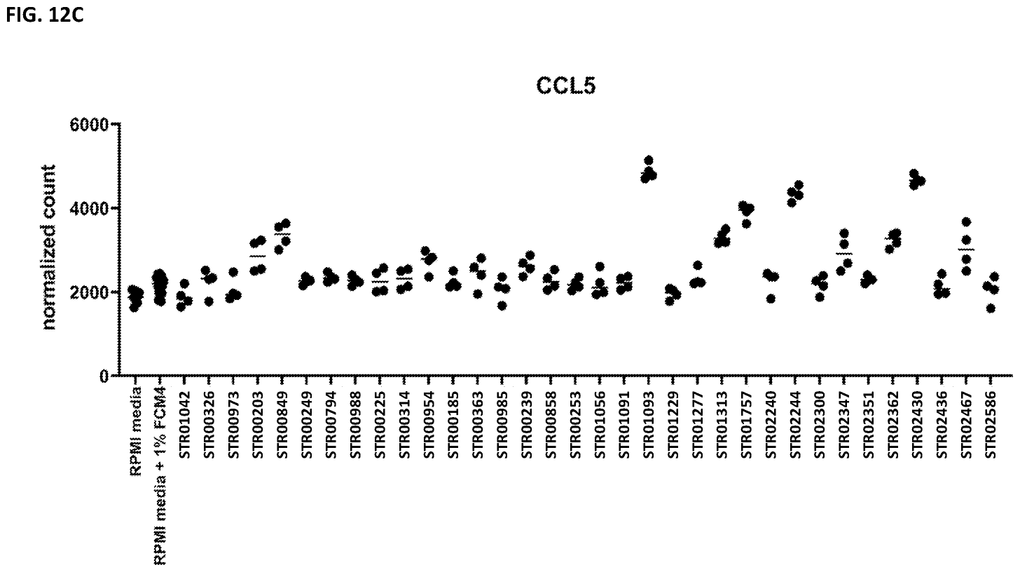

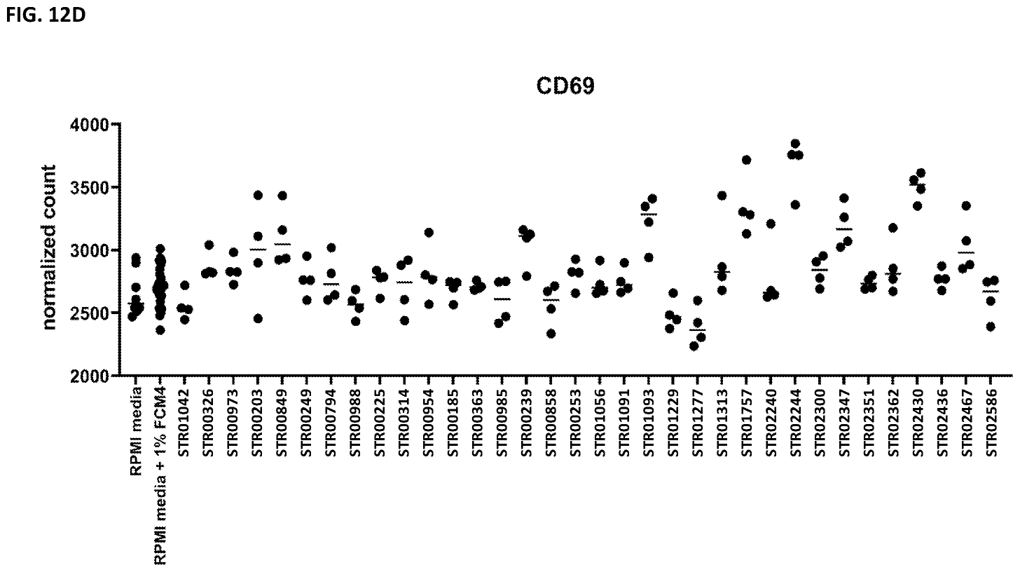

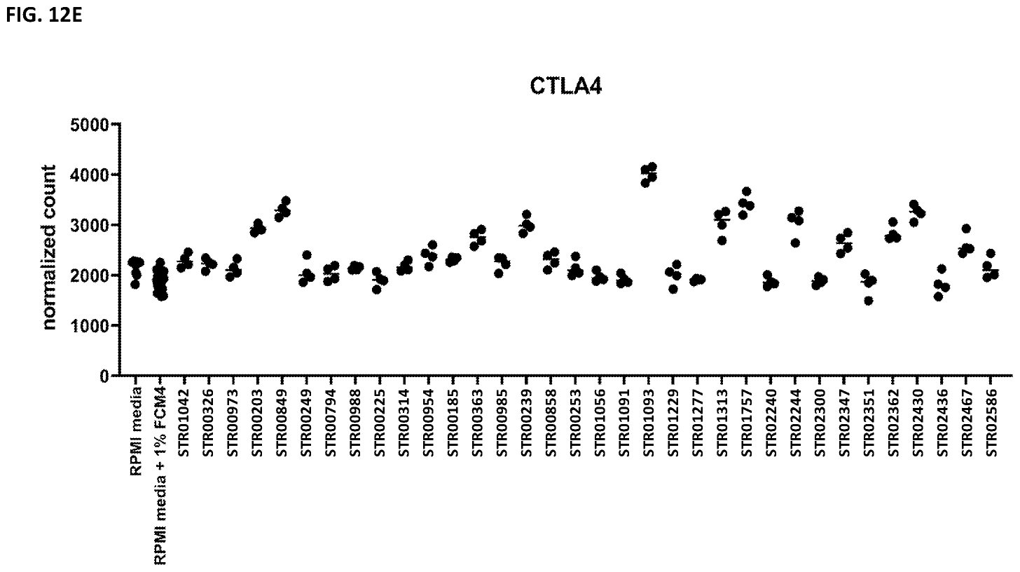

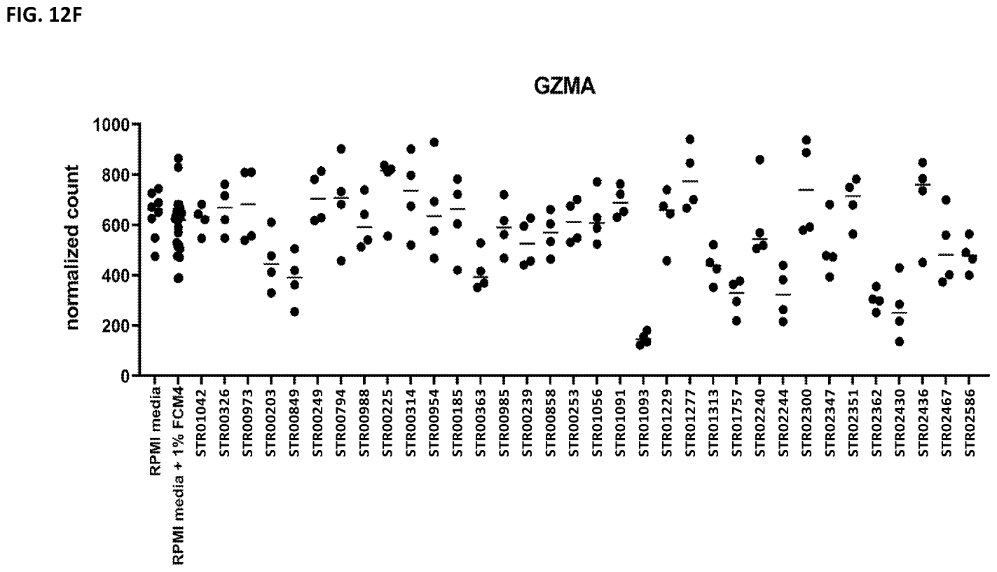

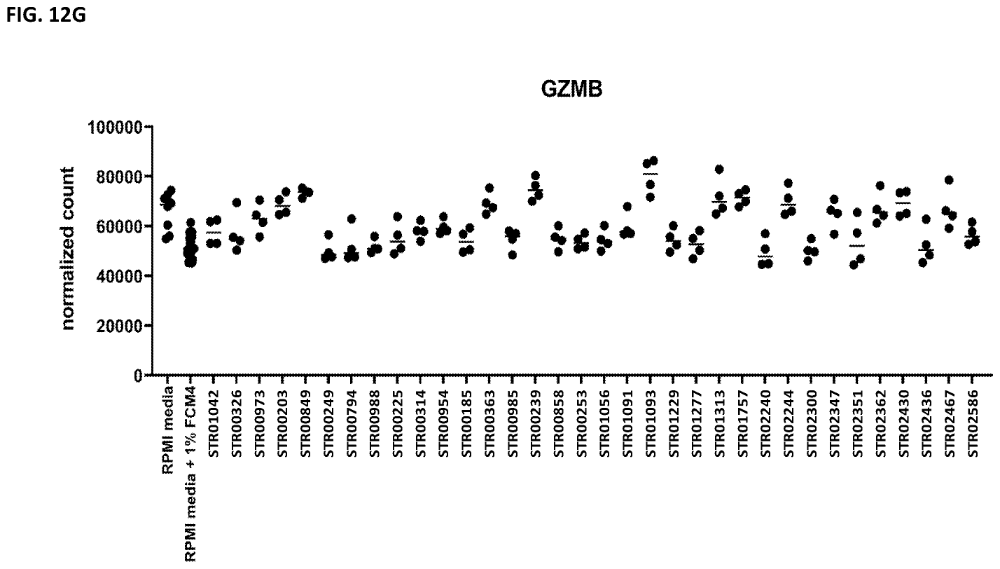

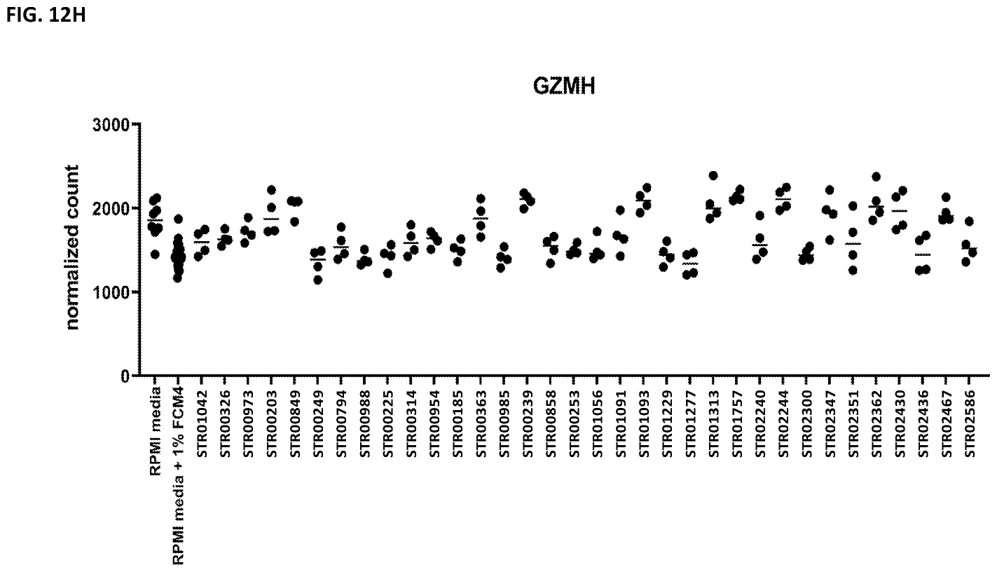

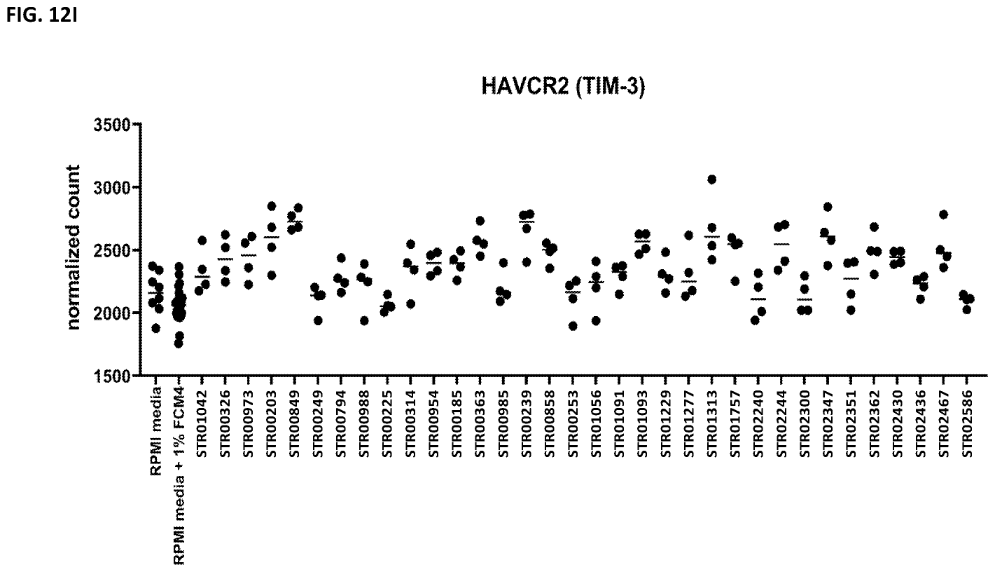

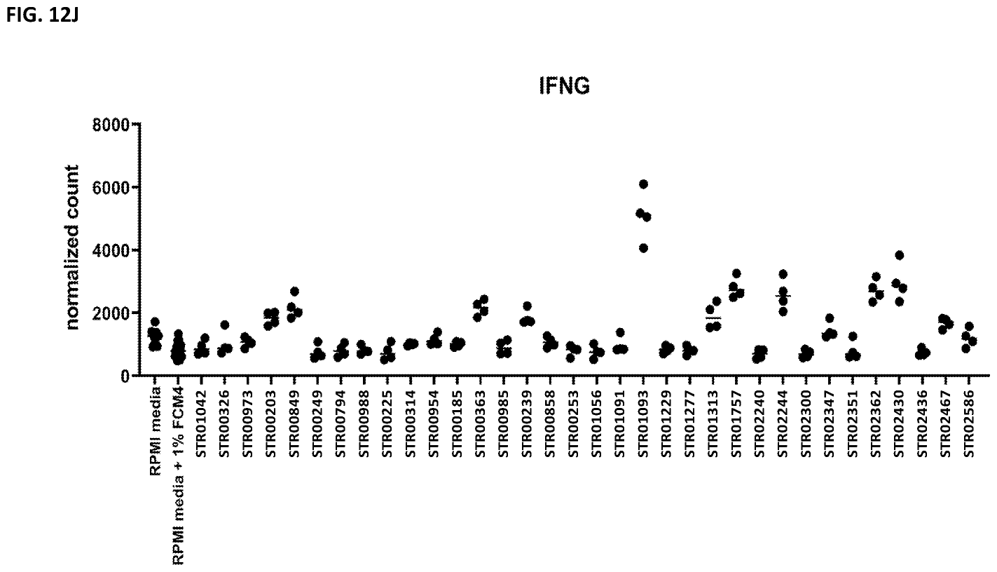

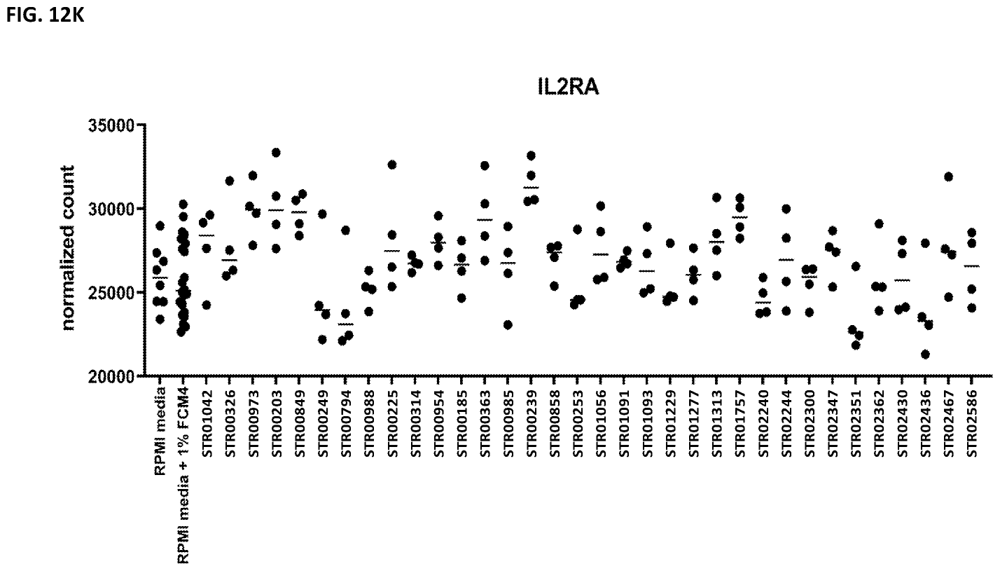

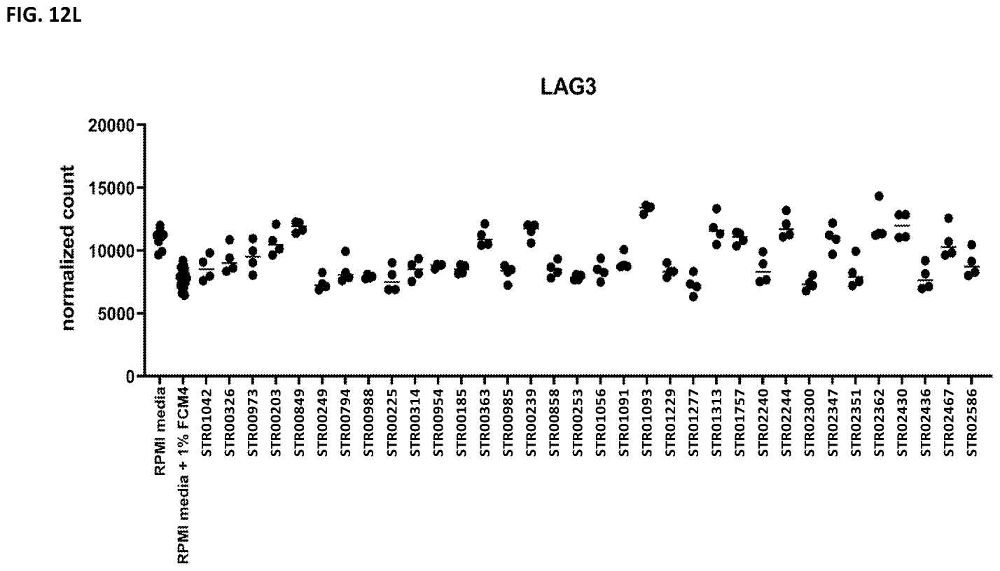

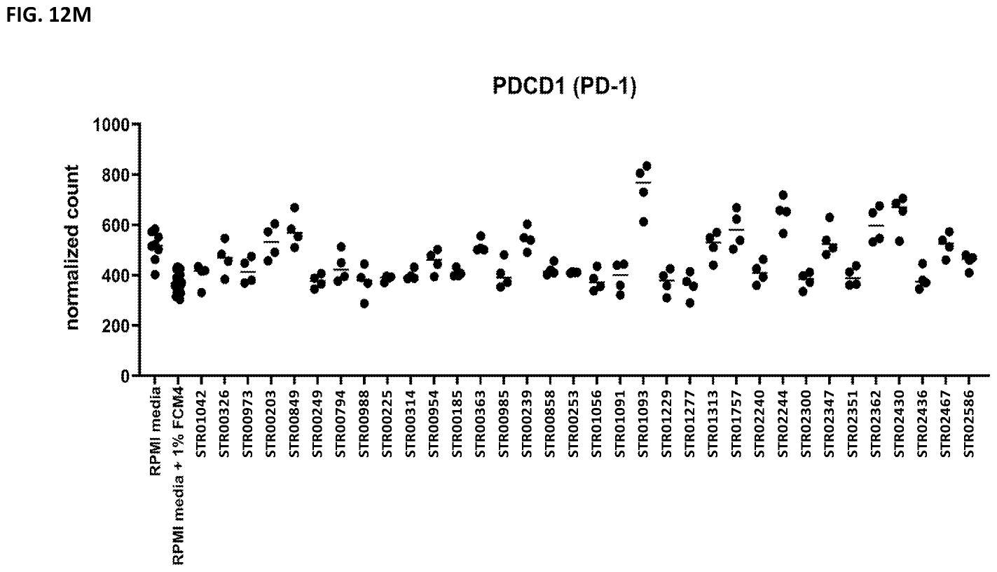

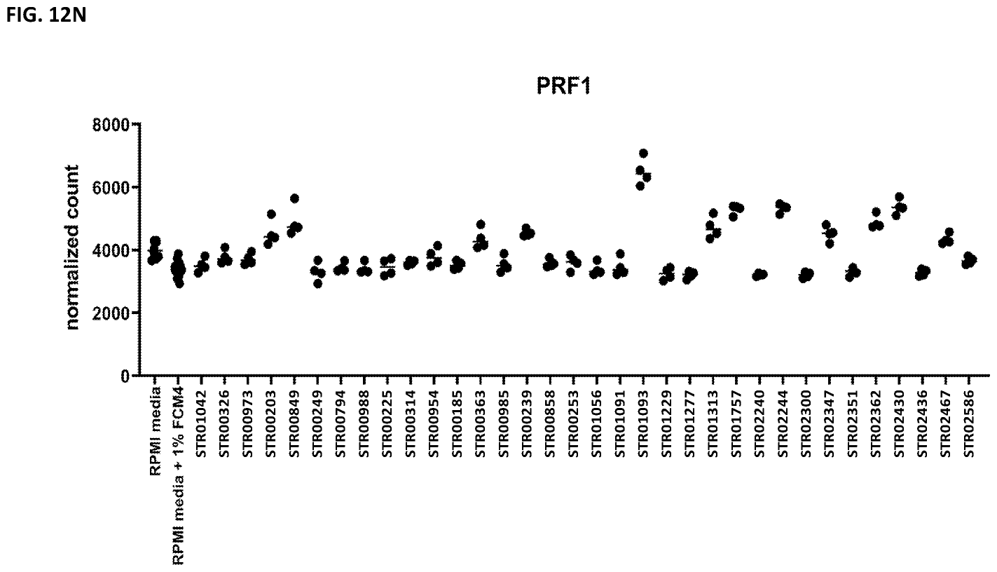

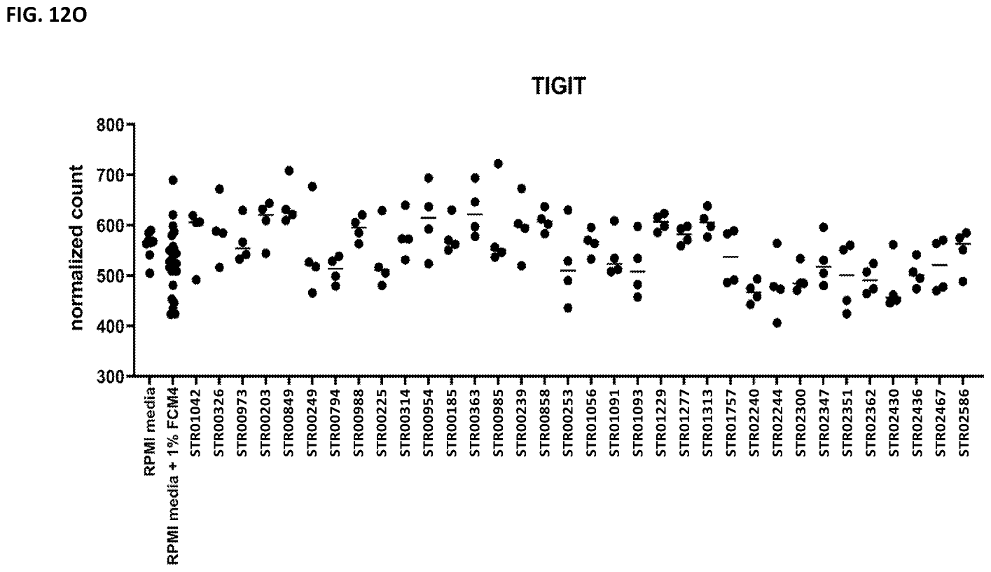

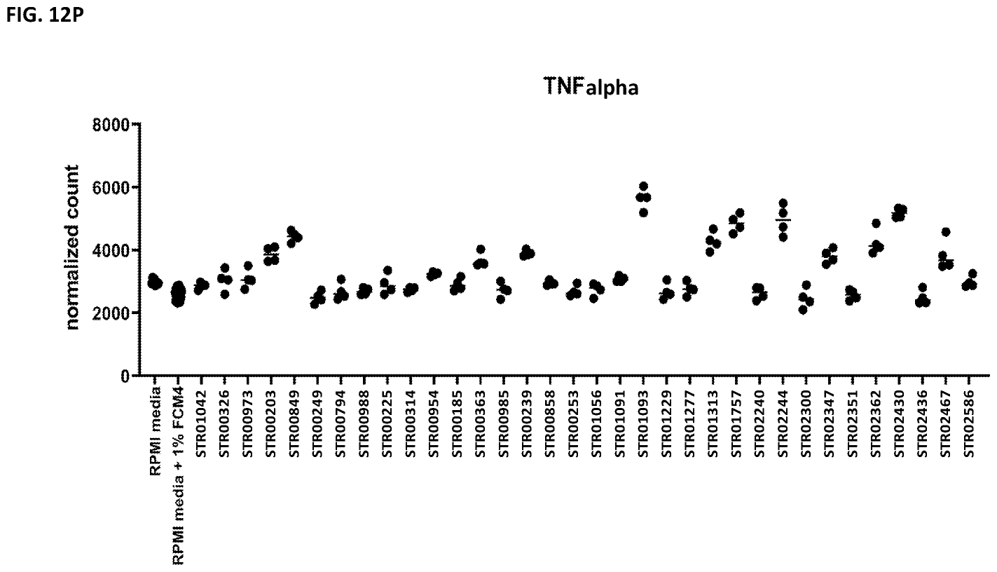

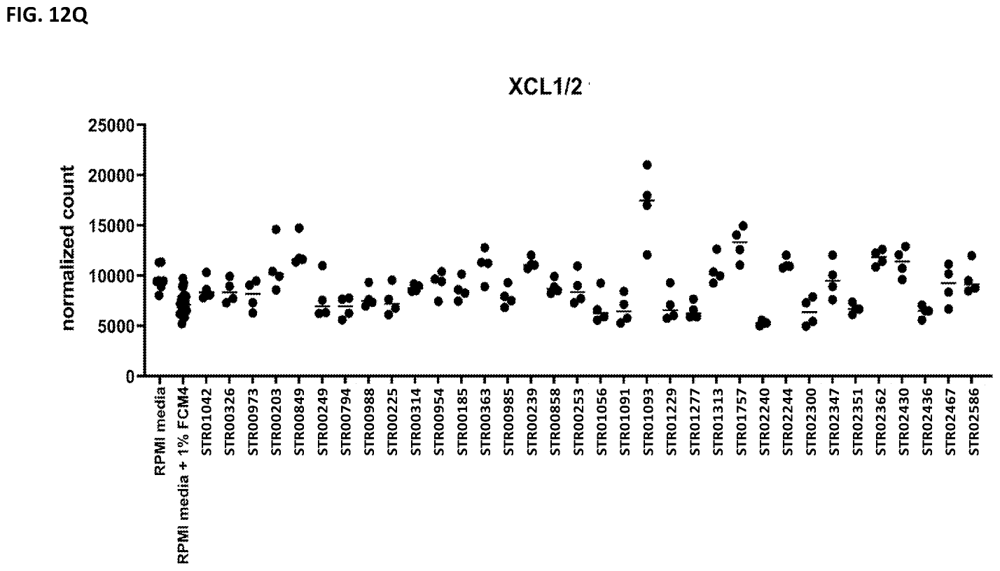

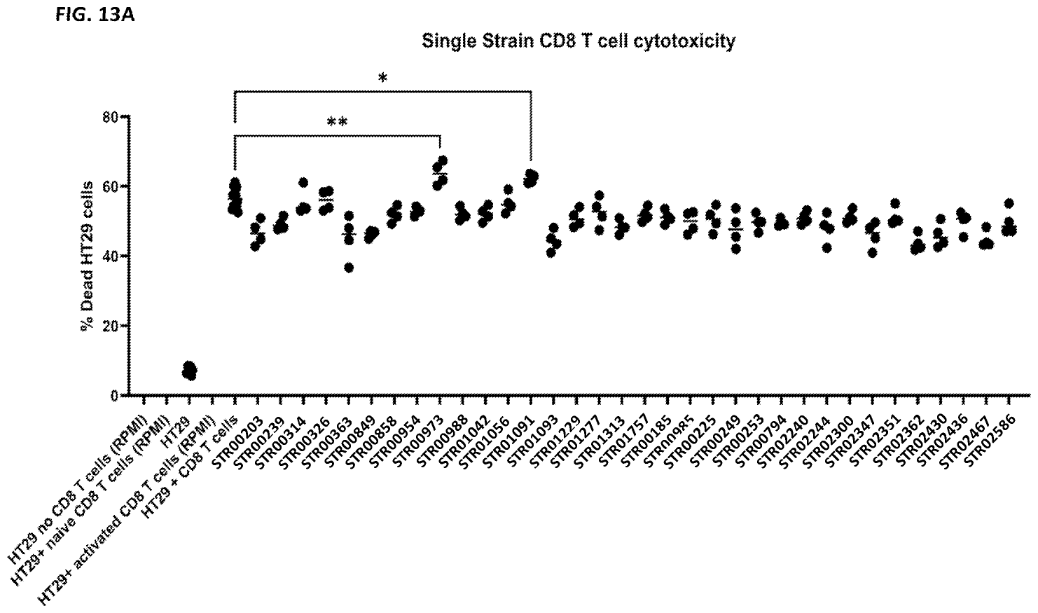

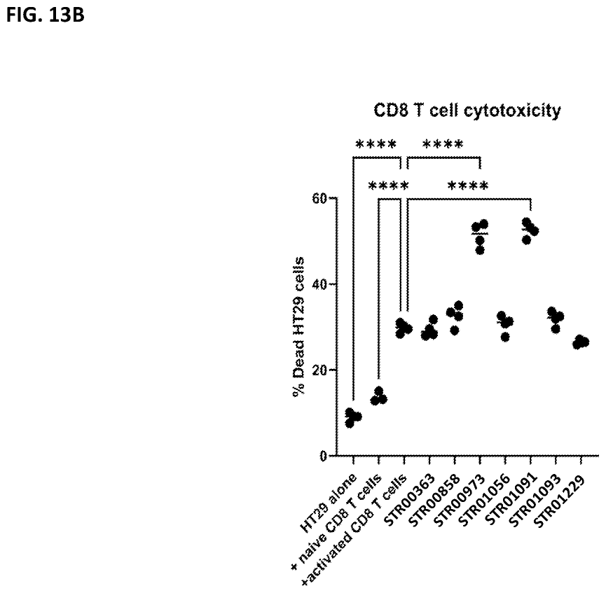

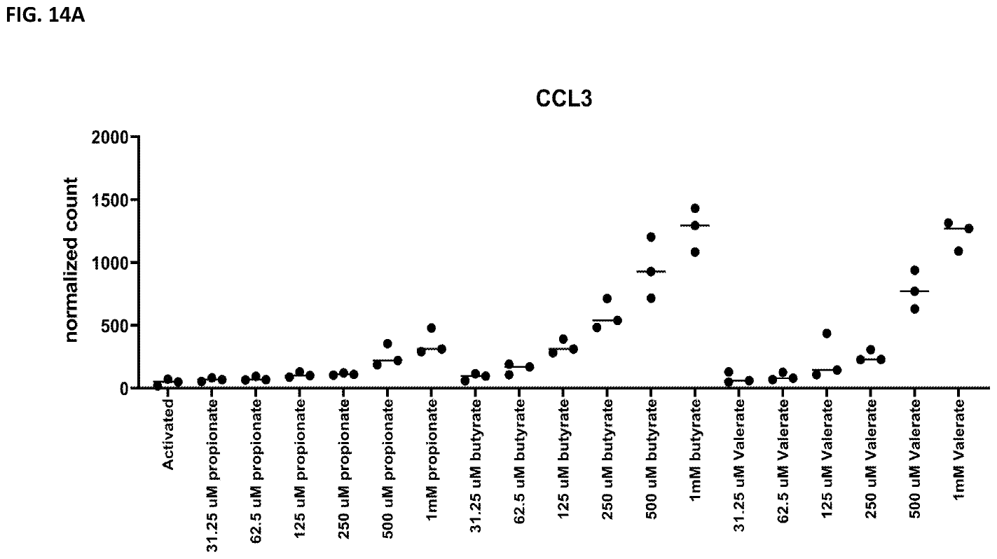

The relationship between single species, short chain fatty acids, and CD8 gene expression data was analyzed. Several single species analyzed using the in vitro CD8 gene expression assay, induced expression of several effector genes such as the CD8 T cell effector cytokines IFNG (IFNγ) and TNFα, cytotoxic molecules granzyme B and perforin, and the activation marker CD69 (FIGS. 12A-Q). CD8 gene expression and cytotoxicity data for 34 single species was specifically assessed. Two experiments, in which the supernatants for each of the experiments were generated in two separate in vitro outgrowths, showed that two species (Murimonas intestini and Faecalicatena orotica) in particular enhanced CD8 cytotoxicity in a co-culture killing assay with a colorectal cancer cell line, HT-29 cells (FIGS. 13A and 13B).

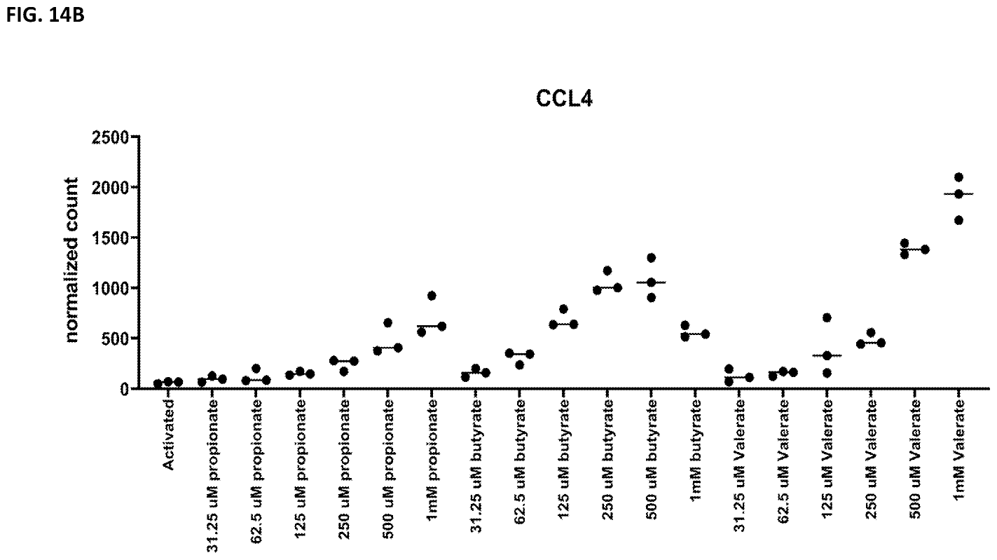

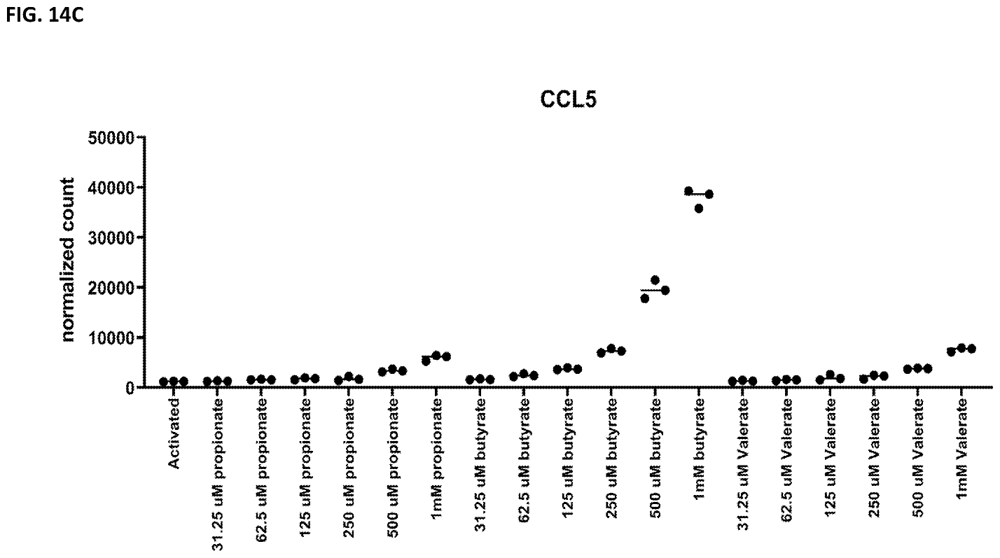

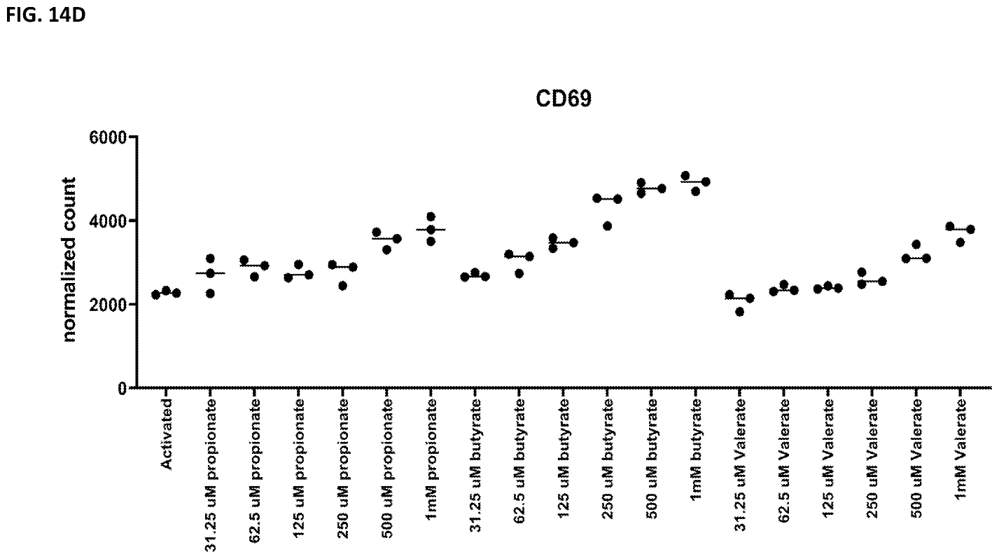

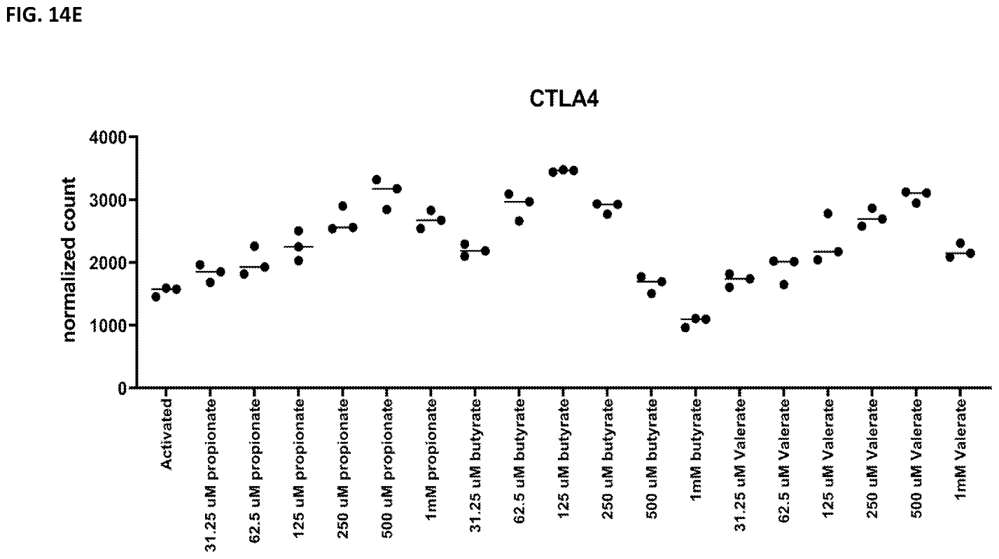

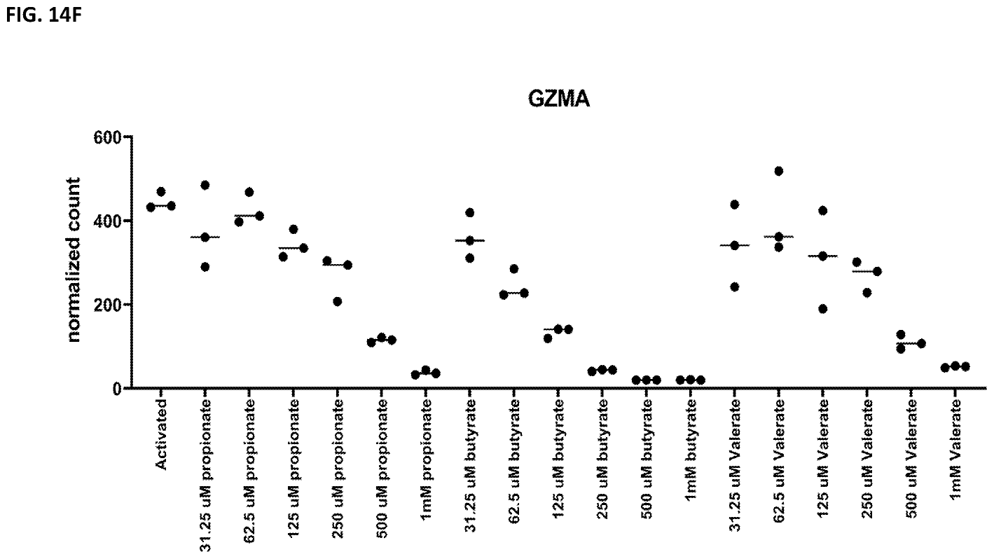

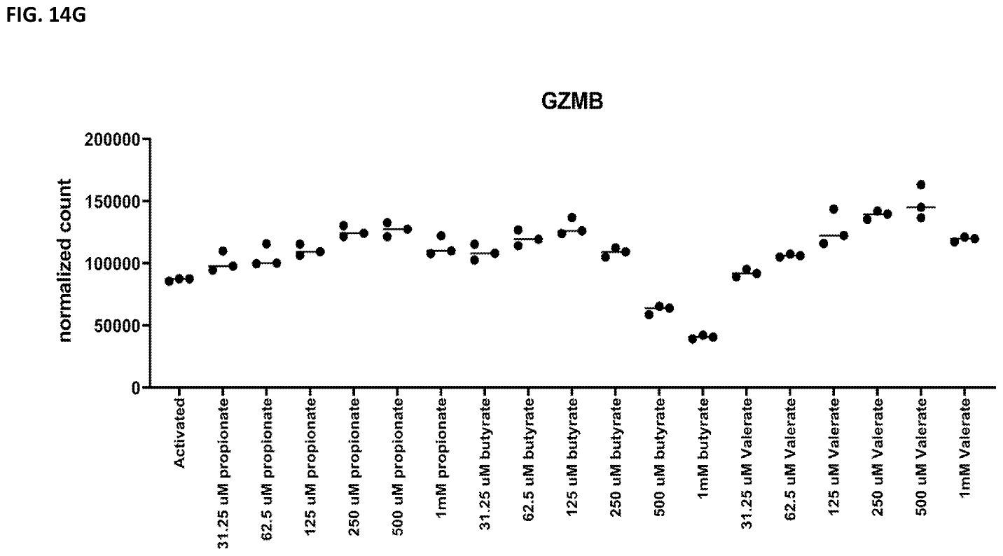

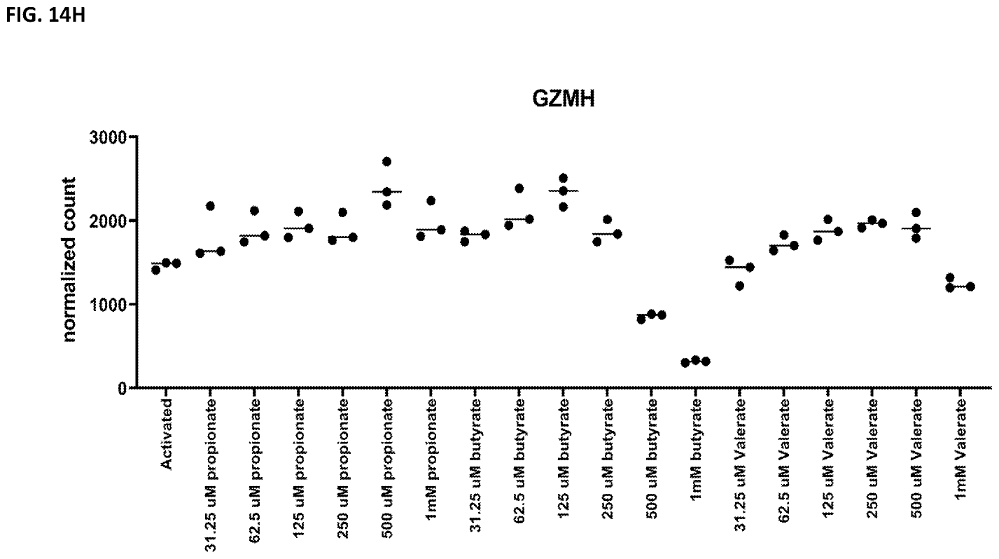

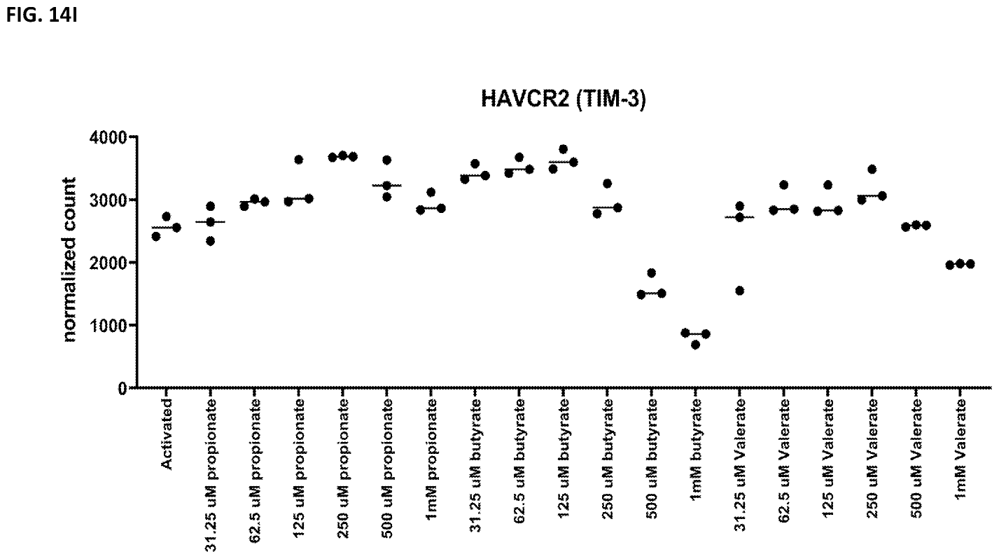

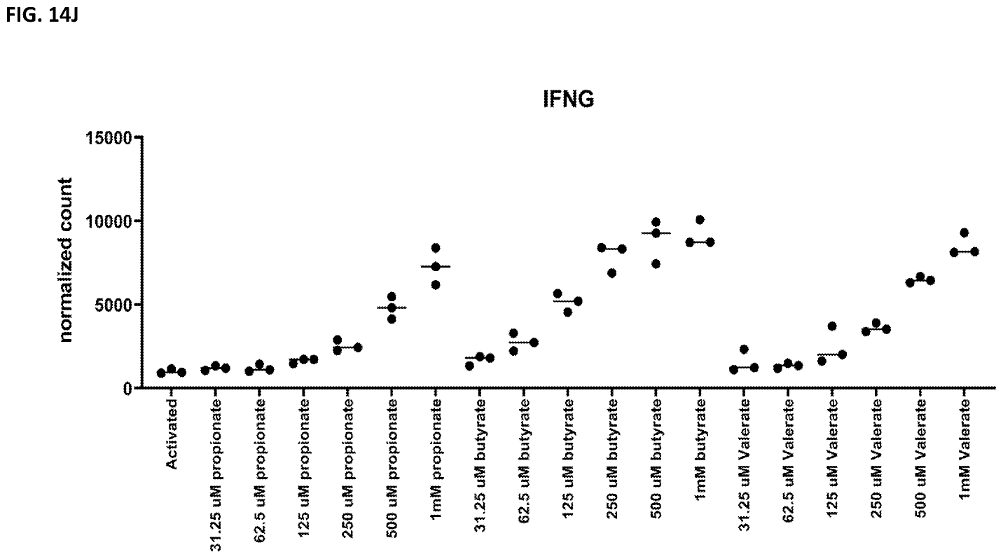

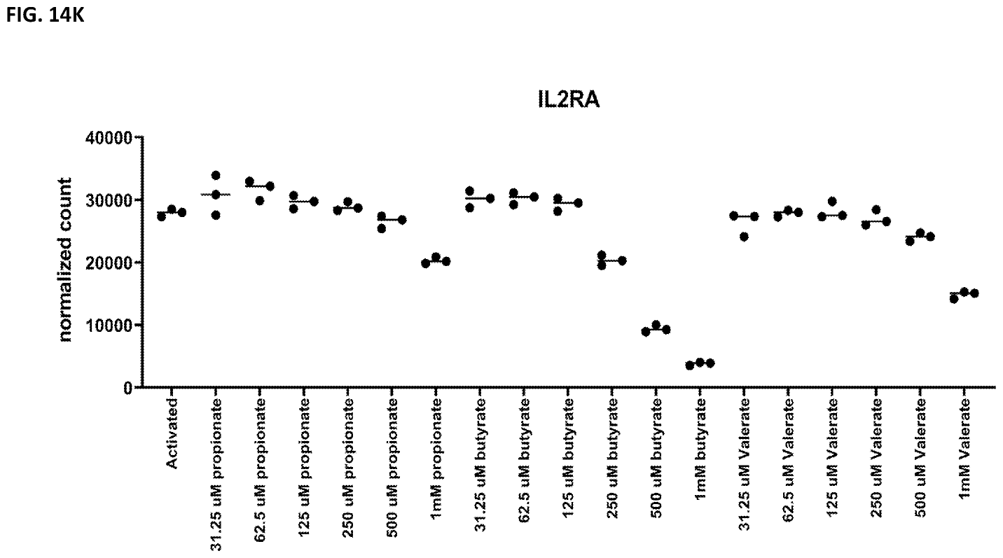

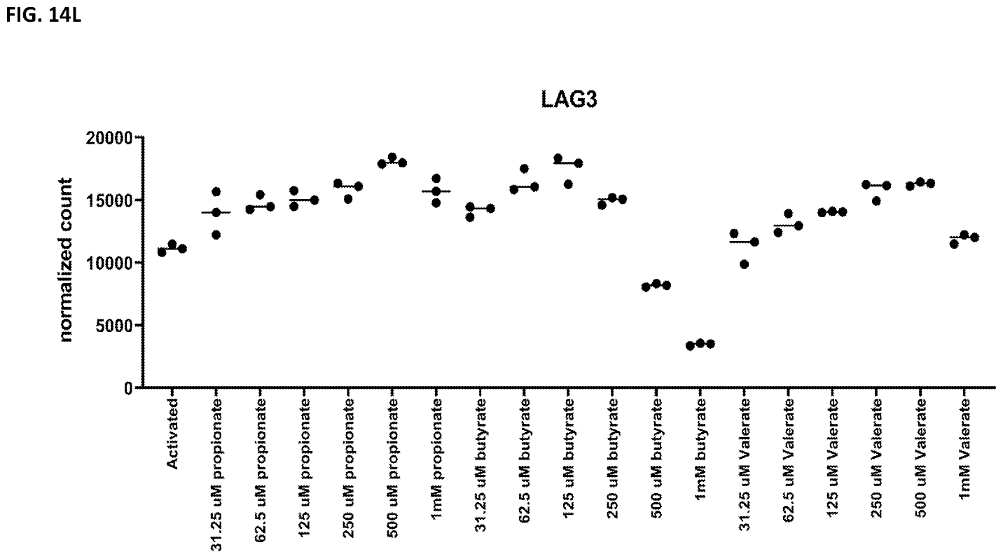

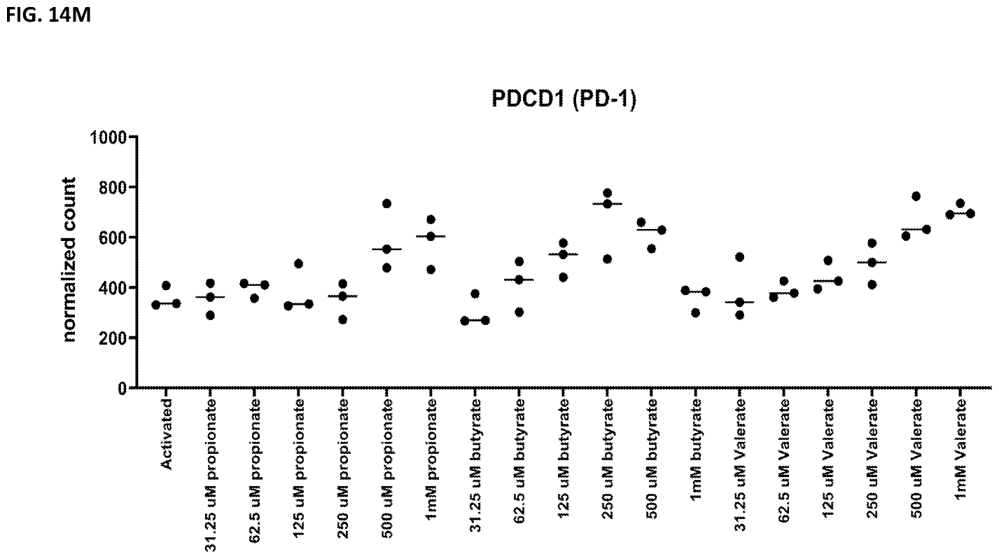

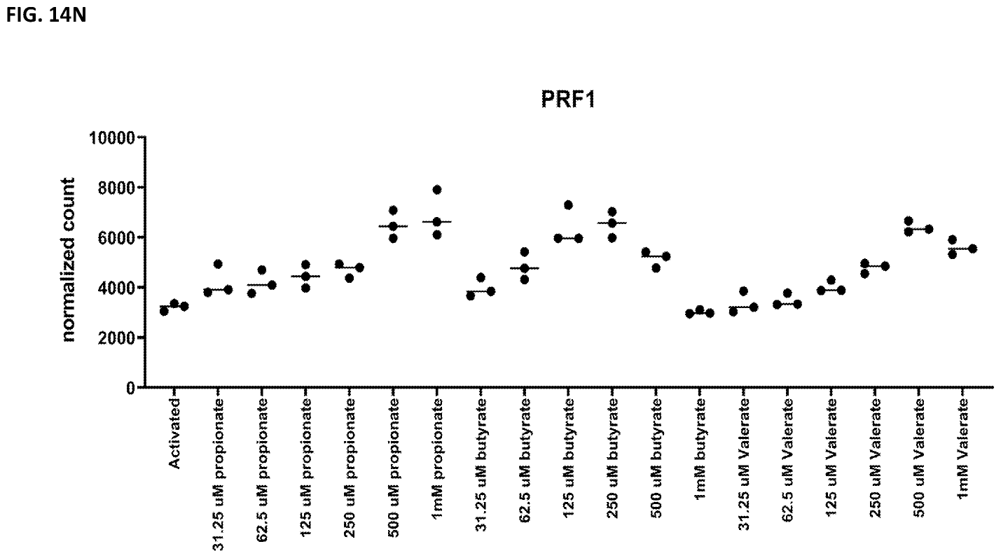

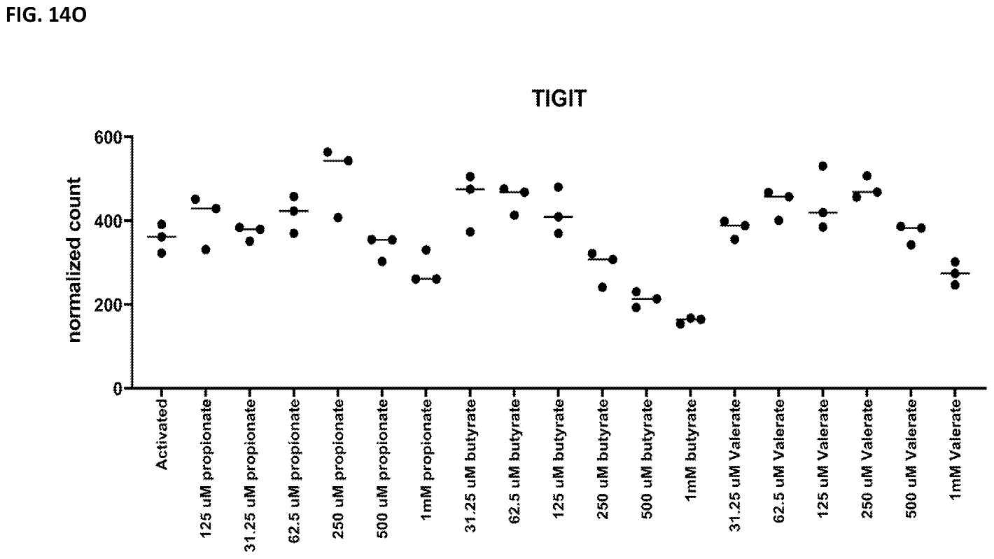

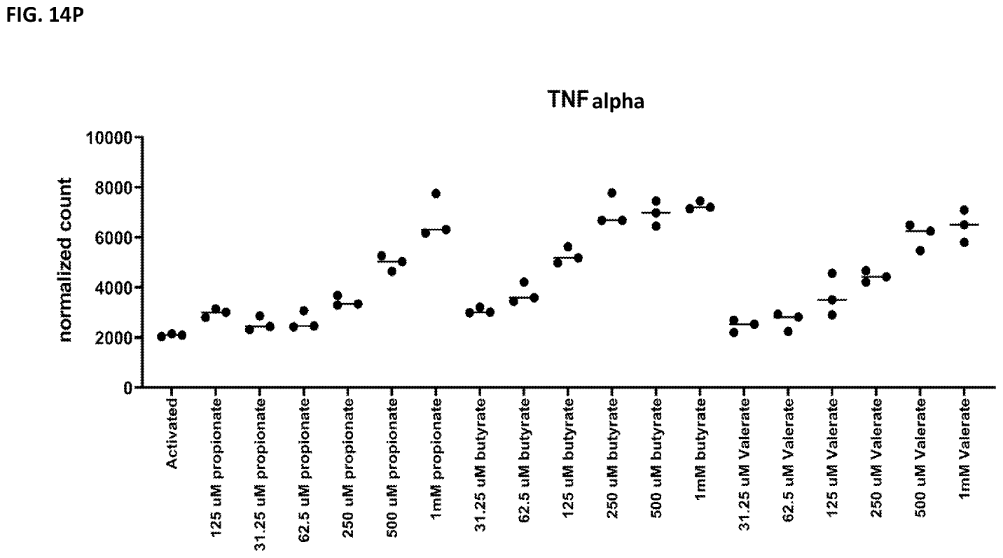

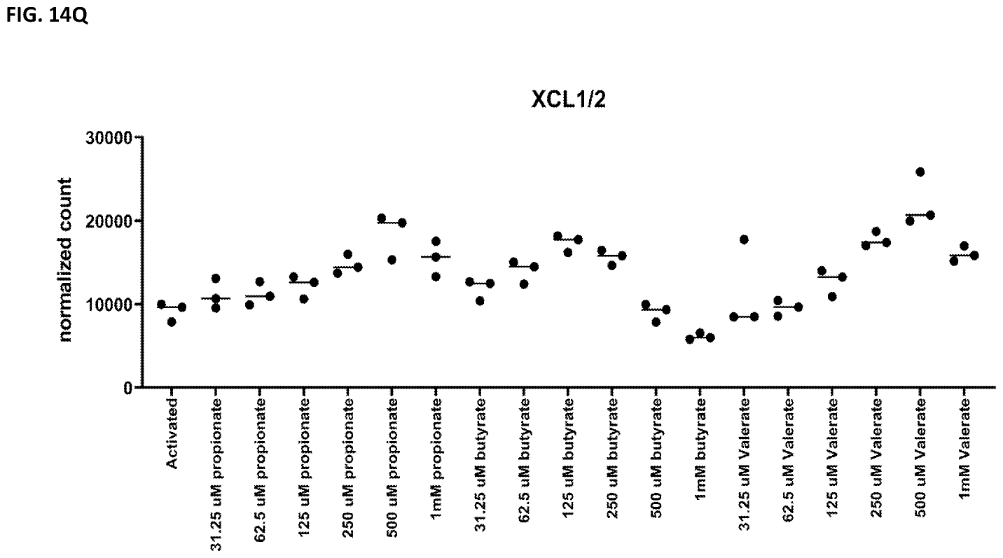

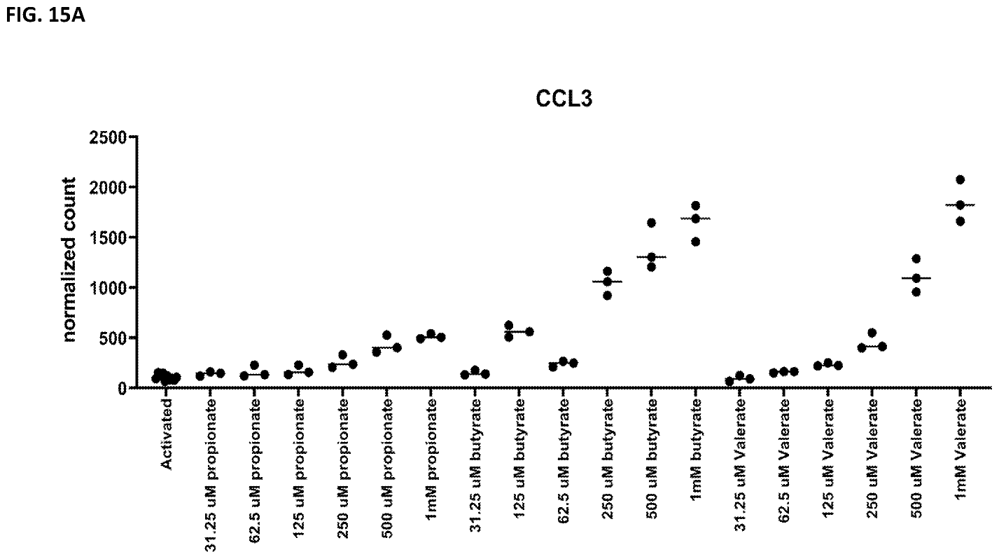

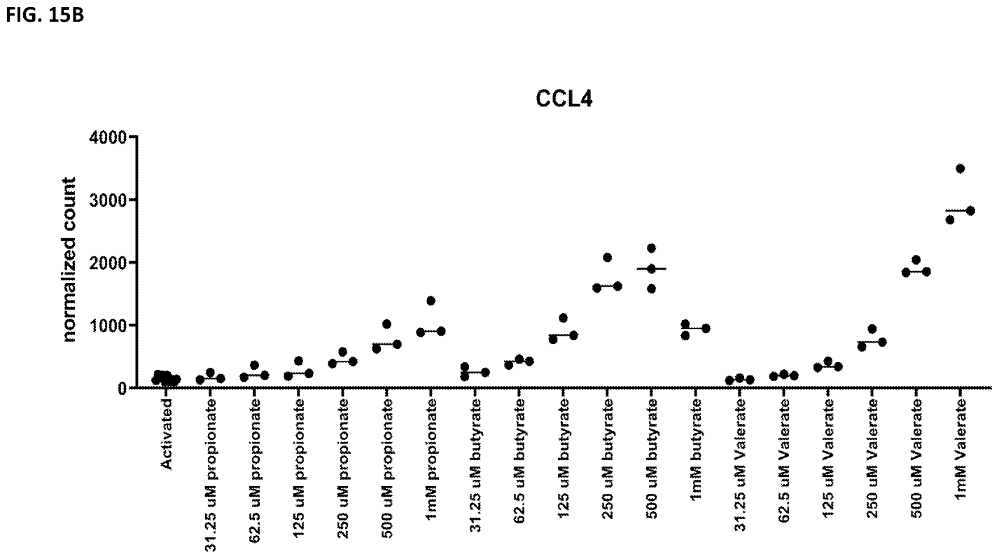

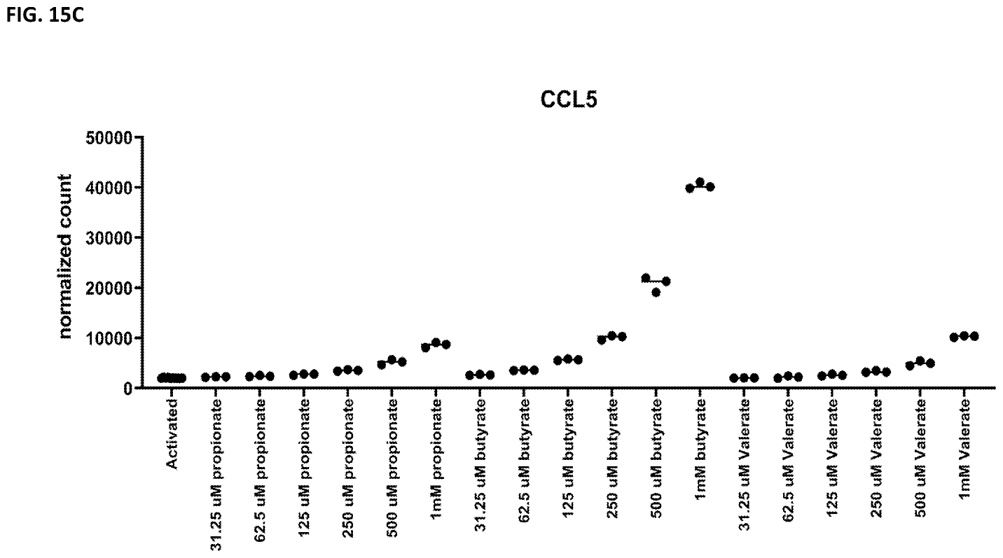

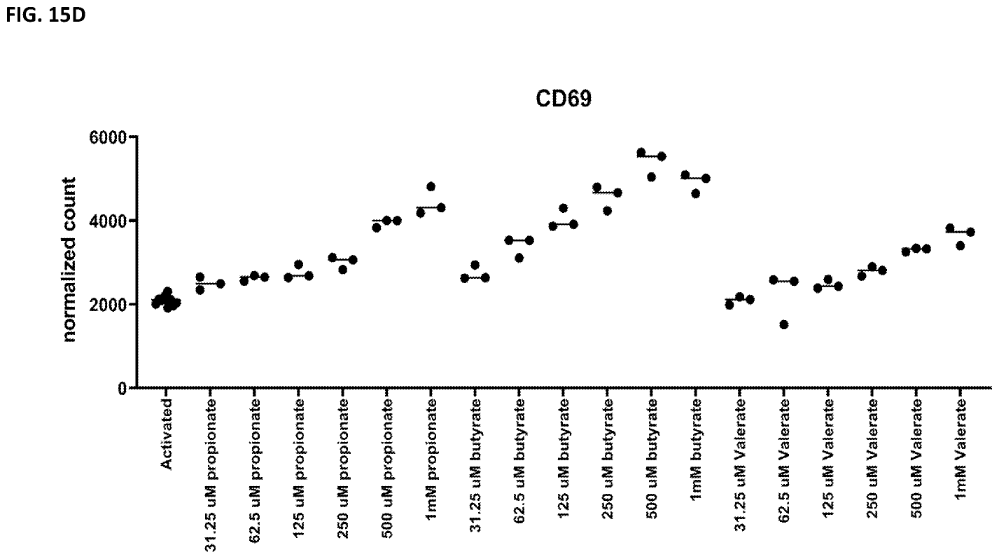

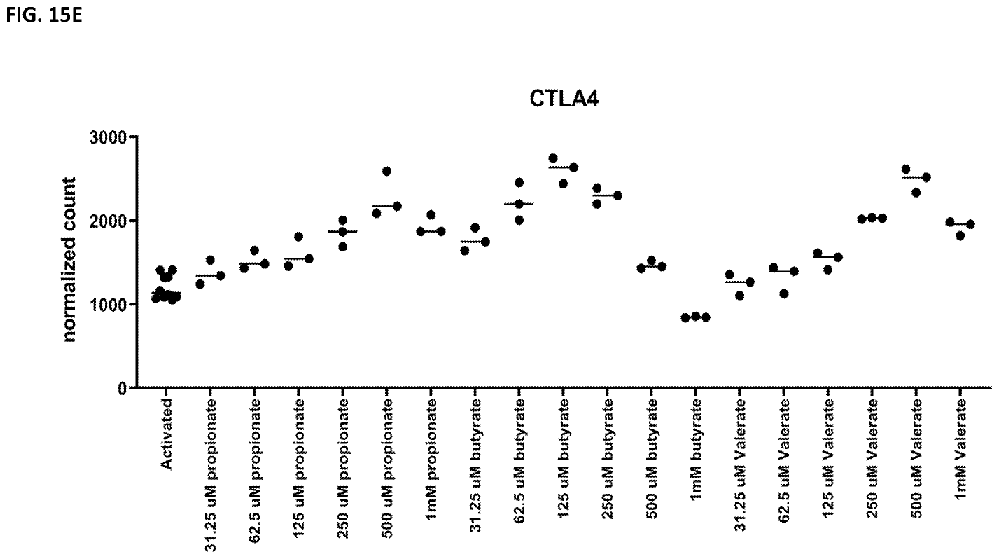

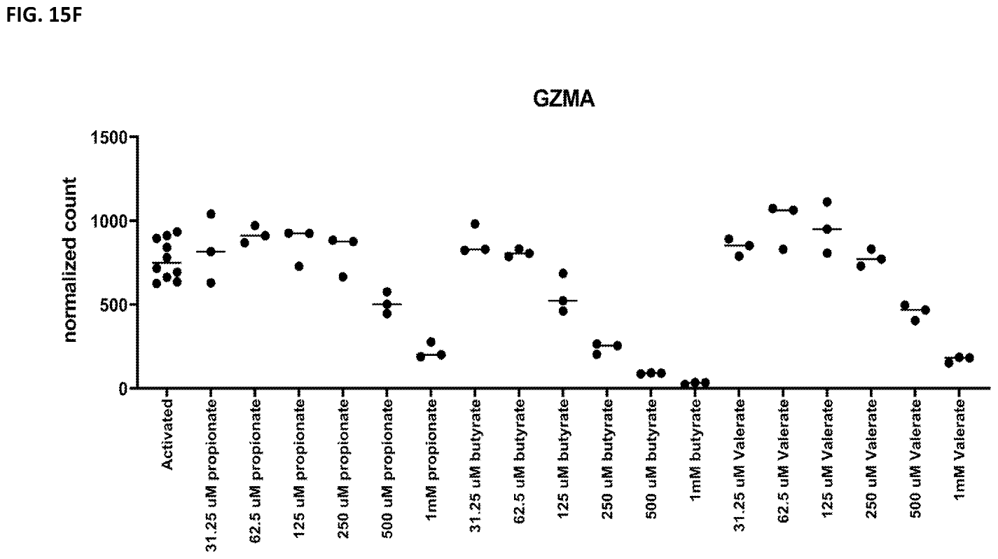

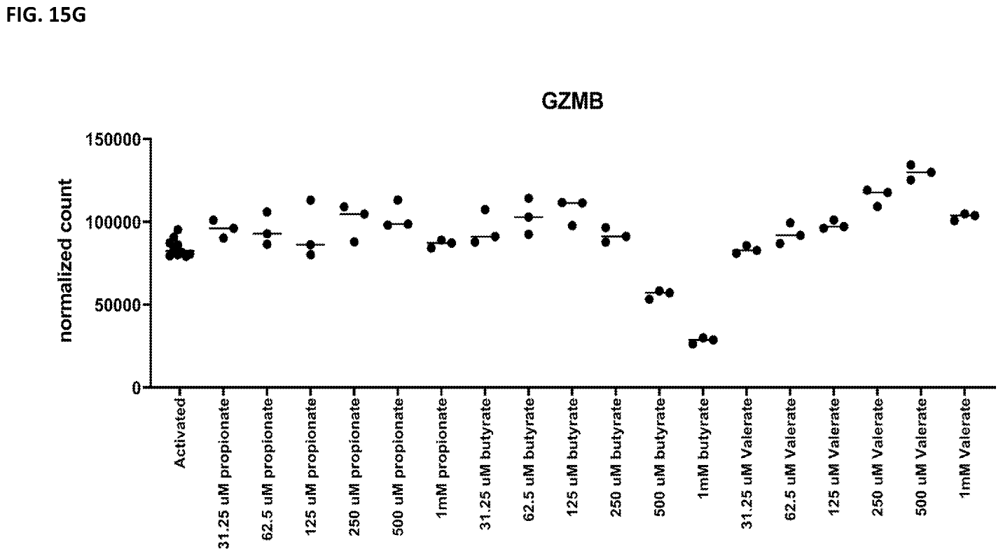

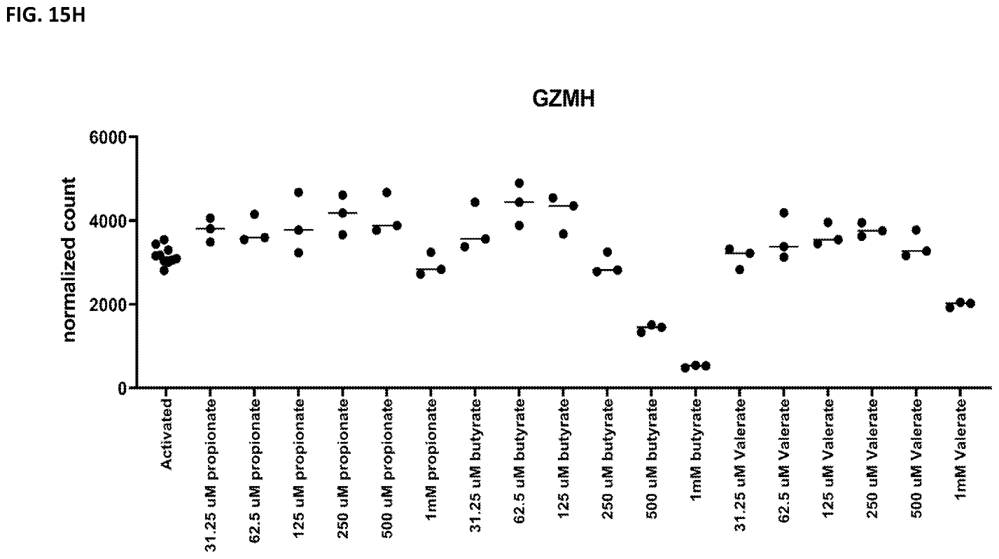

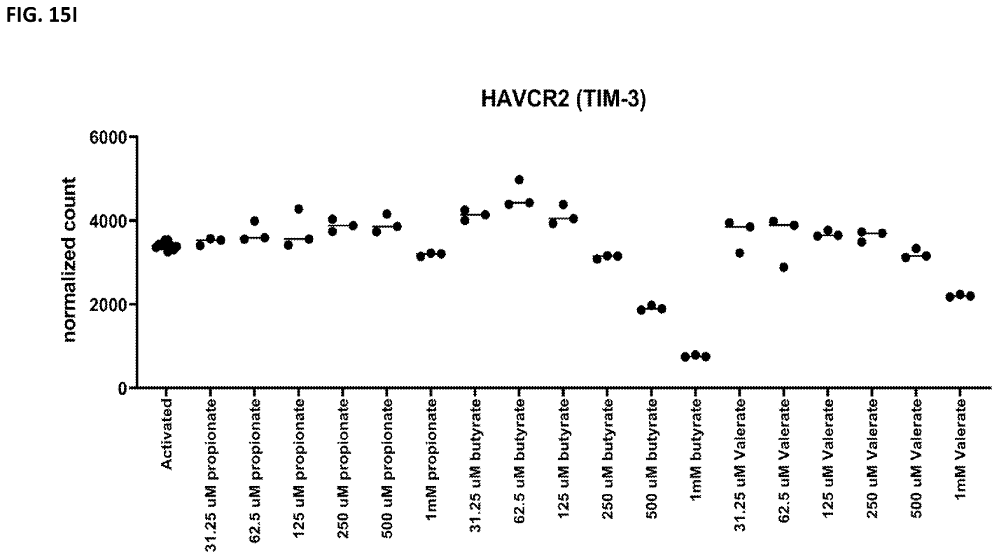

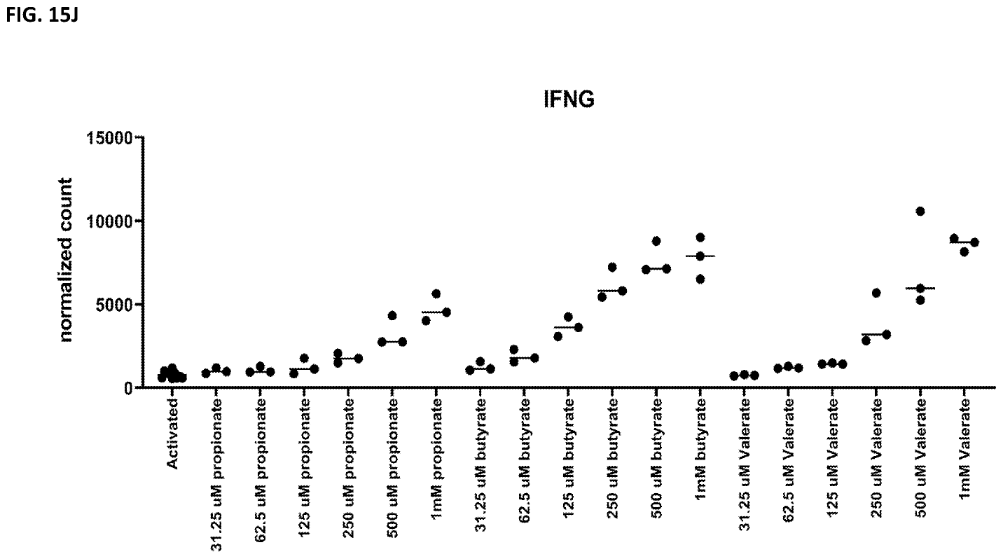

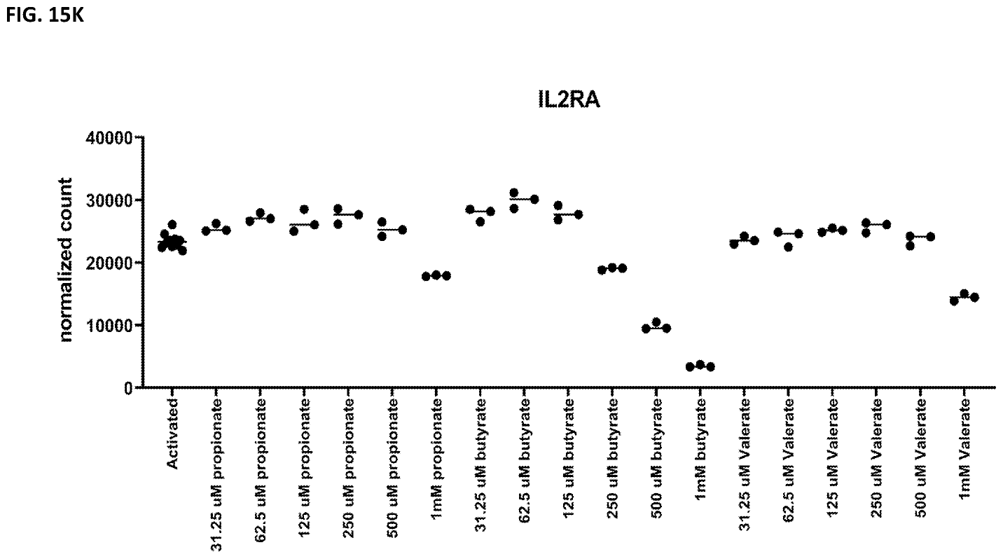

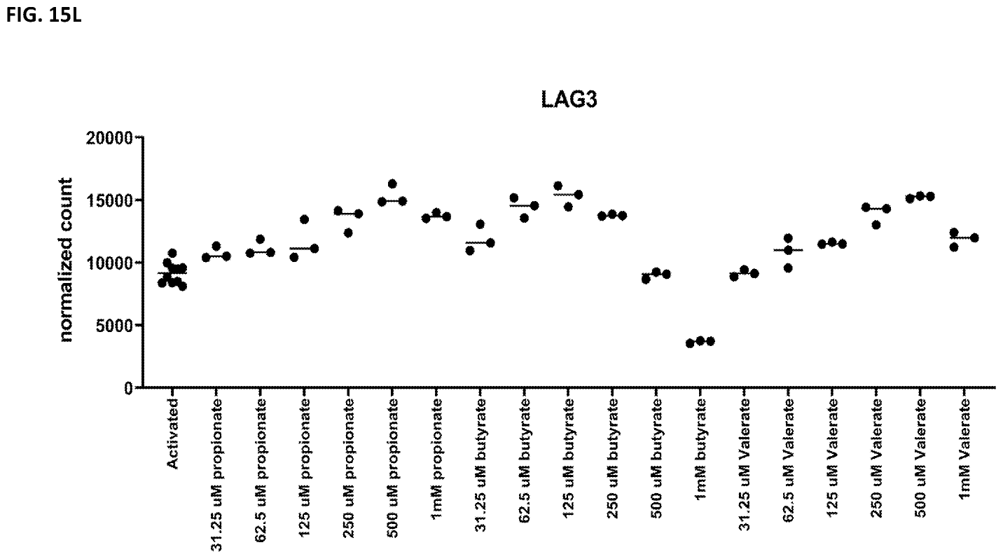

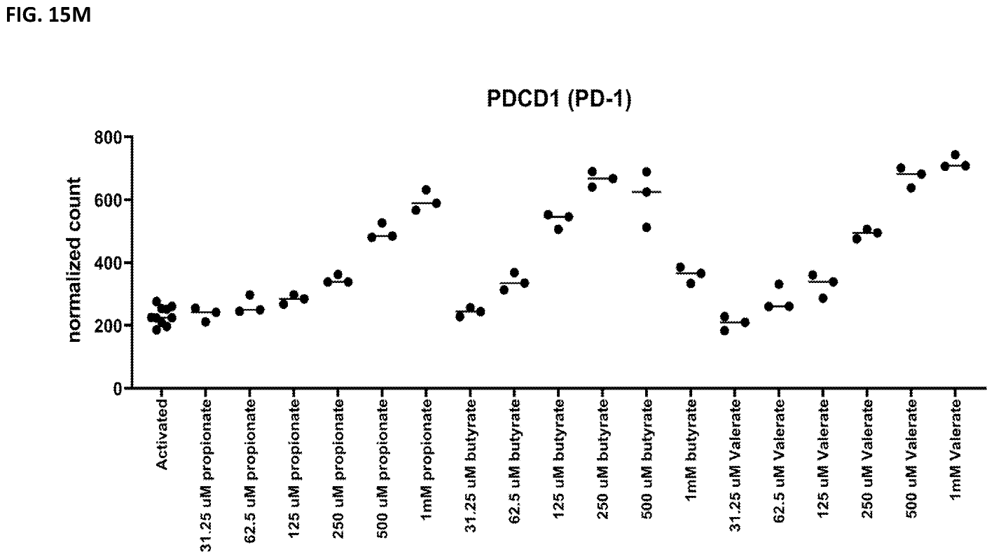

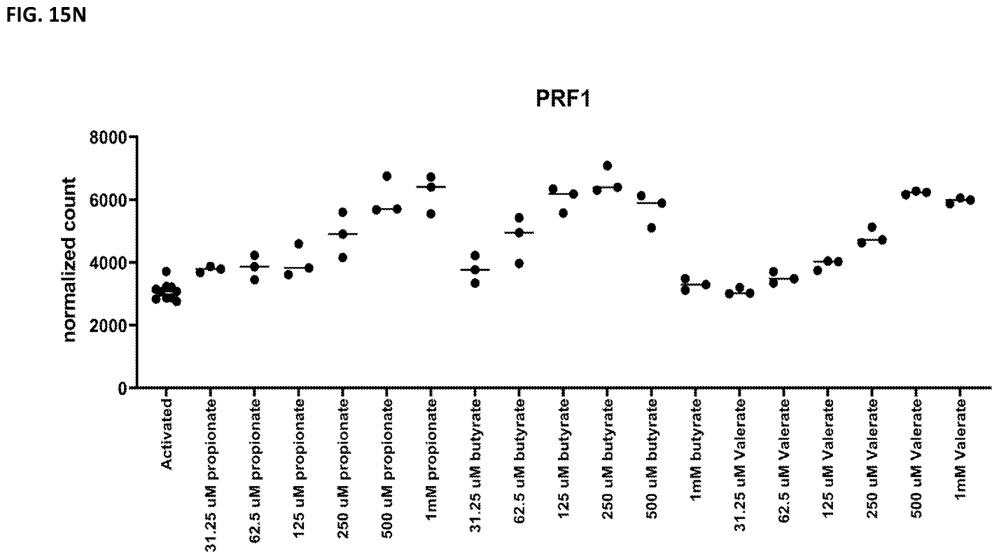

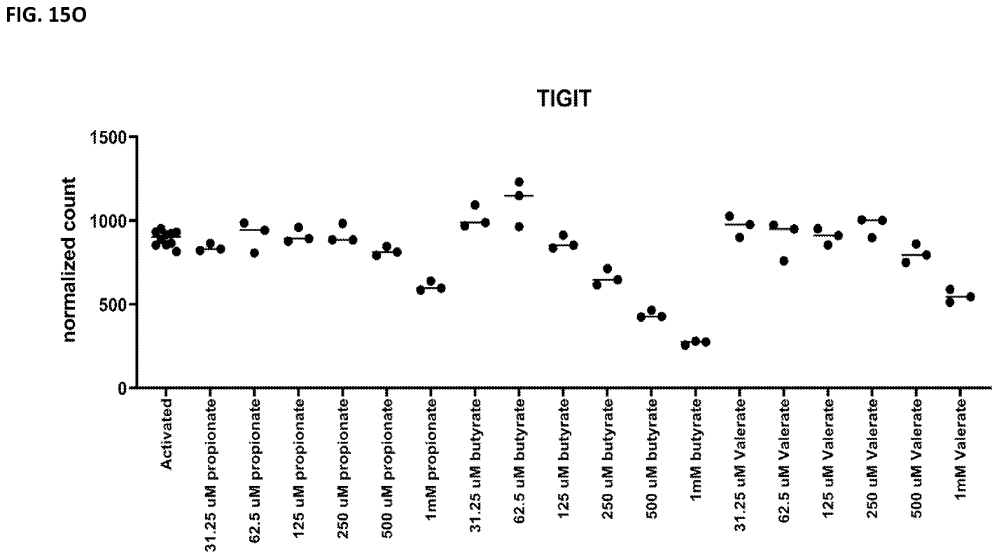

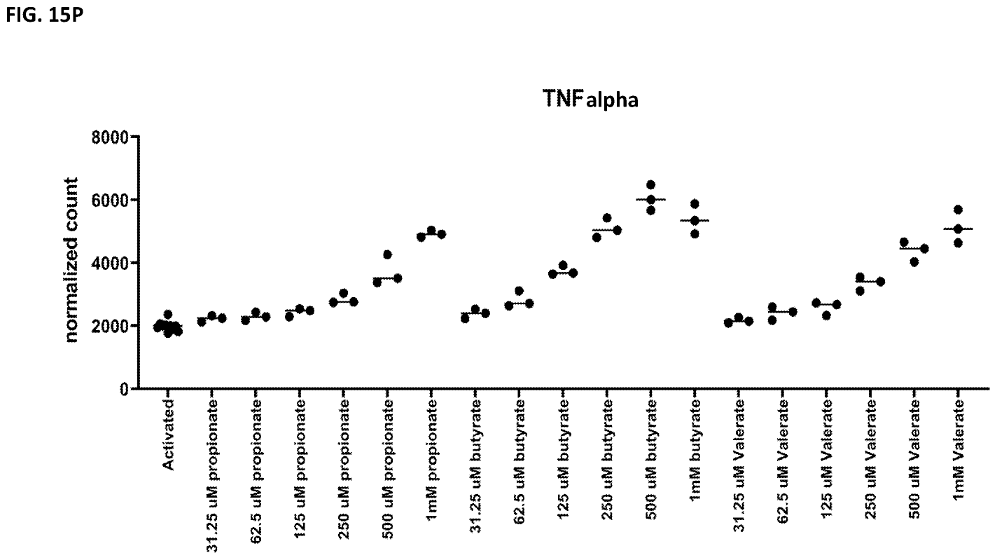

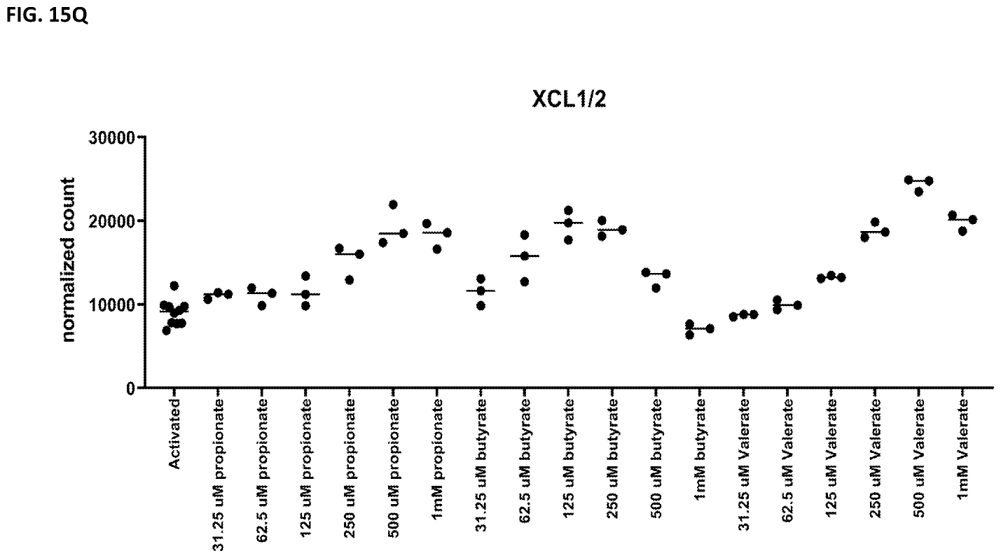

Additionally, data from two different CD8 T cell donors (FIGS. 14A-Q and FIGS. 15A-Q) show a dose-dependent effect on CD8 gene expression [i.e., CCL3, CCL4, CCL5, CD69, CTLA4, GZMA, GZMB, GZMH, HAVCR2 (TIM-3), IFNG, IL2RA LAG3, PDCD1 (PD-1), PRF1, TIGIT, TNFα, XCL 1/2] after treatment with the three SCFAs propionate, butyrate and valerate.

Thus, the results of the in vitro CD8 T cell assay shows that FMTs from responder and non-responder cancer patients to immune checkpoint inhibition therapy can differentially modulate gene expression in CD8 T cells, suggesting a direct microbiome effect on these immune cells. Treating CD8 T cells with FMT supernatants caused changes in expression of activation, immune cell recruitment and inhibitory receptor genes (i.e., CD69, CCL5 and TIM-3, respectively). Analysis of single bacterial strains in the same assays shows that individual strains can also directly affect T cell activation, effector function and cytotoxicity both at the gene expression level, and at the functional level as shown in co-culture assays with the colorectal cancer cell line, HT-29. Finally, testing the SCFAs propionate, butyrate and valerate in the CD8 assay showed a dose-dependent modulation of gene expression in many genes involved in T cell activation, cytotoxicity and effector function, suggesting that the effects on gene expression for bacterial strains and FMTs are at least in part due to bacterial metabolites.

### Example 6: FMT Supernatant CD8 Gene Expression Composite Score (“PC1”) Correlation with Outcome

As many CD8 genes were similarly associated with outcome on a univariate (one-by-one) level analysis, and the expression of several CD8 genes were internally correlated across samples (e.g., CCL5 and TIM3 expression are negatively correlated with each other) a principal component analysis (PCA) was conducted. The raw gene-expression profiles above were transformed into a set of orthogonal composite scores using PCA. Generalized linear regression was then applied to test whether any principal component was associated with outcome.

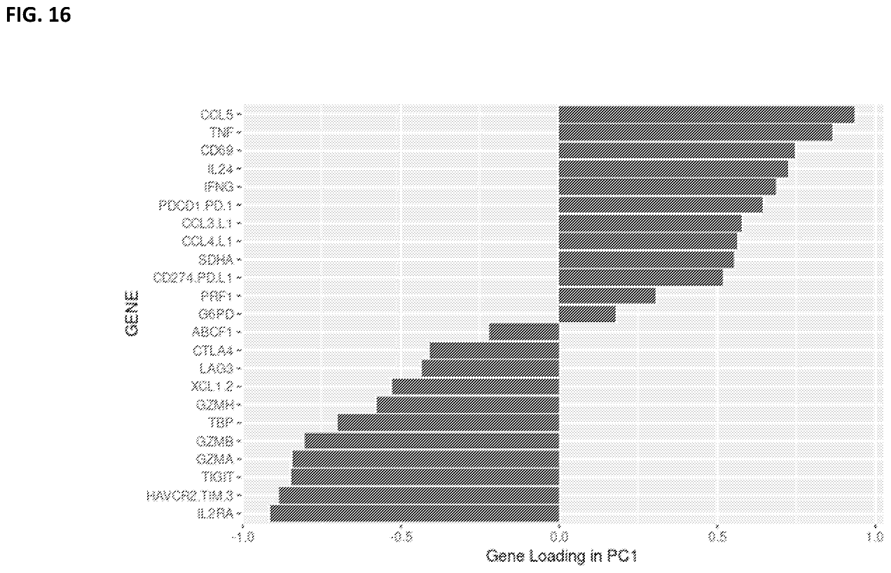

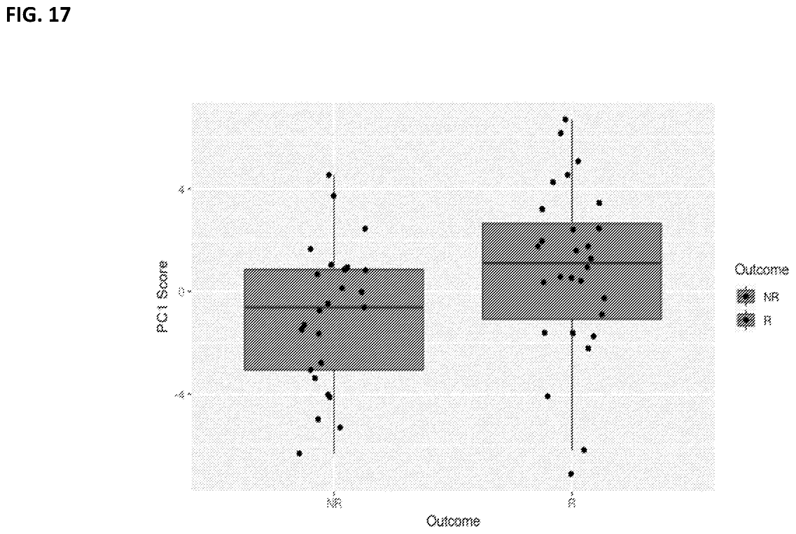

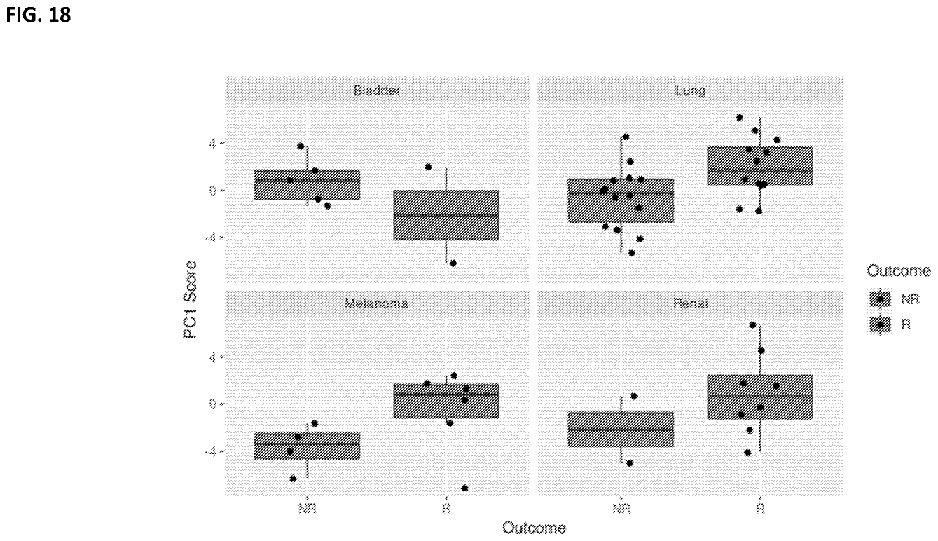

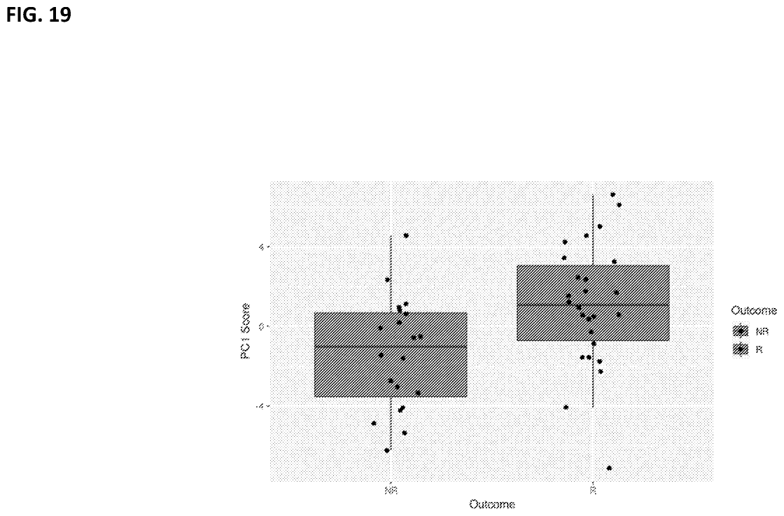

Principal component 1 (PC1) accounted for 44% of the variation present in all genes. PC1 is a composite score involving all genes but the 6 most important genes were: CCL5 and TNFalpha positively loaded and IL2RA, TIM2, TIGIT, and Granzyme A (GZMA) negatively loaded (FIG. 16). PC1 was positively associated with responder status (p=0.04, FIG. 17). PC1 association varied by cancer type with bladder samples containing a signal opposite to the other three cancer types (FIG. 18). When bladder samples are removed the association with outcome increased in strength (p=0.01, FIG. 19).

These results suggest that bacterial metabolic output, captured by culture of FMTs derived from the stool of CPI treated patients, create a coherent CD8 T-cell gene response where multiple activation and effector genes move in concert. In addition, the primary axis of variation (PC1) shared by these genes quantitatively differs among patients who responded to CPI compared with those who did not, implying bacterial metabolism relevant to immune activation differs among these patient populations.

### Example 7: Stool Taxonomic Correlates with PC1

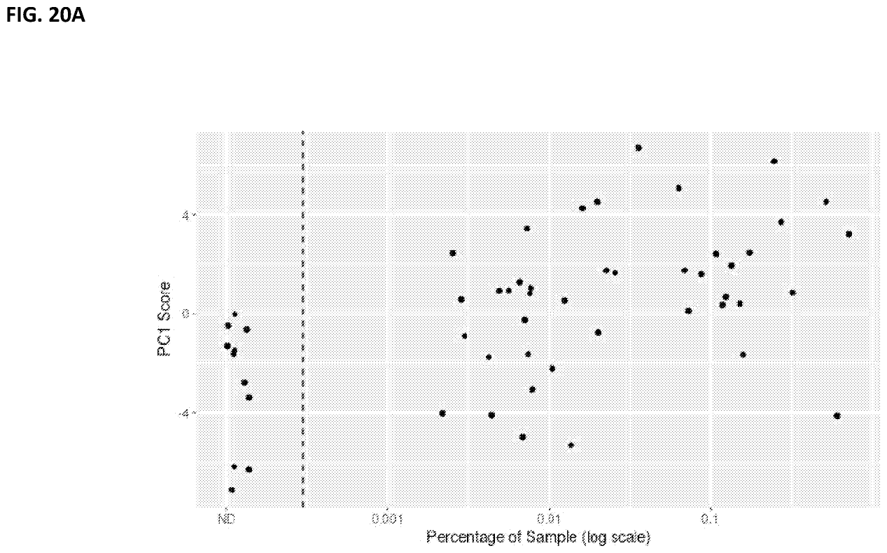

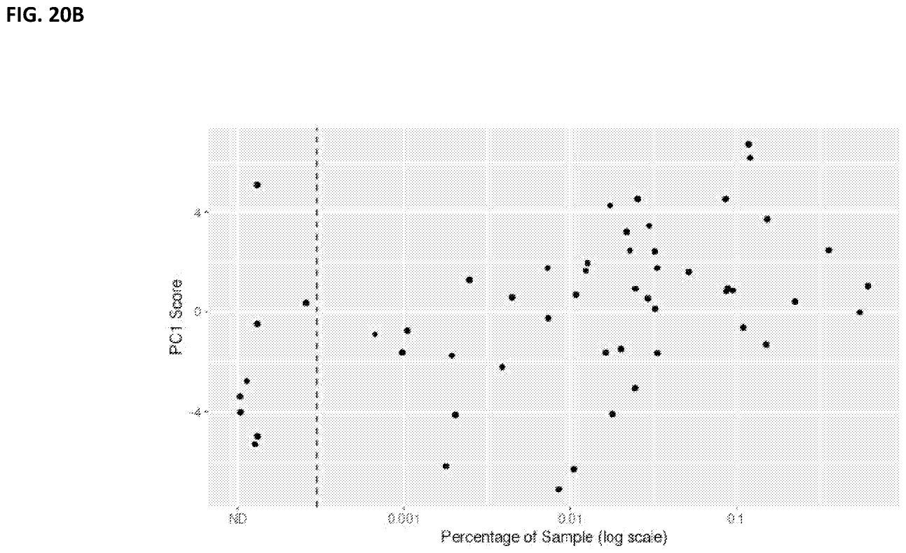

Applying a spearman rank test demonstrates the abundance of certain taxa within patient stool are associated with the PC1 score derived from FMT outgrowths as described in Example 5 and Example 6 (Table 17). Specifically, the abundance of two species Intestinimonas butyriciproducens (FIG. 20A) and Anaerotruncus colihominis (FIG. 20B) were very significantly correlated with PC1 across patients (Intestinimonas butyriciproducens: rho=0.54; p-value=2.6e-5; q-value=0.013; Anaerotruncus colihominis: rho=0.49; p-value=1.7e-4; q-value=0.044). Without wishing to be bound by theory, the significant correlation indicates that each species contributes to the CD8 signature. Additional species also showed a high correlation with PC1 across patients, and were also demonstrated to activate gene pathways useful for treating cancer (e.g., anti-inflammatory) and/or enhance CD8 cytotoxicity with a cancer cell line (e.g., Faecalicatena orotica). This finding implies that increasing abundances of these taxa within a complex community of bacteria, as is found in stool or FMTs, improves the overall ability of a complex community to modulate CD8+ T-cells activity into a state we have demonstrated is correlated with CPI response in patients. Notably, some of these taxa (Lachnospiraceae_NG18 sp7, Negativibacillus sp1, Ruminococcaceae_NG18 sp7, and Coriobacteriaceae_NG1) are associated with both PC1/modulation of CD8+ T-cells and response to CPI therapy.

TABLE 17
Stool taxonomic correlates with PC1

| | Taxa | | | Level_specidic | |
|---|---|---|---|---|---|
| Taxa | Level | spearman.p | spearman.rho | FDR | Global_qval |
| Intestinimonas_butyriciproducens | Species | 2.55747E−05 | 0.543943195 | 0.013119821 | 0.019127402 |
| Intestinimonas | Genus | 4.94248E−05 | 0.527466365 | 0.01057691 | 0.019127402 |
| Anaerotruncus_colihominis | Species | 0.000174662 | 0.493300283 | 0.044800686 | 0.045062679 |
| Lachnospiraceae_NG17_sp3 | Species | 0.000960588 | −0.440601881 | 0.143786765 | 0.144627623 |
| Lachnospiraceae_NG29_sp1 | Species | 0.001121144 | 0.435378156 | 0.143786765 | 0.144627623 |
| Lachnospiraceae_NG29 | Genus | 0.001121144 | 0.435378156 | 0.119962447 | 0.144627623 |
| Fusicatenibacter_sp1 | Species | 0.002568004 | 0.405863148 | 0.203596452 | 0.19466764 |
| Dysosmobacter_welbionis | Species | 0.002591175 | 0.408321239 | 0.203596452 | 0.19466764 |
| Oxalobacter_formigenes | Species | 0.002778119 | 0.402920178 | 0.203596452 | 0.19466764 |
| Oxalobacter | Genus | 0.002778119 | 0.402920178 | 0.174924217 | 0.19466764 |
| Oxalobacteraceae | Family | 0.002778119 | 0.402920178 | 0.130571604 | 0.19466764 |
| Emergencia_timonensis | Species | 0.003269612 | 0.39673953 | 0.209663839 | 0.19466764 |
| Emergencia | Genus | 0.003269612 | 0.39673953 | 0.174924217 | 0.19466764 |
| Dysosmobacter | Genus | 0.006257586 | 0.37284309 | 0.242554343 | 0.345955133 |
| Lachnoclostridium | Genus | 0.006800589 | 0.367420198 | 0.242554343 | 0.350910395 |
| Eisenbergiella_massiliensis | Species | 0.007565859 | 0.362923205 | 0.402548306 | 0.357266814 |
| Lachnoclostridium_edouardi | Species | 0.007846946 | 0.361370513 | 0.402548306 | 0.357266814 |
| Prochlorotrichaceae_NG20_sp1 | Species | 0.01001542 | 0.350789566 | 0.467082772 | 0.407996585 |
| Prochlorotrichaceae_NG20 | Genus | 0.01001542 | 0.350789566 | 0.306185699 | 0.407996585 |
| Butyricicoccus | Genus | 0.012377923 | 0.341316842 | 0.331109429 | 0.479025603 |
| Blautia_coccoides_group | Species | 0.015832609 | 0.329945209 | 0.510152091 | 0.513135436 |
| Coriobacteriaceae_NG1 | Genus | 0.01663508 | −0.327611724 | 0.359472893 | 0.513135436 |
| Anaerotruncus | Genus | 0.016797799 | 0.327150295 | 0.359472893 | 0.513135436 |
| Intestinimonas_timonensis | Species | 0.01695503 | 0.326708028 | 0.510152091 | 0.513135436 |
| Angelakisella_sp1 | Species | 0.017171658 | −0.326104374 | 0.510152091 | 0.513135436 |
| Alistipes_putredinis | Species | 0.019150901 | 0.320869774 | 0.510152091 | 0.513135436 |
| Collinsella_bouchesdurhonensis | Species | 0.019378922 | −0.320296612 | 0.510152091 | 0.513135436 |
| Bacteroides_plebeius | Species | 0.019627656 | 0.319677869 | 0.510152091 | 0.513135436 |
| Clostridium_hylemonae | Species | 0.01975156 | 0.319372123 | 0.510152091 | 0.513135436 |
| Christensenellaceae_NG16_sp1 | Species | 0.021213724 | 0.315881554 | 0.510152091 | 0.513135436 |
| Christensenellaceae_NG16 | Genus | 0.021213724 | 0.315881554 | 0.412703362 | 0.513135436 |
| Lawsonibacter_sp7 | Species | 0.022615005 | 0.312722189 | 0.510152091 | 0.513135436 |
| Coprococcus_catus | Species | 0.022953537 | −0.311983769 | 0.510152091 | 0.513135436 |
| Coriobacteriaceae_NG5_sp1 | Species | 0.023747023 | 0.310288294 | 0.510152091 | 0.513135436 |
| Peptoniphilaceae_NG4_sp2 | Species | 0.023779256 | 0.310220434 | 0.510152091 | 0.513135436 |
| Christensenella_minuta | Species | 0.023866764 | 0.310036593 | 0.510152091 | 0.513135436 |
| Coprobacter_secundus_group | Species | 0.025333184 | −0.307037223 | 0.519836933 | 0.515996955 |
| Gabonia | Genus | 0.025333184 | −0.307037223 | 0.436385531 | 0.515996955 |
| Anaerotignum | Genus | 0.026509401 | 0.304735047 | 0.436385531 | 0.526109659 |
| Erysipelotrichia_NO11_NF1 | Family | 0.033432674 | −0.292693167 | 0.559350727 | 0.646922234 |
| Desulfovibrio | Genus | 0.034943868 | 0.290345175 | 0.534141989 | 0.657778548 |
| Bacteroides_gallinarum | Species | 0.03748788 | −0.286575881 | 0.678922043 | 0.657778548 |
| Negativibacillus_sp1 | Species | 0.038699248 | 0.284854903 | 0.678922043 | 0.657778548 |
| Faecalicatena_orotica | Species | 0.04053956 | 0.282323476 | 0.678922043 | 0.657778548 |
| Lachnospiraceae_NG18_sp7 | Species | 0.042900325 | 0.279210999 | 0.678922043 | 0.657778548 |
| Bittarella | Genus | 0.04330321 | 0.278693945 | 0.575984458 | 0.657778548 |
| Bittarella_massiliensis | Species | 0.043493604 | 0.278450969 | 0.678922043 | 0.657778548 |
| Peptostreptococcaceae | Family | 0.044224038 | −0.277526816 | 0.559350727 | 0.657778548 |
| Intestinibacter_Paraclostridium_Terrisporo- | Genus | 0.045986532 | −0.275347286 | 0.575984458 | 0.657778548 |
| bacter_Candidatus_Dorea_Paeniclostridium_Romboutsia | | | | | |
| Anaerotignum_lactatifermentans | Species | 0.046239118 | 0.275040553 | 0.678922043 | 0.657778548 |
| Bacteroides_cellulosilyticus | Species | 0.046865725 | 0.274285488 | 0.678922043 | 0.657778548 |
| Cuneatibacter_caecimuris | Species | 0.047506224 | 0.27352215 | 0.678922043 | 0.657778548 |
| Alistipes | Genus | 0.047755303 | 0.273227569 | 0.575984458 | 0.657778548 |
| Marvinbryantia_sp5 | Species | 0.047853306 | −0.273112005 | 0.678922043 | 0.657778548 |
| Ruminococcaceae_NG18_sp7 | Species | 0.048305355 | −0.272581442 | 0.678922043 | 0.657778548 |
| Lawsonibacter_sp3 | Species | 0.049816551 | 0.27083664 | 0.678922043 | 0.657778548 |

Taxa (taxonomic group), Taxa Level (Taxonomic level), spearman.p (p-value from spearman correlation), spearman.rho (Rho value from spearman correlation, positive means positively associated with PC1), Level_specidic FDR (FDR corrected q-values within that taxonomic level), Global_qval (FDR corrected q-values across all taxonomic levels)

TABLE A
Species associated with SEQ ID Nos.

| Species | SEQ ID Nos. |
|---|---|
| Massilimaliae_timonensis | 1 |
| Frisingicoccus_sp2 | 9 |
| Alistipes_putredinis | 14 |
| Denitrobacterium_detoxificans | 56 |
| Marvinbryantia_sp8 | 62 |
| Anaerotignum_sp2 | 63 |
| Ruminococcaceae_NG15_sp3 | 69 |
| Neglecta_timonensis | 121 |
| [Anaerotruncus]_rubiinfantis | 124 |
| Prevotellamassilia_timonensis | 126 |
| Massilimaliae_massiliensis | 130 |
| Muricomes_sp1 | 133 |
| Gordonibacter_pamelaeae | 162 |
| Peptoniphilaceae_NG4_sp2 | 172 |
| Ruminococcaceae_NG33_sp1 | 184 |
| Peptoniphilaceae_NG4_sp4 | 185 |
| Ruthenibacterium_sp1 | 186 |
| Monoglobus_pectinilyticus | 196 |
| Corynebacterium_durum | 259 |
| Christensenellaceae_NG5_sp2 | 509 |
| Cuneatibacter_caecimuris | 516 |
| Angelakisella_massiliensis | 538 |
| Massilimaliae_sp1 | 594 |
| [Clostridium]_minihomine | 745 |
| [Allisonella]_histaminiformans | 774 |
| [Merdimonas]_faecis | 1010 |
| Murimonas_intestini | 1621 |
| Prochlorotrichaceae_NG20_sp1 | 1661 |
| Clostridium_disporicum | 1757 |
| Flavonifractor_plautii | 1758 |
| Turicibacter_sanguinis | 1759 |
| Cellulosilyticum_lentocellum | 1760 |
| Erysipelatoclostridium_ramosum | 1761 |
| Dorea_longicatena | 1762 |
| [Clostridium]_aldenense | 1763 |
| [Clostridium]_bolteae | 1764 |
| [Clostridium]_innocuum | 1765 |
| Eisenbergiella_tayi | 1766 |
| Blautia_obeum | 1767 |
| [Clostridium]_scindens | 1768 |
| Eubacterium_callanderi | 1769 |
| Terrisporobacter_petrolearius | 1770 |
| Faecalicatena_contorta_strain | 1771 |
| Escherichia_fergusonii | 1772 |
| Bifidobacterium_dentium | 1773 |
| [Clostridium]_spiroforme | 1774 |
| Roseburia_hominis | 1775 |
| Holdemania_filiformis | 1776 |
| [Clostridium]_symbiosum | 1777 |
| Oscillibacter_valericigenes | 1778 |
| [Clostridium]_leptum | 1779 |
| Actinomycetaceae_NG7_sp1 | 1011, 1012, 1013, 1014, 1015, 1016, 1017 |
| Anaerostipes_hadrus | 1018, 1019, 1020, 1021, 1022, 1023, 1024, 1025, |
| | 1026, 1027, 1028, 1029, 1030, 1031, 1032, 1033, |
| | 1034, 1035, 1036, 1037, 1038, 1039, 1040, 1041, |
| | 1042, 1043, 1044, 1045, 1046, 1047, 1048, 1049, |
| | 1050, 1051, 1052, 1053, 1054, 1055, 1056, 1057 |
| Anaerotignum_lactatifermentans | 1058, 1059, 1060, 1061, 1062, 1063, 1064, 1065 |
| Anaerotruncus_colihominis | 1066, 1067, 1068, 1069, 1070, 1071, 1072, 1073, |
| | 1074, 1075, 1076, 1077, 1078, 1079, 1080, 1081, |
| | 1082, 1083, 1084, 1085 |
| Bacteroides_cellulosilyticus | 1086, 1087, 1088, 1089, 1090, 1091, 1092, 1093, |
| | 1094, 1095, 1096, 1097, 1098, 1099, 1100, 1101, |
| | 1102, 1103, 1104, 1105, 1106 |
| Parabacteroides_merdae | 11, 35, 78, 117, 137 |
| Bacteroides_coprocola | 1107, 1108, 1109, 1110, 1111, 1112 |
| Bacteroides_faecis | 1113, 1114, 1115, 1116, 1117, 1118, 1119, 1120, |
| | 1121, 1122, 1123, 1124, 1125, 1126, 1127 |
| Bacteroides_gallinarum | 1128, 1129 |
| Bacteroides_massiliensis | 1130, 1131, 1132, 1133, 1134, 1135 |
| Bacteroides_plebeius | 1136, 1137, 1138, 1139, 1140, 1141, 1142, 1143, |
| | 1144, 1145, 1146, 1147, 1148, 1149, 1150, 1151, |
| | 1152, 1153, 1154, 1155 |
| Bifidobacterium_animalis | 1156, 1157, 1158, 1159, 1160, 1161, 1162, 1163, |
| | 1164, 1165, 1166, 1167, 1168, 1169, 1170, 1171, |
| | 1172, 1173, 1174, 1175, 1176, 1177, 1178, 1179, |
| | 1180, 1181, 1182, 1183, 1184, 1185, 1186, 1187, |
| | 1188, 1189, 1190, 1191, 1192, 1193, 1194, 1195, |
| | 1196, 1197, 1198, 1199, 1200, 1201, 1202, 1203, |
| | 1204, 1205, 1206, 1207, 1208, 1209, 1210, 1211, |
| | 1212, 1213, 1214, 1215, 1216, 1217, 1218 |
| Intestinibacter_bartlettii | 12, 13, 140, 157 |
| Blautia_coccoides_group | 1219, 1220, 1221, 1222, 1223, 1224, 1225, 1226, |
| | 1227, 1228, 1229, 1230, 1231, 1232, 1233, 1234, |
| | 1235, 1236, 1237, 1238, 1239, 1240, 1241, 1242, |
| | 1243, 1244, 1245, 1246, 1247, 1248, 1249, 1250, |
| | 1251, 1252, 1253, 1254, 1255, 1256, 1257, 1258, |
| | 1259, 1260, 1261, 1262, 1263, 1264, 1265, 1266, |
| | 1267, 1268, 1269, 1270, 1271, 1272 |
| Ruminococcaceae_NG12_sp1 | 122, 174 |
| [Romboutsia]_timonensis | 127, 128, 539, 540 |
| Butyricicoccus_sp2 | 1273, 1274 |
| Christensenella_minuta | 1275, 1276, 1277, 1278, 1279, 1280, 1281, 1282 |
| Christensenella_timonensis | 1283, 1284, 1285 |
| Christensenellaceae_NG16_sp1 | 1286, 1287, 1288 |
| Collinsella_bouchesdurhonensis | 1289, 1290, 1291, 1292 |
| Lactonifactor_longoviformis | 129, 158, 176, 541, 578, 592 |
| Desulfovibrio_desulfuricans | 1293, 1294, 1295, 1296 |
| Dialister_invisus | 1297, 1298, 1299 |
| Eisenbergiella_massiliensis | 1300, 1301, 1302, 1303, 1304, 1305, |
| | 1306, 1307, 1308 |
| Emergencia_timonensis | 1309, 1310, 1311, 1312, 1313, 1314, 1315, 1316, |
| | 1317, 1318, 1319, 1320 |
| Neobitarella_massiliensis | 132, 179 |
| Faecalibacterium_prausnitzii | 1321, 1322, 1323, 1324, 1325, 1326, 1327, 1328, |
| | 1329, 1330, 1331, 1332, 1333, 1334, 1335, 1336, |
| | 1337, 1338, 1339, 1340, 1341, 1342, 1343, 1344, |
| | 1345, 1346, 1347, 1348, 1349 |
| Fusicatenibacter_sp1 | 1350, 1351, 1352, 1353, 1354, 1355 |
| Gemmiger_sp2 | 1356, 1357, 1358, 1359, 1360, 1361, 1362 |
| Granulicatella_adiacens | 1363, 1364, 1365, 1366, 1367, 1368 |
| Intestinimonas_butyriciproducens | 1369, 1370, 1371, 1372, 1373, 1374, 1375, 1376, |
| | 1377, 1378, 1379, 1380, 1381, 1382, 1383, 1384, |
| | 1385, 1386, 1387, 1388, 1389, 1390, 1391, 1392, |
| | 1393, 1394 |
| Intestinimonas_massiliensis | 1395, 1396, 1397, 1398, 1399, 1400, 1401, 1402 |
| Intestinimonas_timonensis | 1403, 1404 |
| Lachnoclostridium_edouardi | 1405, 1406, 1407, 1408, 1409, 1410 |
| Lachnospira_pectinoschiza | 1411, 1412, 1413, 1414, 1415, 1416 |
| Lachnospiraceae_NG17_sp3 | 1417, 1418 |
| Lachnospiraceae_NG18_sp7 | 1419, 1420, 1421, 1422, 1423, 1424, 1425, 1426 |
| Lactobacillus_delbrueckii | 1427, 1428, 1429, 1430, 1431, 1432, 1433, 1434, |
| | 1435, 1436, 1437, 1438, 1439, 1440, 1441, 1442, |
| | 1443, 1444, 1445, 1446, 1447, 1448, 1449, 1450, |
| | 1451, 1452, 1453, 1454, 1455, 1456, 1457, 1458, |
| | 1459, 1460, 1461, 1462, 1463, 1464, 1465, 1466, |
| | 1467, 1468, 1469, 1470, 1471, 1472, 1473, 1474, |
| | 1475, 1476, 1477, 1478, 1479, 1480, 1481, 1482, |
| | 1483, 1484, 1485, 1486, 1487, 1488, 1489, 1490, |
| | 1491, 1492, 1493, 1494, 1495, 1496, 1497, 1498, |
| | 1499, 1500, 1501, 1502, 1503, 1504, 1505 |
| Anaerofustis_stercorihominis | 15, 72, 150, 183 |
| Lactococcus_lactis | 1506, 1507, 1508, 1509, 1510, 1511, 1512, 1513, |
| | 1514, 1515, 1516, 1517, 1518, 1519, 1520, 1521, |
| | 1522, 1523, 1524, 1525, 1526, 1527, 1528, 1529, |
| | 1530, 1531, 1532, 1533, 1534, 1535, 1536, 1537, |
| | 1538, 1539, 1540, 1541, 1542, 1543, 1544, 1545, |
| | 1546, 1547, 1548, 1549, 1550, 1551, 1552, 1553, |
| | 1554, 1555, 1556, 1557, 1558, 1559, 1560, 1561, |
| | 1562, 1563, 1564, 1565, 1566, 1567, 1568, 1569, |
| | 1570, 1571, 1572, 1573, 1574, 1575, 1576, 1577, |
| | 1578, 1579, 1580, 1581, 1582, 1583, 1584, 1585, |
| | 1586, 1587, 1588, 1589, 1590, 1591, 1592, 1593, |
| | 1594, 1595, 1596, 1597, 1598, 1599, 1600, 1601, |
| | 1602, 1603 |
| Collinsella_intestinalis | 16, 17, 79, 81, 134 |
| Lawsonibacter_sp3 | 1604, 1605, 1606, 1607 |
| Lawsonibacter_sp7 | 1608, 1609, 1610, 1611, 1612, 1613, 1614, 1615 |
| Marvinbryantia_sp5 | 1616, 1617, 1618, 1619, 1620 |
| Odoribacter_splanchnicus | 1622, 1623, 1624, 1625, 1626, 1627, 1628, 1629, |
| | 1630, 1631, 1632, 1633, 1634, 1635, 1636, 1637, |
| | 1638, 1639, 1640, 1641, 1642, 1643, 1644, 1645, |
| | 1646, 1647, 1648 |
| Alistipes_indistinctus | 163, 165 |
| Oxalobacter_formigenes | 1649, 1650, 1651, 1652, 1653 |
| Prevotella_buccalis | 1654, 1655, 1656, 1657, 1658, 1659, 1660 |
| Propionibacterium_freudenreichii | 1662, 1663, 1664, 1665, 1666, 1667, 1668, 1669, |
| | 1670, 1671, 1672, 1673, 1674, 1675, 1676, 1677, |
| | 1678, 1679, 1680, 1681, 1682, 1683, 1684, 1685, |
| | 1686, 1687, 1688, 1689, 1690, 1691, 1692, 1693, |
| | 1694, 1695 |
| Roseburia_faecis | 1696, 1697, 1698, 1699, 1700, 1701, |
| | 1702, 1703, 1704 |
| Roseburia_intestinalis | 1705, 1706, 1707, 1708, 1709, 1710, 1711, 1712, |
| | 1713, 1714, 1715, 1716, 1717, 1718, 1719, 1720, |
| | 1721, 1722, 1723, 1724, 1725, 1726, 1727, 1728, |
| | 1729, 1730, 1731, 1732 |
| Ruthenibacterium_lactatiformans | 1733, 1734, 1735, 1736, 1737, 1738, 1739, 1740, |
| | 1741, 1742, 1743, 1744, 1745, 1746, |
| | 1747, 1748, 1749 |
| Scardovia_wiggsiae | 1750, 1751, 1752, 1753, 1754 |
| Slackia_isoflavoniconvertens | 1755, 1756 |
| Collinsella_stercoris | 18, 103 |
| Blautia_luti_group | 187, 189, 200, 201, 325, 326, 327, 328, 409, 429, |
| | 431, 432, 433, 437, 454, 458, 465, 467, 468, 475, |
| | 511, 522, 523, 524, 525, 526, 527, 528, 529, 574, |
| | 579, 581, 586, 587, 595, 596, 599 |
| Ruminococcaceae_NG13_sp1 | 188, 455 |
| Fusicatenibacter_sp2 | 190, 193, 453, 478, 502, 503, 504, 505, 506, 507, |
| | 564, 565 |
| Agathobaculum_butyriciproducens_group | 191, 195, 391, 392, 393, 394, 395, 396, 397, 398, |
| | 413, 452, 457, 466, 469, 471, 472, 473, 474, 476, |
| | 479, 480, 481, 589, 597 |
| [Ruminococcus]_bromii | 192, 381, 382, 383, 414, 415, 425, 428, 430, 434, |
| | 435, 436, 439, 441, 444, 577, 600 |
| Streptococcus_parasanguinis | 194, 217, 224, 234, 235, 236, 248, 257, 258, 273, |
| | 292, 307, 308, 310, 311, 312, 314, 319, 322, 334, |
| | 346, 347, 356, 360, 361, 362, 369, 443, 447, 463, |
| | 494, 554, 555, 573 |
| Streptococcus_australis | 197, 198, 237, 238, 270, 271, 272, 306, 313, 316, |
| | 336, 337, 338, 421, 483, 484, 486, 497, 498, 499, |
| | 501, 508, 510, 547, 548, 550, 551, 552 |
| Lachnospiraceae_NG30_sp3 | 199, 440, 464, 591 |
| Blautia_wexlerae | 2, 3, 6, 10, 19, 20, 21, 22, 23, 24, 73, 80, 82, 83, |
| | 84, 86, 87, 89, 91, 92, 93, 94, 96, 97, 98, 99, 100, |
| | 101, 104, 105, 106, 107, 109, 110, 111, 112, 114, |
| | 116, 123, 135, 136, 139, 143, 145, 146, 148, 156, |
| | 168, 169, 170, 178, 180, 182, 601, 602, 603, 606, |
| | 614, 615, 616, 617, 618, 619, 676, 691, 693, 696, |
| | 697, 699, 700, 702, 704, 705, 706, 709, 710, 711, |
| | 713, 714, 715, 716, 717, 718, 720, 721, 722, 723, |
| | 724, 725, 727, 730, 735, 746, 747, 748, 750, 751, |
| | 752, 757, 759, 761, 762, 763, 769, 770, 772 |
| [Lactobacillus]_fermentum | 202, 203, 204, 205, 267, 268, 269, 343, 344, 377, |
| | 378, 379, 400, 401, 402, 403, 487, 488, 489, 490, |
| | 491, 492, 493, 512, 513, 514, 515, 520, 584, 588 |
| Bacteroides_dorei_group | 206, 207, 208, 209, 210, 215, 223, 249, 250, 251, |
| | 252, 260, 261, 262, 285, 416, 419, 420, 422, 423, |
| | 426, 427, 451, 459, 482, 485, 517, 518, 519, 549, |
| | 556, 557, 558, 559, 562, 563, 590 |
| Streptococcus_gordonii | 211, 231, 232, 233, 289, 293, 321, 339, 340, 341, |
| | 342, 353, 374, 495, 496, 500, 546 |
| [Atopobium]_parvulum | 212, 282, 284, 287, 609, 627, 628, 629 |
| Desulfovibrio_fairfieldensis | 213, 245, 331, 332 |
| Schaalia_odontolytica | 214, 216, 255, 263, 283, 294, 299, 315, |
| | 384, 521, 543 |
| Bacteroides_intestinalis | 218, 410, 418, 438, 442, 445, 449, 450, 456, 460, |
| | 566, 583 |
| Rothia_mucilaginosa | 219, 246, 295, 296, 297, 298, 300, 301, 302, 303, |
| | 304, 330, 349, 352, 357, 358, 359, 363, 365, 366, |
| | 368, 370, 372, 373, 380, 385, 620, 621, 630, 631, |
| | 632, 633, 634, 635, 636, 637, 638, 642, 644, 645, |
| | 646, 647, 648, 649, 650, 651, 652, 653, 654, 655, |
| | 660, 661 |
| Streptococcus_salivarius_group | 220, 221, 222, 226, 277, 286, 309, 329, 371, 375, |
| | 399, 424, 530, 531, 553 |
| Streptococcus_infantis_group | 225, 227, 228, 229, 230, 239, 240, 241, 242, 243, |
| | 244, 253, 254, 264, 290, 291, 305, 317, 318, 320, |
| | 333, 335, 348, 354, 355, 364, 367, 537, 544, 545 |
| Actinomyces_graevenitzii | 247, 265, 387 |
| Pseudoflavonifractor_capillosus | 25, 26 |
| Schaalia_sp2 | 256, 266 |
| Alistipes_finegoldii | 27, 31, 32, 149 |
| [Ruminococcus]_gnavus | 274, 275, 276, 278, 279, 280, 281, 388, 417, 446, |
| | 448, 461, 532, 533, 534, 535, 536, 567, 568, 569, |
| | 570, 571, 572, 575, 576, 580, 593 |
| Bilophila_wadsworthia | 28, 30, 41 |
| Alistipes_shahii | 29, 76 |
| Bittarella_massiliensis | 324, 542 |
| Barnesiella_intestinihominis | 33, 160, 167 |
| Leuconostoc_gelidum | 34, 55, 120 |
| Rothia_dentocariosa | 345, 350, 351, 386 |
| Alistipes_obesi | 36, 166 |
| Sutterella_wadsworthensis | 37, 38, 39, 67, 173 |
| Bacteroides_caccae | 389, 390, 462, 560, 585 |
| Blautia_faecis | 4, 7, 144, 147, 154, 155, 159, 171, 177 |
| [Adlercreutzia]_equolifaciens_group | 40, 68, 70 |
| Blautia_sp1 | 404, 405, 406, 407, 408 |
| [Coprococcus]_catus | 411, 412 |
| Bifidobacterium_catenulatum_group | 42, 43, 44, 45, 46, 47, 48, 50, 51, 52, 57, 58, 59, |
| | 64, 65, 66, 74, 75, 88, 90, 138, 141, 142, 161 |
| Ruminococcaceae_NG18_sp1 | 49, 102, 113, 288, 470, 477 |
| Lachnospiraceae_NG29_sp1 | 5, 8, 151 |
| [Extibacter]_muris | 53, 77, 119, 125, 175 |
| Alistipes_onderdonkii | 54, 60, 164, 323, 376, 582 |
| Clostridium_saudiense | 561, 598 |
| Acetatifactor_sp1 | 604, 607, 675, 707, 708, 712, 719, 726, 728, 729, |
| | 753, 760, 768, 771 |
| Blautia_caecimuris | 605, 733, 754, 756, 765 |
| Campylobacter_concisus | 608, 624, 626, 639, 656, 657, 658, 659, 668, 669, |
| | 670, 671, 672, 673, 674 |
| Anaeromassilibacillus_sp3 | 61, 181 |
| Prevotella_copri | 610, 611, 612, 677, 678, 679, 680, 682, 683, 684, |
| | 685, 686, 687, 689, 692, 694, 695, 698, 701, 731 |
| Holdemania_filiformis | 613, 681, 736, 749 |
| Hungatella_sp2 | 622, 623, 734 |
| Holdemania_massiliensis | 625, 764 |
| Anaerobutyricum_hallii | 640, 641, 688, 690, 703, 737, 738, 739, 740, 741, |
| | 742, 743, 744, 755, 766, 773 |
| Akkermansia_muciniphila | 643, 662, 663, 664, 665, 666, 667, 767 |
| Harryflintia_acetispora | 71, 118, 131, 152 |
| Dysosmobacter_welbionis | 732, 758 |
| [Clostridium]_asparagiforme_group | 775, 776, 777, 778, 779, 780, 781, 782, 783, 784, |
| | 785, 786, 787, 788, 789 |
| [Clostridium]_hylemonae | 790, 791, 792, 793, 794, 795, 796 |
| [Coprobacter]_secundus_group | 797, 798, 799 |
| [Faecalicatena]_orotica | 800, 801, 802, 803, 804, 805, 806, 807, |
| | 808, 809, 810 |
| [Lactobacillus]_paracasei | 811, 812, 813, 814, 815, 816, 817, 818, 819, 820, |
| | 821, 822, 823, 824, 825, 826, 827, 828, 829, 830, |
| | 831, 832, 833, 834, 835, 836, 837, 838, 839, 840, |
| | 841, 842, 843, 844, 845, 846, 847, 848, 849, 850, |
| | 851, 852, 853, 854, 855, 856, 857, 858, 859, 860, |
| | 861, 862, 863, 864, 865, 866, 867, 868, 869, 870, |
| | 871, 872, 873, 874, 875, 876, 877, 878, 879, 880, |
| | 881, 882, 883, 884, 885, 886, 887, 888, 889, 890, |
| | 891, 892, 893, 894, 895, 896, 897, 898, 899, 900, |
| | 901, 902, 903, 904, 905, 906, 907, 908, 909, 910, |
| | 911, 912, 913, 914, 915, 916, 917, 918, 919, 920, |
| | 921, 922, 923, 924, 925, 926, 927, 928, 929, 930 |
| Hungatella_sp1 | 85, 95, 108, 115, 153 |
| [Lactobacillus]_reuteri | 931, 932, 933, 934, 935, 936, 937, 938, 939, 940, |
| | 941, 942, 943, 944, 945, 946, 947, 948, 949, 950, |
| | 951, 952, 953, 954, 955, 956, 957, 958, 959, 960, |
| | 961, 962, 963, 964, 965, 966, 967, 968, 969, 970, |
| | 971, 972, 973, 974, 975, 976, 977, 978, 979, 980, |
| | 981, 982, 983, 984, 985, 986, 987, 988, 989, 990, |
| | 991, 992, 993, 994, 995, 996, 997, 998, 999, |
| | 1000, 1001, 1002, 1003, 1004, 1005, 1006, 1007, |
| | 1008, 1009 |

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that may be cited throughout this disclosure are hereby expressly incorporated by reference in their entirety for any purpose, as are the references cited therein.

What is claimed is:

1. A therapeutic composition comprising an effective amount of a purified or isolated population of bacteria selected from Intestinimonas butyriciproducens and Anaerotruncus colihominis or bacteria having a 16S rDNA sequence having at least 97%, 98%, 98.7% or 99% sequence identity to any one 16S rDNA sequence thereof.

2. The therapeutic composition of claim 1, wherein the 16S rDNA sequence of Intestinimonas butyriciproducens is selected from SEQ ID NOs: 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, and 1394, and the 16S rDNA sequence of Anaerotruncus colihominis is selected from SEQ ID NOs: 1066, 1067, 1068, 1069, 1070, 1071, 1072, 1073, 1074, 1075, 1076, 1077, 1078, 1079, 1080, 1081, 1082, 1083, 1084, and 1085.

3. A therapeutic composition comprising an effective amount of a purified or isolated population of bacteria selected from Murimonas intestini and Faecalicatena orotica or bacteria having a 16S rDNA sequence having at least 97%, 98%, 98.7% or 99% sequence identity to any one 16S rDNA sequence thereof.

4. The therapeutic composition of claim 1, wherein the 16S rDNA sequence of Murimonas intestini is SEQ ID NO: 1621 and the 16S rDNA sequence of Faecalicatena orotica is selected from SEQ ID NOs: 800, 801, 802, 803, 804, 805, 806, 807, 808, 809, and 810.

5. A therapeutic composition comprising an effective amount of a purified or isolated population of bacteria comprising one or more bacteria selected from Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans, or bacteria having 16S rDNA having at least 94.5%, 95%, 97%, 98%, 98.7% or 99% sequence identity to any one of the 16S rDNA thereof.

6. The therapeutic composition of claim 5, wherein the 16S rDNA sequence of Anaerostipes hadrus is selected from SEQ ID NOs: 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, and 1057, the 16S rDNA sequence of Christensenella timonensis is selected from SEQ ID NOs: 1283, 1284, and 1285, the 16S rDNA sequence of Desulfovibrio desulfuricans is selected from SEQ ID NOs: 1293, 1294, 1295, and 1296, the 16S rDNA sequence of Dialister invisus is selected from SEQ ID NOs: 1297, 1298, and 1299, the 16S rDNA sequence of Lachnoclostridium edouardi is selected from SEQ ID NOs: 1405, 1406, 1407, 1408, 1409, and 1410, the 16S rDNA sequence of Lachnospira pectinoschiza is selected from SEQ ID NOs: 1411, 1412, 1413, 1414, 1415, and 1416, the 16S rDNA sequence of Lachnospiraceae NG18_sp7 is selected from SEQ ID NOs: 1419, 1420, 1421, 1422, 1423, 1424, 1425, and 1426, the 16S rDNA sequence of Lawsonibacter sp7 is selected from SEQ ID NOs: 1608, 1609, 1610, 1611, 1612, 1613, 1614, and 1615, the 16S rDNA sequence of Merdimonas faecis is SEQ ID NO: 1010, the 16S rDNA sequence of Odoribacter splanchnicus is selected from SEQ ID NOs: 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, and 1648, the 16S rDNA sequence of Roseburia faecis is selected from SEQ ID NOs: 1696, 1697, 1698, 1699, 1700, 1701, 1702, 1703, and 1704, the 16S rDNA sequence of Roseburia intestinalis is selected from SEQ ID NOs: 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732, or the 16S rDNA sequence of Ruthenibacterium lactatiformans is selected from SEQ ID NOs: 1733, 1734, 1735, 1736, 1737, 1738, 1739, 1740, 1741, 1742, 1743, 1744, 1745, 1746, 1747, 1748, and 1749.

7. The therapeutic composition of claim 5, wherein the bacteria comprises one or more bacteria selected from Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof.

8. The therapeutic composition of claim 7, wherein the 16S rDNA sequence of Anaerostipes hadrus is selected from SEQ ID NOs: 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, and 1057, the 16S rDNA sequence of Dialister invisus is selected from SEQ ID NOs: 1297, 1298, and 1299, the 16S rDNA sequence of Merdimonas faecis is SEQ ID NO: 1010, the 16S rDNA sequence of Roseburia faecis is selected from SEQ ID NOs: 1696, 1697, 1698, 1699, 1700, 1701, 1702, 1703, and 1704, or the 16S rDNA sequence of Roseburia intestinalis is selected from SEQ ID NOs: 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

9. The therapeutic composition of claim 5, wherein the bacteria comprises one or more bacteria selected from Desulfovibrio desulfuricans, Roseburia intestinalis, or bacteria having 16S rDNA having at least 94.5%, 95%, 97%, 98%, 98.7% or 99% sequence identity to any one of the 16S rDNA thereof.

10. The therapeutic composition of claim 9, wherein the 16S rDNA sequence of Desulfovibrio desulfuricans is selected from SEQ ID NOs: 1293, 1294, 1295, and 1296 or the 16S rDNA sequence of Roseburia intestinalis is selected from SEQ ID NOs: 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

11. The therapeutic composition of claim 5, wherein the bacteria comprises one or more bacteria selected from Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof.

12. The therapeutic composition of claim 11, wherein the 16S rDNA sequence of Lachnoclostridium edouardi is selected from SEQ ID NOs: 1405, 1406, 1407, 1408, 1409, and 1410, the 16S rDNA sequence of Lachnospira pectinoschiza is selected from SEQ ID NOs: 1411, 1412, 1413, 1414, 1415, and 1416, the 16S rDNA sequence of Lawsonibacter sp7 is selected from SEQ ID NOs: 1608, 1609, 1610, 1611, 1612, 1613, 1614, and 1615, the 16S rDNA sequence of Odoribacter splanchnicus is selected from SEQ ID NOs: 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, and 1648, or the 16S rDNA sequence of Ruthenibacterium lactatiformans is selected from SEQ ID NOs: 1733, 1734, 1735, 1736, 1737, 1738, 1739, 1740, 1741, 1742, 1743, 1744, 1745, 1746, 1747, 1748, and 1749.

13. A therapeutic composition comprising an effective amount of a purified or isolated population of bacteria comprising one or more bacteria selected from Anaerotignum lactatifermentans, Anaerotruncus colihominis, Angelakisella sp1, Bacteroides cellulosilyticus, Bacteroides gallinarum, Bacteroides plebeius, Blautia coccoides group, Christensenella minuta, Christensenellaceae NG16_sp1, Clostridium hylemonae, Collinsella bouchesdurhonensis, Coprobacter secundus group, Eisenbergiella massiliensis, Emergencia timonensis, Faecalicatena orotica, Fusicatenibacter sp1, Intestinimonas butyriciproducens, Intestinimonas timonensis, Lachnospiraceae NG17_sp3, Lawsonibacter sp3, Oxalobacter formigenes, Peptoniphilaceae NG4_sp2, Prochlorotrichaceae NG20_sp1, or bacteria having a 16S rDNA sequence having at least 97%, 98%, 98.7% or 99% sequence identity to any one 16S rDNA sequence thereof.

14. The therapeutic composition of claim 13, wherein the 16S rDNA sequence of Anaerotignum lactatifermentans is selected from SEQ ID NOs: 1058, 1059, 1060, 1061, 1062, 1063, 1064, and 1065, the 16S rDNA sequence of Anaerotruncus colihominis is selected from SEQ ID NOs: 1066, 1067, 1068, 1069, 1070, 1071, 1072, 1073, 1074, 1075, 1076, 1077, 1078, 1079, 1080, 1081, 1082, 1083, 1084, and 1085, the 16S rDNA sequence of Bacteroides cellulosilyticus is selected from SEQ ID NOs: 1086, 1087, 1088, 1089, 1090, 1091, 1092, 1093, 1094, 1095, 1096, 1097, 1098, 1099, 1100, 1101, 1102, 1103, 1104, 1105, and 1106, the 16S rDNA sequence of Bacteroides gallinarum is selected from SEQ ID NOs: 1128 and 1129, the 16S rDNA sequence of Bacteroides plebeius is selected from SEQ ID NOs: 1136, 1137, 1138, 1139, 1140, 1141, 1142, 1143, 1144, 1145, 1146, 1147, 1148, 1149, 1150, 1151, 1152, 1153, 1154, and 1155, the 16S rDNA sequence of Blautia coccoides group is selected from SEQ ID NOs: 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, and 1272, the 16S rDNA sequence of Christensenella minuta is selected from SEQ ID NOs: 1275, 1276, 1277, 1278, 1279, 1280, 1281, and 1282, the 16S rDNA sequence of Christensenellaceae NG16_sp1 is selected from SEQ ID NOs: 1286, 1287, and 1288, the 16S rDNA sequence of Clostridium hylemonae is selected from SEQ ID NOs: 790, 791, 792, 793, 794, 795, and 796, the 16S rDNA sequence of Collinsella bouchesdurhonensis is selected from SEQ ID NOs: 1289, 1290, 1291, and 1292, the 16S rDNA sequence of Coprobacter secundus group is selected from SEQ ID NOs: 797, 798, and 799, the 16S rDNA sequence of Eisenbergiella massiliensis is selected from SEQ ID NOs: 1300, 1301, 1302, 1303, 1304, 1305, 1306, 1307, and 1308, the 16S rDNA sequence of Emergencia timonensis is selected from SEQ ID NOs: 1309, 1310, 1311, 1312, 1313, 1314, 1315, 1316, 1317, 1318, 1319, and 1320, the 16S rDNA sequence of Faecalicatena orotica is selected from SEQ ID NOs: 800, 801, 802, 803, 804, 805, 806, 807, 808, 809, and 810, the 16S rDNA sequence of Fusicatenibacter sp1 is selected from SEQ ID NOs: 1350, 1351, 1352, 1353, 1354, and 1355, the 16S rDNA sequence of Intestinimonas butyriciproducens is selected from SEQ ID NOs: 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, and 1394, the 16S rDNA sequence of Intestinimonas timonensis is selected from SEQ ID NOs: 1403 and, 1404, the 16S rDNA sequence of Lachnospiraceae NG17_sp3 is selected from SEQ ID NOs: 1417 and 1418, the 16S rDNA sequence of Lawsonibacter sp3 is selected from SEQ ID NOs: 1604, 1605, 1606, and 1607, the 16S rDNA sequence of Oxalobacter formigenes is selected from SEQ ID NOs: 1649, 1650, 1651, 1652, and 1653, the 16S rDNA sequence of Peptoniphilaceae NG4_sp2 is SEQ ID NO: 172, or the 16S rDNA sequence of Prochlorotrichaceae NG20_sp1 is SEQ ID NO: 1661.

15. The therapeutic composition of any of claims 1-14, wherein the therapeutic composition comprises two, three, four, five, six, seven, eight, nine, ten, eleven, twelve, thirteen, fourteen, fifteen, sixteen, seventeen, eighteen or more bacterial species.

16. The therapeutic composition of any of claims 1-15, wherein the therapeutic composition does not comprise one or more bacteria selected from Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

17. The therapeutic composition of claim 16, wherein the 16S rDNA sequence of Actinomycetaceae_NG7 sp1 is selected from SEQ ID NOs: 1011, 1012, 1013, 1014, 1015, 1016, and 1017, the 16S rDNA sequence of Allisonella histaminiformans is SEQ ID NO: 774, the 16S rDNA sequence of Bacteroides coprocola is selected from SEQ ID NOs: 1107, 1108, 1109, 1110, 1111, and 1112, the 16S rDNA sequence of Bacteroides faecis is selected from SEQ ID NOs: 1113, 1114, 1115, 1116, 1117, 1118, 1119, 1120, 1121, 1122, 1123, 1124, 1125, 1126, and 1127, the 16S rDNA sequence of Bacteroides massiliensis is selected from SEQ ID NOs: 1130, 1131, 1132, 1133, 1134, and 1135, the 16S rDNA sequence of Bifidobacterium animalis is selected from SEQ ID NOs: 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, and 1218, the 16S rDNA sequence of Clostridium asparagiforme group is selected from SEQ ID NOs: 775, 776, 777, 778, 779, 780, 781, 782, 783, 784, 785, 786, 787, 788, and 789, the 16S rDNA sequence of Faecalibacterium prausnitzii is selected from SEQ ID NOs: 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349, the 16S rDNA sequence of Gemmiger sp2 is selected from SEQ ID NOs: 1356, 1357, 1358, 1359, 1360, 1361, and 1362, the 16S rDNA sequence of Granulicatella adiacens is selected from SEQ ID NOs: 1363, 1364, 1365, 1366, 1367, and 1368, the 16S rDNA sequence of Intestinimonas massiliensis is selected from SEQ ID NOs: 1395, 1396, 1397, 1398, 1399, 1400, 1401, and 1402, the 16S rDNA sequence of Lactobacillus delbrueckii is selected from SEQ ID NOs: 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, and 1505, the 16S rDNA sequence of Lactobacillus reuteri is selected from SEQ ID NOs: 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, and 1009, the 16S rDNA sequence of Lactococcus lactis is selected from SEQ ID NOs: 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, and 1603, the 16S rDNA sequence of Marvinbryantia sp5 is selected from SEQ ID NOs: 1616, 1617, 1618, 1619, and 1620, the 16S rDNA sequence of Prevotella buccalis is selected from SEQ ID NOs: 1654, 1655, 1656, 1657, 1658, 1659, and 1660, the 16S rDNA sequence of Propionibacterium freudenreichii is selected from SEQ ID NOs: 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, and 1695, the 16S rDNA sequence of Scardovia wiggsiae is selected from SEQ ID NOs: 1750, 1751, 1752, 1753, and 1754, or the 16S rDNA sequence of Slackia isoflavoniconvertens is selected from SEQ ID NOs: 1755 and 1756.

18. The therapeutic composition of any of claims 1-15, wherein the therapeutic composition does not comprise Allisonella histaminiformans, Bacteroides faecis, Bacteroides massiliensis, Desulfovibrionaceae NG5_sp1, Gemmiger sp2, Intestinimonas massiliensis, Marvinbryantia sp5, Propionibacterium freudenreichii, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

19. The therapeutic composition of claim 18, wherein the 16S rDNA sequence of Allisonella histaminiformans is SEQ ID NO: 774, the 16S rDNA sequence of Bacteroides faecis is selected from SEQ ID NOs: 1113, 1114, 1115, 1116, 1117, 1118, 1119, 1120, 1121, 1122, 1123, 1124, 1125, 1126, and 1127, the 16S rDNA sequence of Bacteroides massiliensis is selected from SEQ ID NOs: 1130, 1131, 1132, 1133, 1134, and 1135, the 16S rDNA sequence of Gemmiger sp2 is selected from SEQ ID NOs: 1356, 1357, 1358, 1359, 1360, 1361, and 1362, the 16S rDNA sequence of Intestinimonas massiliensis is selected from SEQ ID NOs: 1395, 1396, 1397, 1398, 1399, 1400, 1401, and 1402, the 16S rDNA sequence of Marvinbryantia sp5 is selected from SEQ ID NOs: 1616, 1617, 1618, 1619, and 1620, the 16S rDNA sequence of Propionibacterium freudenreichii is selected from SEQ ID NOs: 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, and 1695, or the 16S rDNA sequence of Slackia isoflavoniconvertens is selected from SEQ ID NOs: 1755 and 1756.

20. The therapeutic composition of any of claims 1-15, wherein the therapeutic composition does not comprise Bifidobacterium animalis, Faecalibacterium prausnitzii, or bacteria having 16S rDNA having at least 94.5%, 95%, 97%, 98%, 98.7% or 99% sequence identity to any one of the 16S rDNA thereof.

21. The therapeutic composition of claim 20, wherein the 16S rDNA sequence of Bifidobacterium animalis is selected from SEQ ID NOs: 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, and 1218, or the 16S rDNA sequence of Faecalibacterium prausnitzii is selected from SEQ ID NOs: 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349.

22. The therapeutic composition of any of claims 1-15, wherein the therapeutic composition does not comprise Granulicatella adiacens, Lactococcus lactis, Scardovia wiggsiae, or bacteria having 16S rDNA having at least 94.5%, 95%, 97%, 98%, 98.7% or 99% sequence identity to any one of the 16S rDNA thereof.

23. The therapeutic composition of claim 22, wherein the 16S rDNA sequence of Granulicatella adiacens is selected from SEQ ID NOs: 1363, 1364, 1365, 1366, 1367, and 1368, the 16S rDNA sequence of Lactococcus lactis is selected from SEQ ID NOs: 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, and 1603, or the 16S rDNA sequence of Scardovia wiggsiae is selected from SEQ ID NOs: 1750, 1751, 1752, 1753, and 1754.

24. A therapeutic composition comprising an effective amount of a purified or isolated population of bacteria that have 16S rDNA sequence identity of at least 94.5% to 16S rDNA sequences of species belonging to one or more of the families Sutterellaceae, Coriobacteriaceae, Akkermansiaceae, Desulfovibrionaceae, Odoribacteraceae, or Rikenellaceae.

25. The therapeutic composition of claim 24, wherein the bacteria have 16S rDNA sequence identity of at least 95%, 97%, 98%, 98.7% or 99% to 16S rDNA sequences of species belonging to one or more of the families Sutterellaceae, Coriobacteriaceae, Akkermansiaceae, Desulfovibrionaceae, Odoribacteraceae, or Rikenellaceae.

26. The therapeutic composition of claim 24 or 25, wherein the therapeutic composition comprises bacteria belonging to two, three, four or more families.

27. A therapeutic composition comprising an effective amount of a purified or isolated population of bacteria comprising one or more bacteria selected from Akkermansia muciniphila, Dysosmobacter welbionis, Anaerobutyricum hallii, Blautia caecimuris, Holdemania filiformis, Acetatifactor sp1, Ruminococcaceae NG14 sp1, Hungatella sp2, Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Anaerostipes hadrus, Christensenella timonensis, Desulfovibrio desulfuricans, Dialister invisus, Lachnoclostridium edouardi, Lachnospira pectinoschiza, Lachnospiraceae NG18_sp7, Lawsonibacter sp7, Merdimonas faecis, Negativibacillus sp1, Odoribacter splanchnicus, Roseburia faecis, Roseburia intestinalis, Ruminococcaceae NG11_sp5, Ruthenibacterium lactatiformans, or bacteria having 16S rDNA having at least 94.5%, 95%, 97%, 98%, 98.7% or 99% sequence identity to any one of the 16S rDNA thereof.

28. The therapeutic composition of claim 27, wherein the bacteria comprises one or more bacteria selected from Akkermansia muciniphila, Dysosmobacter welbionis, Anaerobutyricum hallii, Blautia caecimuris, Holdemania filiformis, or bacteria having 16S rDNA having at least 94.5%, 95%, 97%, 98%, 98.7% or 99% sequence identity to any one of the 16S rDNA thereof.

29. The therapeutic composition of claim 27, wherein the bacteria comprises one or more bacteria selected from Acetatifactor sp1, Blautia wexlerae, Ruminococcaceae NG14 sp1, Hungatella sp2, Holdemania filiformis, or bacteria having 16S rDNA having at least 94.5%, 95%, 97%, 98%, 98.7% or 99% sequence identity to any one of the 16S rDNA thereof.

30. The therapeutic composition of claim 27, wherein the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof.

31. The therapeutic composition of claim 27, wherein the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, Desulfovibrio desulfuricans, Roseburia intestinalis, or bacteria having 16S rDNA having at least 94.5%, 95%, 97%, 98%, 98.7% or 99% sequence identity to any one of the 16S rDNA thereof.

32. The therapeutic composition of claim 27, wherein the bacteria comprises one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof.

33. A therapeutic composition comprising a purified population of bacteria, wherein the purified or isolated population of bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 601, 602, 603, 604, 605, 606, 607, 613, 614, 615, 616, 617, 618, 619, 622, 623, 640, 641, 643, 662, 663, 664, 665, 666, 667, 675, 676, 681, 688, 690, 691, 693, 696, 697, 699, 700, 702, 703, 704, 705, 706, 707, 708, 709, 710, 711, 712, 713, 714, 715, 716, 717, 718, 719, 720, 721, 722, 723, 724, 725, 726, 727, 728, 729, 730, 732, 733, 734, 735, 736, 737, 738, 739, 740, 741, 742, 743, 744, 746, 747, 748, 749, 750, 751, 752, 753, 754, 755, 756, 757, 758, 759, 760, 761, 762, 763, 765, 766, 767, 768, 769, 770, 771, 772 773, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1283, 1284, 1285, 1293, 1294, 1295, 1296, 1297, 1298, 1299, 1405, 1406, 1407, 1408, 1409, 1410, 1411, 1412, 1413, 1414, 1415, 1416, 1419, 1420, 1421, 1422, 1423, 1424, 1425, 1426, 1608, 1609, 1610, 1611, 1612, 1613, 1614, 1615, 1010, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749.

34. The therapeutic composition of claim 33, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 605, 613, 640, 641, 643, 662, 663, 664, 665, 666, 667, 681, 688, 690, 703, 732, 733, 736, 737, 738, 739, 740, 741, 742, 743, 744, 749, 754, 755, 756, 758, 765, 766, 767 and 773.

35. The therapeutic composition of claim 33, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 601, 602, 603, 604, 606, 607, 613, 614, 615, 616, 617, 618, 619, 622, 623, 675, 676, 681, 691, 693, 696, 697, 699, 700, 702, 704, 705, 706, 707, 708, 709, 710, 711, 712, 713, 714, 715, 716, 717, 718, 719, 720, 721, 722, 723, 724, 725, 726, 727, 728, 729, 730, 734, 735, 736, 746, 747, 748, 749, 750, 751, 752, 753, 757, 759, 760, 761, 762, 763, 768, 769, 770, 771 and 772.

36. The therapeutic composition of claim 33, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180, 182, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1297, 1298, 1299, 1010, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

37. The therapeutic composition of claim 33, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179, 184, 1293, 1294, 1295, 1296, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

38. The therapeutic composition of claim 33, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183, 186, 1405, 1406, 1407, 1408, 1409, 1410, 1411, 1412, 1413, 1414, 1415, 1416, 1608, 1609, 1610, 1611, 1612, 1613, 1614, 1615, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1733, 1734, 1735, 1736, 1737, 1738, 1739, 1740, 1741, 1742, 1743, 1744, 1745, 1746, 1747, 1748, and 1749.

39. The therapeutic composition of any of claims 27-38, wherein the therapeutic composition comprises two, three, four, five, six, seven, eight, nine, ten, eleven, twelve, thirteen, fourteen, fifteen, sixteen, seventeen, eighteen or more bacterial species.

40. The therapeutic composition of any of claims 24-39, wherein the therapeutic composition does not comprise one or more bacteria selected from Holdemania massiliensis, Rothia mucilaginosa, Hungatella sp2, Prevotella copri, Campylobacter concisus, [Atopobium]parvulum, Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, [Atopobium] parvulum, [Ruminococcus] gnavus, Bacteroides dorei group, Blautia sp1, Schaalia sp2, Actinomycetaceae_NG7 sp1, Allisonella histaminiformans, Bacteroides coprocola, Bacteroides faecis, Bacteroides massiliensis, Bifidobacterium animalis, Clostridium asparagiforme group, Desulfovibrionaceae NG5_sp1, Faecalibacterium prausnitzii, Gemmiger sp2, Granulicatella adiacens, Intestinimonas massiliensis, Lactobacillus delbrueckii, Lactobacillus paracasei, Lactobacillus reuteri, Lactococcus lactis, Marvinbryantia sp5, Prevotella buccalis, Propionibacterium freudenreichii, Ruminococcaceae NG18_sp7, Scardovia wiggsiae, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

41. The therapeutic composition of any of claims 24-39, wherein the therapeutic composition does not comprise Holdemania massiliensis, Rothia mucilaginosa, Hungatella sp2, Prevotella copri, Campylobacter concisus, or bacteria having 16S rDNA having at least 94.5%, 95%, 97%, 98%, 98.7% or 99% sequence identity to any one of the 16S rDNA thereof.

42. The therapeutic composition of any of claims 24-39, wherein the therapeutic composition does not comprise Rothia mucilaginosa, [Atopobium] parvulum, Holdemania massiliensis, Prevotella copri, Campylobacter concisus, or bacteria having 16S rDNA having at least 94.5%, 95%, 97%, 98%, 98.7% or 99% sequence identity to any one of the 16S rDNA thereof.

43. The therapeutic composition of any of claims 24-39, wherein the therapeutic composition does not comprise Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, Bifidobacterium animalis, Faecalibacterium prausnitzii, or bacteria having 16S rDNA having at least 94.5%, 95%, 97%, 98%, 98.7% or 99% sequence identity to any one of the 16S rDNA thereof.

44. The therapeutic composition of any of claims 24-39, wherein the therapeutic composition does not comprise [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, Allisonella histaminiformans, Bacteroides faecis, Bacteroides massiliensis, Desulfovibrionaceae NG5_sp1, Gemmiger sp2, Intestinimonas massiliensis, Marvinbryantia sp5, Propionibacterium freudenreichii, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

45. The therapeutic composition of any of claims 24-39, wherein the therapeutic composition does not comprise [Atopobium] parvulum, [Ruminococcus] gnavus, Bacteroides dorei group, Blautia sp1, Schaalia sp2, Granulicatella adiacens, Lactococcus lactis, Scardovia wiggsiae, or bacteria having 16S rDNA having at least 94.5%, 95%, 97%, 98%, 98.7% or 99% sequence identity to any one of the 16S rDNA thereof.

46. The therapeutic composition of any of claims 24-39, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 608, 609, 610, 611, 612, 620, 621, 622, 623, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734, 764, 1011, 1012, 1013, 1014, 1015, 1016, 1017, 774, 1107, 1108, 1109, 1110, 1111, 1112, 1113, 1114, 1115, 1116, 1117, 1118, 1119, 1120, 1121, 1122, 1123, 1124, 1125, 1126, 1127, 1130, 1131, 1132, 1133, 1134, 1135, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 775, 776, 777, 778, 779, 780, 781, 782, 783, 784, 785, 786, 787, 788, 789, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1356, 1357, 1358, 1359, 1360, 1361, 1362, 1363, 1364, 1365, 1366, 1367, 1368, 1395, 1396, 1397, 1398, 1399, 1400, 1401, 1402, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1616, 1617, 1618, 1619, 1620, 1654, 1655, 1656, 1657, 1658, 1659, 1660, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1750, 1751, 1752, 1753, 1754, 1755, and 1756.

47. The therapeutic composition of any of claims 24-39, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 610, 611, 612, 620, 621, 622, 623, 624, 625, 626, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731, 734 and 764.

48. The therapeutic composition of any of claims 24-39, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 608, 609, 610, 611, 612, 620, 621, 624, 625, 626, 627, 628, 629, 630, 631, 632, 633, 634, 635, 636, 637, 638, 639, 642, 644, 645, 646, 647, 648, 649, 650, 651, 652, 653, 654, 655, 656, 657, 658, 659, 660, 661, 668, 669, 670, 671, 672, 673, 674, 677, 678, 679, 680, 682, 683, 684, 685, 686, 687, 689, 692, 694, 695, 698, 701, 731 and 764.

49. The therapeutic composition of any of claims 24-39, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538, 582, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, o1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349.

50. The therapeutic composition of any of claims 24-39, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599, 600, 774, 1113, 1114, 1115, 1116, 1117, 1118, 1119, 1120, 1121, 1122, 1123, 1124, 1125, 1126, 1127, 1130, 1131, 1132, 1133, 1134, 1135, 1356, 1357, 1358, 1359, 1360, 1361, 1362, 1395, 1396, 1397, 1398, 1399, 1400, 1401, 1402, 1616, 1617, 1618, 1619, 1620, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1755, and 1756.

51. The therapeutic composition of any of claims 24-39, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590, 593, 1363, 1364, 1365, 1366, 1367, 1368, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1750, 1751, 1752, 1753, and 1754.

52. The therapeutic composition of any of claims 1-51, further comprising at least one anticancer agent.

53. The therapeutic composition of claim 52, wherein the anticancer agent is a checkpoint inhibitor.

54. The therapeutic composition of claim 53, wherein the checkpoint inhibitor is selected from an anti-PD-1 antibody, an anti-CTLA-4 antibody, an anti-PD-L1 antibody or combinations thereof.

55. The therapeutic composition of claim 54, wherein the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT O11, MPDL3280A, MEDI-4736, MSB-0020718C, AUR-012, LAG-3, OX40 inhibitors, OX40L inhibitors, TIGIT inhibitors or STI-A1010, or combinations thereof.

56. The therapeutic composition of claim 52, wherein the anticancer agent is a chemotherapy agent.

57. The therapeutic composition of any of claims 52-56, wherein the anticancer agent is a combination of a checkpoint inhibitor and a chemotherapy agent.

58. The therapeutic composition of any of claims 1-57, wherein each of the purified or isolated populations of bacteria is present in the composition at a concentration of at least about 1×10² viable colony forming units (CFU).

59. The therapeutic composition of any of claims 1-58, wherein each purified or isolated population of bacteria is present in the composition at a concentration of about 1×10² to 1×10⁹ viable CFU.

60. The therapeutic composition of any of claims 1-59, wherein a fraction of the purified or isolated population of bacteria comprises a spore-forming bacteria.

61. The therapeutic composition of any of claims 1-60, wherein a fraction of the purified or isolated population of bacteria is in spore form.

62. The therapeutic composition of any of claims 1-60, wherein a fraction of the purified or isolated population of bacteria is in vegetative form.

63. The therapeutic composition of any of claims 1-60, wherein the composition further comprises a pharmaceutically acceptable excipient.

64. The therapeutic composition of any of claims 1-63, wherein the composition is formulated for delivery to the intestine.

65. The therapeutic composition of any of claims 1-64, wherein the composition is enterically coated.

66. The therapeutic composition of any of claims 1-65, wherein the composition is formulated for oral administration.

67. The therapeutic composition of any of claims 1-66, wherein the composition is formulated into a food or beverage.

68. The therapeutic composition of any of claims 1-67, wherein the composition can reduce the rate of tumor growth in an animal model or in a mammalian subject.

69. The therapeutic composition of any of claims 1-68, wherein the composition can reduce the rate of tumor growth in an animal model or in a mammalian subject when administered concomitantly with, prior to, or subsequent to the administration of at least one anticancer agent.

70. The therapeutic composition of any of claims 1-69, wherein the composition can increase the number of active CD8 T cells in a tumor of an animal model or of a mammalian subject when administered concomitantly with, prior to, or subsequent to the administration of at least one anticancer agent.

71. The therapeutic composition of any one of claims 1-70, wherein the composition is formulated for multiple administrations.

72. The therapeutic composition of any one of claims 1-58, wherein each of the populations of bacteria is present in the composition at a concentration of at least 1×10³ viable CFU.

73. A method of treating or preventing or reducing risk of a cancer in a mammalian subject in need thereof comprising administering to the subject a therapeutic composition according to any one of claims 1-72.

74. The method of claim 73, wherein the bacteria comprises one or more bacteria selected from Bifidobacterium catenulatum group, Blautia wexlerae, Hungatella sp1, Intestinibacter bartlettii, Lachnospiraceae NG31 sp1, Lactonifactor longoviformis, Leuconostoc gelidum, Sutterella wadsworthensis, Anaerostipes hadrus, Dialister invisus, Merdimonas faecis, Roseburia faecis, Roseburia intestinalis, Ruminococcaceae NG11_sp5, or bacteria having 16S rDNA having at least 94.5%, 95%, 97%, 98%, 98.7% or 99% sequence identity to any one of the 16S rDNA thereof.

75. The method of claim 74, wherein the cancer is a bladder cancer.

76. The method of claim 73, wherein the bacteria comprises one or more bacteria selected from [Extibacter] muris, Alistipes onderdonkii, Collinsella intestinalis, Collinsella stercoris, Muricomes sp1, Neobitarella massiliensis, Prevotellamassilia sp2, Ruminococcaceae NG33 sp1, Desulfovibrio desulfuricans, Roseburia intestinalis, or bacteria having 16S rDNA having at least 94.5%, 95%, 97%, 98%, 98.7% or 99% sequence identity to any one of the 16S rDNA thereof.

77. The method of claim 76, wherein the cancer is a non-small cell lung cancer.

78. The method of claim 73, wherein the bacteria comprises one or more bacteria selected from [Adlercreutzia] equolifaciens group, [Anaerotruncus] rubiinfantis, [Romboutsia] timonensis, Alistipes finegoldii, Alistipes indistinctus, Alistipes obesi, Alistipes putredinis, Alistipes shahii, Anaerofustis stercorihominis, Anaeromassilibacillus sp3, Barnesiella intestinihominis, Bilophila wadsworthia, Blautia faecis, Coriobacteriaceae NG5 sp1, Frisingicoccus sp3, Gordonibacter pamelaeae, Harryflintia acetispora, Lachnospiraceae NG29 sp1, Marvinbryantia sp8, Massilimaliae massiliensis, Massilimaliae timonensis, Neglecta timonensis, Parabacteroides merdae, Peptoniphilaceae NG4 sp1, Peptoniphilaceae NG4 sp3, Pseudoflavonifractor capillosus, Ruminococcaceae NG12 sp1, Ruminococcaceae NG15 sp3, Ruminococcaceae NG17 sp3, Ruthenibacterium sp1, Lachnoclostridium edouardi, Lachnospira pectinoschiza, Lawsonibacter sp7, Odoribacter splanchnicus, Ruthenibacterium lactatiformans, or bacteria having 16S rDNA having at least 94.5%, 95%, 97%, 98%, 98.7% or 99% sequence identity to any one of the 16S rDNA thereof.

79. The method of claim 78, wherein the cancer is a metastatic melanoma.

80. The method of claim 73, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 2, 3, 6, 10, 12, 13, 19, 20, 21, 22, 23, 24, 34, 37, 38, 39, 42, 43, 44, 45, 46, 47, 48, 50, 51, 52, 55, 57, 58, 59, 64, 65, 66, 67, 73, 74, 75, 80, 82, 83, 84, 85, 86, 87, 88, 89, 90, 91, 92, 93, 94, 95, 96, 97, 98, 99, 100, 101, 104, 105, 106, 107, 108, 109, 110, 111, 112, 114, 115, 116, 120, 123, 129, 135, 136, 138, 139, 140, 141, 142, 143, 145, 146, 148, 153, 156, 157, 158, 161, 168, 169, 170, 173, 176, 178, 180, 182, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1297, 1298, 1299, 1010, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

81. The method of claim 80, wherein the cancer is a bladder cancer.

82. The method of claim 73, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 16, 17, 18, 53, 54, 60, 77, 79, 81, 103, 119, 125, 132, 133, 134, 164, 175, 179, 184, 1293, 1294, 1295, 1296, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, and 1732.

83. The method of claim 82, wherein the cancer is a non-small cell lung cancer.

84. The method of claim 73, wherein the bacteria comprises one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 1, 4, 5, 7, 8, 9, 11, 14, 15, 25, 26, 27, 28, 29, 30, 31, 32, 33, 35, 36, 40, 41, 61, 62, 68, 69, 70, 71, 72, 76, 78, 117, 118, 121, 122, 124, 127, 128, 130, 131, 137, 144, 147, 149, 150, 151, 152, 154, 155, 159, 160, 162, 163, 165, 166, 167, 171, 174, 177, 181, 183, 186, 1405, 1406, 1407, 1408, 1409, 1410, 1411, 1412, 1413, 1414, 1415, 1416, 1608, 1609, 1610, 1611, 1612, 1613, 1614, 1615, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1733, 1734, 1735, 1736, 1737, 1738, 1739, 1740, 1741, 1742, 1743, 1744, 1745, 1746, 1747, 1748, and 1749.

85. The method of claim 84, wherein the cancer is a metastatic melanoma.

86. The method of any of claims 73-85, wherein the therapeutic composition does not comprise Alistipes onderdonkii, Angelakisella massiliensis, Christensenellaceae NG5 sp2, Cuneatibacter caecimuris, Desulfovibrio fairfieldensis, Ruminococcaceae NG13 sp1, Ruminococcaceae NG16 sp1, Bifidobacterium animalis, Faecalibacterium prausnitzii, or bacteria having 16S rDNA having at least 94.5%, 95%, 97%, 98%, 98.7% or 99% sequence identity to any one of the 16S rDNA thereof.

87. The method of claim 86, wherein the cancer is a bladder cancer.

88. The method of any of claims 73-85, wherein the therapeutic composition does not comprise [Coprococcus] catus, [Lactobacillus] fermentum, [Romboutsia] timonensis, [Ruminococcus] bromii, [Ruminococcus] gauvreauii, Actinomyces graevenitzii, Agathobaculum butyriciproducens group, Bacteroides caccae, Bacteroides intestinalis, Bittarella massiliensis, Blautia luti group, Clostridium saudiense, Corynebacterium durum, Fusicatenibacter sp2, Lachnospiraceae NG30 sp3, Massilimaliae sp1, Monoglobus pectinilyticus, Rothia dentocariosa, Rothia mucilaginosa, Ruminococcaceae NG17 sp1, Schaalia odontolytica, Streptococcus australis, Streptococcus gordonii, Streptococcus infantis group, Streptococcus parasanguinis, Streptococcus salivarius group, Allisonella histaminiformans, Bacteroides faecis, Bacteroides massiliensis, Desulfovibrionaceae NG5_sp1, Gemmiger sp2, Intestinimonas massiliensis, Marvinbryantia sp5, Propionibacterium freudenreichii, Slackia isoflavoniconvertens, Thermodesulfobiaceae NG1_sp1, or bacteria having 16S rDNA having at least 94.5%, 95%, 97%, 98%, 98.7% or 99% sequence identity to any one of the 16S rDNA thereof.

89. The method of claim 88, wherein the cancer is a non-small cell lung cancer.

90. The method of any of claims 73-85, wherein the therapeutic composition does not comprise [Atopobium] parvulum, [Ruminococcus] gnavus, Bacteroides dorei group, Blautia sp1, Schaalia sp2, Granulicatella adiacens, Lactococcus lactis, Scardovia wiggsiae, or bacteria having 16S rDNA having at least 94.5%, 95%, 97%, 98%, 98.7% or 99% sequence identity to any one of the 16S rDNA thereof.

91. The method of claim 90, wherein the cancer is a metastatic melanoma.

92. The method of any of claims 73-85, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 188, 213, 245, 323, 331, 332, 376, 455, 509, 516, 538, 582, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, o1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, and 1349.

93. The method of claim 92, wherein the cancer is a bladder cancer.

94. The method of any of claims 73-85, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 187, 189, 190, 191, 192, 193, 194, 195, 196, 197, 198, 199, 200, 201, 202, 203, 204, 205, 211, 214, 216, 217, 218, 219, 220, 221, 222, 224, 225, 226, 227, 228, 229, 230, 231, 232, 233, 234, 235, 236, 237, 238, 239, 240, 241, 242, 243, 244, 246, 247, 248, 253, 254, 255, 257, 258, 259, 263, 264, 265, 267, 268, 269, 270, 271, 272, 273, 277, 283, 286, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 324, 325, 326, 327, 328, 329, 330, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 377, 378, 379, 380, 381, 382, 383, 384, 385, 386, 387, 389, 390, 391, 392, 393, 394, 395, 396, 397, 398, 399, 400, 401, 402, 403, 409, 410, 411, 412, 413, 414, 415, 418, 421, 424, 425, 428, 429, 430, 431, 432, 433, 434, 435, 436, 437, 438, 439, 440, 441, 442, 443, 444, 445, 447, 449, 450, 452, 453, 454, 456, 457, 458, 460, 462, 463, 464, 465, 466, 467, 468, 469, 471, 472, 473, 474, 475, 476, 478, 479, 480, 481, 483, 484, 486, 487, 488, 489, 490, 491, 492, 493, 494, 495, 496, 497, 498, 499, 500, 501, 502, 503, 504, 505, 506, 507, 508, 510, 511, 512, 513, 514, 515, 520, 521, 522, 523, 524, 525, 526, 527, 528, 529, 530, 531, 537, 539, 540, 542, 543, 544, 545, 546, 547, 548, 550, 551, 552, 553, 554, 555, 560, 561, 564, 565, 566, 573, 574, 577, 579, 581, 583, 584, 585, 586, 587, 588, 589, 591, 594, 595, 596, 597, 598, 599, 600, 774, 1113, 1114, 1115, 1116, 1117, 1118, 1119, 1120, 1121, 1122, 1123, 1124, 1125, 1126, 1127, 1130, 1131, 1132, 1133, 1134, 1135, 1356, 1357, 1358, 1359, 1360, 1361, 1362, 1395, 1396, 1397, 1398, 1399, 1400, 1401, 1402, 1616, 1617, 1618, 1619, 1620, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1755, and 1756.

95. The method of claim 94, wherein the cancer is non-small cell lung cancer.

96. The method of any of claims 73-85, wherein the therapeutic composition does not comprise one or more bacteria having a 16S rDNA sequence that is at least 85%, at least 90%, at least 95%, at least 96%, at least 97%, at least 98%, at least 99%, or 100% identical to a 16S rDNA sequence set forth in SEQ ID NOs: 206, 207, 208, 209, 210, 212, 215, 223, 249, 250, 251, 252, 256, 260, 261, 262, 266, 274, 275, 276, 278, 279, 280, 281, 282, 284, 285, 287, 388, 404, 405, 406, 407, 408, 416, 417, 419, 420, 422, 423, 426, 427, 446, 448, 451, 459, 461, 482, 485, 517, 518, 519, 532, 533, 534, 535, 536, 549, 556, 557, 558, 559, 562, 563, 567, 568, 569, 570, 571, 572, 575, 576, 580, 590, 593, 1363, 1364, 1365, 1366, 1367, 1368, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1750, 1751, 1752, 1753, and 1754.

97. The method of claim 96, wherein the cancer is metastatic melanoma.

98. The method of any one of claims 73-97, further comprising administering at least one anticancer agent to the subject.

99. The method of claim 98, wherein the anticancer agent is a checkpoint inhibitor.

100. The method of claim 99, wherein the checkpoint inhibitor is selected from an anti-PD-1 antibody, an anti-CTLA-4 antibody, an anti-PD-L1 antibody or combinations thereof.

101. The method of claim 90, wherein the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT O11, MPDL3280A, MEDI-4736, MSB-0020718C, AUR-012, LAG-3, OX40 inhibitors, OX40L inhibitors, TIGIT inhibitors, STI-A1010 or combinations thereof.

102. The method of claim 98, wherein the anticancer agent comprises a chemotherapy agent.

103. The method of any of claims 98-102, wherein the anticancer agent is a combination of a checkpoint inhibitor and a chemotherapy agent.

104. The method of any of claims 73-103, wherein the purified or isolated population of bacteria is administered at a concentration of at least about 1×10² viable colony forming units.

105. The method of any of claims 73-104, wherein the purified or isolated population of bacteria is administered at a concentration of at a concentration of about 1×10² to 1×10⁹ viable colony forming units.

106. The method of any of claims 73-105, wherein a fraction of the purified or isolated population of bacteria comprises a spore-forming bacteria.

107. The method of any of claims 73-105, wherein a fraction of the purified or isolated population of bacteria is in spore form.

108. The method of any of claims 73-105, wherein a fraction of the purified or isolated population of bacteria is in vegetative form.

109. The method of any of claims 73-108, wherein the composition further comprises a pharmaceutically acceptable excipient.

110. The method of any of claims 73-109, wherein the composition is formulated for delivery to the intestine.

111. The method of any of claims 73-110, wherein the composition is enterically coated.

112. The method of any of claims 73-111, wherein the composition is formulated for oral administration.

113. The method of any of claims 73-112, wherein the composition is formulated into a food or beverage.

114. The method of any of claims 73-113, wherein the mammalian subject is a human.

115. The method of any of claims 73-114, wherein the cancer is selected from a metastatic melanoma, melanoma of the skin, non-small cell lung cancer, a kidney cancer, a bladder cancer, a head and neck cancer, a Merkel cell skin cancer (Merkel cell carcinoma), or a Hodgkin lymphoma.

116. The method of claim 115, wherein the cancer is a metastatic melanoma.

117. The method of claim 115, wherein the cancer is a non-small cell lung cancer.

118. The method of claim 115, wherein the cancer is a bladder cancer.

119. The method of any of claims 73-118, wherein prior to administration of the population of bacteria the subject is subjected to antibiotic treatment and/or a bowel cleanse.

120. The method of any one of claims 73-119, wherein the subject has previously been treated for the cancer.

121. The method of claim 120, wherein the subject has been determined to be a non-responder to the previous treatment.

122. The method of claim 120 or 121, wherein the subject has been determined to have a toxic response to the previous treatment.

123. The method of any one of claims 120-122, wherein the previous treatment comprises immune checkpoint blockade monotherapy or immune checkpoint blockade combination therapy.

124. The method of any one of claims 73-123, wherein the cancer is recurrent cancer.

125. A method of identifying a mammalian subject as a candidate for immune checkpoint therapy in combination with adjuvant microbiome therapy to treat a cancer, the method comprising:
a) obtaining a microbiome sample from the subject,
b) determining the prevalence and/or abundance of the family of bacteria in the microbiome sample, and
c) determining that the subject is a candidate for treatment if the microbiome sample comprises the bacteria of any one of claims 1-72.

126. The method of any of claim 125, wherein the subject is determined to be a candidate for immune checkpoint inhibitor therapy.

127. The method of claim 125 or 126, wherein the immune checkpoint therapy comprises immune checkpoint blockade monotherapy or immune checkpoint blockade combination therapy.

128. The method of any of claims 125-127, wherein the treatment further comprises a chemotherapy.

129. The method of any of claims 125-128, wherein the mammalian subject is a human.

130. The method of any of claims 125-129, wherein the cancer is selected from a metastatic melanoma, a melanoma of the skin, a non-small cell lung cancer, a kidney cancer, a bladder cancer, a head and neck cancer, a Merkel cell skin cancer (Merkel cell carcinoma), or a Hodgkin lymphoma.

131. The method of claim 130, wherein the cancer is a metastatic melanoma.

132. The method of claim 130, wherein the cancer is a non-small cell lung cancer.

133. The method of claim 130, wherein the cancer is a bladder cancer.

134. A method of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises the bacteria of any one of claims 1-72.

135. A therapeutic composition derived from fecal matter from a donor identified using the method of claim 134.

136. The therapeutic composition of claim 135, further comprising a pharmaceutically acceptable excipient.

137. The therapeutic composition of claim 135 or 136, wherein the therapeutic composition comprises bacteria that are in vegetative and/or spore form.

138. The therapeutic composition of any one of claims 135-137, wherein the therapeutic composition further comprises a checkpoint inhibitor.

139. The therapeutic composition of claim 138, wherein the checkpoint inhibitor is selected from anti-PD-1 antibody, an anti-CTLA-4 antibody, an anti-PD-L1 antibody or combinations thereof.

140. The therapeutic composition of claim 138, wherein the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT O11, MPDL3280A, MEDI-4736, MSB-0020718C, AUR-012, LAG-3, OX40 inhibitors, OX40L inhibitors, TIGIT inhibitors, STI-A1010 or combinations thereof.

141. A method of treating or preventing or reducing risk of a cancer in a mammalian subject comprising administering to the subject a therapeutic composition of any one of claims 135-140.

142. A method of treating or preventing or reducing risk of a cancer comprising administering at least one anticancer treatment to a subject determined to have a microbiome sample comprising the bacteria of any one of claims 1-72.

143. A method comprising evaluating a microbiome profile in a sample from a subject for the bacteria of any one of claims 1-72.

144. The method of claim 143, wherein the method further comprises comparing the microbiome profile to a control microbiome.

145. The method of claim 144, wherein the control microbiome comprises a microbiome sample from a subject determined to be a responder to an anticancer treatment.

146. The method of claim 145, wherein the comparing comprises comparison of the bacteria from claim 143 to the control microbiome from a subject determined to be a responder to an anticancer treatment.

147. The method of claim 144, wherein the control microbiome comprises a microbiome sample from a subject determined to be a non-responder to an anticancer treatment.

148. The method of claim 147, wherein the comparing comprises comparison of the bacteria from claim 143 to the control microbiome from a subject determined to be a non-responder to an anticancer treatment.
